(19) United States
(12) Patent Application Publication
(10) Pub. No.: US 2025/0020999 A1
(43) Pub. Date: Jan. 16, 2025

(54) RESIST COMPOSITION AND PATTERN FORMING PROCESS

(71) Applicant: Shin-Etsu Chemical Co., Ltd., Tokyo (JP)

(72) Inventors: Jun Hatakeyama, Joetsu-shi (JP); Masahiro Fukushima, Joetsu-shi (JP); Tomomi Watanabe, Joetsu-shi (JP)

(73) Assignee: Shin-Etsu Chemical Co., Ltd., Tokyo (JP)

(21) Appl. No.: 18/760,338

(22) Filed: Jul. 1, 2024

(30) Foreign Application Priority Data
Jul. 11, 2023 (JP) 2023-113631
Jan. 9, 2024 (JP) 2024-001147

(51) Int. Cl. G03F 7/004; C08F 212/14; C08F 220/18; C08F 220/22; G03F 7/038; G03F 7/039

(52) U.S. Cl. CPC G03F 7/0045; C08F 212/24; C08F 220/1807; C08F 220/22; G03F 7/0382; G03F 7/0392

(57) ABSTRACT

The resist composition has high sensitivity, improved LWR and CDU, high contrast, excellent resolution. A wide process margin can be obtained by using a sulfonium salt or an iodonium salt containing an arylsulfonic acid anion having an aromatic group substituted with a bromine atom or an iodine atom as an acid generator. The resist composition comprising an acid generator containing an onium salt having the following formula (1).

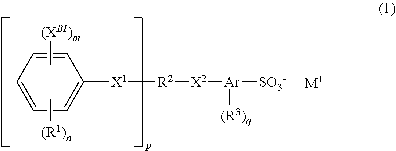

### CROSS-REFERENCE TO RELATED APPLICATIONS

This non-provisional application claims priority under 35 U.S.C. § 119(a) on Patent Application Nos. 2023-113631 and 2024-001147 filed in Japan on Jul. 11, 2023 and Jan. 9, 2024, respectively, the entire contents of which are hereby incorporated by reference.

### TECHNICAL FIELD

The present invention relates to a resist composition and a pattern forming process.

### BACKGROUND ART

To meet the demand for higher integration density and higher operating speed of LSIs, the effort to reduce the pattern rule is in rapid progress. As the use of 5G high-speed communications and artificial intelligence (AI) is widely spreading, high-performance devices are needed for their processing. As the most advanced miniaturization technology, mass production of microelectronic devices at the 5-nm node by the lithography using extreme ultraviolet (EUV) having a wavelength of 13.5 nm has been implemented. Studies are made on the application of EUV lithography to 3-nm node devices of the next generation and 2-nm node devices of the next-but-one generation. IMEC in Belgium announced its successful development of 1-nm and 0.7-nm node devices.

With the miniaturization of the pattern, edge roughness (LWR) of line patterns and critical dimensional uniformity (CDU) of hole patterns and dot patterns have been regarded as problems. The influence of uneven distribution and aggregation of a base polymer and an acid generator and the influence of acid diffusion have been pointed out. Furthermore, LWR and CDU tend to increase as the resist film is thinned, and deterioration of LWR and CDU due to thinning with progress of miniaturization has become a serious problem.

In EUV resist compositions, it is necessary to simultaneously achieve high sensitivity, high resolution, and low LWR. If the acid diffusion distance is shortened, the LWR and CDU are improved, but sensitivity is reduced. For example, by lowering the post exposure bake (PEB) temperature, the LWR and CDU are improved, but sensitivity is reduced. Even if the amount of quencher added is increased, the LWR and CDU are improved, but sensitivity is reduced. It is necessary to break a trade-off relationship between sensitivity and LWR.

Resist compositions added with an onium salt having an anion having an iodine atom or a bromine atom as an acid generator have been proposed (Patent Documents 1 to 5). By having an iodine atom with high EUV absorption or a bromine atom with high ionization efficiency, the efficiency of decomposition of the acid generator during exposure is increased, and sensitivity is increased. The amount of absorption of photons increases, and physical contrast can be increased.

The health effects of perfluoroalkyl compounds (PFAS) have been pointed out, and there is a movement to place restrictions on the production and sale of PFAS compounds in European REACH. Many compounds, including PFAS, are currently used in the context of semiconductor lithography. For example, a material containing the compound is used as a surfactant, an acid generator, or the like.

### CITATION LIST

Patent Document 1: JP-A 2018-159744
Patent Document 2: JP-A 2018-005224
Patent Document 3: JP-A 2018-025789
Patent Document 4: JP-A 2019-003175
Patent Document 5: JP-A 2023-021084

### SUMMARY OF THE INVENTION

It is desired to develop a resist composition that has higher sensitivity than previous resist compositions and is capable of improving the LWR of line patterns and the CDU of hole patterns.

The present invention has been made in view of the above circumstances, and an object of the present invention is to provide a resist composition which exhibits higher sensitivity and improved LWR and CDU regardless of whether it is of positive or negative tone, and a pattern forming process using the resist composition.

As a result of intensive studies to achieve the above object, the present inventors have found that a resist composition having high sensitivity, improved LWR and CDU, high contrast, excellent resolution, and a wide process margin can be obtained by using a sulfonium salt or an iodonium salt containing an arylsulfonic acid anion having an aromatic group substituted with a bromine atom or an iodine atom as an acid generator, and have completed the present invention.

That is, the present invention provides the following resist composition and pattern forming process.

1. A resist composition comprising an acid generator containing an onium salt having the following formula (1):

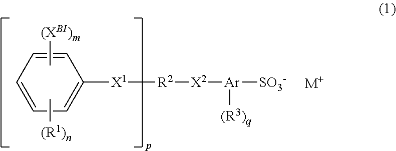

wherein m is an integer of 1 to 5, n is an integer of 0 to 4, provided that 1≤m+n≤5, p is an integer of 1 to 3, q is an integer of 1 to 4 when Ar is a (q+2)-valent group derived from benzene, and an integer of 1 to 6 when Ar is a (q+2)-valent group derived from naphthalene,
X^{BI} is a bromine atom or an iodine atom,
Ar is a (q+2)-valent group derived from benzene or naphthalene,
X¹ and X² are each a single bond, an ether bond, an ester bond, a sulfonic acid ester bond, an amide bond, a urethane bond, a urea bond, a carbonate bond, or a C₁-C₆ alkanediyl group, and the alkanediyl group may contain at least one selected from an ether bond and an ester bond, however, when p=1, X^{BI} is an iodine atom and Ar is a (q+2)-valent group derived from benzene, X¹ is not *—C(═O)—O—, and when p=1, X^{BI} is an iodine atom, X¹ and R² are each a single bond and Ar is a (q+2)-valent group derived from benzene, X² is not *—C(═O)—O—, and when p=1, X² and R² are each a single bond and Ar is a (q+2)-valent group derived from benzene, X¹ is not —O—C(═O)—** and a carbonate bond, and when p=1, X¹ is a single bond and Ar is a (q+2)-valent group derived from benzene, X² is not —O—C(═O)—** and a carbonate bond, * represents a bond with a benzene ring in the formula, and ** represents a bond with Ar,
R¹ is a hydroxy group, a carboxy group, a fluorine atom, a chlorine atom, an amino group, a C₁-C₂₀ hydrocarbyl group, a C₁-C₂₀ hydrocarbyloxy group, a C₂-C₂₀ hydrocarbyloxycarbonyl group, a C₂-C₂₀ hydrocarbylcarbonyloxy group, —N(R^{1A})—C(═O)—R^{1B}, N(R^{1A})—C(═O)—O—R^{1B}, or —N(R^{1A})—S(═O)₂—R^{1B}, and the hydrocarbyl group, the hydrocarbyloxy group, the hydrocarbyloxycarbonyl group, and the hydrocarbylcarbonyloxy group may contain at least one selected from a fluorine atom, a chlorine atom, a bromine atom, an iodine atom, a hydroxy group, an amino group, an ester bond, and an ether bond, R^{1A} is a hydrogen atom or a C₁-C₆ saturated hydrocarbyl group, and the saturated hydrocarbyl group may contain a halogen atom, a hydroxy group, a C₁-C₆ saturated hydrocarbyloxy group, a C₂-C₆ saturated hydrocarbylcarbonyl group, or a C₂-C₆ saturated hydrocarbylcarbonyloxy group, R^{1B} is a C₁-C₆ aliphatic hydrocarbyl group or a C₆-C₁₂ aryl group, which may contain a halogen atom, a hydroxy group, a C₁-C₆ saturated hydrocarbyloxy group, a C₂-C₆ saturated hydrocarbylcarbonyl group, or a C₂-C₆ saturated hydrocarbylcarbonyloxy group,
R² is a single bond or a C₁-C₂₈ (p+1)-valent hydrocarbon group, and the hydrocarbon group may contain at least one selected from an oxygen atom, a nitrogen atom, a sulfur atom, and a halogen atom,
R³ is a fluorine atom, a trifluoromethoxy group, a nitro group, a C₁-C₁₂ saturated hydrocarbyl group, or a C₆-C₁₀ aryl group, and
M⁺ is a sulfonium cation or an iodonium cation.

2. The resist composition of item 1, wherein X¹ and X² are groups other than a single bond, and R² is a group other than a single bond.

3. The resist composition of item 1 or 2, further comprising a base polymer.

4. The resist composition of item 3, wherein the base polymer contains repeat units having the following formula (a1) or (a2):

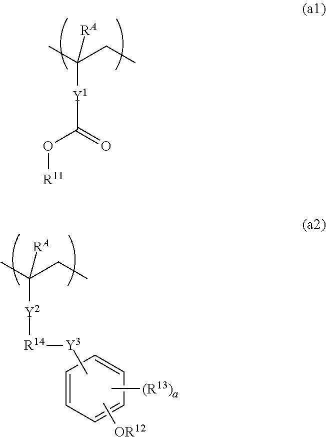

wherein R^{A} is each independently a hydrogen atom or a methyl group,
Y¹ is a single bond, a phenylene group, a naphthylene group, or a C₁-C₁₂ linking group containing at least one moiety selected from an ester bond, an ether bond, and a lactone ring,
Y² is a single bond or an ester bond,
Y³ is a single bond, an ether bond, or an ester bond,
R¹¹ and R¹² are each independently an acid labile group,
R¹³ is a C₁-C₄ saturated hydrocarbyl group, a halogen atom, a C₂-C₅ saturated hydrocarbylcarbonyl group, a cyano group, or a C₂-C₅ saturated hydrocarbyloxycarbonyl group,
R¹⁴ is a single bond or a C₁-C₆ alkanediyl group which may contain an ether bond or an ester bond, and
a is an integer of 0 to 4.

5. The resist composition of item 4, which is a chemically amplified positive resist composition.

6. The resist composition of item 3, wherein the base polymer is free of an acid labile group.

7. The resist composition of item 6, which is a chemically amplified negative resist composition.

8. The resist composition of any one of items 1 to 7, further comprising an organic solvent.

9. The resist composition of any one of items 1 to 8, further comprising a quencher.

10. The resist composition of any one of items 1 to 9, further comprising a surfactant.

11. A pattern forming process comprising the steps of:
applying the resist composition of any one of items 1 to 10 onto a substrate to form a resist film thereon,
exposing the resist film to high-energy radiation, and
developing the exposed resist film in a developer.

12. The pattern forming process of item 11, wherein the high-energy radiation is ArF excimer laser having a wavelength of 193 nm, KrF excimer laser having a wavelength of 248 nm, electron beam (EB), or EUV having a wavelength of 3 to 15 nm.

### Advantageous Effects of Invention

A resist film containing, as an acid generator, a sulfonium salt or an iodonium salt containing an arylsulfonic acid anion having an aromatic group substituted with a bromine atom or an iodine atom is characterized by high absorption of EUV light and suppression of acid diffusion. This makes it possible to prevent a reduction of resolution due to blur by acid diffusion and to improve the LWR and CDU. Accordingly, a resist composition having high sensitivity and improved LWR and CDU is formulated.

### DETAILED DESCRIPTION OF THE INVENTION

### [Resist Composition]

A resist composition of the present invention contains an acid generator containing a sulfonium salt or an iodonium salt containing an arylsulfonic acid anion having an aromatic group substituted with a bromine atom or an iodine atom. The salt has high contrast and small acid diffusion due to absorption of an iodine atom, ionization of a bromine atom, and bulkiness of an aryl acid. This can improve LWR and CDU.

The effect of improving the LWR and CDU by the acid generator used in the present invention is exerted in both positive pattern formation and negative pattern formation by alkaline aqueous solution development and negative pattern formation by organic solvent development.

### [Acid Generator]

An acid generator used in the present invention contains a sulfonium salt or an iodonium salt containing an arylsulfonic acid anion having an aromatic group substituted with a bromine atom or an iodine atom, represented by formula (1).

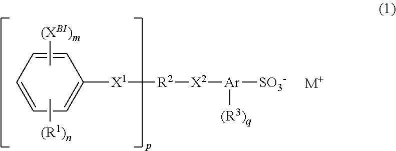

In formula (1), m is an integer of 1 to 5, n is an integer of 0 to 4, provided that 1≤m+n≤5, p is an integer of 1 to 3, q is an integer of 1 to 4 when Ar is a (q+2)-valent group derived from benzene, and an integer of 1 to 6 when Ar is a (q+2)-valent group derived from naphthalene.

In formula (1), X^{BI} is a bromine atom or an iodine atom. Groups X^{BI} may be identical or different when m is 2 or more.

In formula (1), Ar is a (q+2)-valent group derived from benzene or naphthalene. Specifically, Ar is a group obtained by eliminating (q+2) hydrogen atoms from benzene or naphthalene.

In formula (1), X¹ and X² are each a single bond, an ether bond, an ester bond, a sulfonic acid ester bond, an amide bond, a urethane bond, a urea bond, a carbonate bond, or a C₁-C₆ alkanediyl group, and the alkanediyl group may contain at least one selected from an ether bond and an ester bond, however, when p=1, X^{BI} is an iodine atom and Ar is a (q+2)-valent group derived from benzene, X¹ is not *—C(═O)—O—, and when p=1, X^{BI} is an iodine atom, X¹ and R² are each a single bond and Ar is a (q+2)-valent group derived from benzene, X² is not *—C(═O)—O—, and when p=1, X² and R² are each a single bond and Ar is a (q+2)-valent group derived from benzene, X¹ is not —O—C(═O)—** and a carbonate bond, and when p=1, X¹ is a single bond and Ar is a (q+2)-valent group derived from benzene, X² is not —O—C(═O)—** and a carbonate bond, * represents a bond with a benzene ring in the formula, and ** represents a bond with Ar. X¹ and X² are preferably groups other than a single bond, and more preferably an ether bond, an ester bond, an amide bond, a urethane bond, a urea bond, a carbonate bond, or the like.

In formula (1), R¹ is a hydroxy group, a carboxy group, a fluorine atom, a chlorine atom, an amino group, a C₁-C₂₀ hydrocarbyl group, a C₁-C₂₀ hydrocarbyloxy group, a C₂-C₂₀ hydrocarbyloxycarbonyl group, a C₂-C₂₀ hydrocarbylcarbonyloxy group, —N(R^{1A})—C(═O)—R^{1B}, —N(R^{1A})—C(═O)—O—R^{1B} or —N(R^{1A})—S(═O)₂—R^{1B}, and the hydrocarbyl group, the hydrocarbyloxy group, the hydrocarbyloxycarbonyl group, and the hydrocarbylcarbonyloxy group may contain at least one selected from a fluorine atom, a chlorine atom, a bromine atom, an iodine atom, a hydroxy group, an amino group, an ester bond, and an ether bond, R^{1A} is a hydrogen atom or a C₁-C₆ saturated hydrocarbyl group, and the saturated hydrocarbyl group may contain a halogen atom, a hydroxy group, a C₁-C₆ saturated hydrocarbyloxy group, a C₂-C₆ saturated hydrocarbylcarbonyl group, or a C₂-C₆ saturated hydrocarbylcarbonyloxy group, R^{1B} is a C₁-C₁₆ aliphatic hydrocarbyl group or a C₆-C₁₂ aryl group, which may contain a halogen atom, a hydroxy group, a C₁-C₆ saturated hydrocarbyloxy group, a C₂-C₆ saturated hydrocarbylcarbonyl group, or a C₂-C₆ saturated hydrocarbylcarbonyloxy group. Groups R¹ may be identical or different when n and/or p is 2 or more.

The hydrocarbyl group represented by R¹ and the hydrocarbyl moiety in the hydrocarbyloxy group, the hydrocarbyloxycarbonyl group, and the hydrocarbylcarbonyloxy group may be saturated or unsaturated, and may be straight, branched, or cyclic. Examples thereof include C₁-C₂₀ alkyl groups such as a methyl group, an ethyl group, a n-propyl group, an isopropyl group, a n-butyl group, an isobutyl group, a sec-butyl group, a tert-butyl group, a n-pentyl group, a n-hexyl group, a n-octyl group, a n-nonyl group, a n-decyl group, an undecyl group, a dodecyl group, a tridecyl group, a tetradecyl group, a pentadecyl group, a heptadecyl group, an octadecyl group, a nonadecyl group, and an icosyl group; C₃-C₂₀ cyclic saturated hydrocarbyl groups such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; C₂-C₂₀ alkenyl groups such as a vinyl group, a propenyl group, a butenyl group, and a hexenyl group; C₂-C₂₀ alkynyl groups such as an ethynyl group, a propynyl group, and a butynyl group; C₃-C₂₀ cyclic unsaturated aliphatic hydrocarbyl groups such as a cyclohexenyl group and a norbornenyl group; C₆-C₂₀ aryl groups such as a phenyl group, a methylphenyl group, an ethylphenyl group, a n-propylphenyl group, an isopropylphenyl group, a n-butylphenyl group, an isobutylphenyl group, a sec-butylphenyl group, a tert-butylphenyl group, a naphthyl group, a methylnaphthyl group, an ethylnaphthyl group, a n-propylnaphthyl group, an isopropylnaphthyl group, a n-butylnaphthyl group, an isobutylnaphthyl group, a sec-butylnaphthyl group, and a tert-butylnaphthyl group; C₇-C₂₀ aralkyl groups such as a benzyl group and a phenethyl group; and groups obtained by combining the foregoing.

In formula (1), R² is a single bond or a C₁-C₂₈ (p+1)-valent hydrocarbon group, and the hydrocarbon group may contain at least one selected from an oxygen atom, a nitrogen atom, a sulfur atom, and a halogen atom. The hydrocarbon group is a group obtained by eliminating (p+1) hydrogen atoms from a C₁-C₂₈ hydrocarbon group. The hydrocarbon group may be saturated or unsaturated, and may be straight, branched, or cyclic. Examples thereof include C₁-C₂₈ alkanes such as methane, ethane, propane, butane, 2-methylpropane, pentane, 2-methylbutane, hexane, heptane, octane, nonane, decane, undecane, and dodecane; C₃-C₂₈ cyclic saturated hydrocarbons such as cyclopropane, cyclobutane, cyclopentane, cyclohexane, norbornane, and adamantane; C₂-C₂₈ alkenes such as ethylene, propene, 1-butene, 2-butene, and 2-methylpropene; C₃-C₂₈ cyclic unsaturated hydrocarbons such as cyclohexene and norbornene; C₆-C₂₈ aromatic hydrocarbons such as benzene, naphthalene, toluene, xylene, and anthracene; and compounds in which some or all of these hydrogen atoms are substituted with a hydrocarbyl group. R² is preferably a group other than a single bond, and more preferably a group derived from an alkane, a cyclic saturated hydrocarbon which may contain an oxygen atom, or an aromatic hydrocarbon.

In formula (1), R³ is a fluorine atom, a trifluoromethoxy group, a nitro group, a C₁-C₁₂ saturated hydrocarbyl group, or a C₆-C₁₀ aryl group. Groups R³ may be identical or different when q is 2 or more.

Examples of the anion of the salt having formula (1) include those shown below, but are not limited thereto.

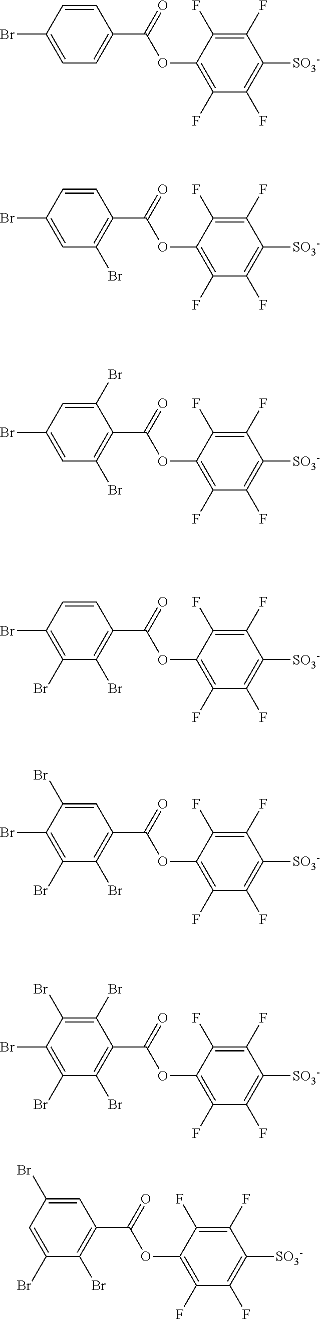

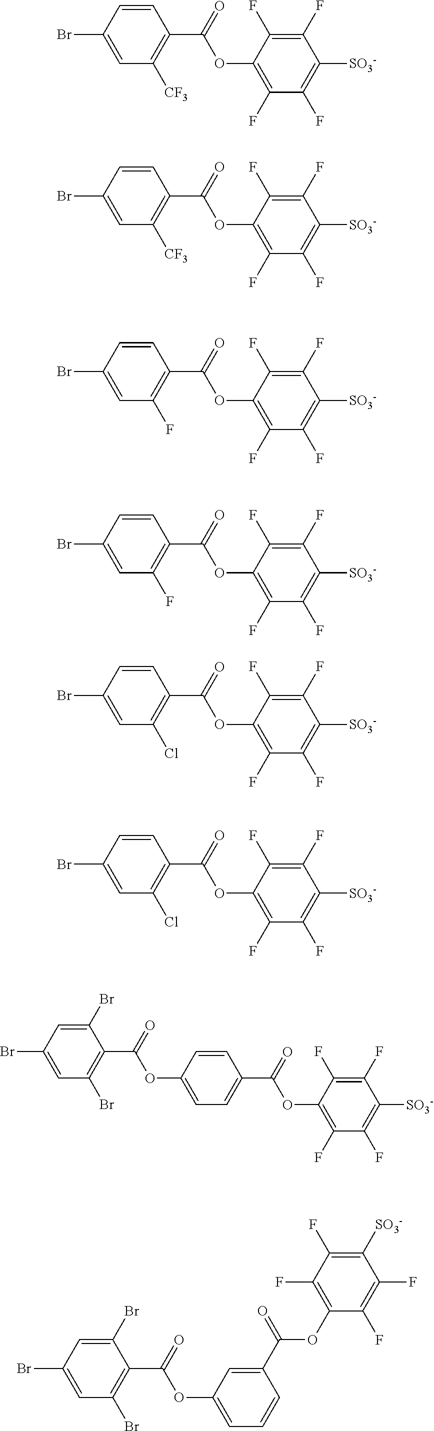

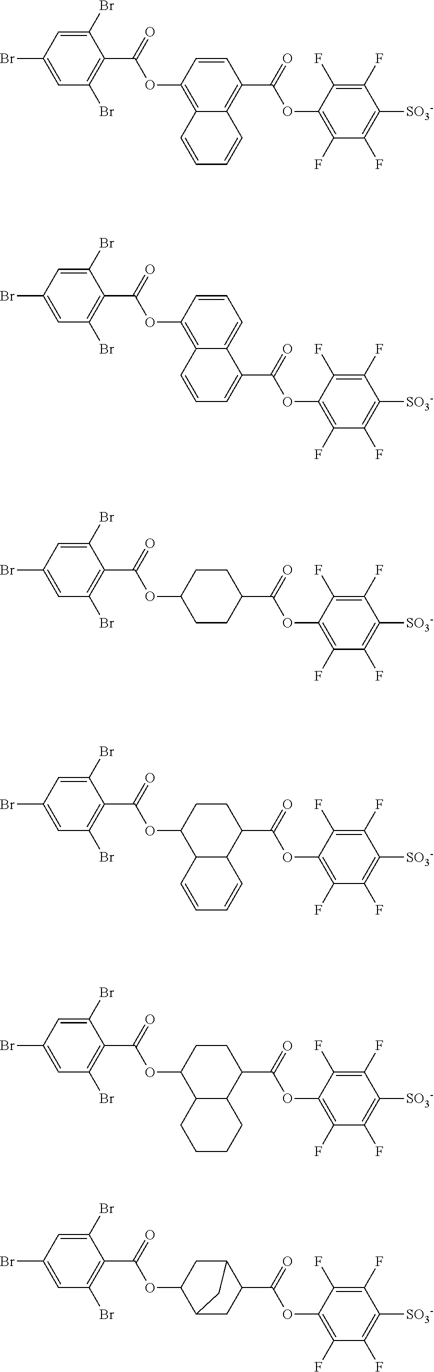

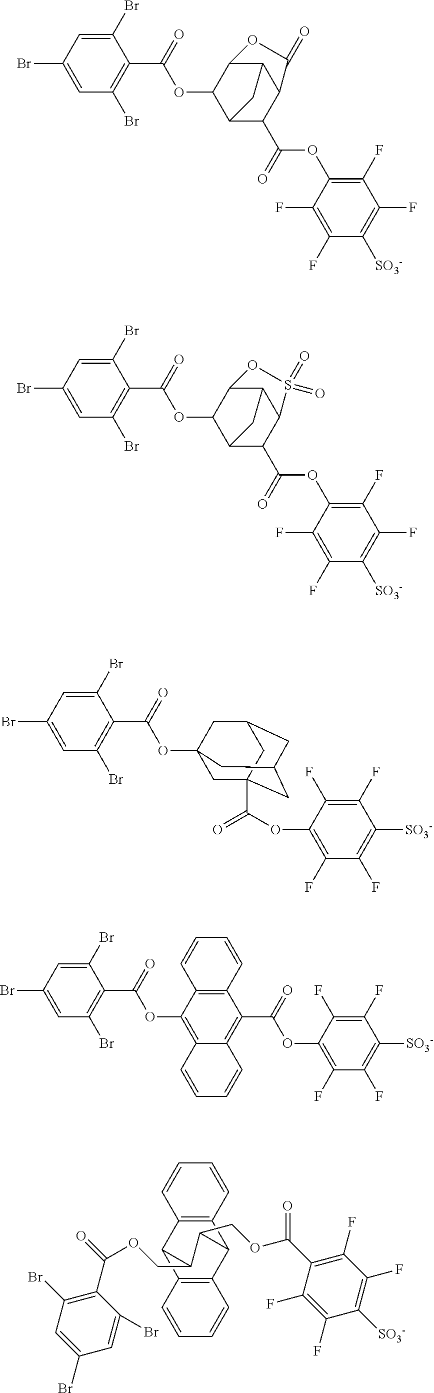

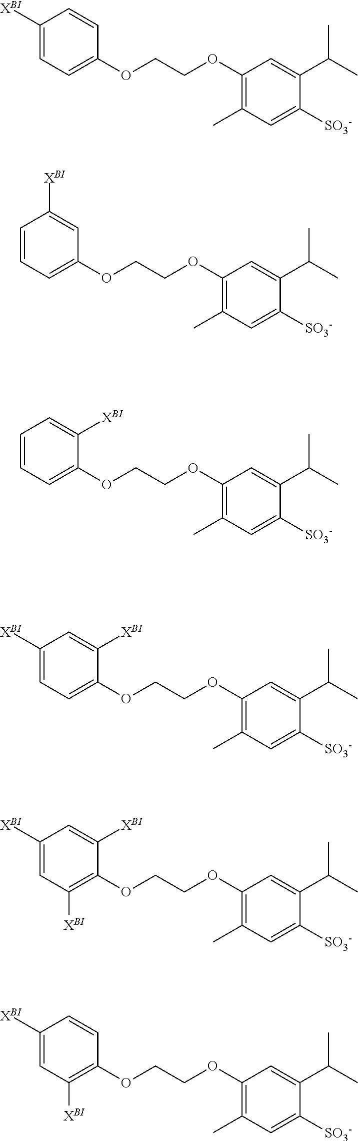

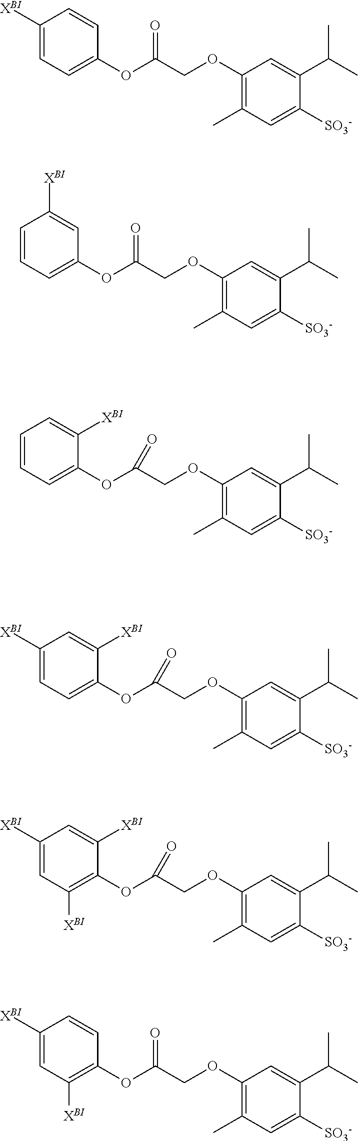

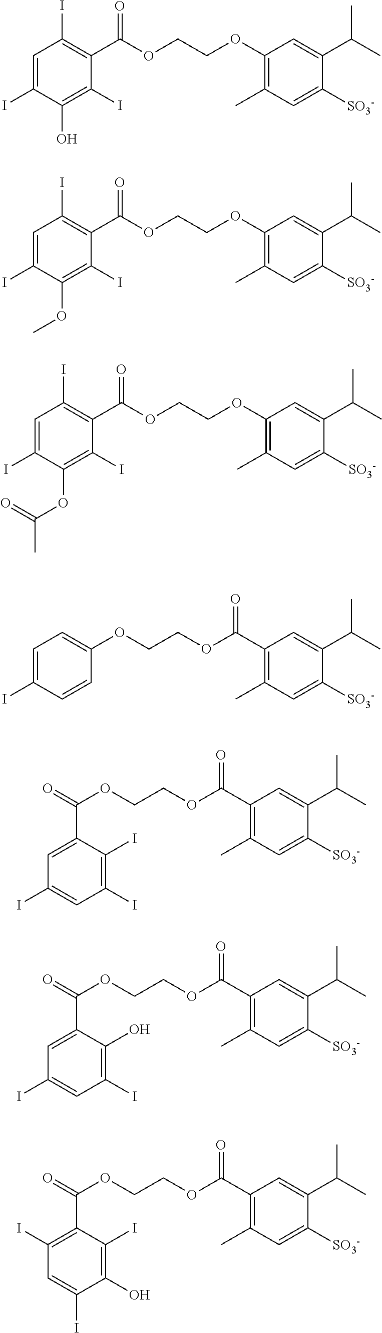

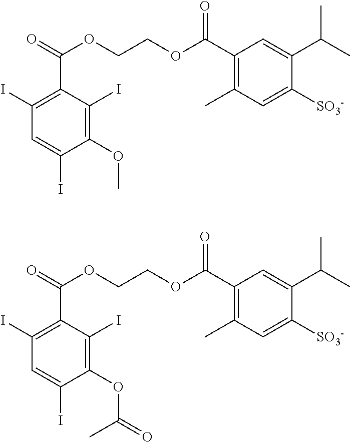

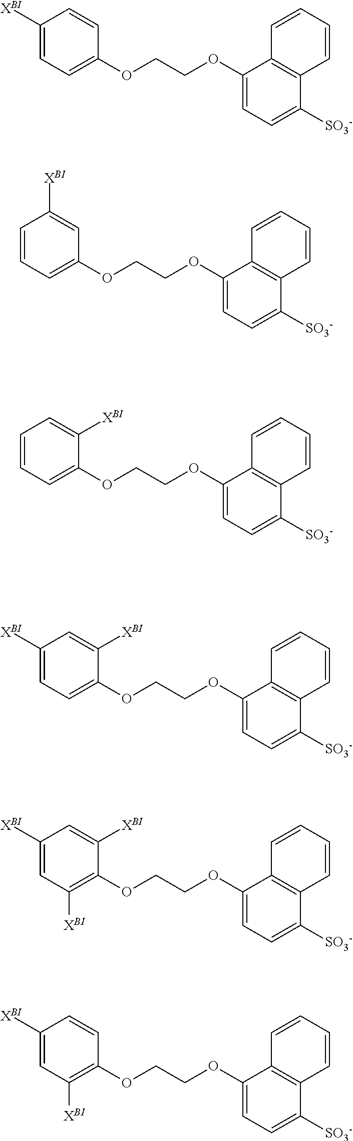

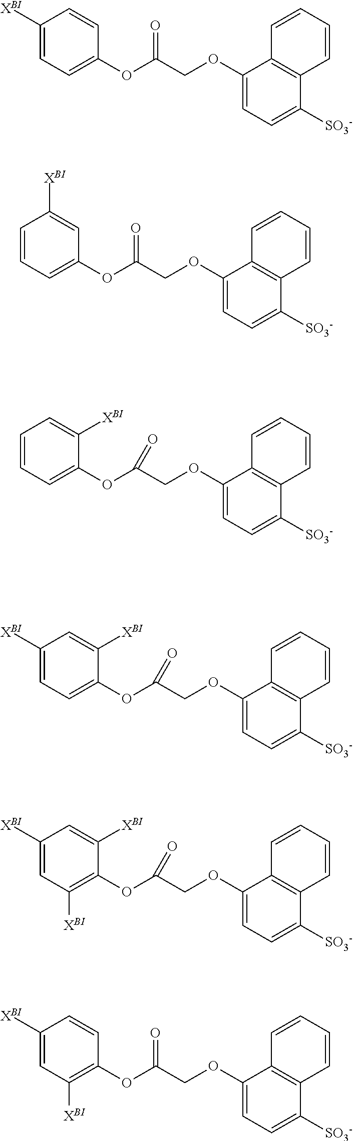

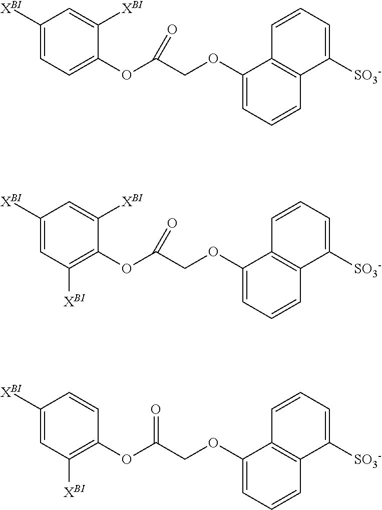

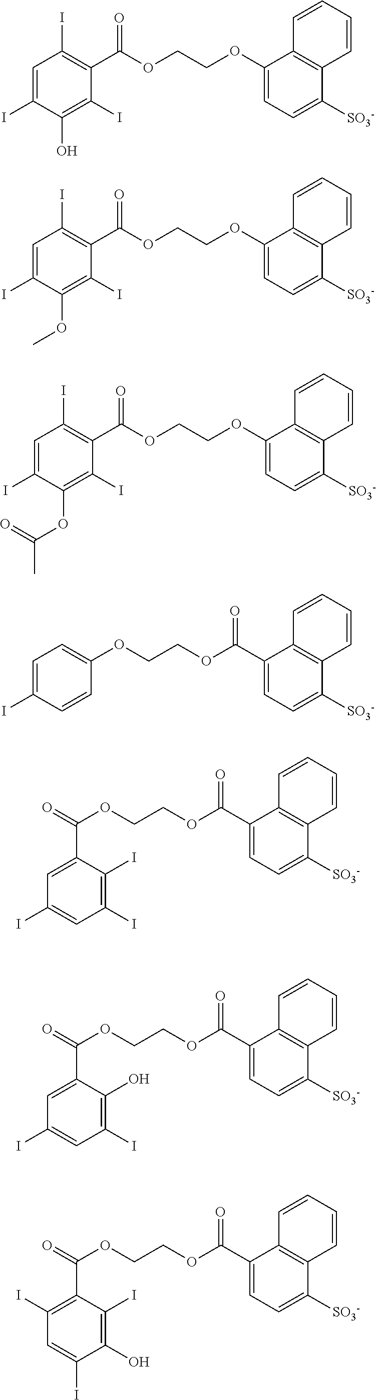

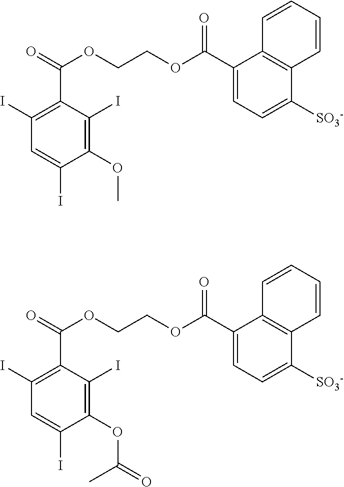

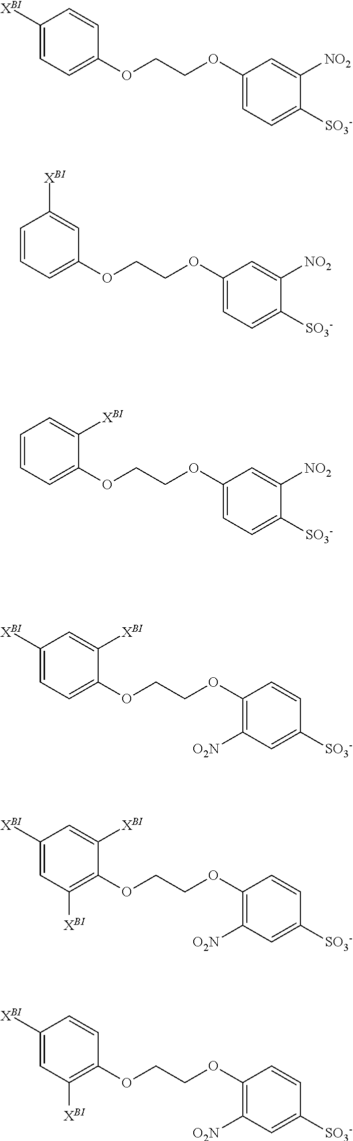

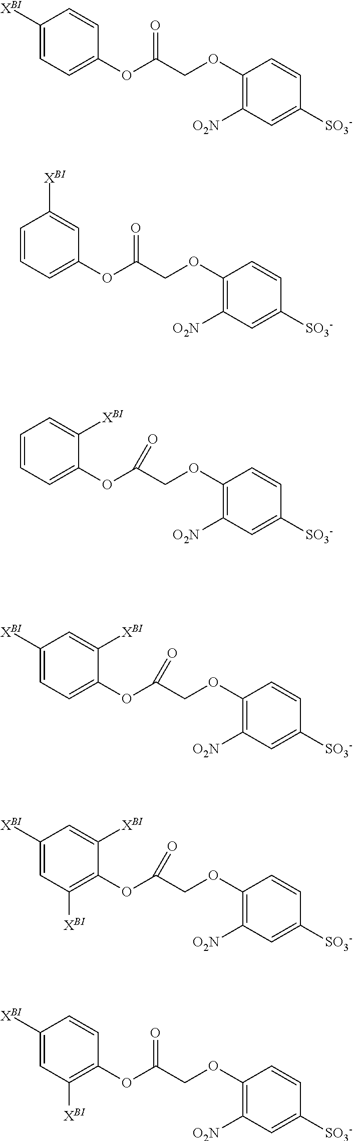

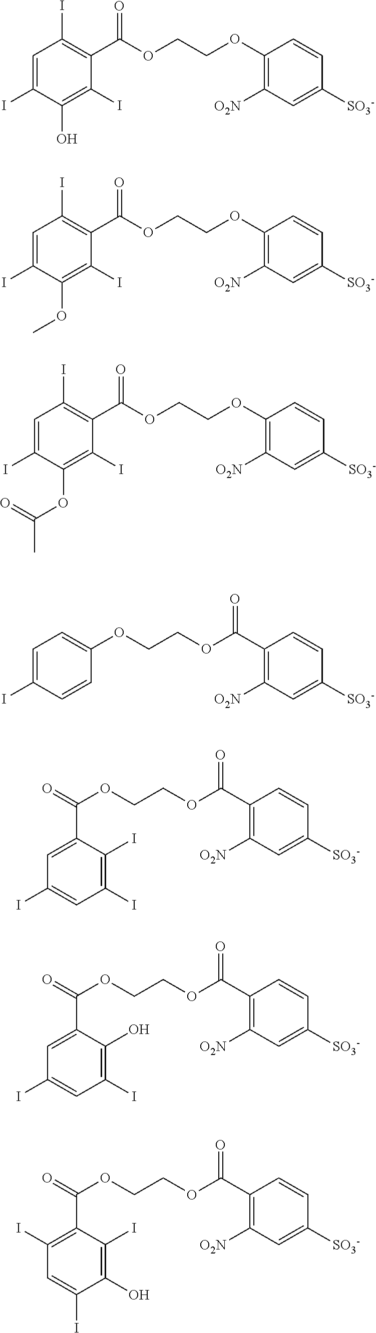

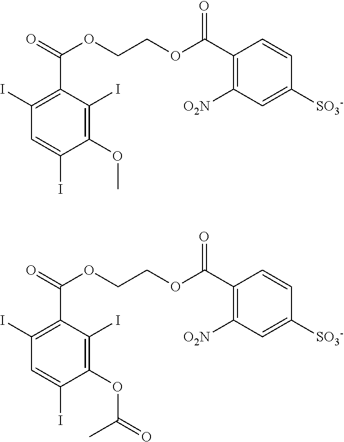

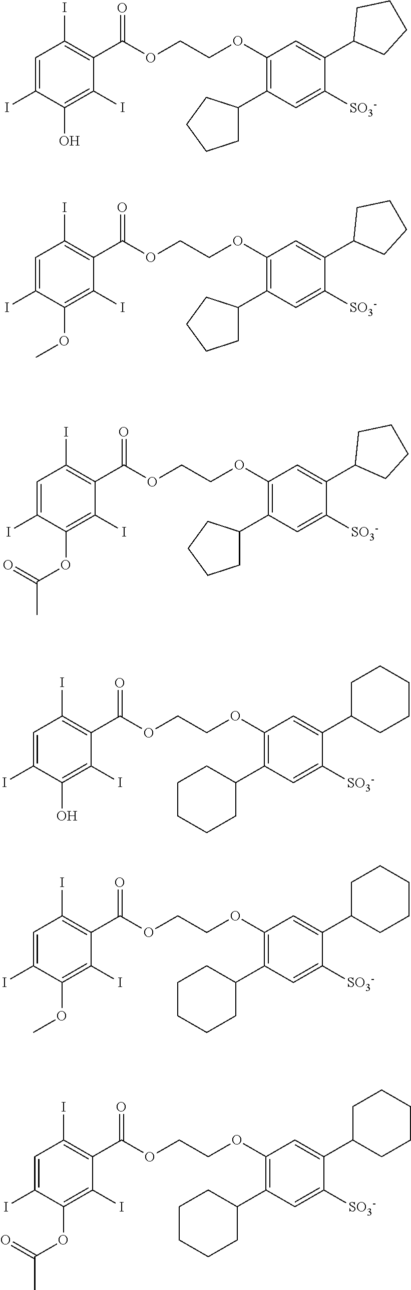

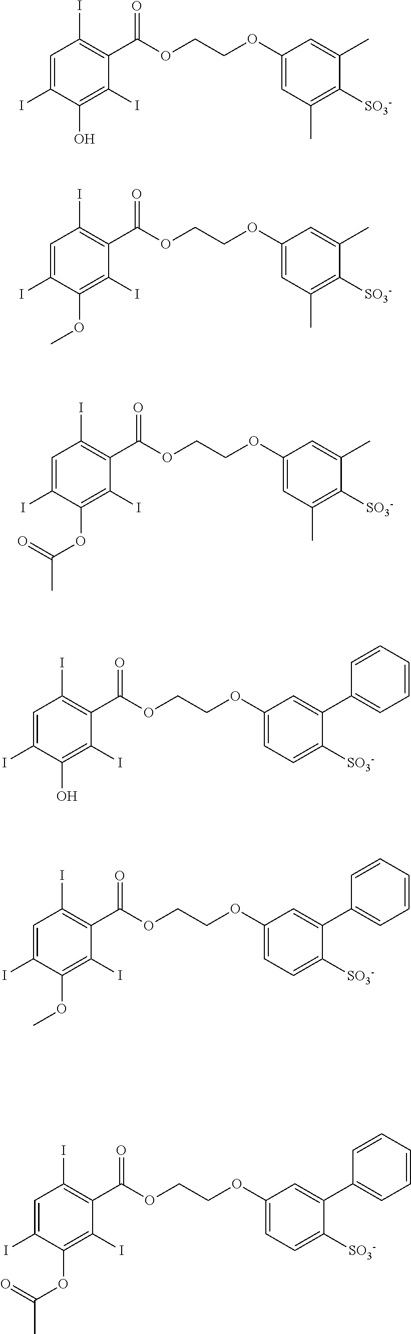

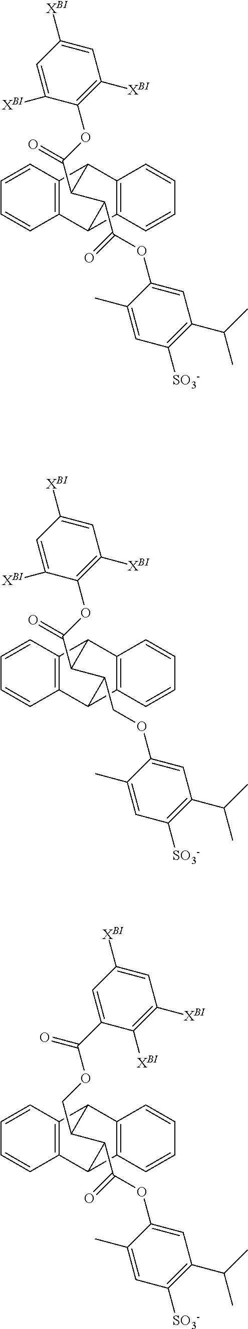

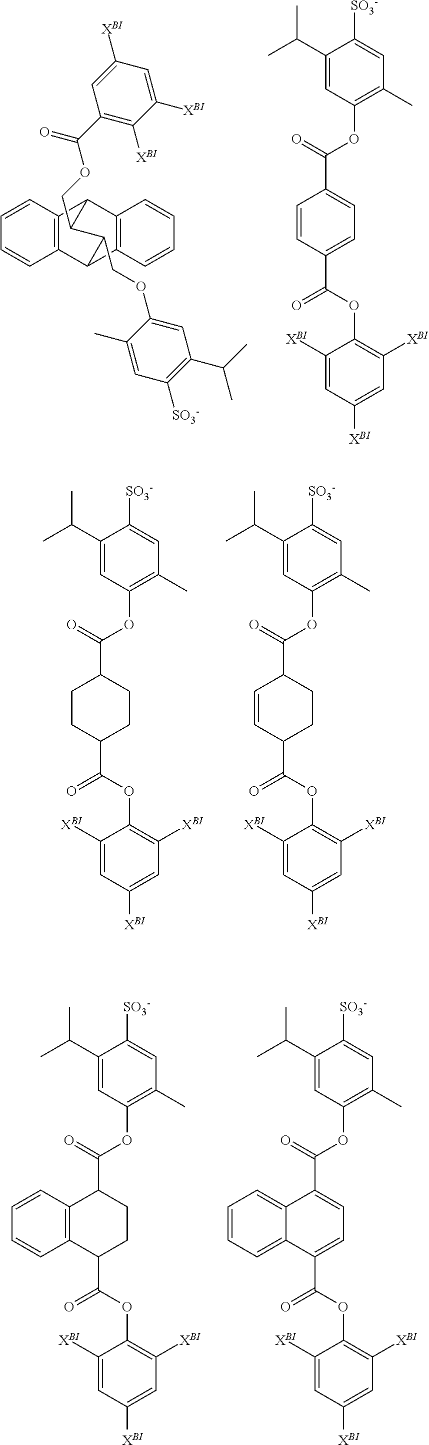

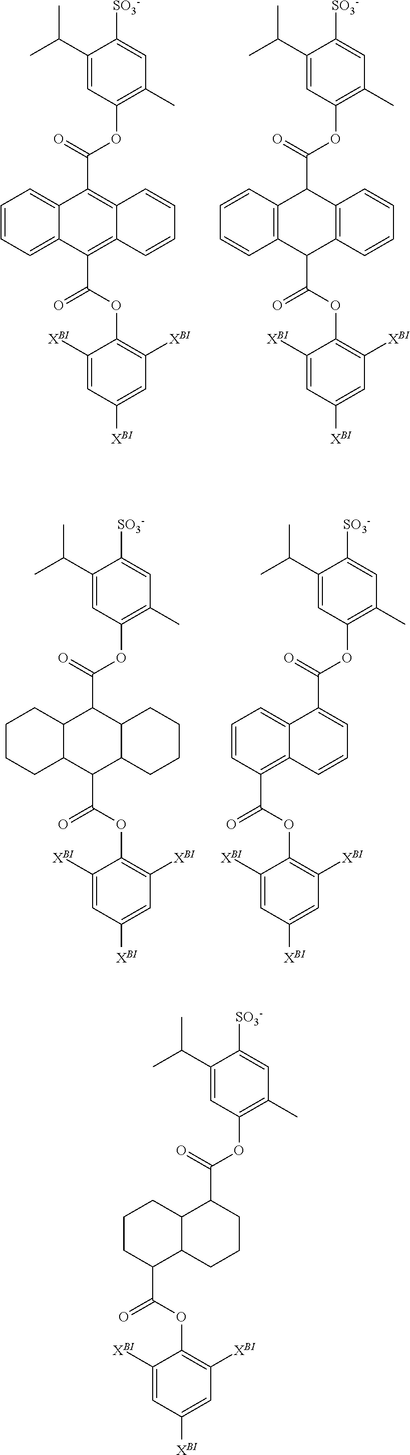

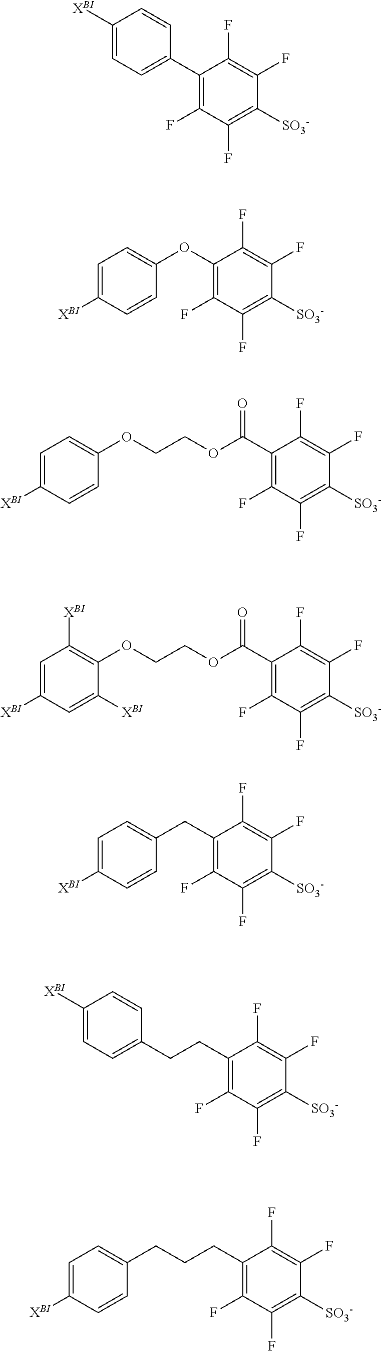

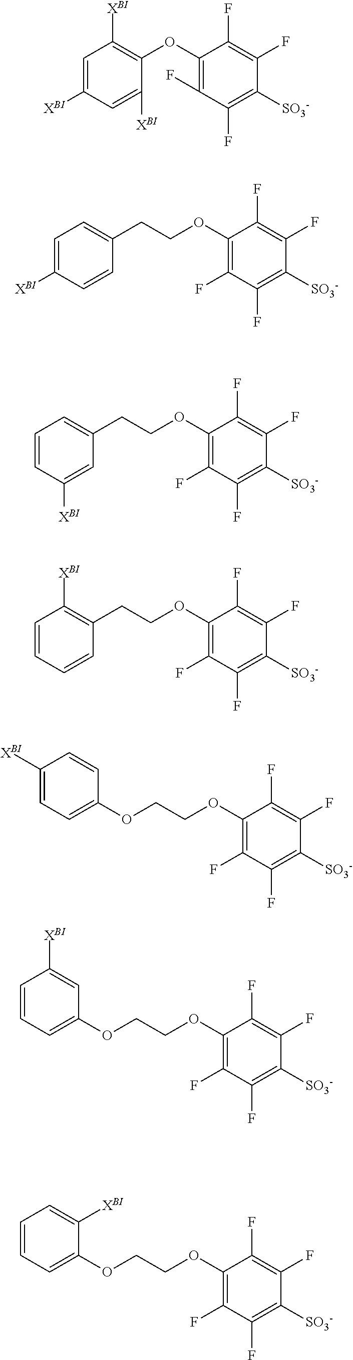

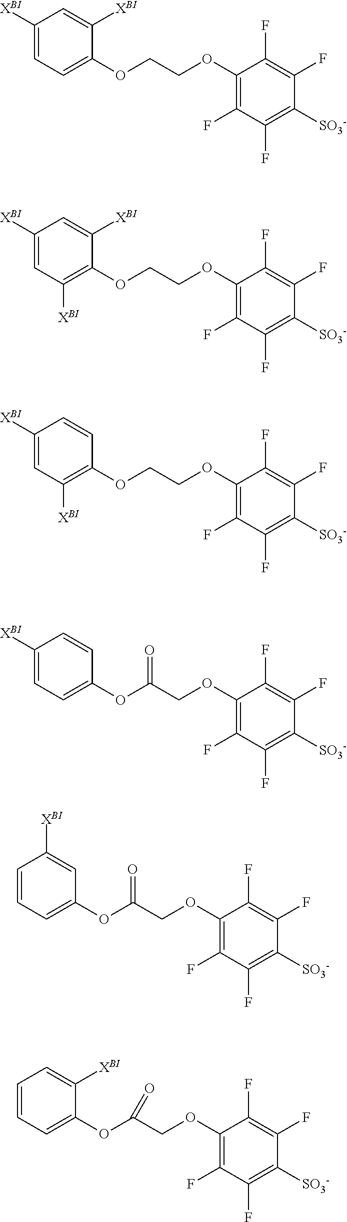

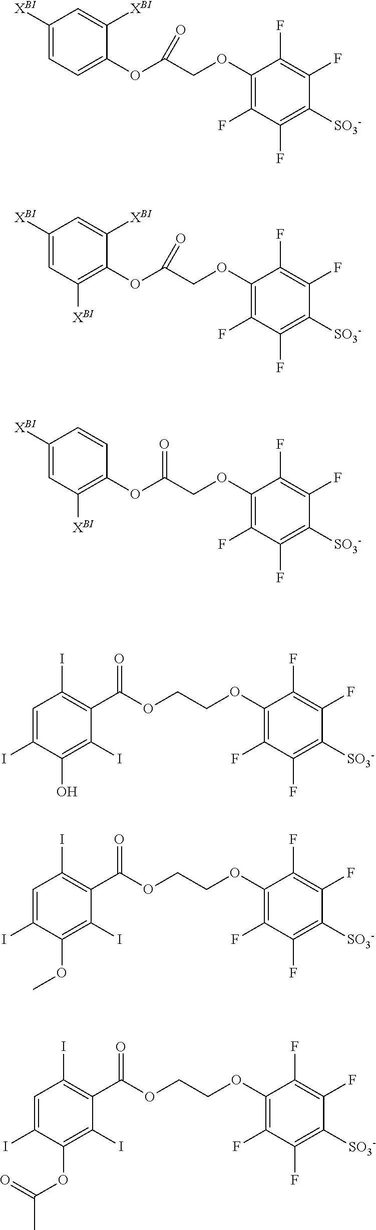

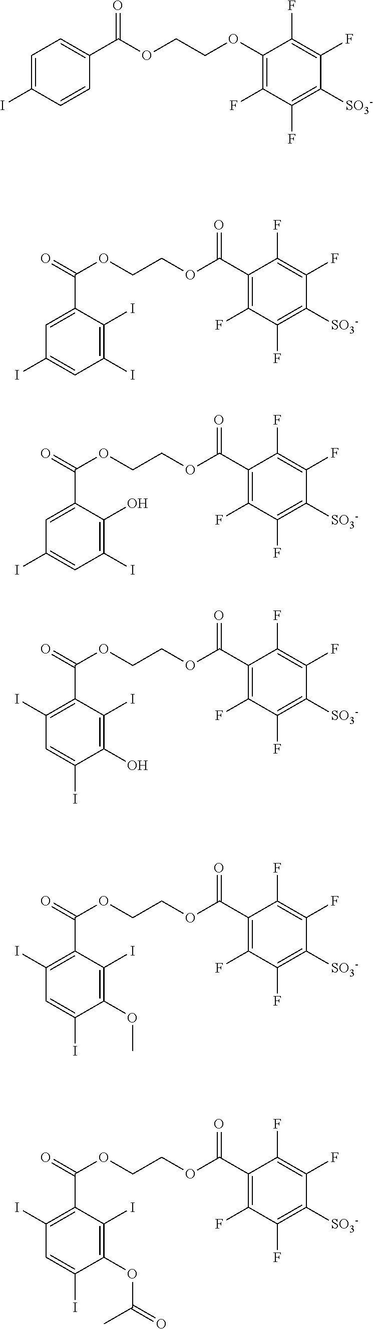

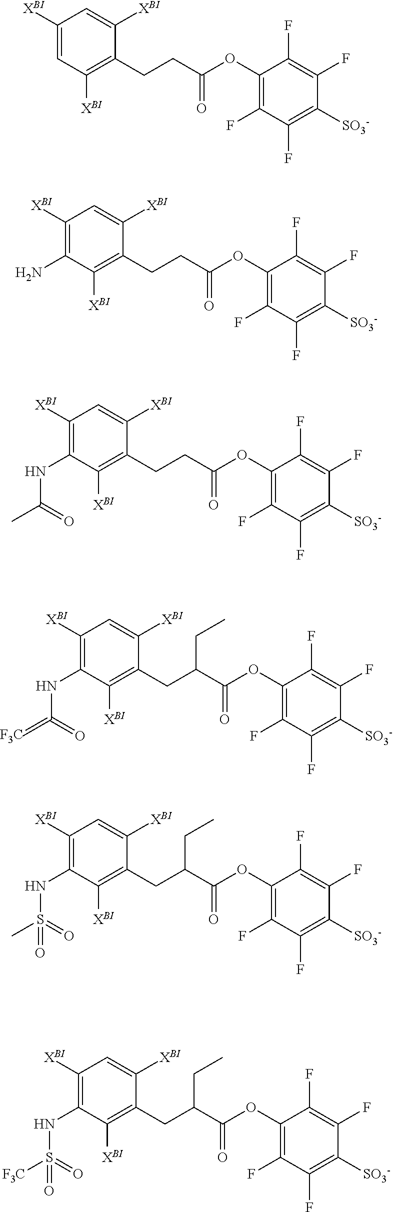

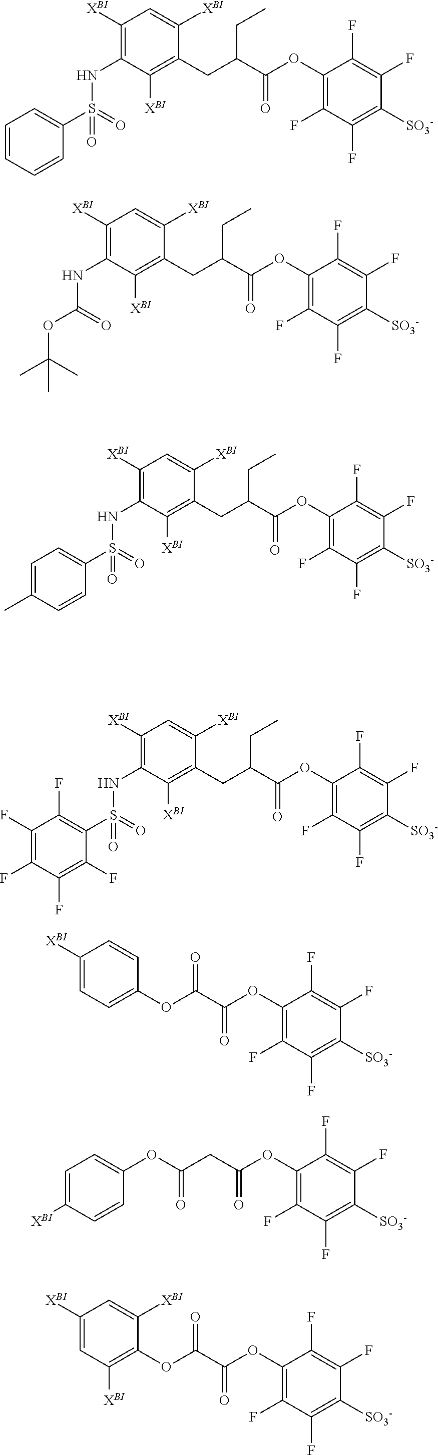

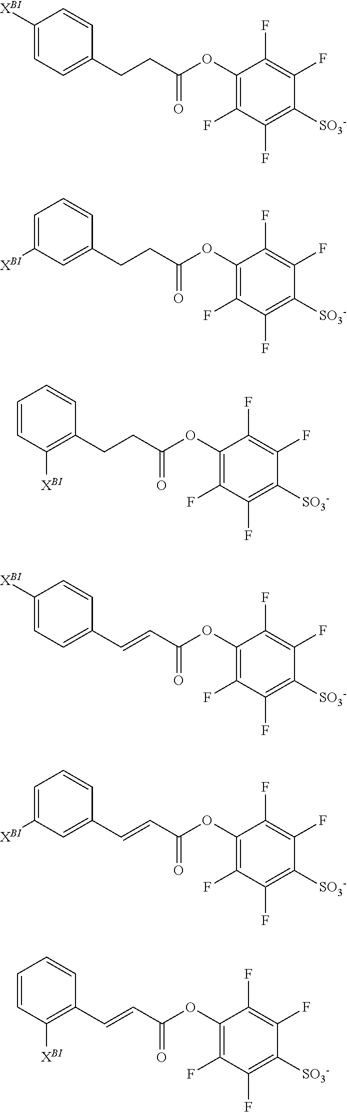

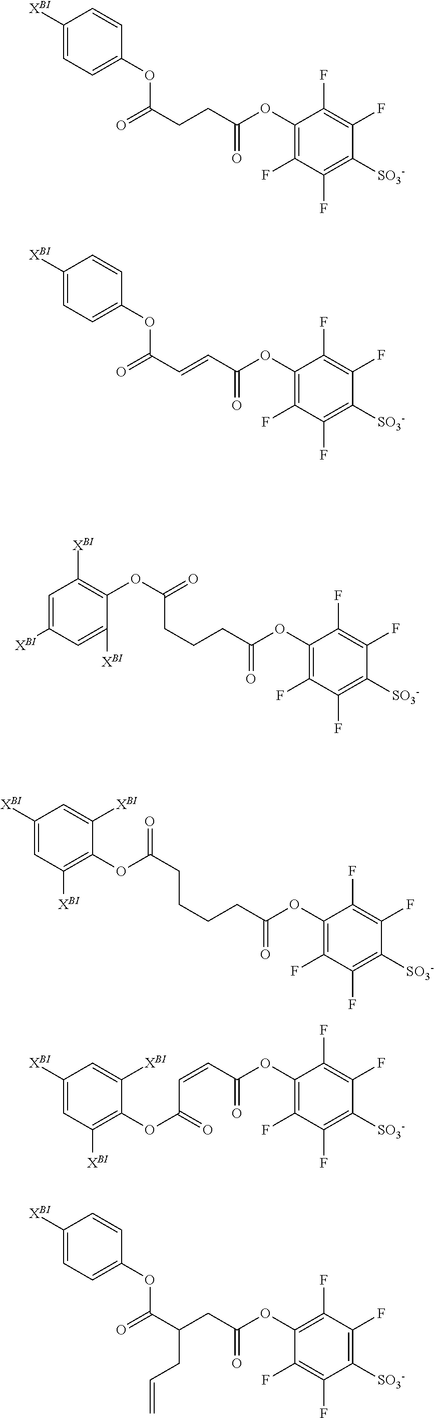

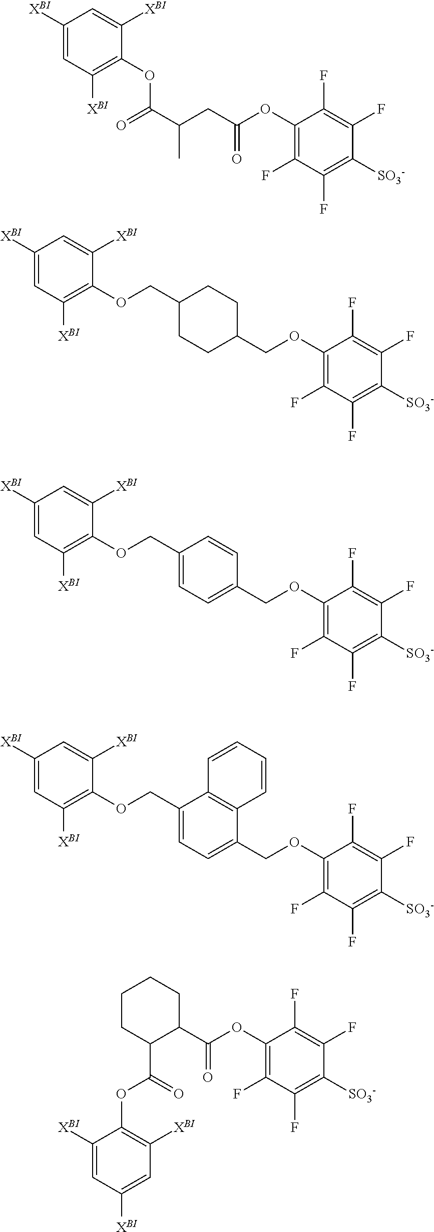

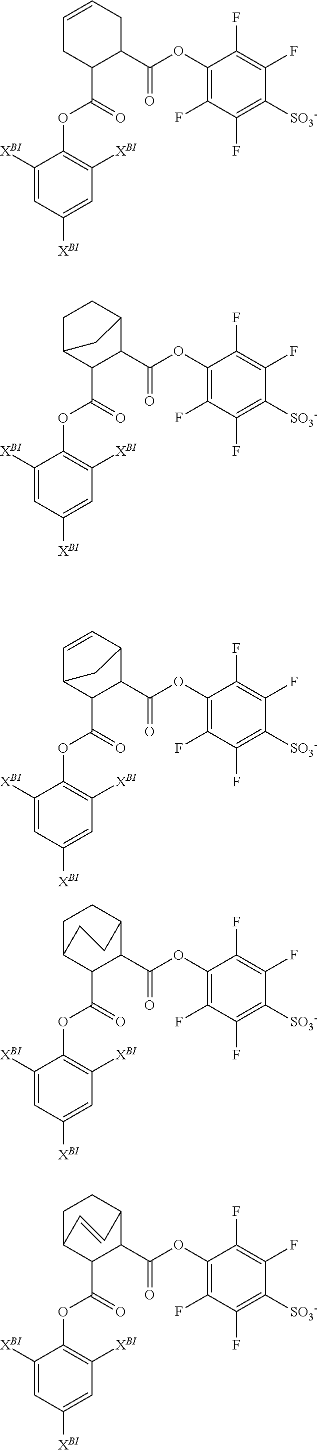

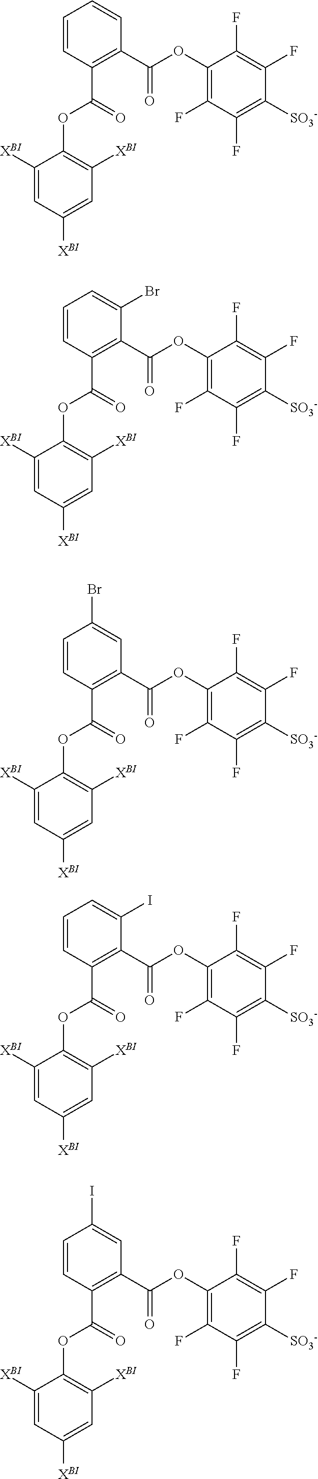

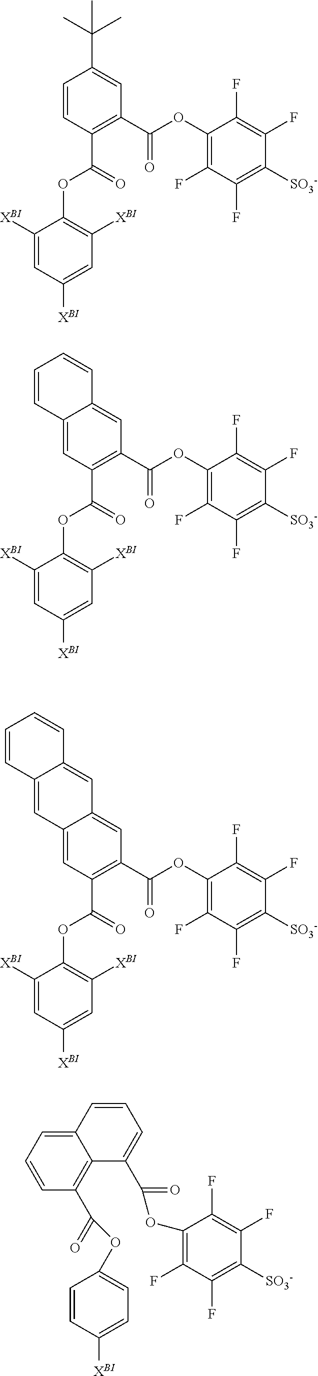

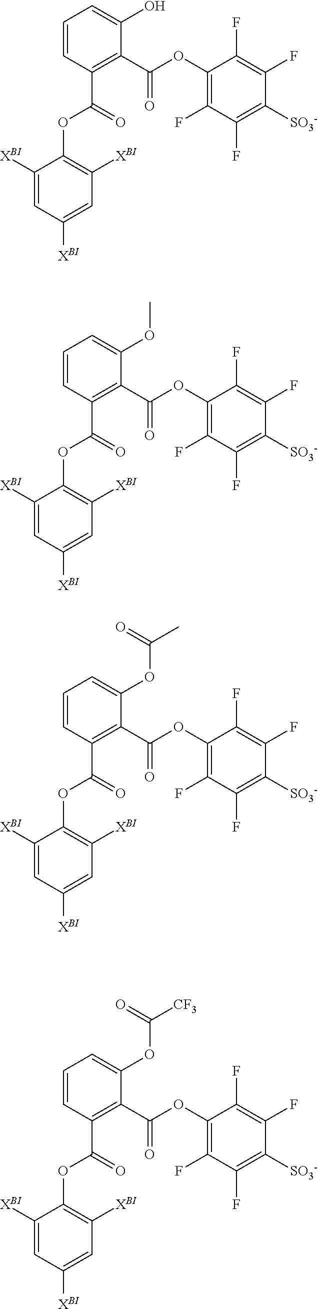

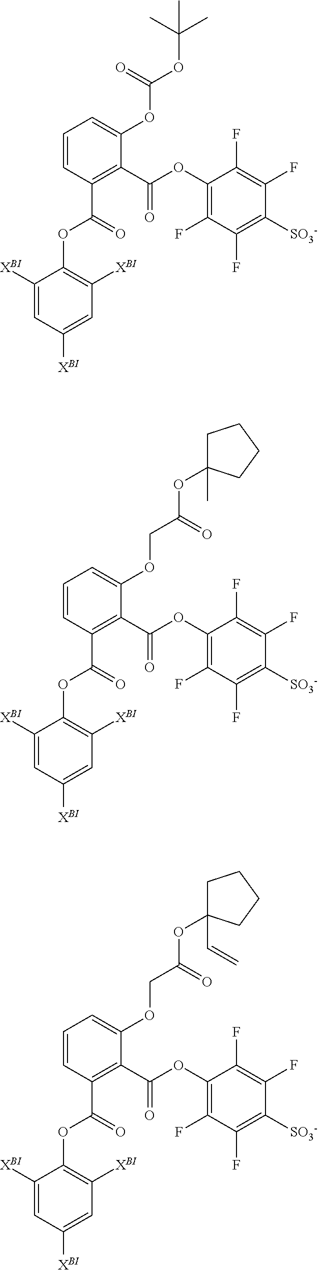

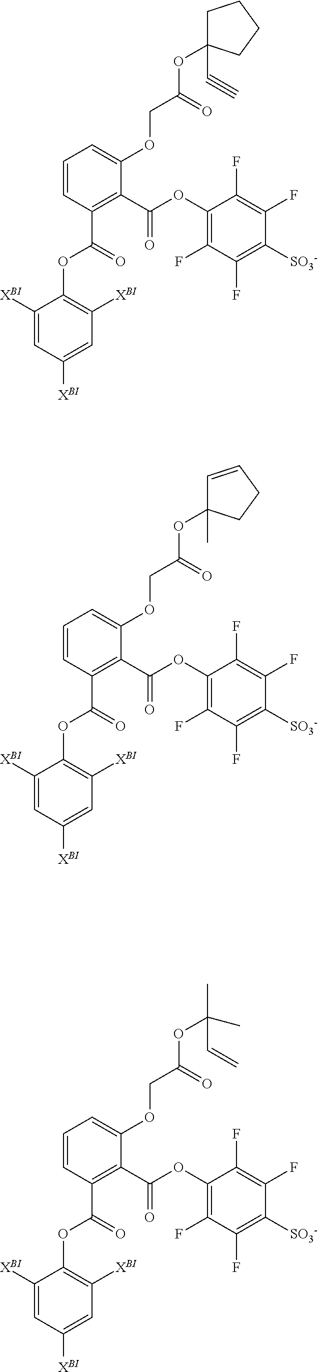

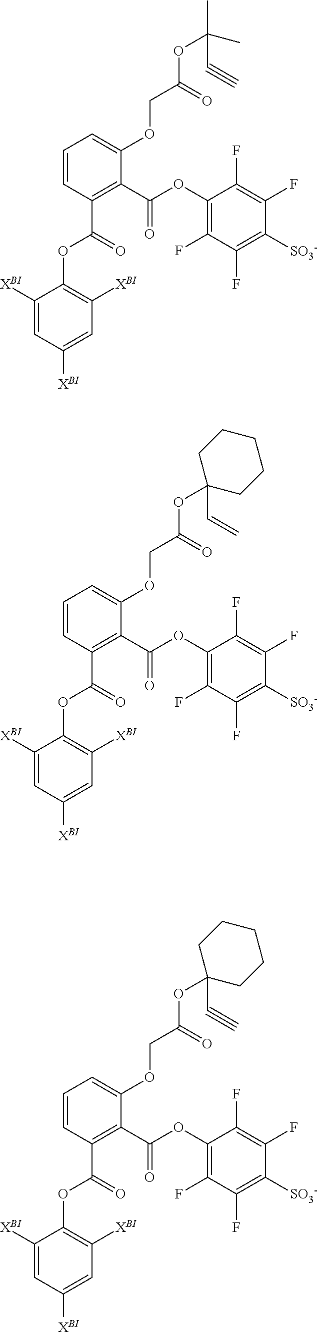

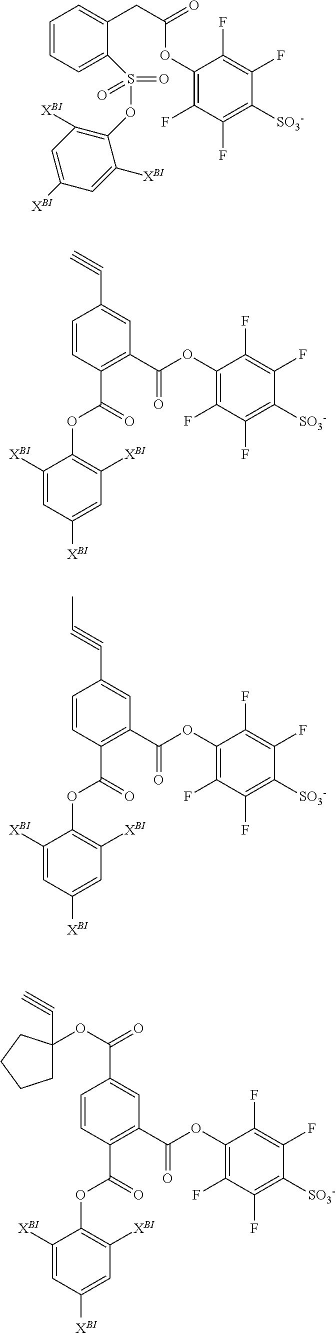

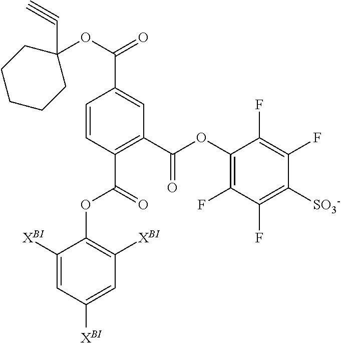

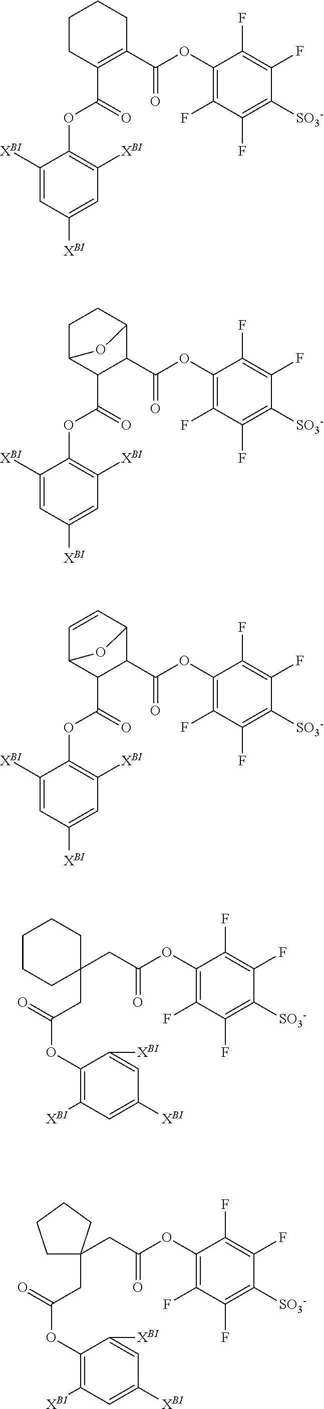

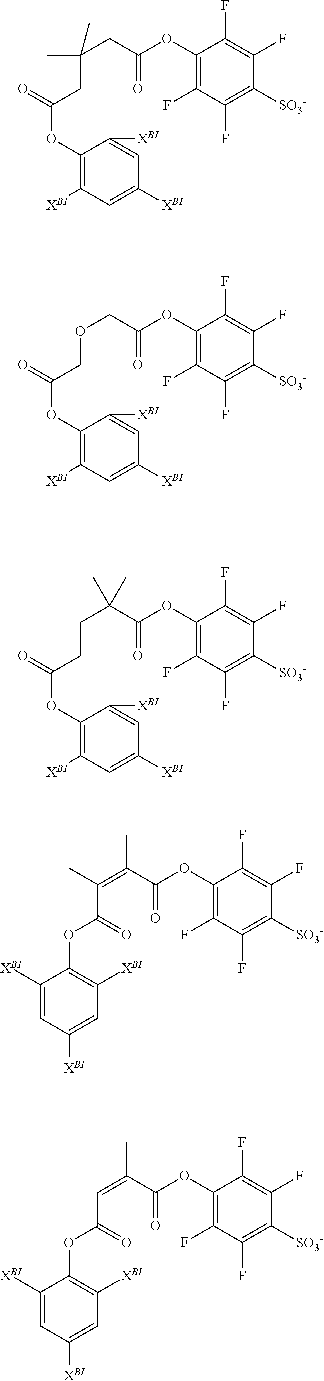

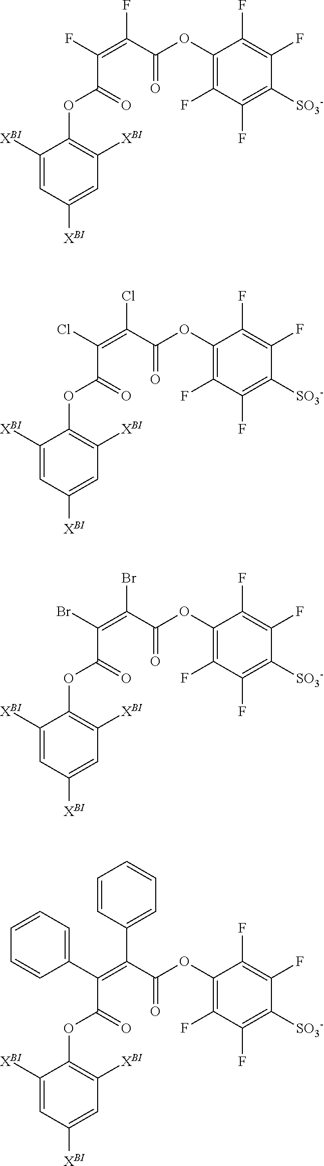

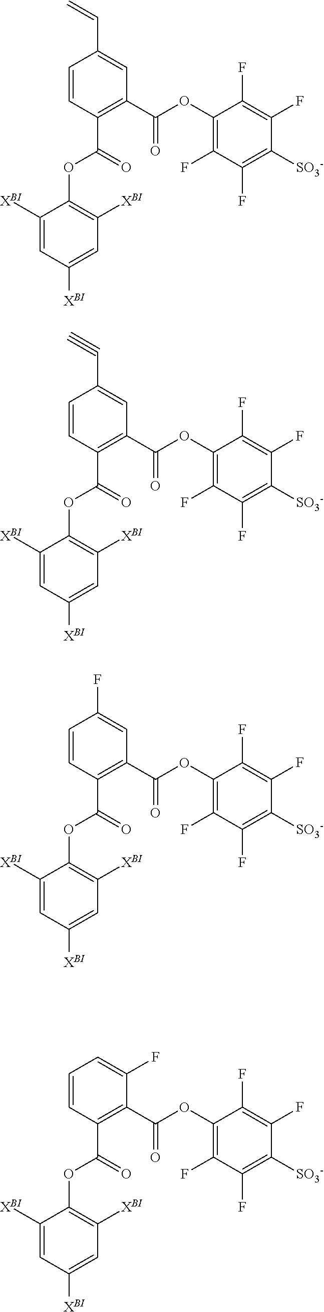

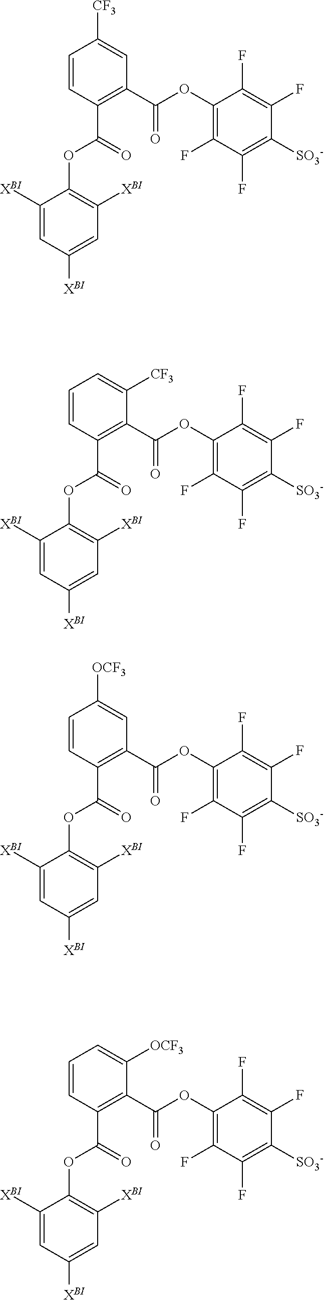

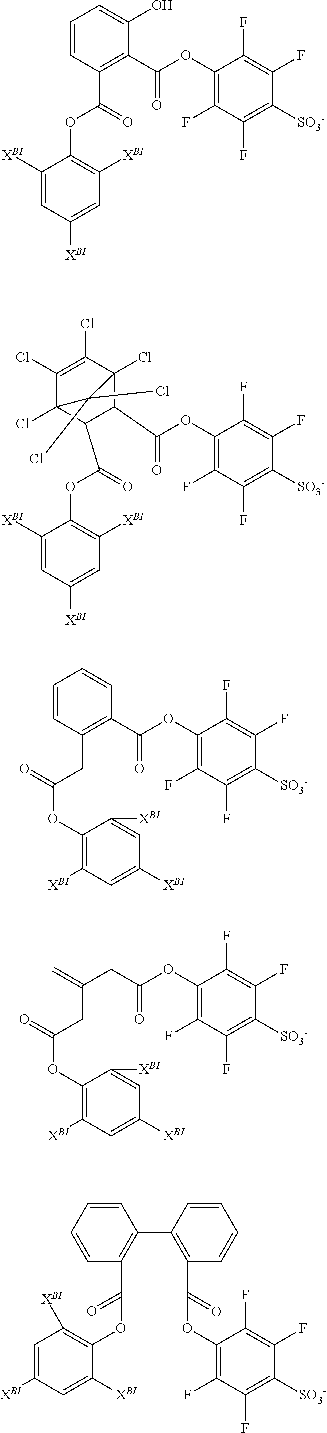

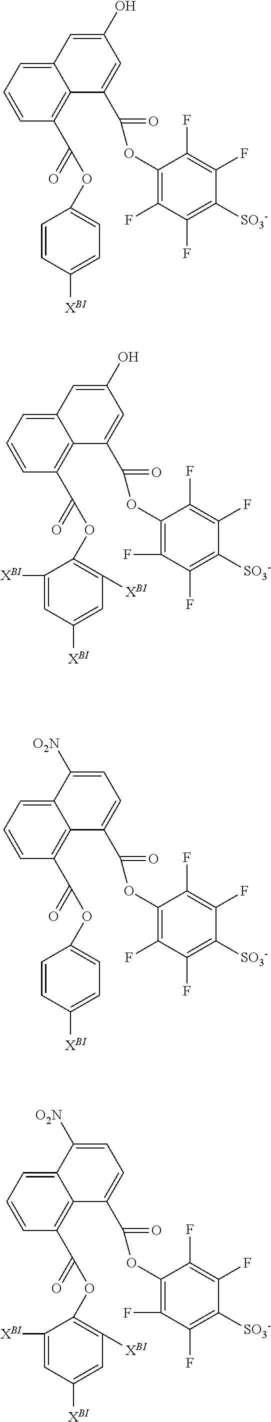

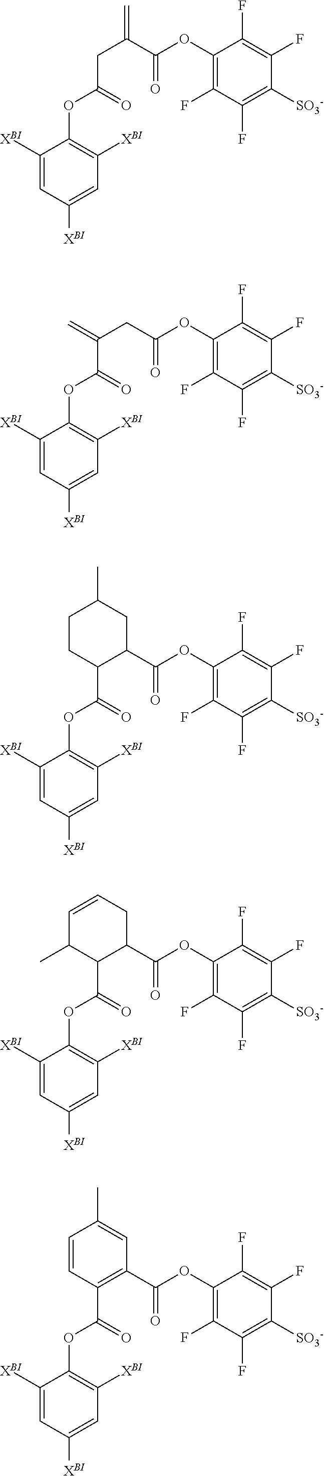

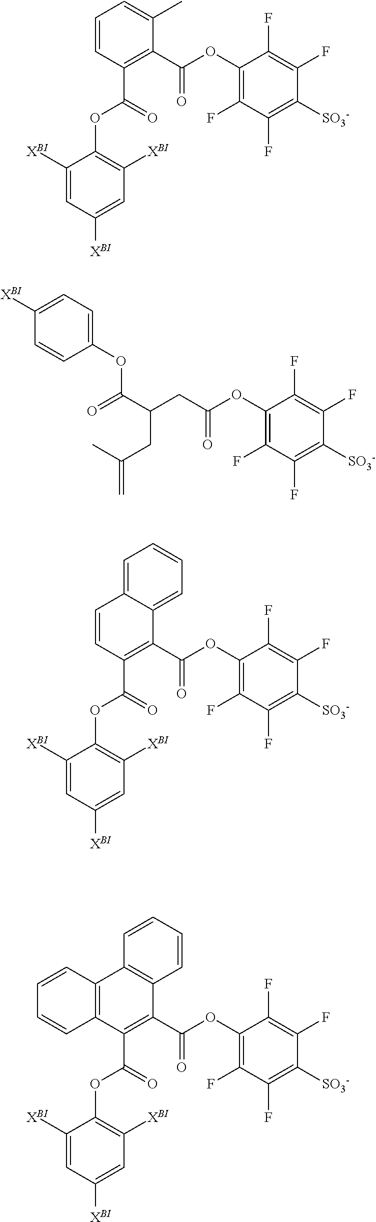

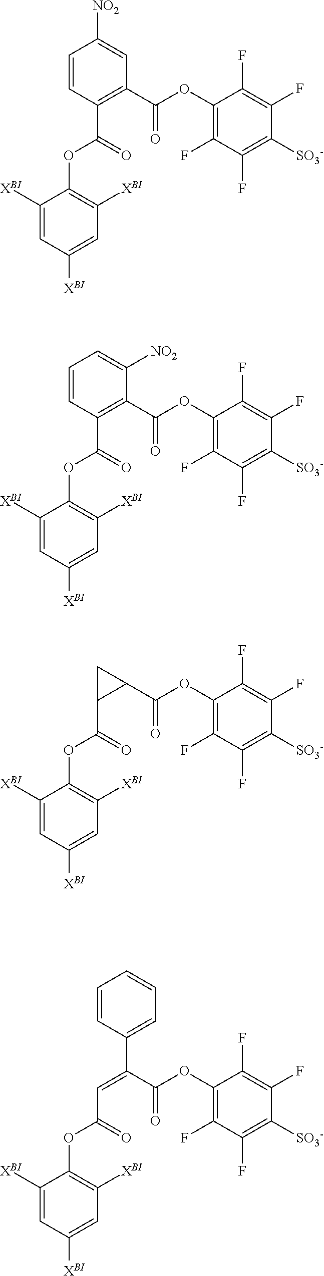

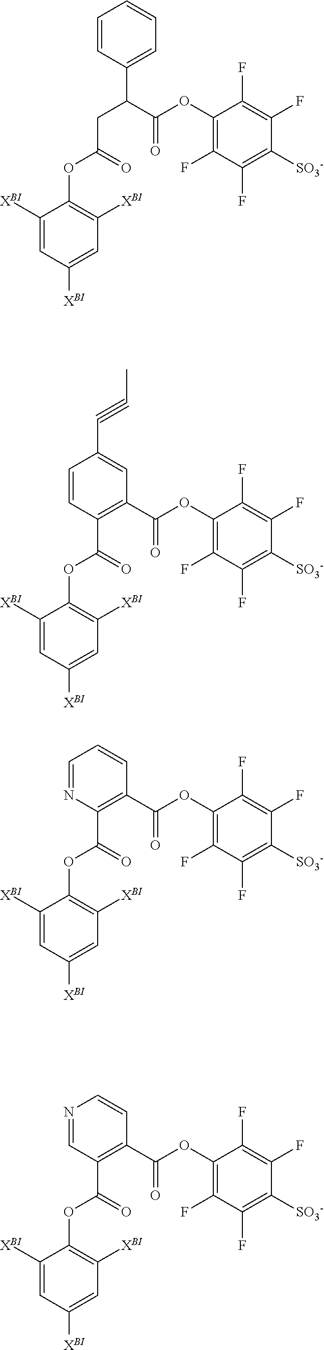

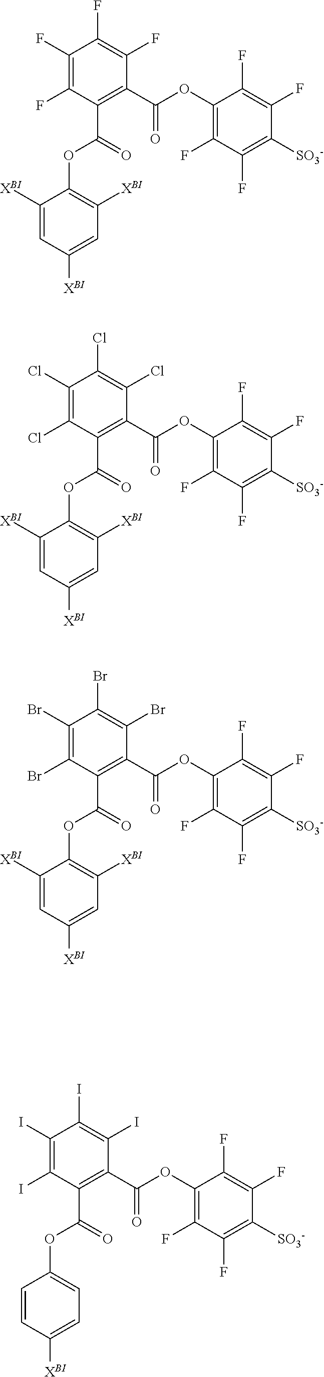

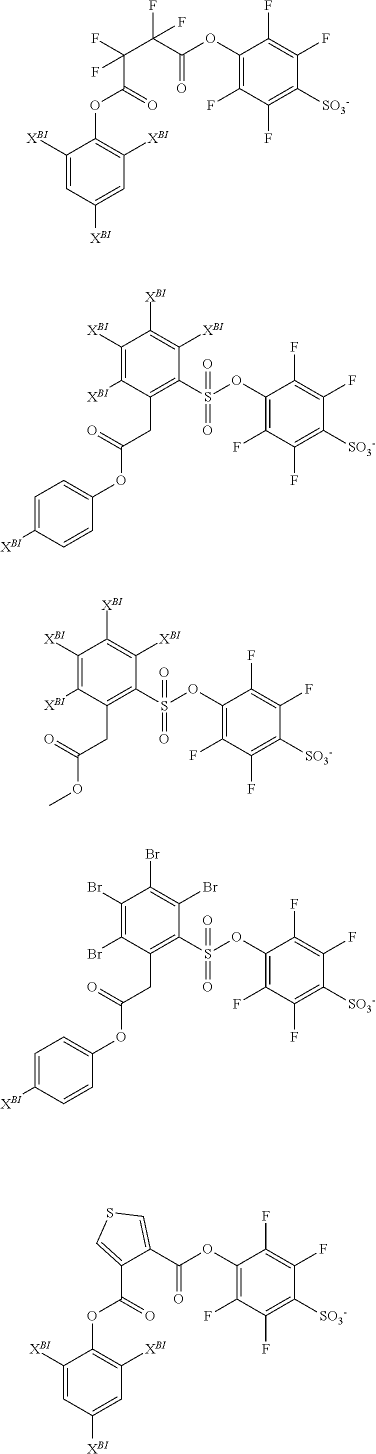

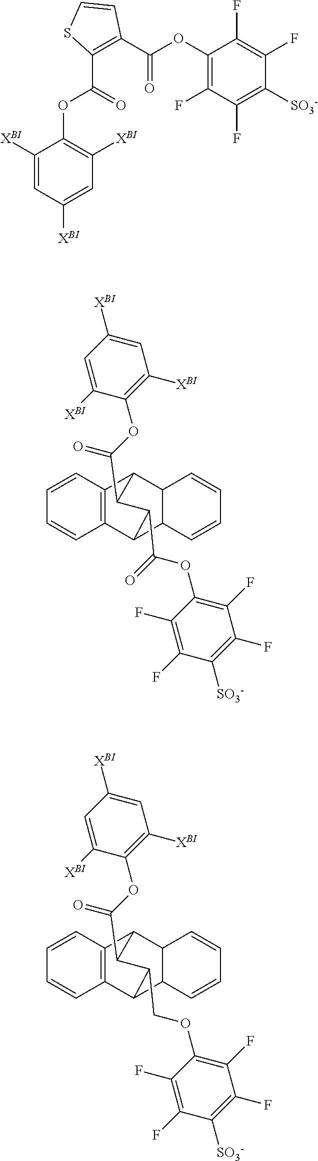

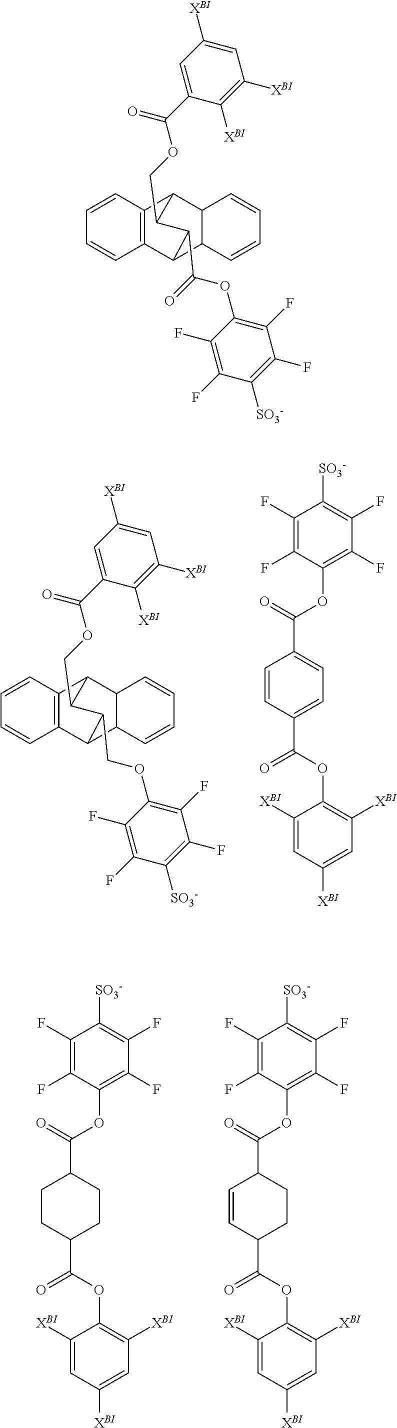

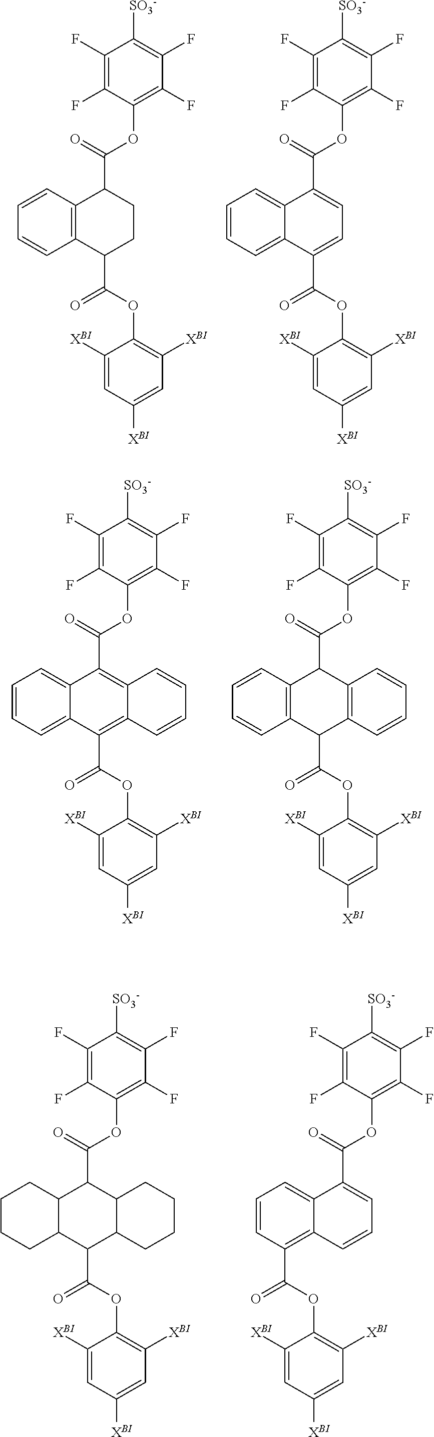

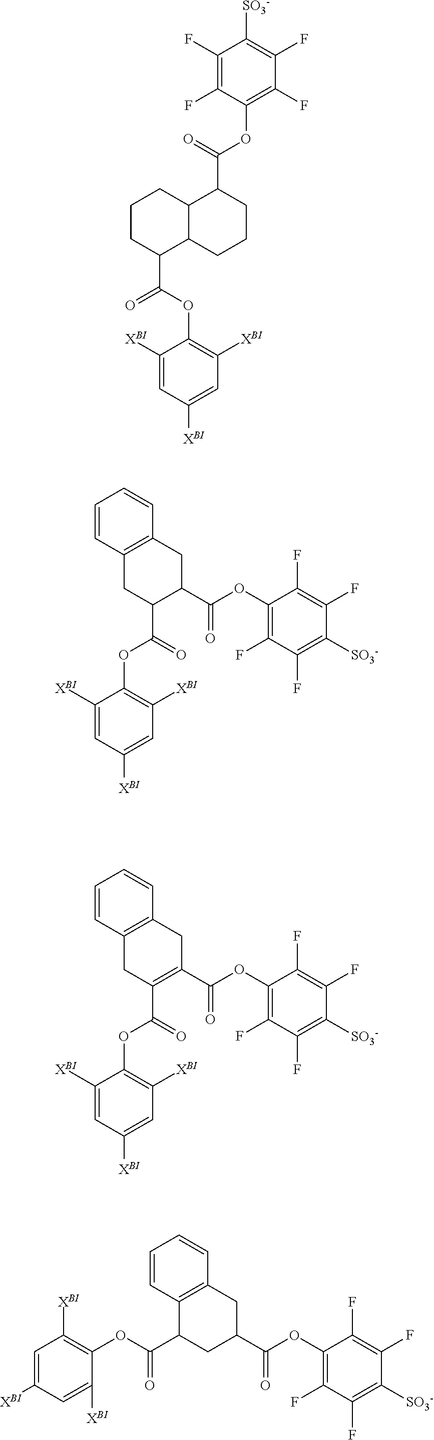

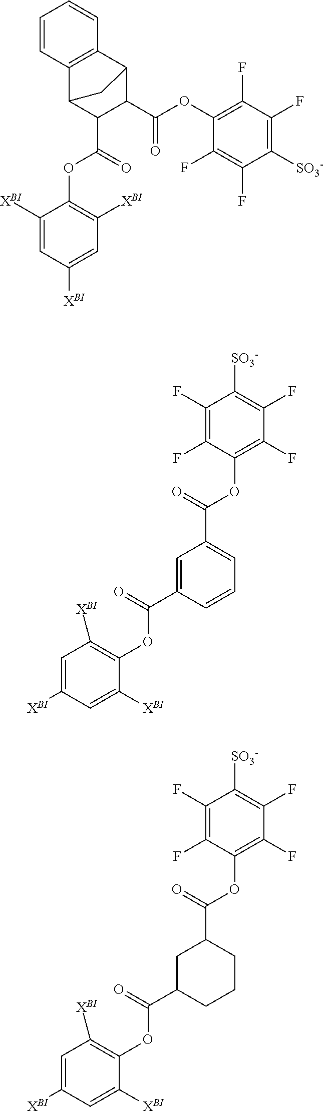

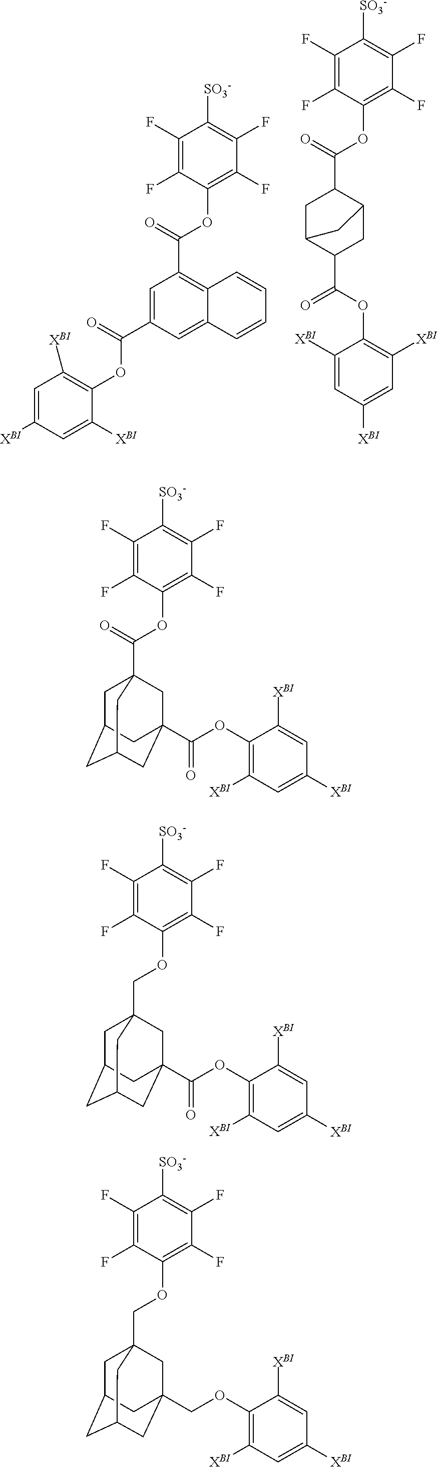

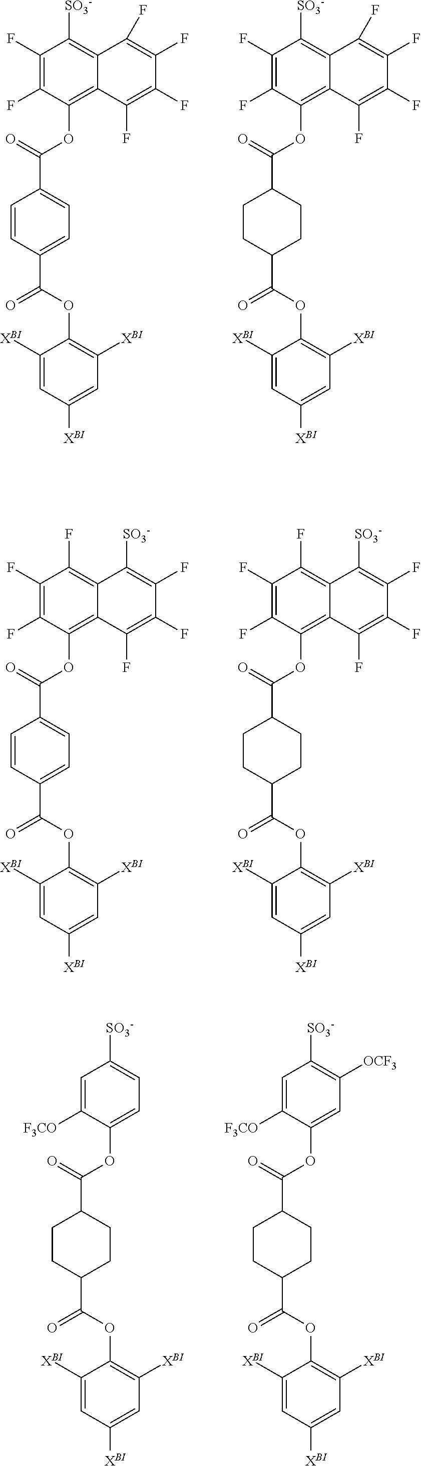

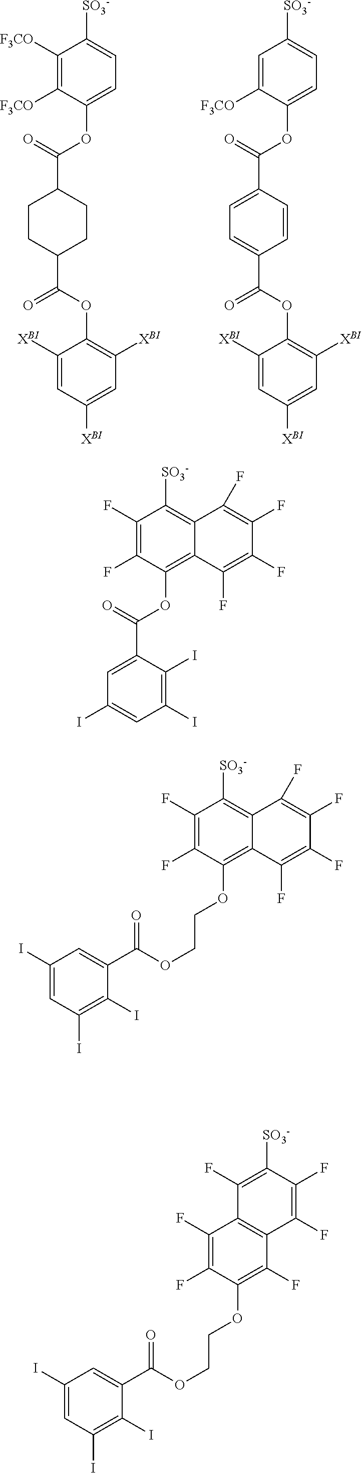

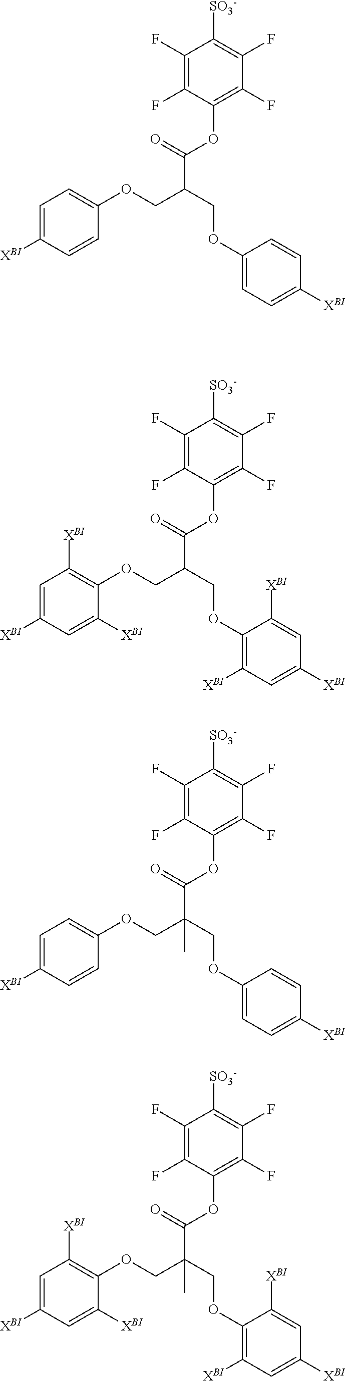

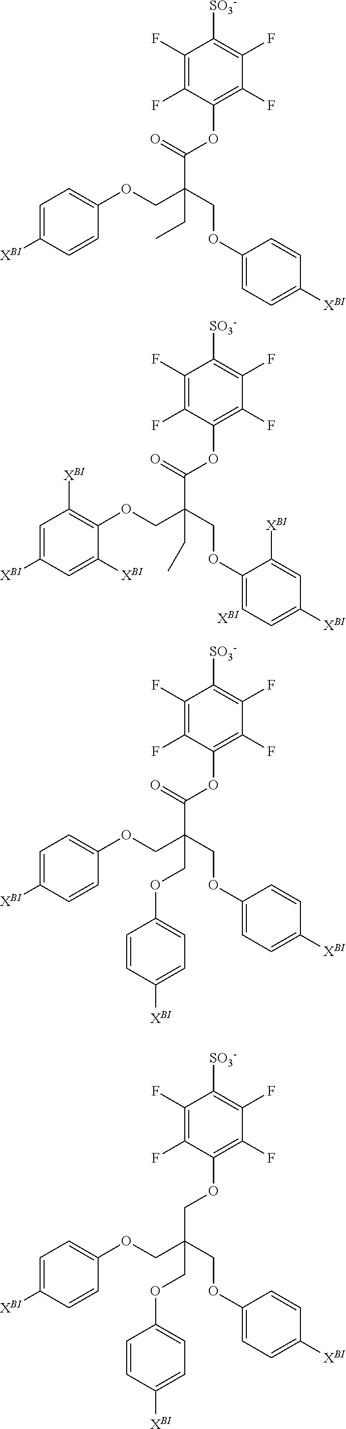

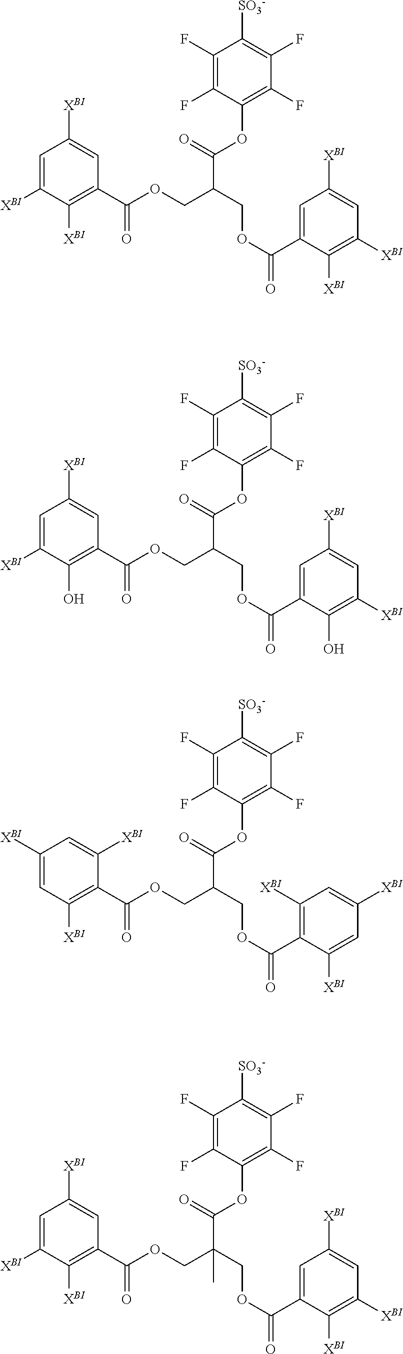

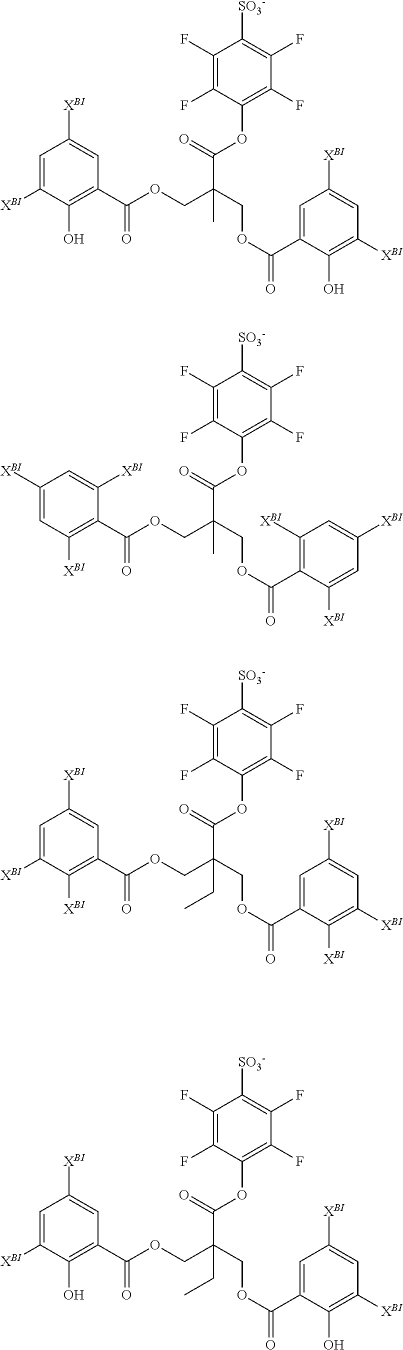

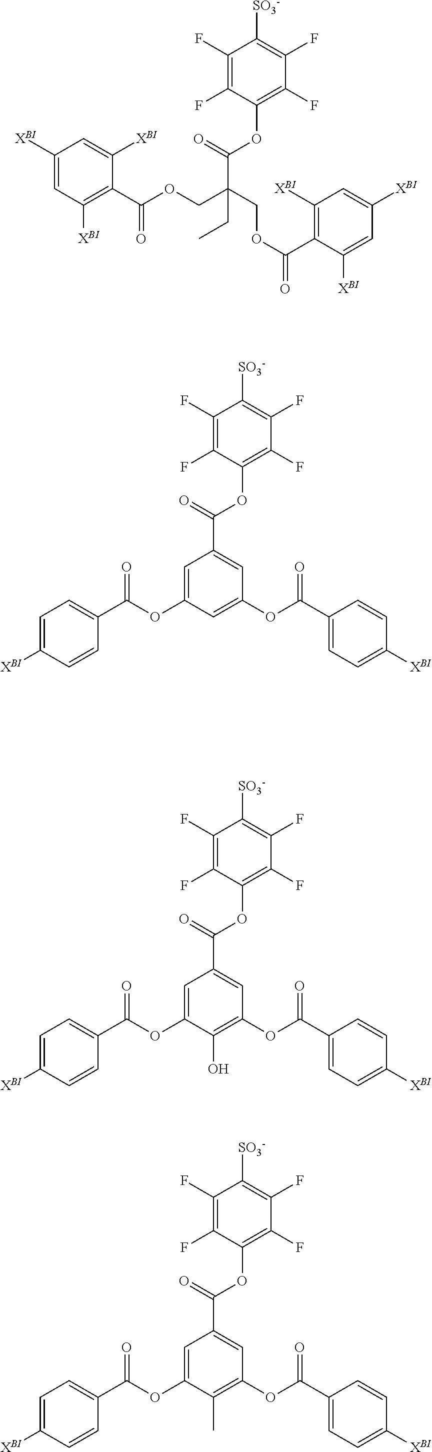

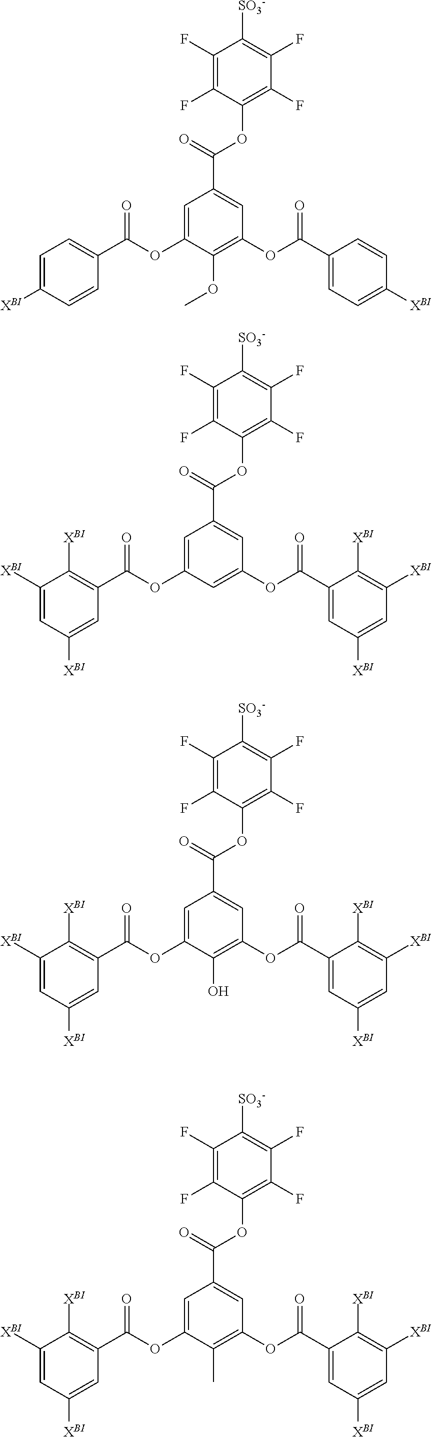

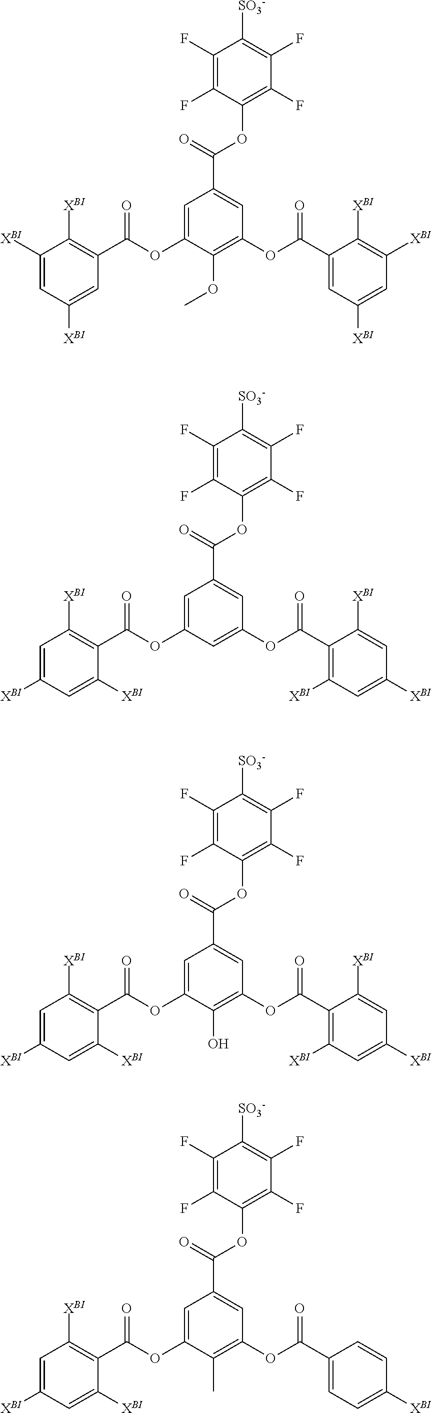

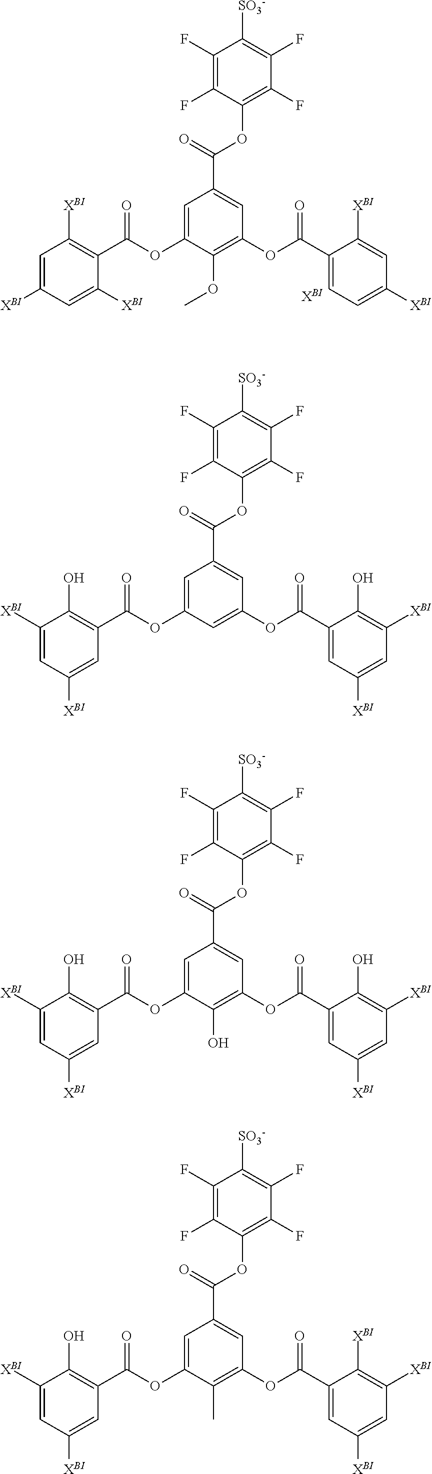

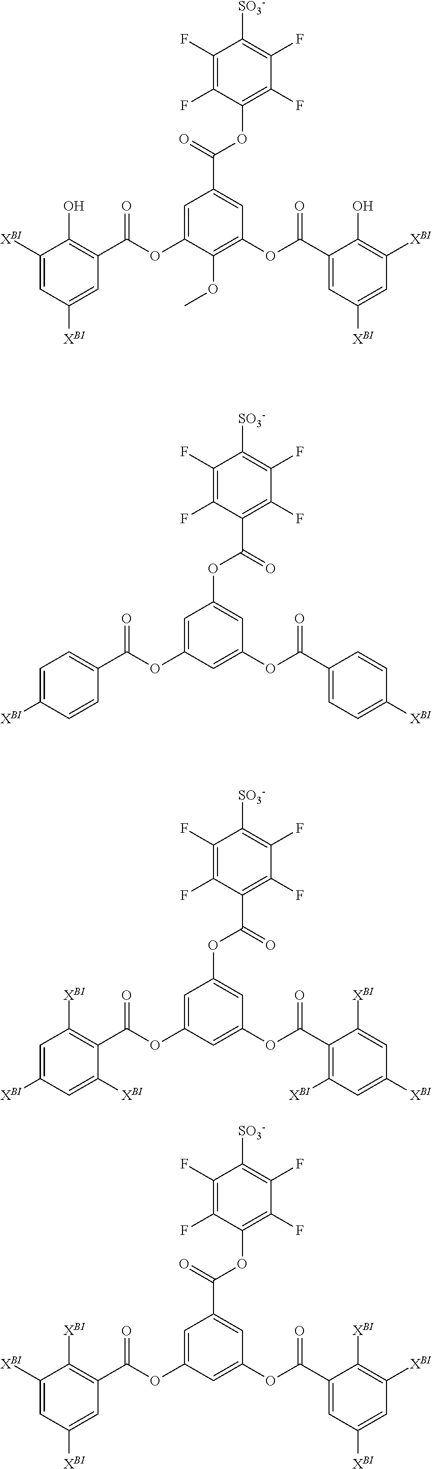

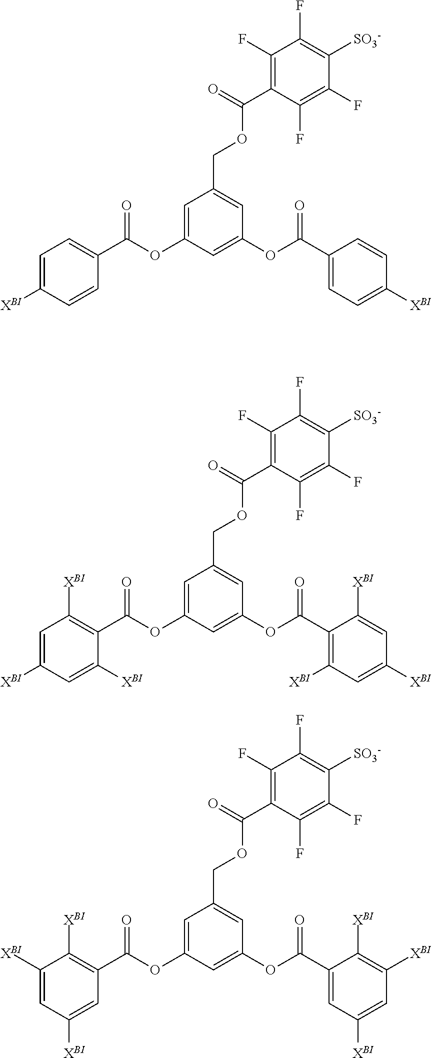

In formula (1), M⁺ is a sulfonium cation or an iodonium cation. The sulfonium cation is preferably one having formula (2), and the iodonium cation is preferably one having formula (3).

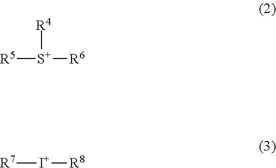

In formulae (2) and (3), R⁴ to R⁸ are each independently a halogen atom, or a C₁-C₂₀ hydrocarbyl group which may contain a heteroatom.

Examples of the halogen atom represented by R⁴ to R⁸ include a fluorine atom, a chlorine atom, a bromine atom, and an iodine atom.

The C₁-C₂₀ hydrocarbyl group represented by R⁴ to R⁸ may be saturated or unsaturated, and may be straight, branched, or cyclic. Examples thereof include C₁-C₂₀ alkyl groups such as a methyl group, an ethyl group, a n-propyl group, an isopropyl group, a n-butyl group, an isobutyl group, a sec-butyl group, a tert-butyl group, a n-pentyl group, a n-hexyl group, a n-octyl group, a n-nonyl group, a n-decyl group, an undecyl group, a dodecyl group, a tridecyl group, a tetradecyl group, a pentadecyl group, a heptadecyl group, an octadecyl group, a nonadecyl group, and an icosyl group; C₃-C₂₀ cyclic saturated hydrocarbyl groups such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; C₂-C₂₀ alkenyl groups such as a vinyl group, a propenyl group, a butenyl group, and a hexenyl group; C₂-C₂₀ alkynyl groups such as an ethynyl group, a propynyl group, and a butynyl group; C₃-C₂₀ cyclic unsaturated aliphatic hydrocarbyl groups such as a cyclohexenyl group and a norbornenyl group; C₆-C₂₀ aryl groups such as a phenyl group, a methylphenyl group, an ethylphenyl group, a n-propylphenyl group, an isopropylphenyl group, a n-butylphenyl group, an isobutylphenyl group, a sec-butylphenyl group, a tert-butylphenyl group, a naphthyl group, a methylnaphthyl group, an ethylnaphthyl group, a n-propylnaphthyl group, an isopropylnaphthyl group, a n-butylnaphthyl group, an isobutylnaphthyl group, a sec-butylnaphthyl group, and a tert-butylnaphthyl group; C₇-C₂₀ aralkyl groups such as a benzyl group and a phenethyl group; and groups obtained by combining the foregoing.

Some or all of hydrogen atoms of the hydrocarbyl group may be substituted with a group containing a heteroatom such as an oxygen atom, a sulfur atom, a nitrogen atom, or a halogen atom, and some of —CH₂— of the hydrocarbyl group may be substituted with a group containing a heteroatom such as an oxygen atom, a sulfur atom, or a nitrogen atom, so that the group may contain a hydroxy group, a fluorine atom, a chlorine atom, a bromine atom, an iodine atom, a cyano group, a nitro group, a mercapto group, a carbonyl group, an ether bond, an ester bond, a sulfonic acid ester bond, a carbonate bond, a lactone ring, a sultone ring, a carboxylic anhydride (—C(═O)—O—C(═O)—), or a haloalkyl group.

R⁴ and R⁵ may bond together to form a ring together with the sulfur atom to which they are bonded. The ring preferably has the following structure.

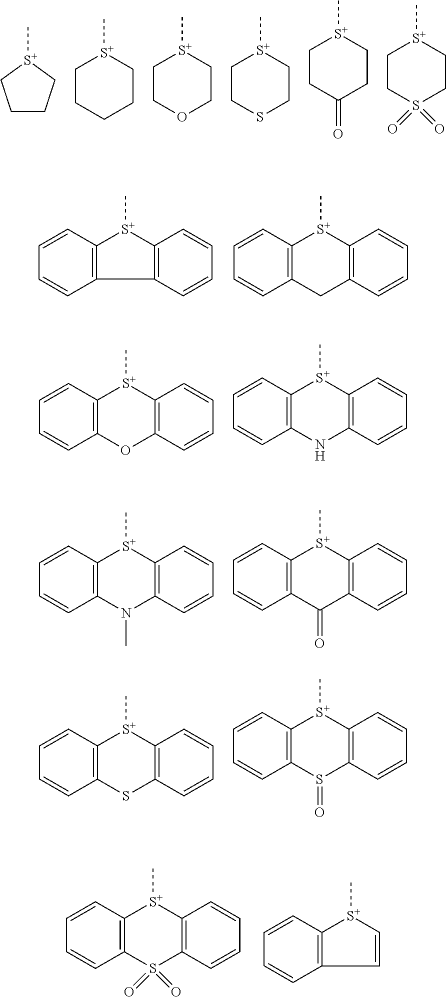

In the formulae, a broken line represents a bond.

Examples of the sulfonium cation represented by M⁺ include those shown below, but are not limited thereto.

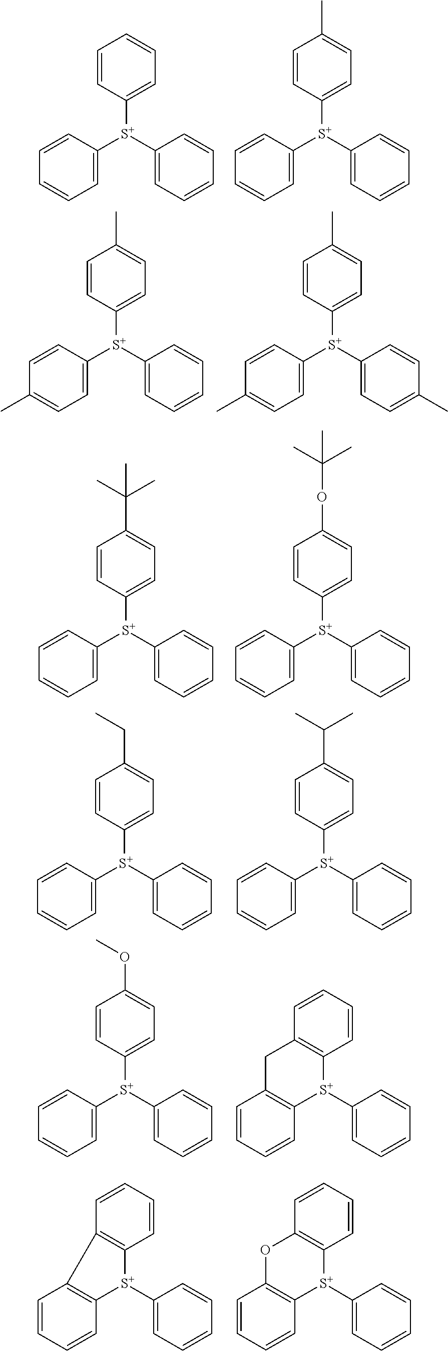

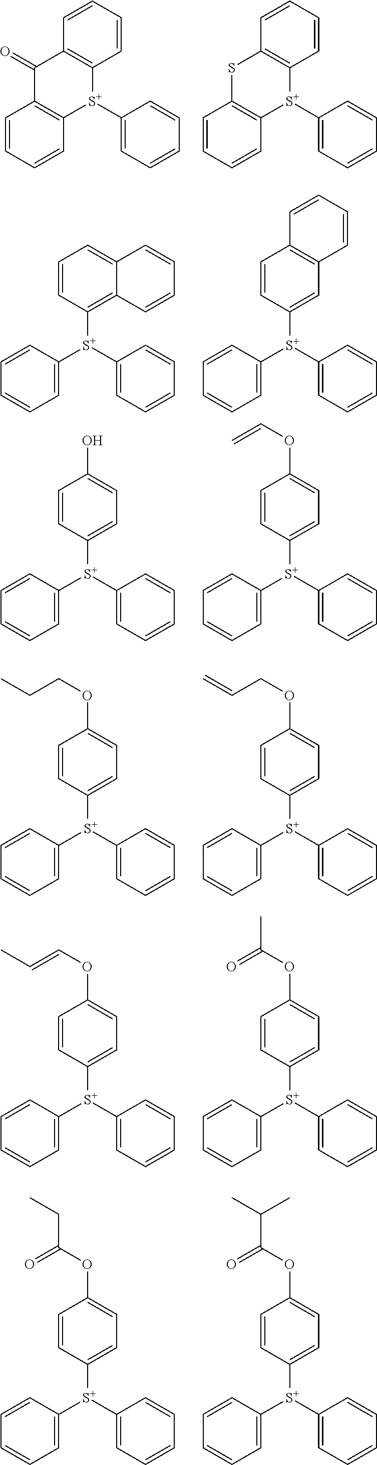

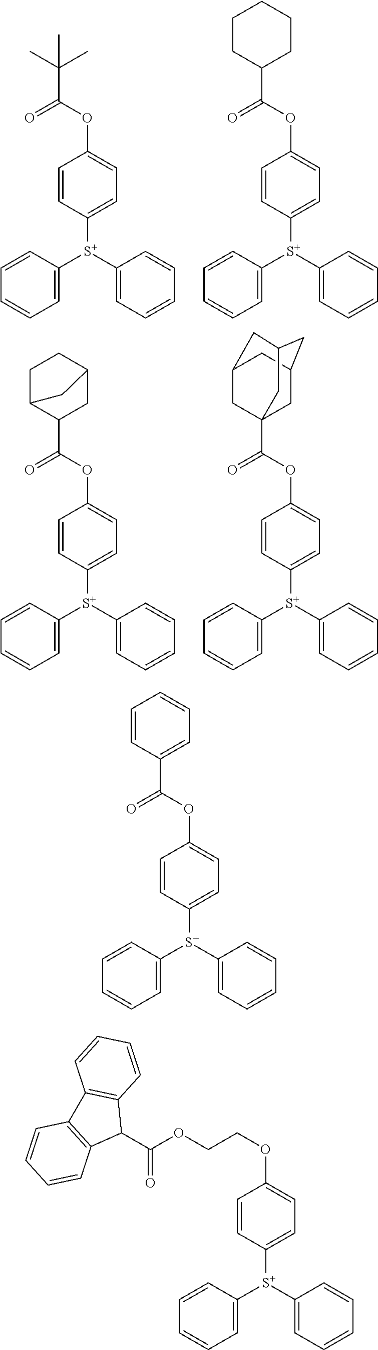 [SMILES: CC(C)(C)C(=O)Oc1ccc([S+](c2ccccc2)c2ccccc2)cc1 ; O=C(Oc1ccc([S+](c2ccccc2)c2ccccc2)cc1)C1CCCCC1 ; O=C(Oc1ccc([S+](c2ccccc2)c2ccccc2)cc1)C1CC2CCC1C2 ; O=C(Oc1ccc([S+](c2ccccc2)c2ccccc2)cc1)C12CC3CC(CC(C3)C1)C2 ; O=C(Oc1ccc([S+](c2ccccc2)c2ccccc2)cc1)c1ccccc1 ; O=C(OCCOc1ccc([S+](c2ccccc2)c2ccccc2)cc1)C1c2ccccc2-c2ccccc21]

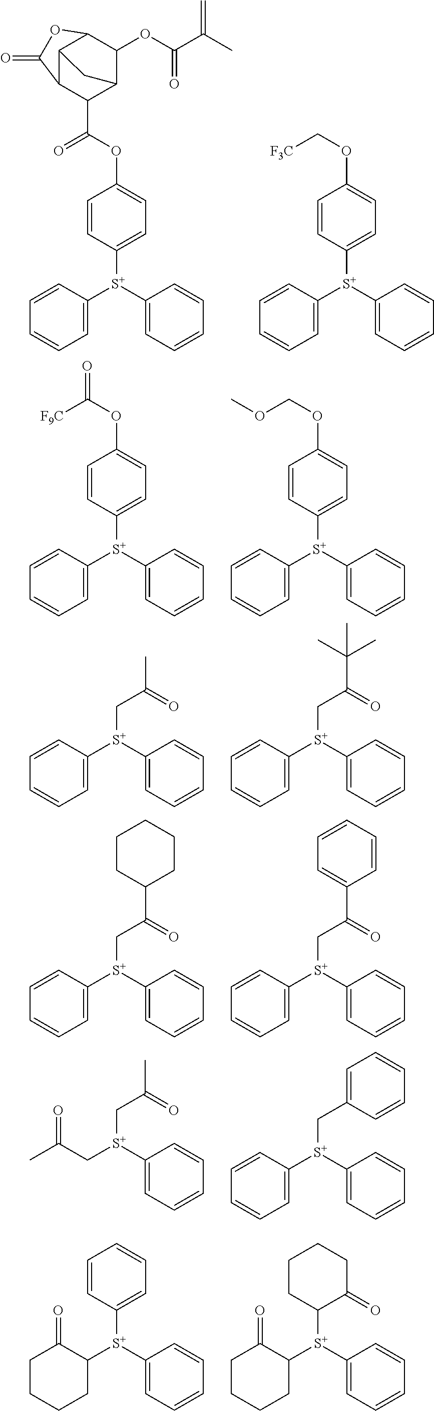

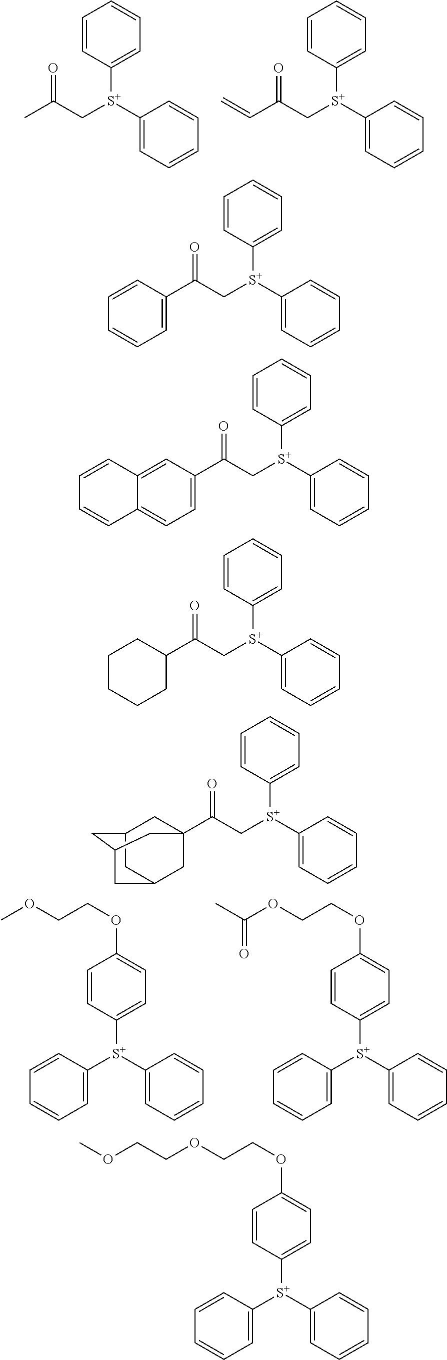

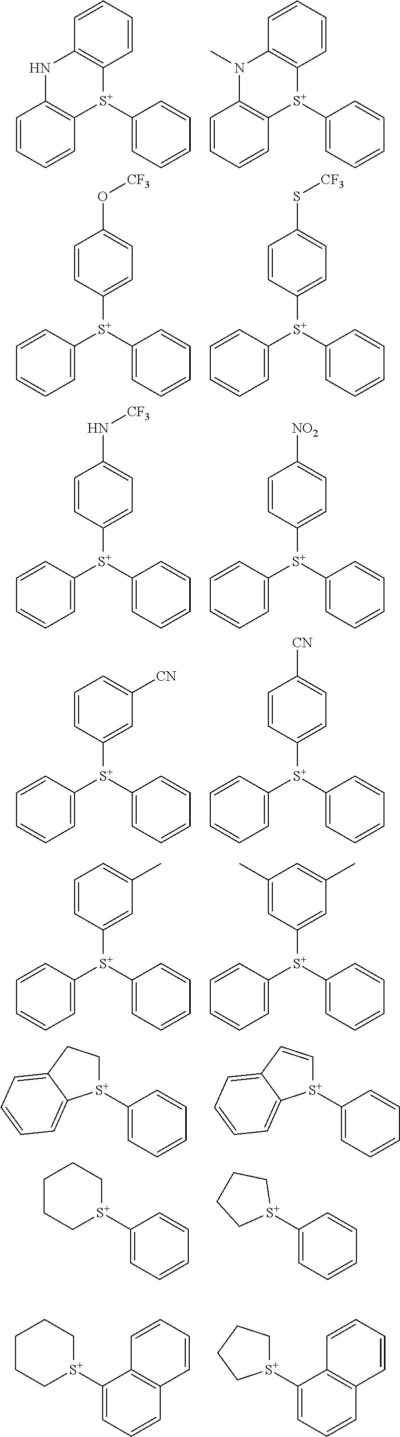

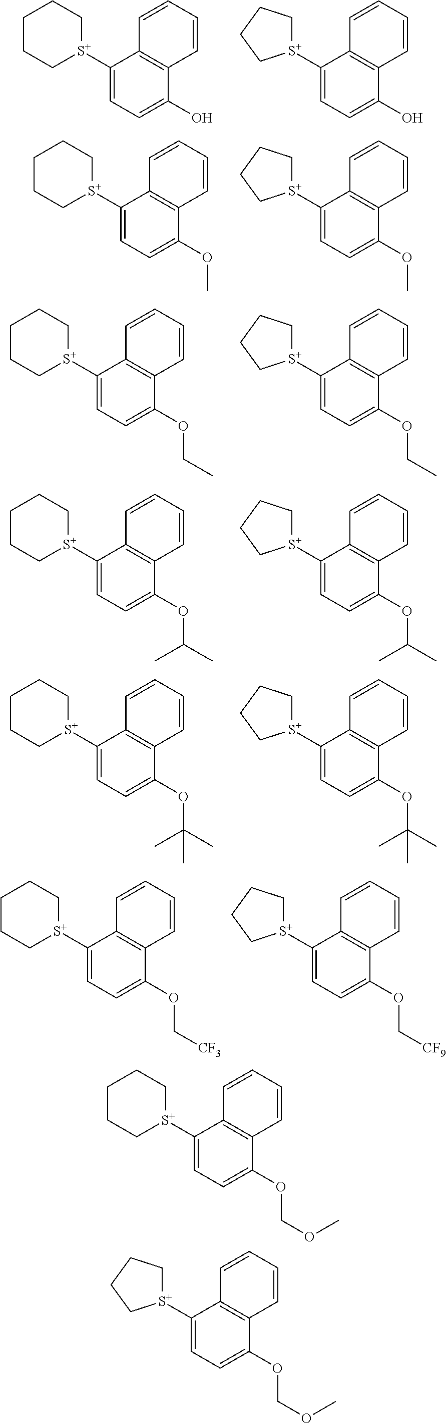

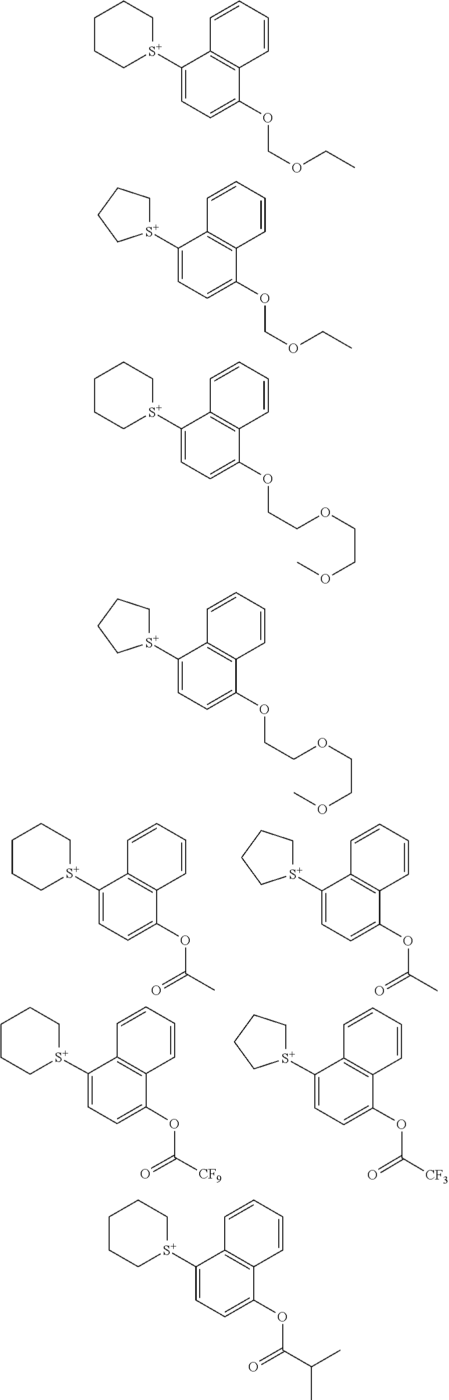

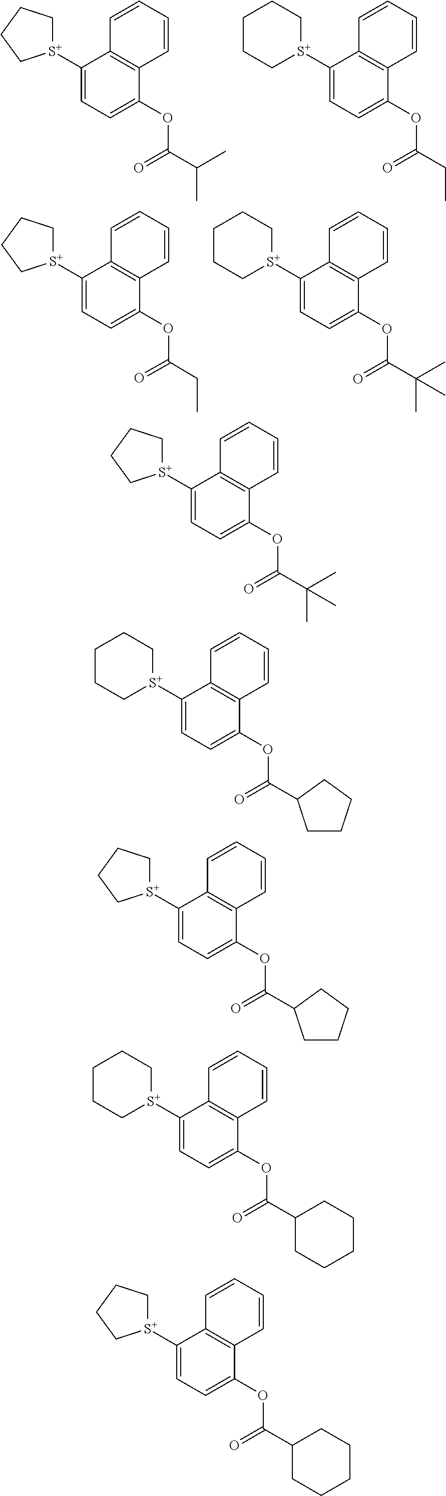

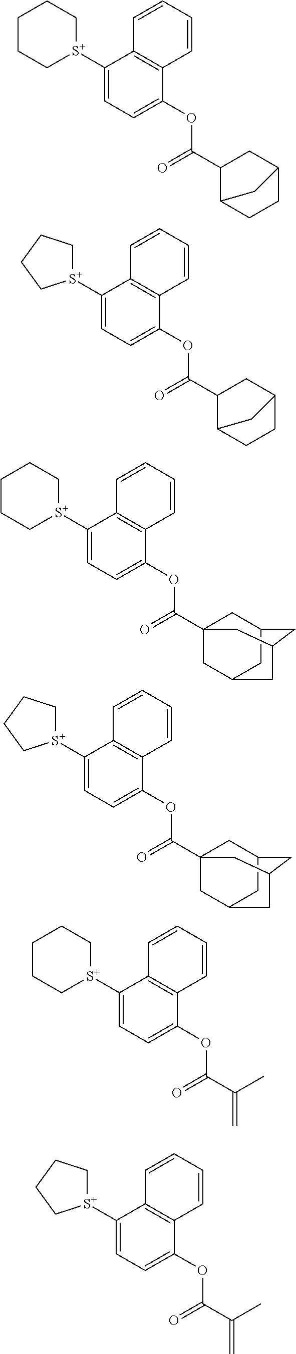 [SMILES: O=C(Oc1ccc([S+]2CCCCC2)c2ccccc12)C1CC2CCC1C2 ; O=C(Oc1ccc([S+]2CCCC2)c2ccccc12)C1CC2CCC1C2 ; O=C(Oc1ccc([S+]2CCCCC2)c2ccccc12)C12CC3CC(CC(C3)C1)C2 ; O=C(Oc1ccc([S+]2CCCC2)c2ccccc12)C12CC3CC(CC(C3)C1)C2 ; C=C(C)C(=O)Oc1ccc([S+]2CCCCC2)c2ccccc12 ; C=C(C)C(=O)Oc1ccc([S+]2CCCC2)c2ccccc12]

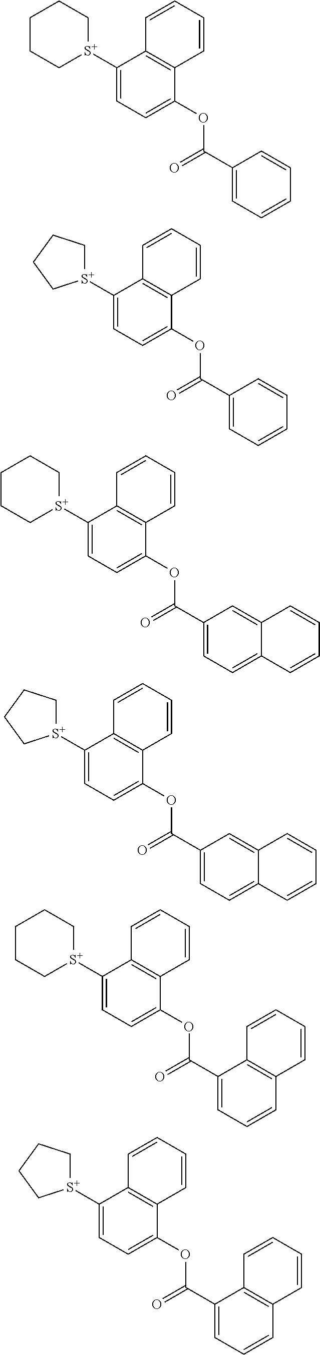 [SMILES: O=C(Oc1ccc([S+]2CCCCC2)c2ccccc12)c1ccccc1 ; O=C(Oc1ccc([S+]2CCCC2)c2ccccc12)c1ccccc1 ; O=C(Oc1ccc([S+]2CCCCC2)c2ccccc12)c1ccc2ccccc2c1 ; O=C(Oc1ccc([S+]2CCCC2)c2ccccc12)c1ccc2ccccc2c1 ; O=C(Oc1ccc([S+]2CCCCC2)c2ccccc12)c1cccc2ccccc12 ; O=C(Oc1ccc([S+]2CCCC2)c2ccccc12)c1cccc2ccccc12]

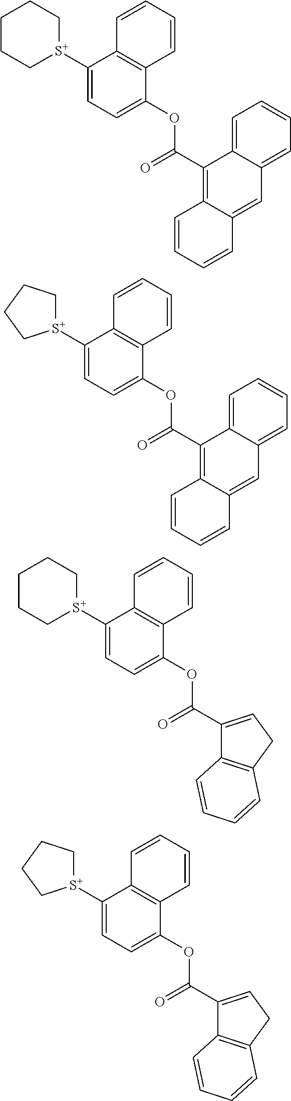 [SMILES: O=C(Oc1ccc([S+]2CCCCC2)c2ccccc12)c1c2ccccc2cc2ccccc12 ; O=C(Oc1ccc([S+]2CCCC2)c2ccccc12)c1c2ccccc2cc2ccccc12 ; O=C(Oc1ccc([S+]2CCCCC2)c2ccccc12)C1=CCc2ccccc21 ; O=C(Oc1ccc([S+]2CCCC2)c2ccccc12)C1=CCc2ccccc21]

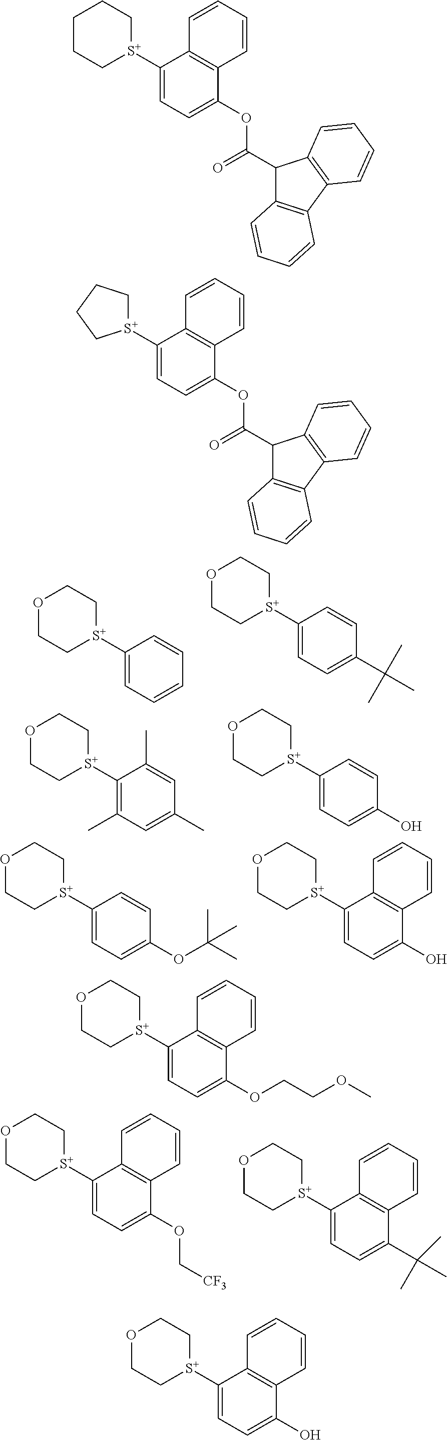

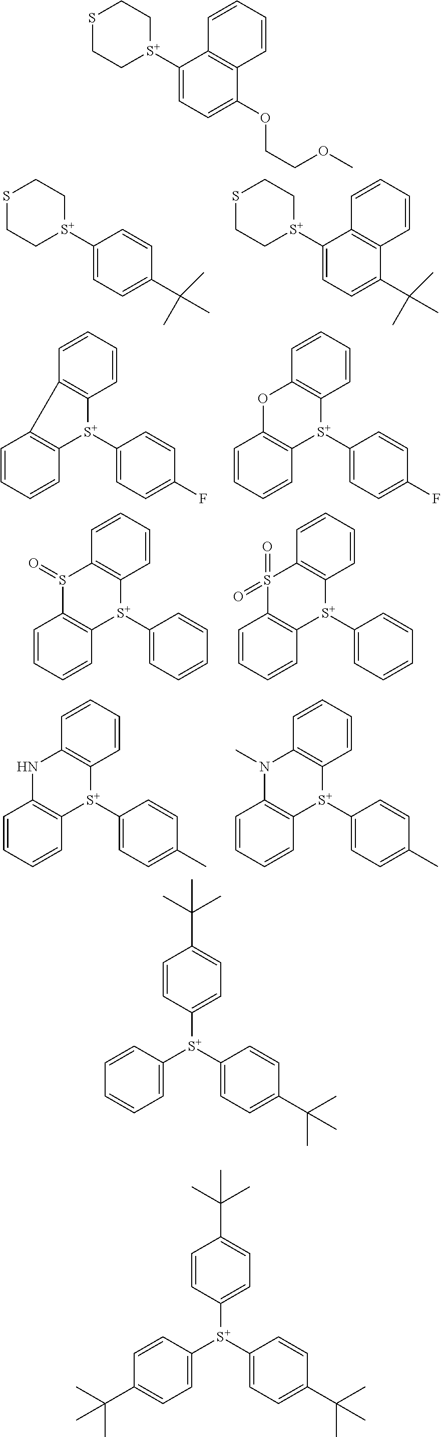

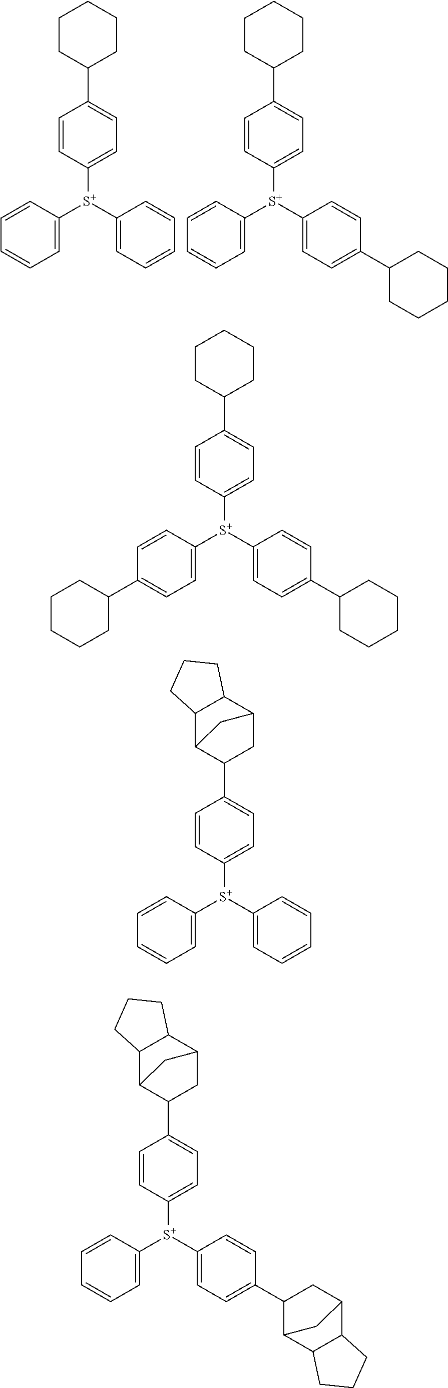 [SMILES: c1ccc([S+](c2ccccc2)c2ccc(C3CCCCC3)cc2)cc1 ; c1ccc([S+](c2ccc(C3CCCCC3)cc2)c2ccc(C3CCCCC3)cc2)cc1 ; c1cc([S+](c2ccc(C3CCCCC3)cc2)c2ccc(C3CCCCC3)cc2)ccc1C1CCCCC1 ; c1ccc([S+](c2ccccc2)c2ccc(C3CC4CC3C3CCCC43)cc2)cc1 ; c1ccc([S+](c2ccc(C3CC4CC3C3CCCC43)cc2)c2ccc(C3CC4CC3C3CCCC43)cc2)cc1]

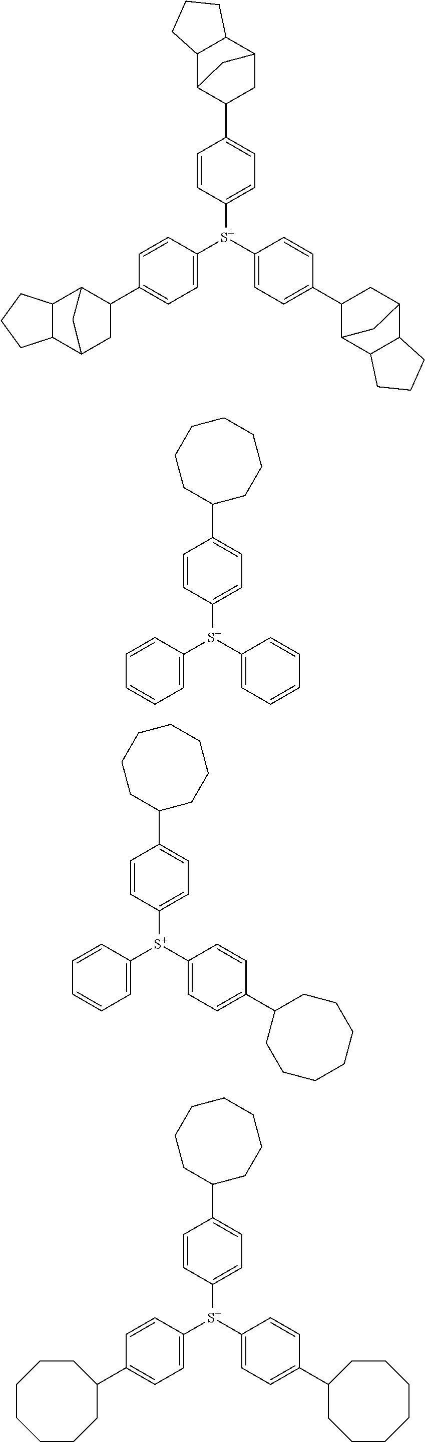 [SMILES: c1cc([S+](c2ccc(C3CC4CC3C3CCCC43)cc2)c2ccc(C3CC4CC3C3CCCC43)cc2)ccc1C1CC2CC1C1CCCC21 ; c1ccc([S+](c2ccccc2)c2ccc(C3CCCCCCC3)cc2)cc1 ; c1ccc([S+](c2ccc(C3CCCCCCC3)cc2)c2ccc(C3CCCCCCC3)cc2)cc1 ; c1cc([S+](c2ccc(C3CCCCCCC3)cc2)c2ccc(C3CCCCCCC3)cc2)ccc1C1CCCCCCC1]

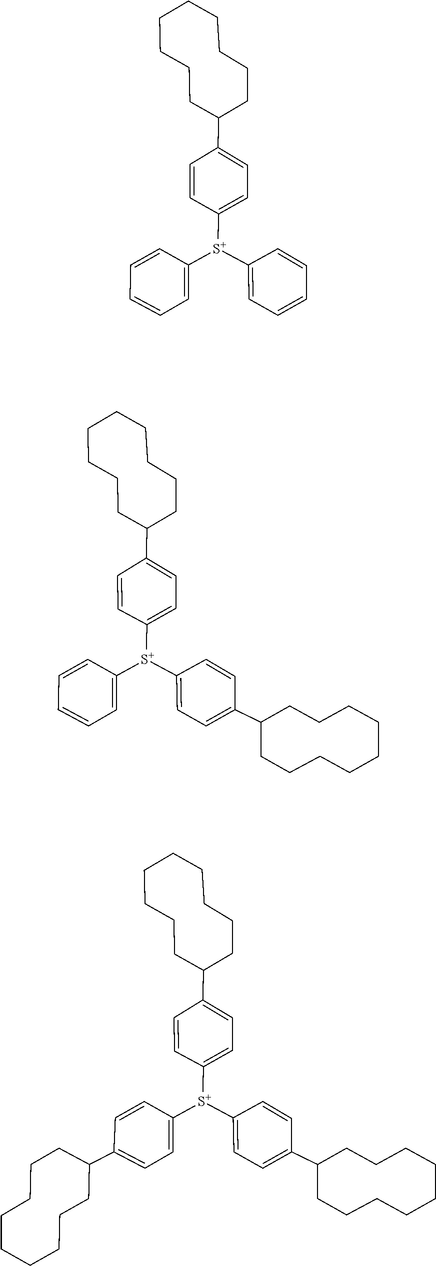 [SMILES: c1ccc([S+](c2ccccc2)c2ccc(C3CCCCCCCCC3)cc2)cc1 ; c1ccc([S+](c2ccc(C3CCCCCCCCC3)cc2)c2ccc(C3CCCCCCCCC3)cc2)cc1 ; c1cc([S+](c2ccc(C3CCCCCCCCC3)cc2)c2ccc(C3CCCCCCCCC3)cc2)ccc1C1CCCCCCCCC1]

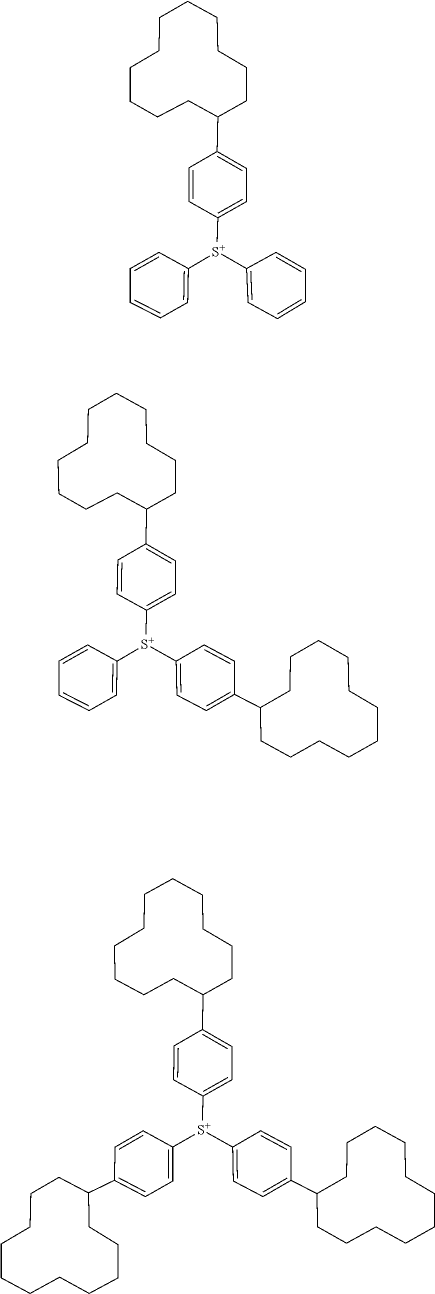 [SMILES: c1ccc([S+](c2ccccc2)c2ccc(C3CCCCCCCCCCC3)cc2)cc1 ; c1ccc([S+](c2ccc(C3CCCCCCCCCCC3)cc2)c2ccc(C3CCCCCCCCCCC3)cc2)cc1 ; c1cc([S+](c2ccc(C3CCCCCCCCCCC3)cc2)c2ccc(C3CCCCCCCCCCC3)cc2)ccc1C1CCCCCCCCCCC1]

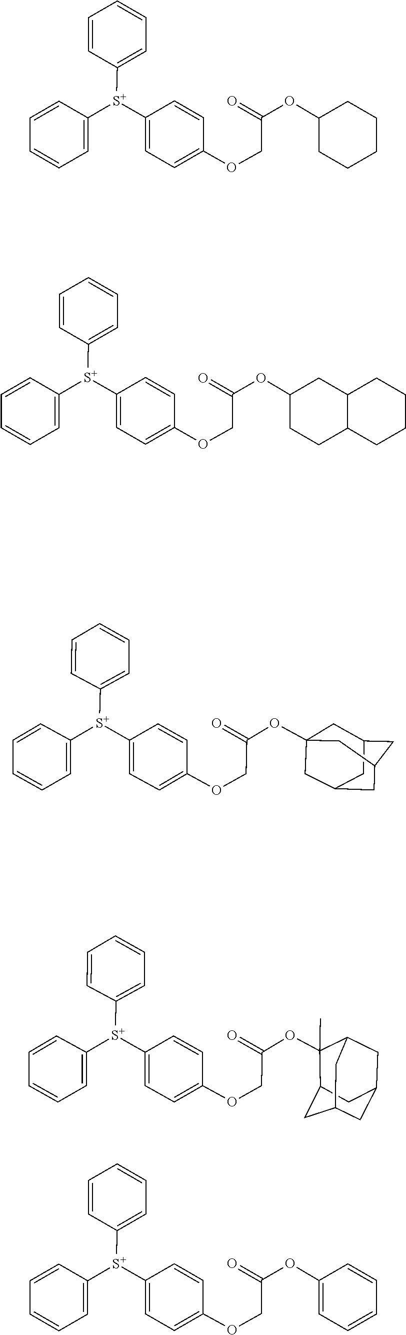 [SMILES: O=C(COc1ccc([S+](c2ccccc2)c2ccccc2)cc1)OC1CCCCC1 ; O=C(COc1ccc([S+](c2ccccc2)c2ccccc2)cc1)OC1CCC2CCCCC2C1 ; O=C(COc1ccc([S+](c2ccccc2)c2ccccc2)cc1)OC12CC3CC(CC(C3)C1)C2 ; CC1(OC(=O)COc2ccc([S+](c3ccccc3)c3ccccc3)cc2)C2CC3CC(C2)CC1C3 ; O=C(COc1ccc([S+](c2ccccc2)c2ccccc2)cc1)Oc1ccccc1]

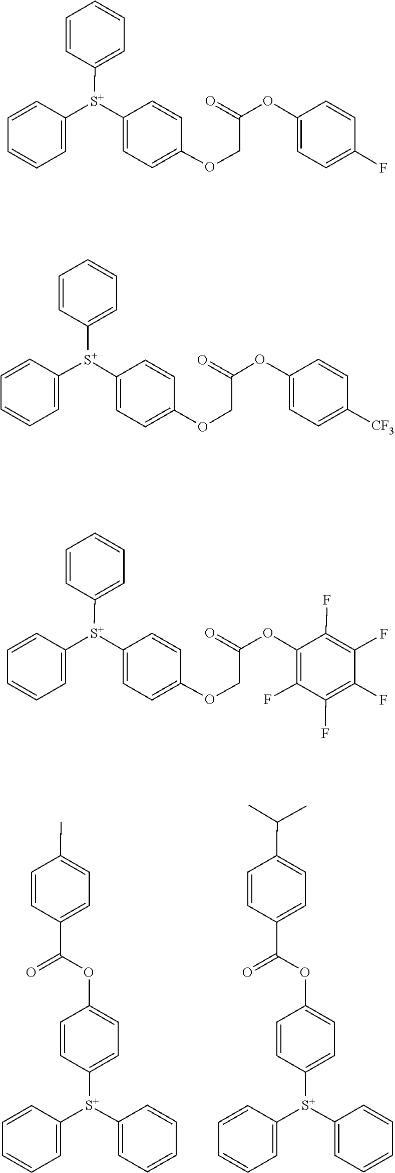 [SMILES: O=C(COc1ccc([S+](c2ccccc2)c2ccccc2)cc1)Oc1ccc(F)cc1 ; O=C(COc1ccc([S+](c2ccccc2)c2ccccc2)cc1)Oc1ccc(C(F)(F)F)cc1 ; O=C(COc1ccc([S+](c2ccccc2)c2ccccc2)cc1)Oc1c(F)c(F)c(F)c(F)c1F ; Cc1ccc(C(=O)Oc2ccc([S+](c3ccccc3)c3ccccc3)cc2)cc1 ; CC(C)c1ccc(C(=O)Oc2ccc([S+](c3ccccc3)c3ccccc3)cc2)cc1]

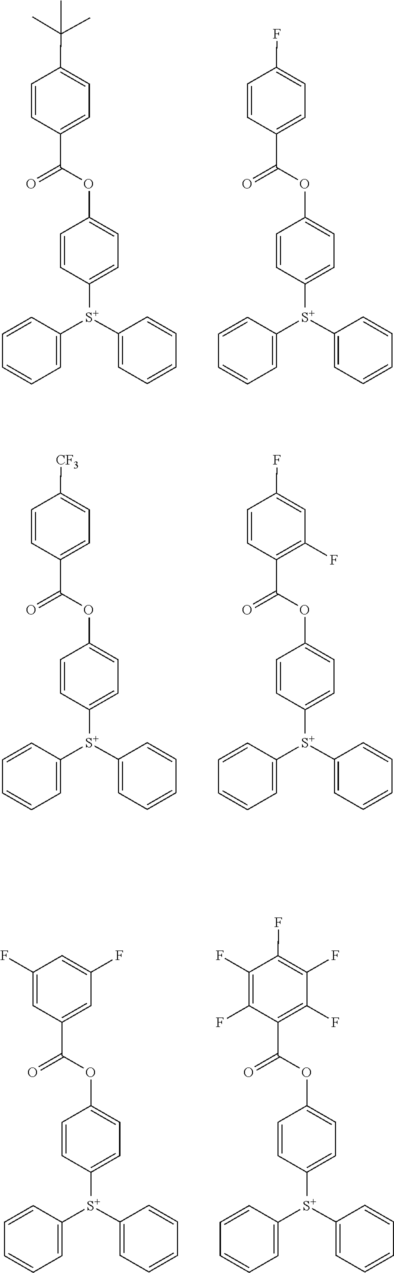 [SMILES: CC(C)(C)c1ccc(C(=O)Oc2ccc([S+](c3ccccc3)c3ccccc3)cc2)cc1 ; O=C(Oc1ccc([S+](c2ccccc2)c2ccccc2)cc1)c1ccc(F)cc1 ; O=C(Oc1ccc([S+](c2ccccc2)c2ccccc2)cc1)c1ccc(C(F)(F)F)cc1 ; O=C(Oc1ccc([S+](c2ccccc2)c2ccccc2)cc1)c1ccc(F)cc1F ; O=C(Oc1ccc([S+](c2ccccc2)c2ccccc2)cc1)c1cc(F)cc(F)c1 ; O=C(Oc1ccc([S+](c2ccccc2)c2ccccc2)cc1)c1c(F)c(F)c(F)c(F)c1F]

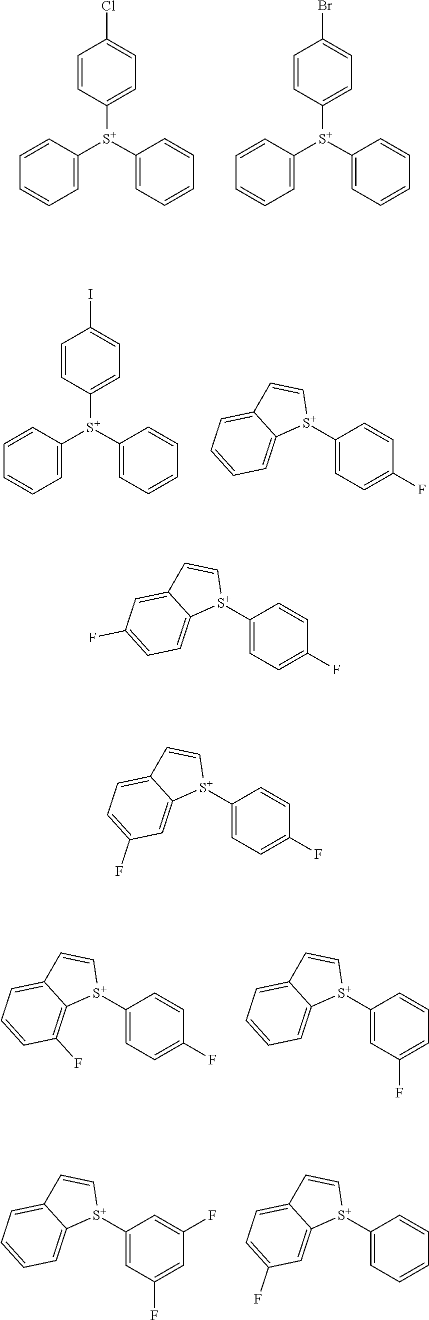

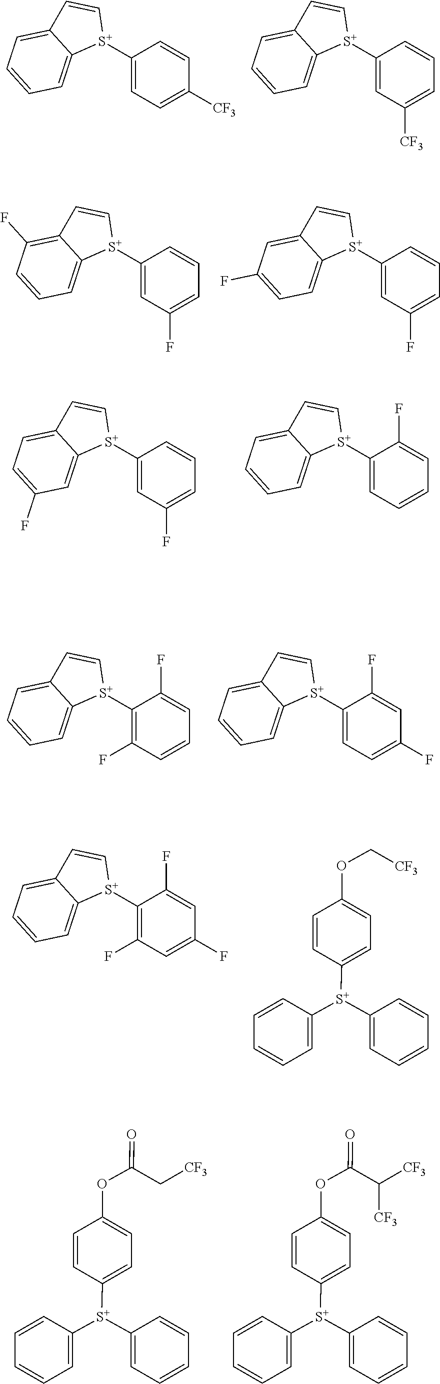

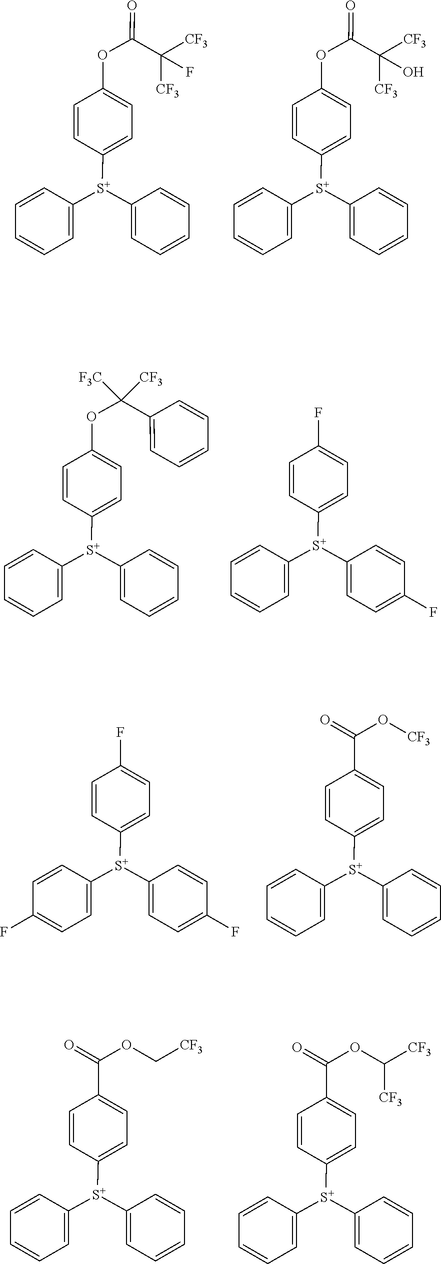

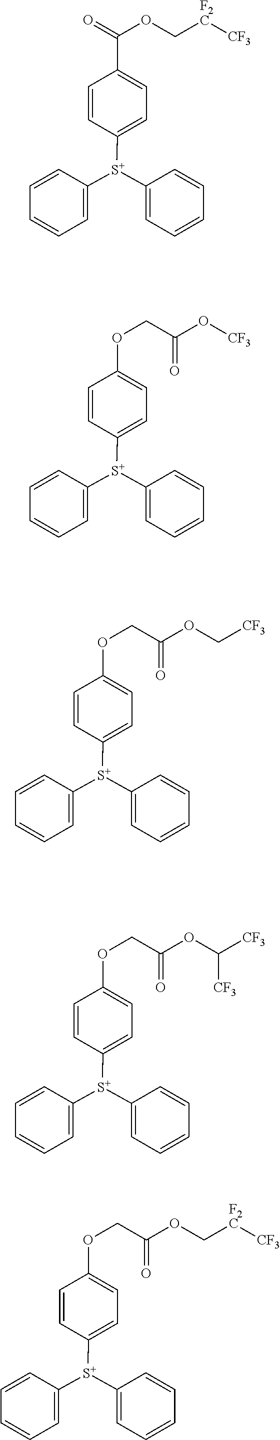 [SMILES: O=C(OCC(F)(F)C(F)(F)F)c1ccc([S+](c2ccccc2)c2ccccc2)cc1 ; O=C(COc1ccc([S+](c2ccccc2)c2ccccc2)cc1)OC(F)(F)F ; O=C(COc1ccc([S+](c2ccccc2)c2ccccc2)cc1)OCC(F)(F)F ; O=C(COc1ccc([S+](c2ccccc2)c2ccccc2)cc1)OC(C(F)(F)F)C(F)(F)F ; O=C(COc1ccc([S+](c2ccccc2)c2ccccc2)cc1)OCC(F)(F)C(F)(F)F]

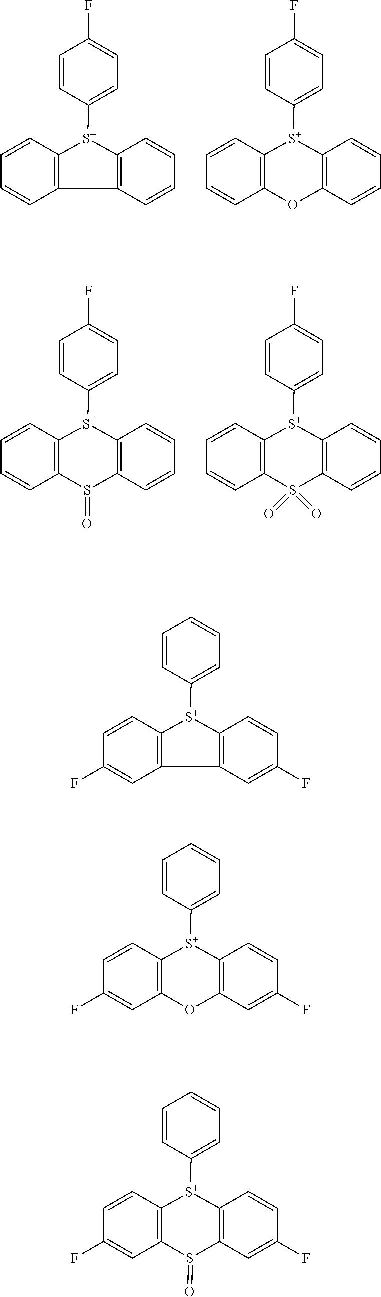

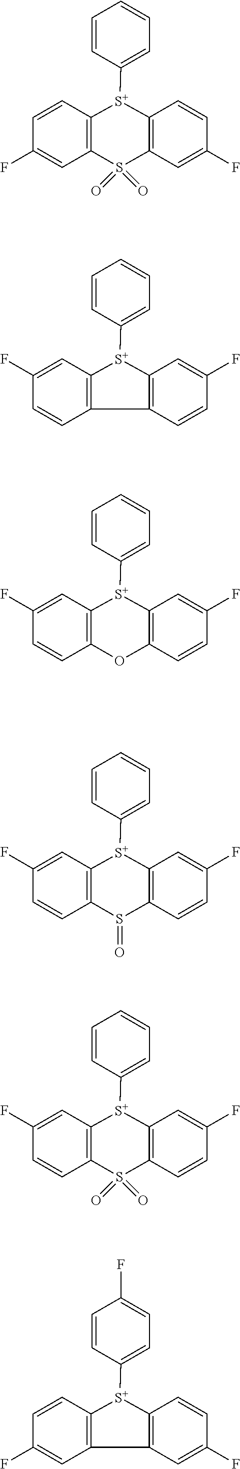 [SMILES: O=S1(=O)c2cc(F)ccc2[S+](c2ccccc2)c2ccc(F)cc21 ; Fc1ccc2c3ccc(F)cc3[s+](-c3ccccc3)c2c1 ; Fc1ccc2c(c1)[S+](c1ccccc1)c1cc(F)ccc1O2 ; O=S1c2ccc(F)cc2[S+](c2ccccc2)c2cc(F)ccc21 ; O=S1(=O)c2ccc(F)cc2[S+](c2ccccc2)c2cc(F)ccc21 ; Fc1ccc(-[s+]2c3ccc(F)cc3c3cc(F)ccc32)cc1]

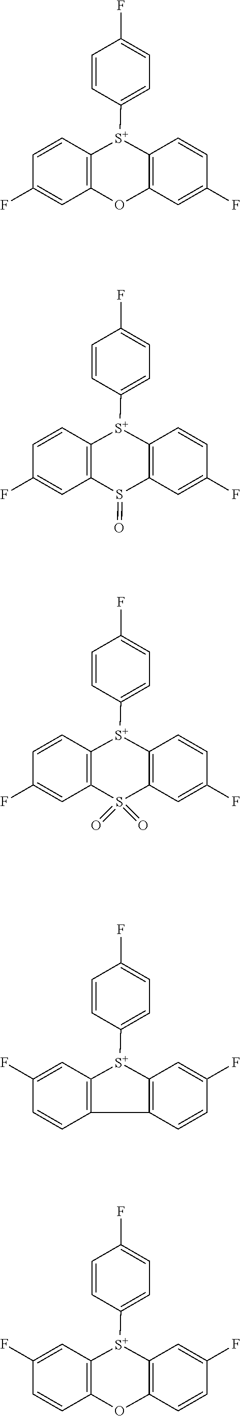 [SMILES: Fc1ccc([S+]2c3ccc(F)cc3Oc3cc(F)ccc32)cc1 ; O=S1c2cc(F)ccc2[S+](c2ccc(F)cc2)c2ccc(F)cc21 ; O=S1(=O)c2cc(F)ccc2[S+](c2ccc(F)cc2)c2ccc(F)cc21 ; Fc1ccc(-[s+]2c3cc(F)ccc3c3ccc(F)cc32)cc1 ; Fc1ccc([S+]2c3cc(F)ccc3Oc3ccc(F)cc32)cc1]

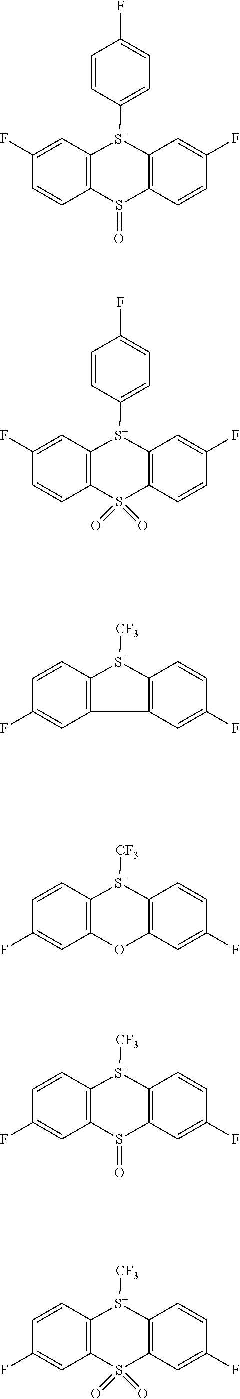 [SMILES: O=S1c2ccc(F)cc2[S+](c2ccc(F)cc2)c2cc(F)ccc21 ; O=S1(=O)c2ccc(F)cc2[S+](c2ccc(F)cc2)c2cc(F)ccc21 ; Fc1ccc2c(c1)c1cc(F)ccc1[s+]2C(F)(F)F ; Fc1ccc2c(c1)Oc1cc(F)ccc1[S+]2C(F)(F)F ; O=S1c2cc(F)ccc2[S+](C(F)(F)F)c2ccc(F)cc21 ; O=S1(=O)c2cc(F)ccc2[S+](C(F)(F)F)c2ccc(F)cc21]

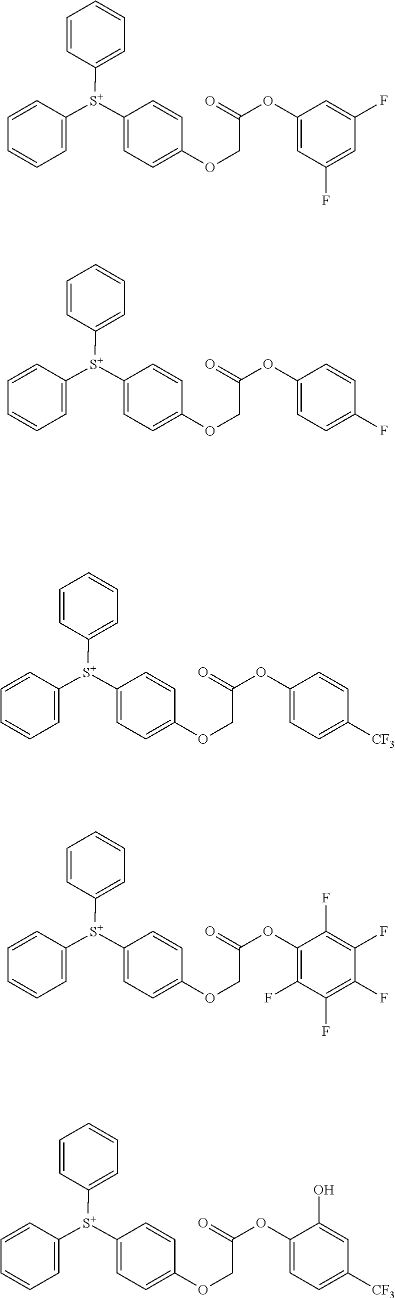 [SMILES: O=C(COc1ccc([S+](c2ccccc2)c2ccccc2)cc1)Oc1cc(F)cc(F)c1 ; O=C(COc1ccc([S+](c2ccccc2)c2ccccc2)cc1)Oc1ccc(F)cc1 ; O=C(COc1ccc([S+](c2ccccc2)c2ccccc2)cc1)Oc1ccc(C(F)(F)F)cc1 ; O=C(COc1ccc([S+](c2ccccc2)c2ccccc2)cc1)Oc1c(F)c(F)c(F)c(F)c1F ; O=C(COc1ccc([S+](c2ccccc2)c2ccccc2)cc1)Oc1ccc(C(F)(F)F)cc1O]

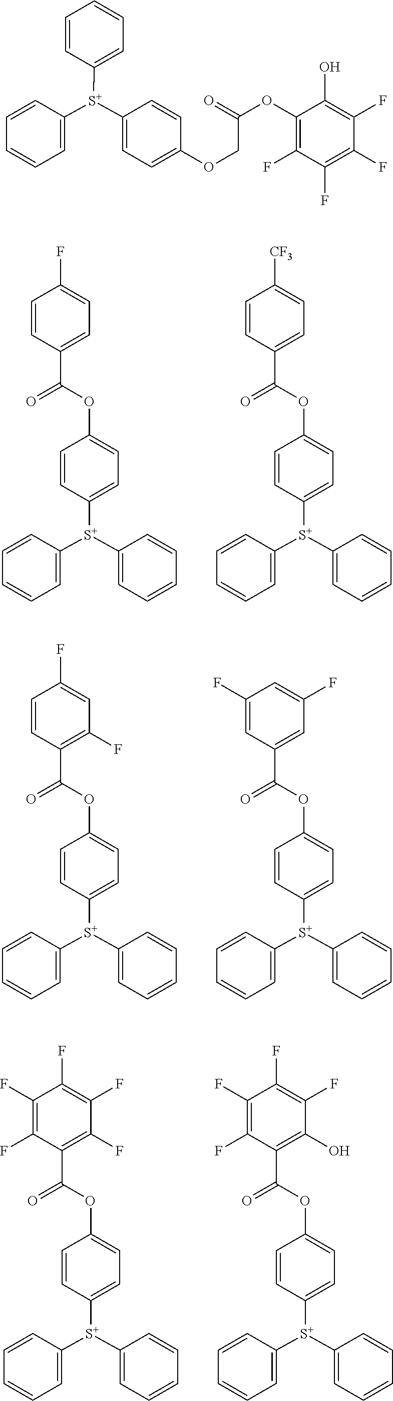

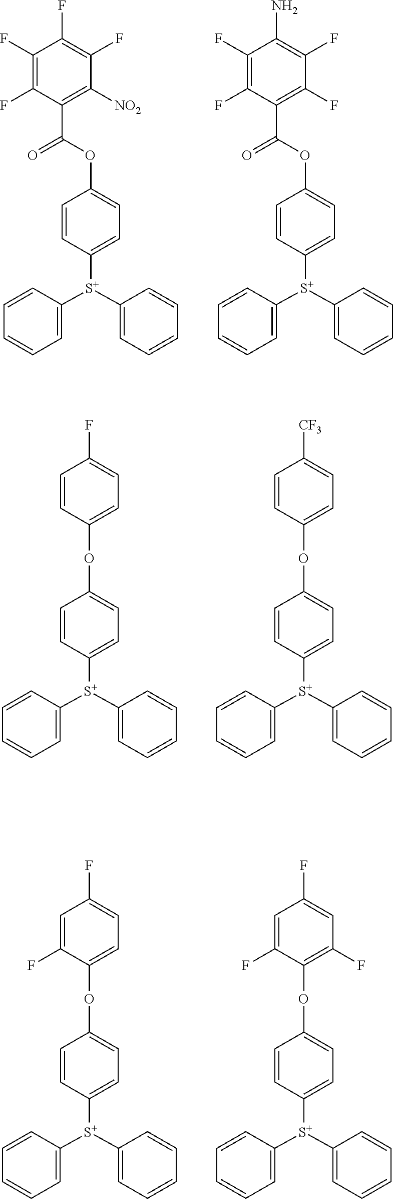 [SMILES: O=C(Oc1ccc([S+](c2ccccc2)c2ccccc2)cc1)c1c(F)c(F)c(F)c(F)c1[N+](=O)[O-] ; Nc1c(F)c(F)c(C(=O)Oc2ccc([S+](c3ccccc3)c3ccccc3)cc2)c(F)c1F ; Fc1ccc(Oc2ccc([S+](c3ccccc3)c3ccccc3)cc2)cc1 ; FC(F)(F)c1ccc(Oc2ccc([S+](c3ccccc3)c3ccccc3)cc2)cc1 ; Fc1ccc(Oc2ccc([S+](c3ccccc3)c3ccccc3)cc2)c(F)c1 ; Fc1cc(F)c(Oc2ccc([S+](c3ccccc3)c3ccccc3)cc2)c(F)c1]

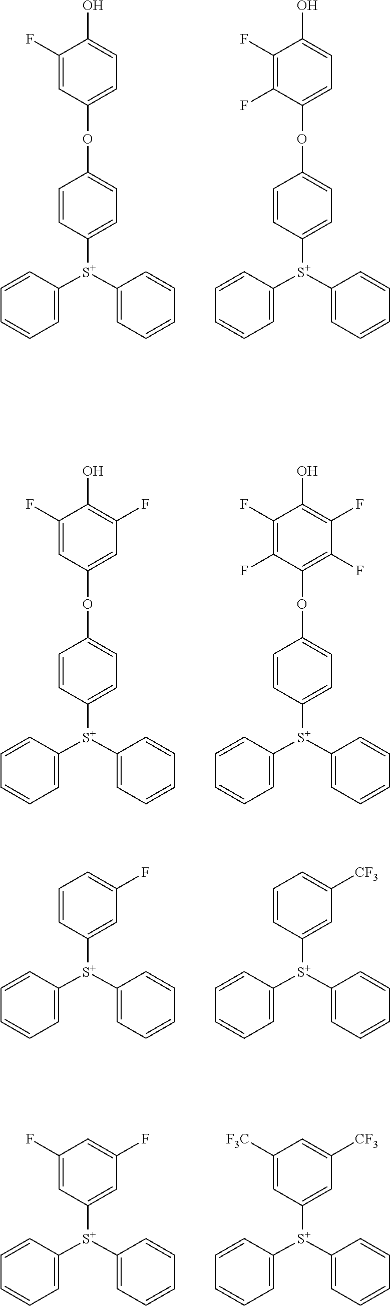

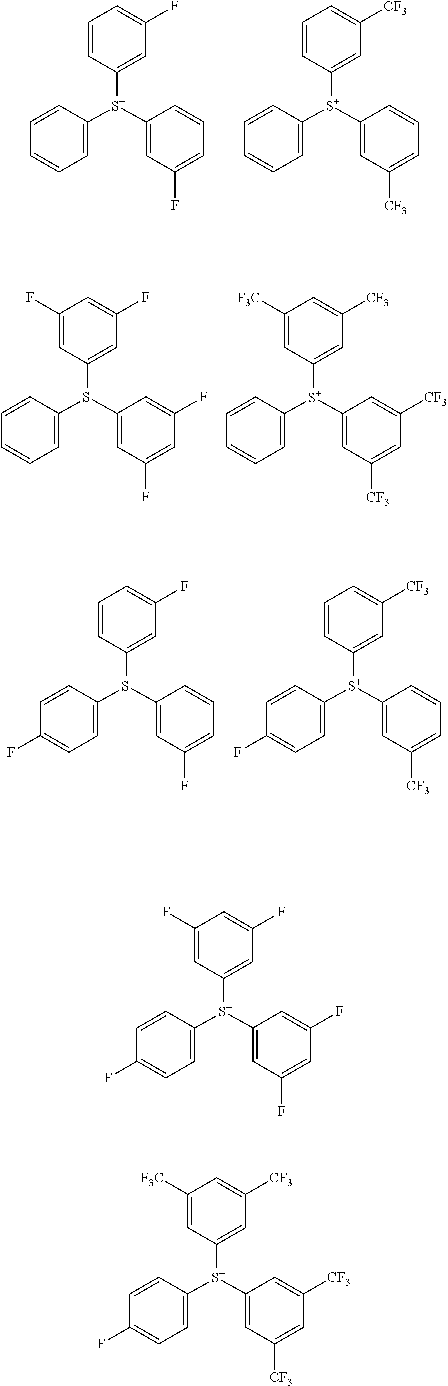

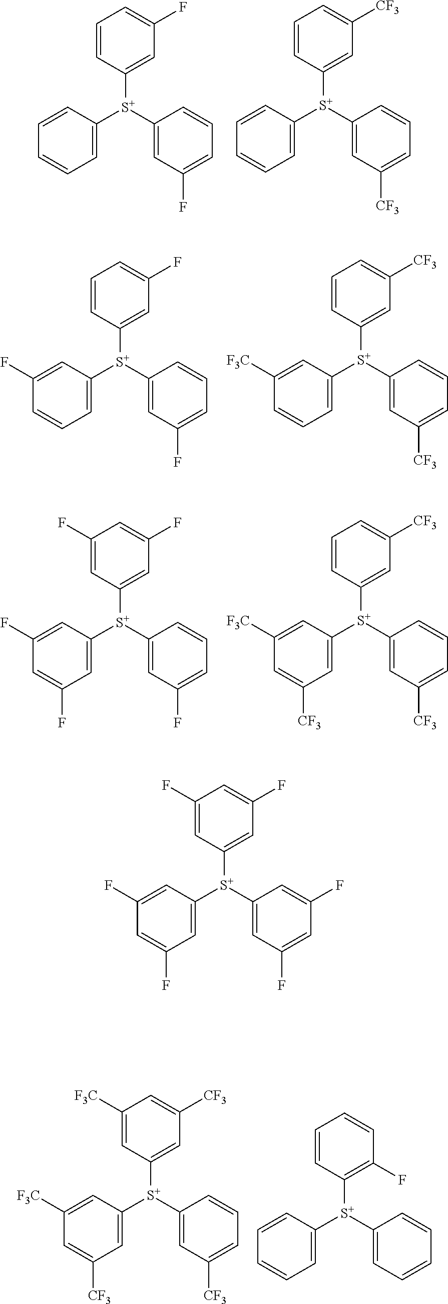

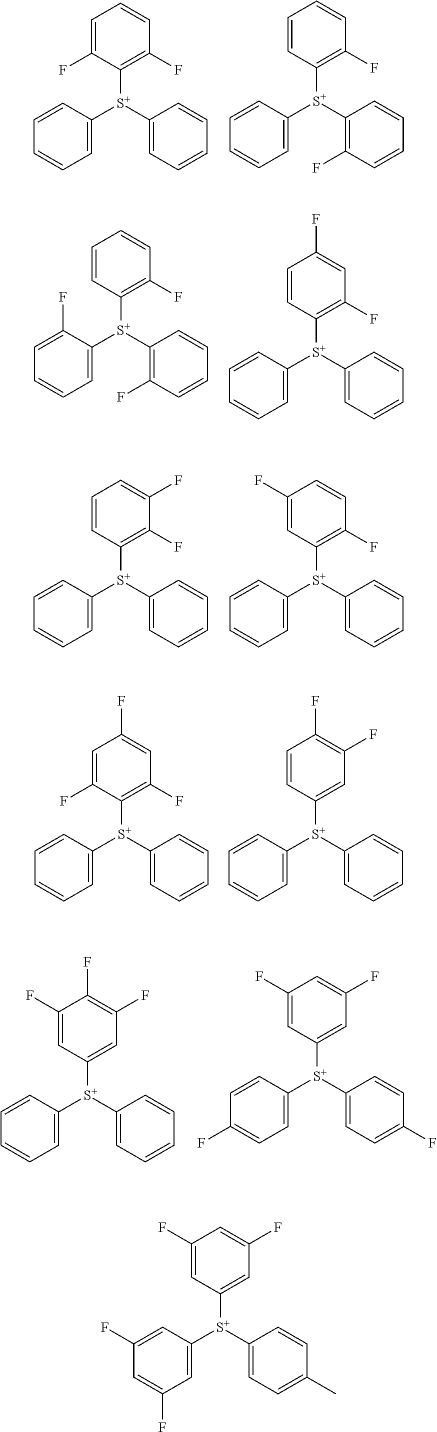

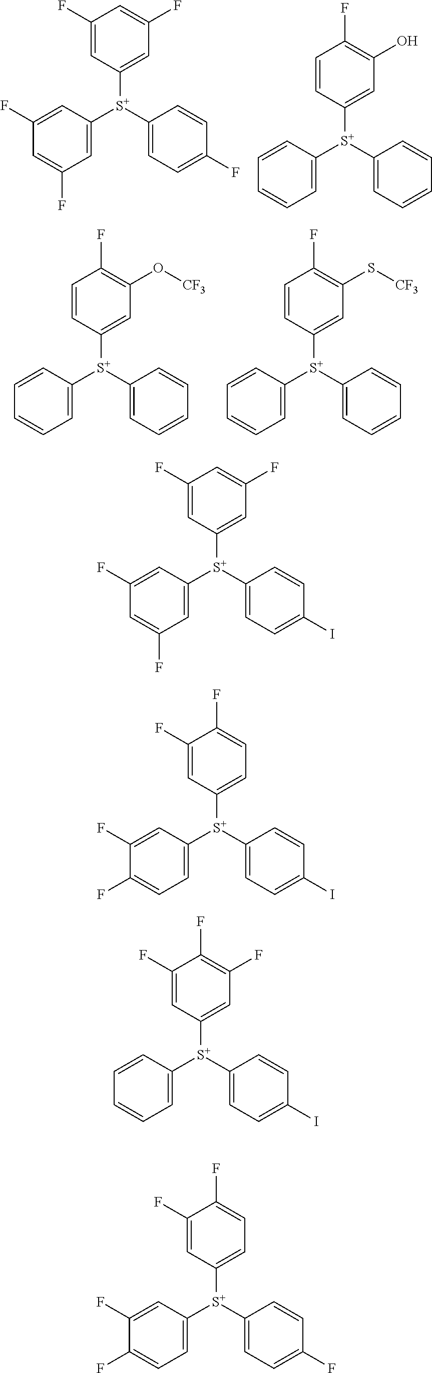

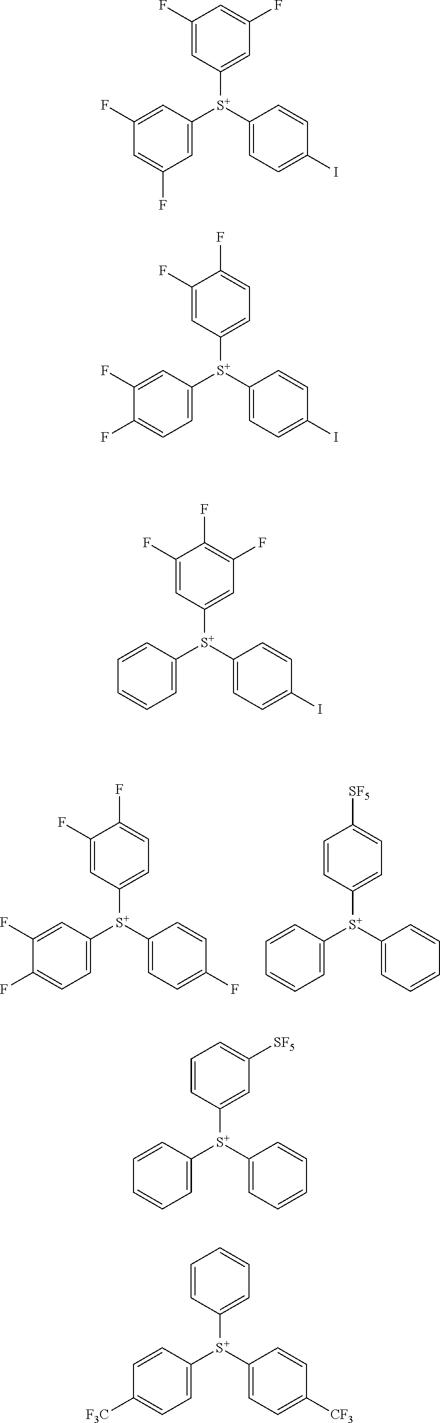

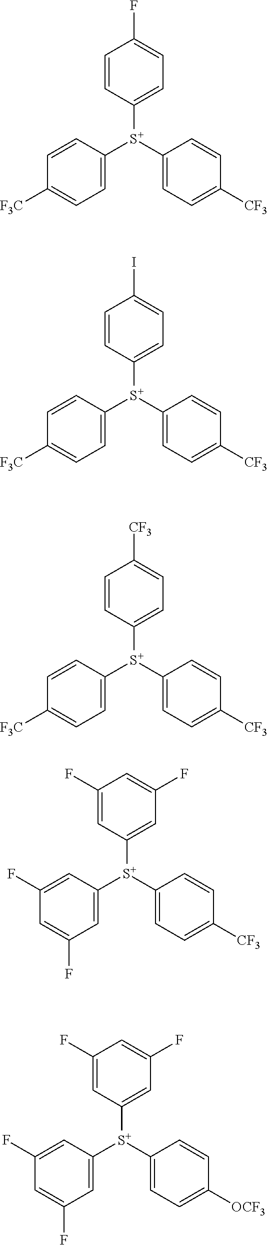 [SMILES: Fc1ccc([S+](c2ccc(C(F)(F)F)cc2)c2ccc(C(F)(F)F)cc2)cc1 ; FC(F)(F)c1ccc([S+](c2ccc(I)cc2)c2ccc(C(F)(F)F)cc2)cc1 ; FC(F)(F)c1ccc([S+](c2ccc(C(F)(F)F)cc2)c2ccc(C(F)(F)F)cc2)cc1 ; Fc1cc(F)cc([S+](c2ccc(C(F)(F)F)cc2)c2cc(F)cc(F)c2)c1 ; Fc1cc(F)cc([S+](c2ccc(OC(F)(F)F)cc2)c2cc(F)cc(F)c2)c1]

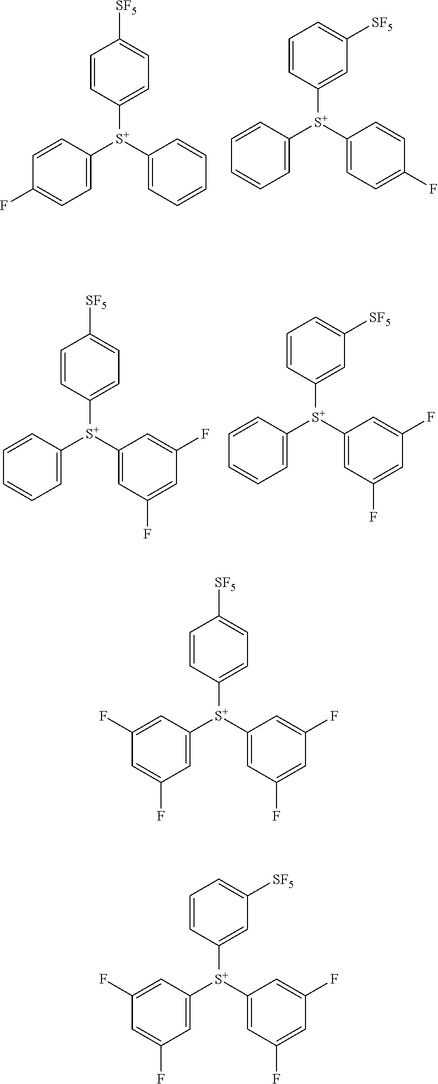

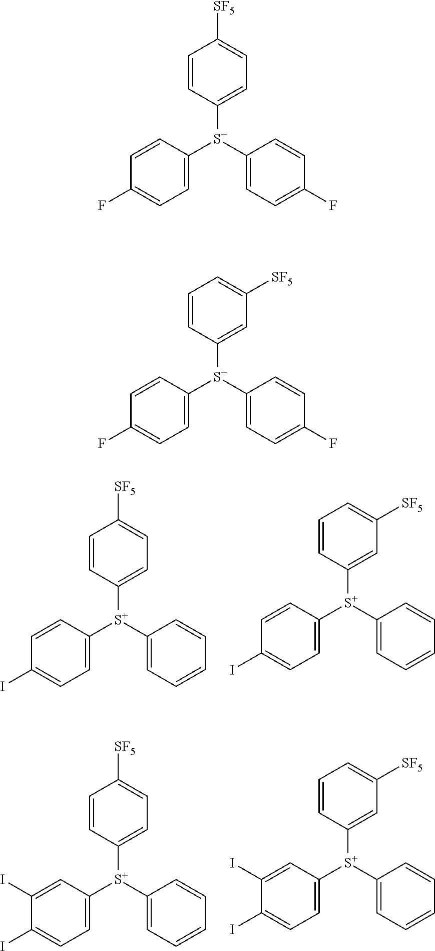

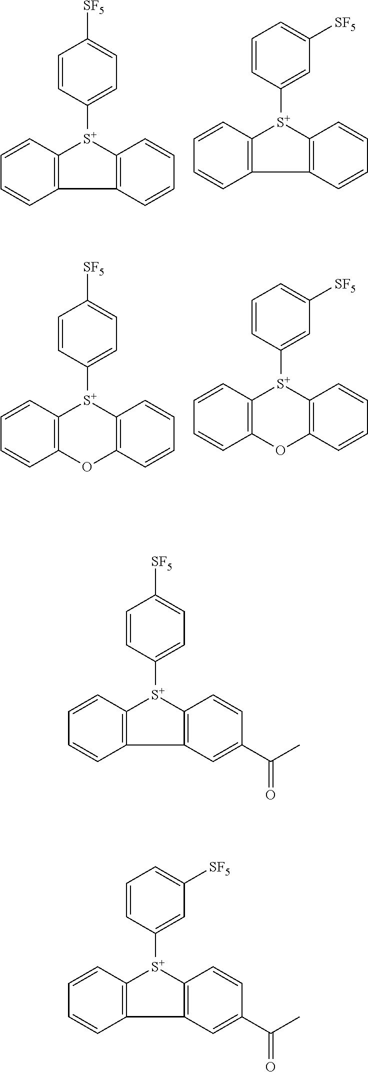

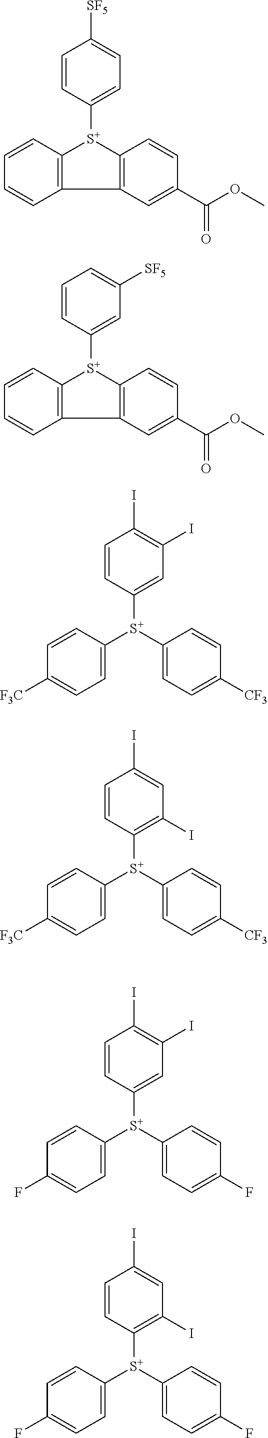

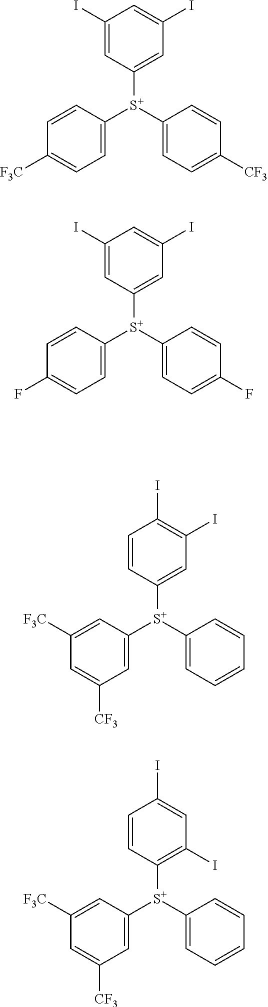 [SMILES: FC(F)(F)c1ccc([S+](c2ccc(C(F)(F)F)cc2)c2cc(I)cc(I)c2)cc1 ; Fc1ccc([S+](c2ccc(F)cc2)c2cc(I)cc(I)c2)cc1 ; FC(F)(F)c1cc([S+](c2ccccc2)c2ccc(I)c(I)c2)cc(C(F)(F)F)c1 ; FC(F)(F)c1cc([S+](c2ccccc2)c2ccc(I)cc2I)cc(C(F)(F)F)c1]

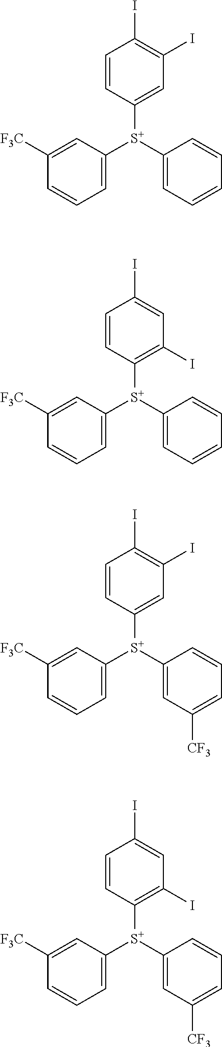 [SMILES: FC(F)(F)c1cccc([S+](c2ccccc2)c2ccc(I)c(I)c2)c1 ; FC(F)(F)c1cccc([S+](c2ccccc2)c2ccc(I)cc2I)c1 ; FC(F)(F)c1cccc([S+](c2cccc(C(F)(F)F)c2)c2ccc(I)c(I)c2)c1 ; FC(F)(F)c1cccc([S+](c2cccc(C(F)(F)F)c2)c2ccc(I)cc2I)c1]

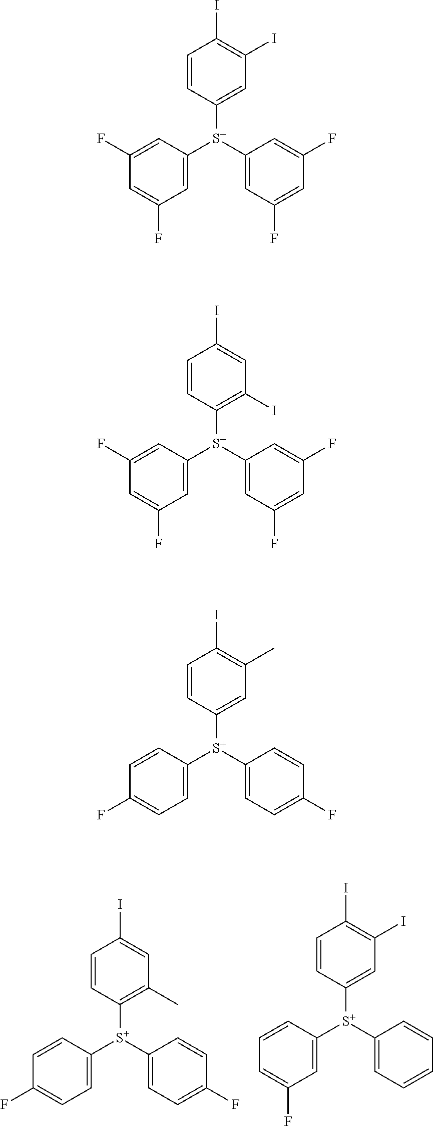 [SMILES: Fc1cc(F)cc([S+](c2cc(F)cc(F)c2)c2ccc(I)c(I)c2)c1 ; Fc1cc(F)cc([S+](c2cc(F)cc(F)c2)c2ccc(I)cc2I)c1 ; Cc1cc([S+](c2ccc(F)cc2)c2ccc(F)cc2)ccc1I ; Cc1cc(I)ccc1[S+](c1ccc(F)cc1)c1ccc(F)cc1 ; Fc1cccc([S+](c2ccccc2)c2ccc(I)c(I)c2)c1]

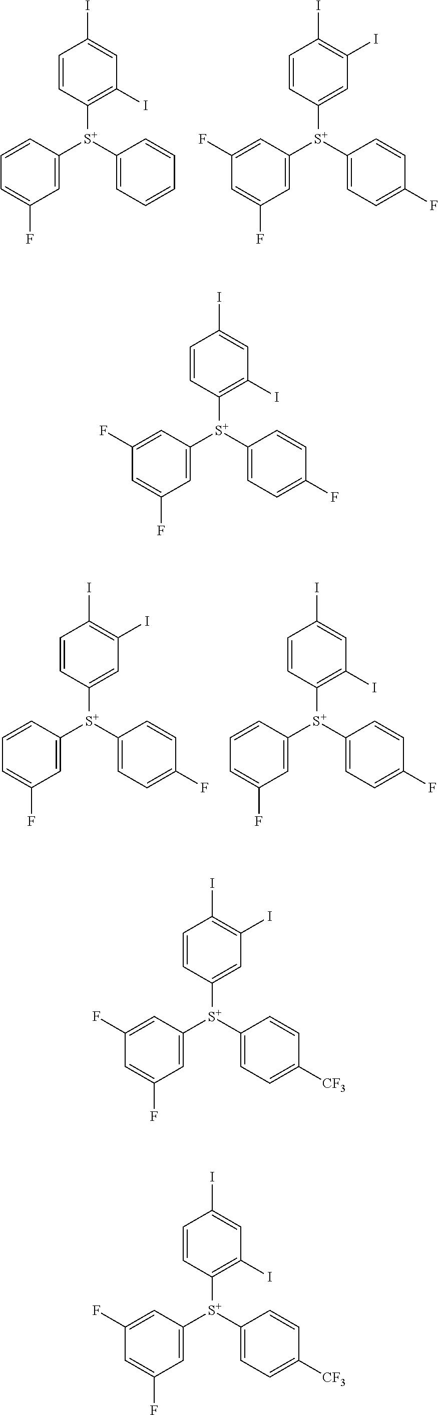

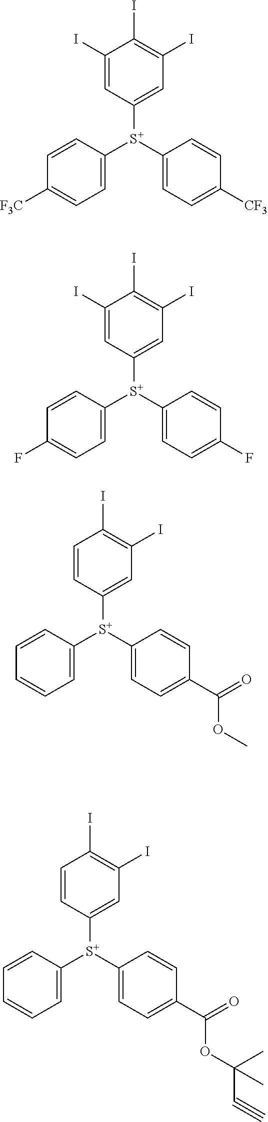 [SMILES: FC(F)(F)c1ccc([S+](c2ccc(C(F)(F)F)cc2)c2cc(I)c(I)c(I)c2)cc1 ; Fc1ccc([S+](c2ccc(F)cc2)c2cc(I)c(I)c(I)c2)cc1 ; COC(=O)c1ccc([S+](c2ccccc2)c2ccc(I)c(I)c2)cc1 ; C#CC(C)(C)OC(=O)c1ccc([S+](c2ccccc2)c2ccc(I)c(I)c2)cc1]

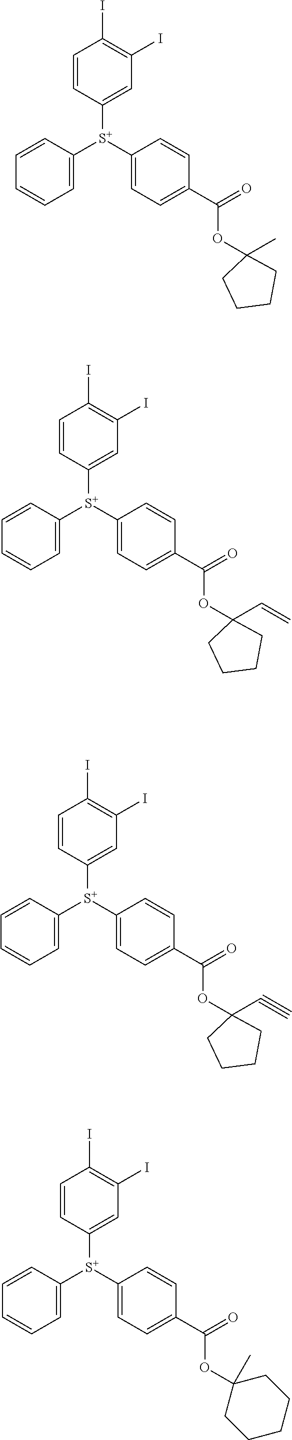 [SMILES: CC1(OC(=O)c2ccc([S+](c3ccccc3)c3ccc(I)c(I)c3)cc2)CCCC1 ; C=CC1(OC(=O)c2ccc([S+](c3ccccc3)c3ccc(I)c(I)c3)cc2)CCCC1 ; C#CC1(OC(=O)c2ccc([S+](c3ccccc3)c3ccc(I)c(I)c3)cc2)CCCC1 ; CC1(OC(=O)c2ccc([S+](c3ccccc3)c3ccc(I)c(I)c3)cc2)CCCCC1]

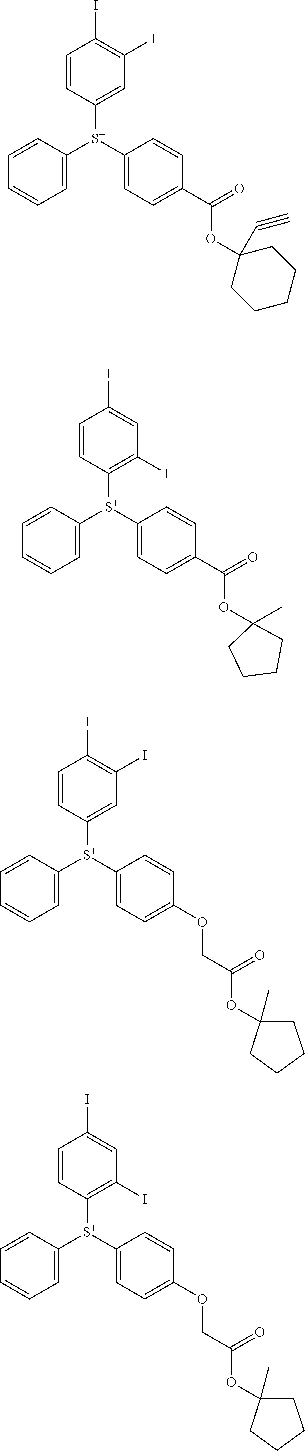 [SMILES: C#CC1(OC(=O)c2ccc([S+](c3ccccc3)c3ccc(I)c(I)c3)cc2)CCCCC1 ; CC1(OC(=O)c2ccc([S+](c3ccccc3)c3ccc(I)cc3I)cc2)CCCC1 ; CC1(OC(=O)COc2ccc([S+](c3ccccc3)c3ccc(I)c(I)c3)cc2)CCCC1 ; CC1(OC(=O)COc2ccc([S+](c3ccccc3)c3ccc(I)cc3I)cc2)CCCC1]

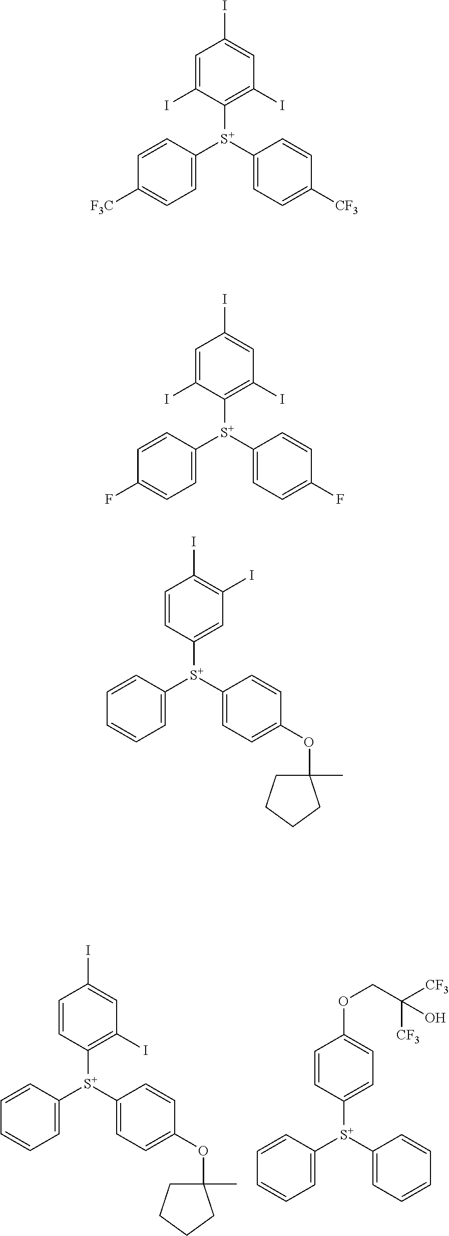 [SMILES: FC(F)(F)c1ccc([S+](c2ccc(C(F)(F)F)cc2)c2c(I)cc(I)cc2I)cc1 ; Fc1ccc([S+](c2ccc(F)cc2)c2c(I)cc(I)cc2I)cc1 ; CC1(Oc2ccc([S+](c3ccccc3)c3ccc(I)c(I)c3)cc2)CCCC1 ; CC1(Oc2ccc([S+](c3ccccc3)c3ccc(I)cc3I)cc2)CCCC1 ; OC(COc1ccc([S+](c2ccccc2)c2ccccc2)cc1)(C(F)(F)F)C(F)(F)F]

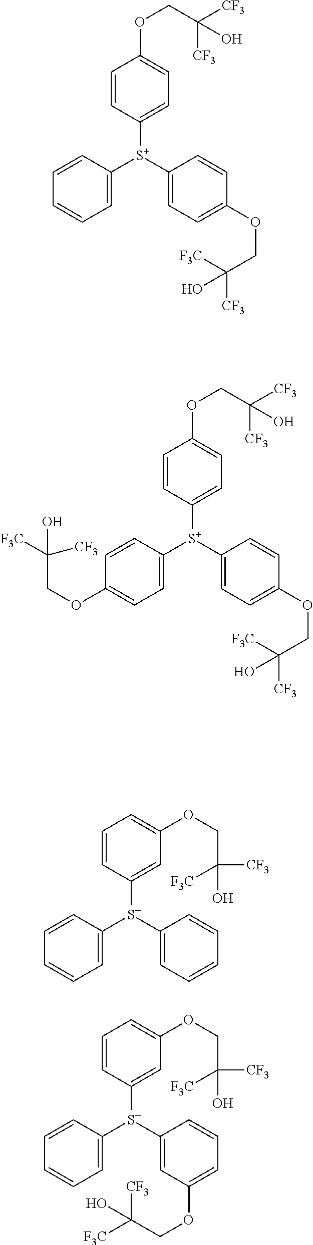 [SMILES: OC(COc1ccc([S+](c2ccccc2)c2ccc(OCC(O)(C(F)(F)F)C(F)(F)F)cc2)cc1)(C(F)(F)F)C(F)(F)F ; OC(COc1ccc([S+](c2ccc(OCC(O)(C(F)(F)F)C(F)(F)F)cc2)c2ccc(OCC(O)(C(F)(F)F)C(F)(F)F)cc2)cc1)(C(F)(F)F)C(F)(F)F ; OC(COc1cccc([S+](c2ccccc2)c2ccccc2)c1)(C(F)(F)F)C(F)(F)F ; OC(COc1cccc([S+](c2ccccc2)c2cccc(OCC(O)(C(F)(F)F)C(F)(F)F)c2)c1)(C(F)(F)F)C(F)(F)F]

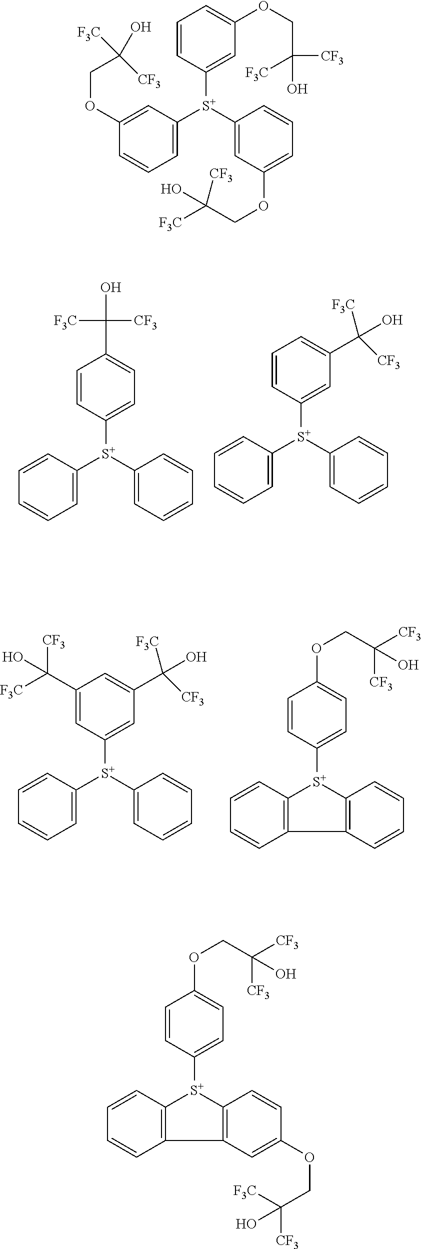 [SMILES: OC(COc1cccc([S+](c2cccc(OCC(O)(C(F)(F)F)C(F)(F)F)c2)c2cccc(OCC(O)(C(F)(F)F)C(F)(F)F)c2)c1)(C(F)(F)F)C(F)(F)F ; OC(c1ccc([S+](c2ccccc2)c2ccccc2)cc1)(C(F)(F)F)C(F)(F)F ; OC(c1cccc([S+](c2ccccc2)c2ccccc2)c1)(C(F)(F)F)C(F)(F)F ; OC(c1cc([S+](c2ccccc2)c2ccccc2)cc(C(O)(C(F)(F)F)C(F)(F)F)c1)(C(F)(F)F)C(F)(F)F ; OC(COc1ccc(-[s+]2c3ccccc3c3ccccc32)cc1)(C(F)(F)F)C(F)(F)F ; OC(COc1ccc(-[s+]2c3ccccc3c3cc(OCC(O)(C(F)(F)F)C(F)(F)F)ccc32)cc1)(C(F)(F)F)C(F)(F)F]

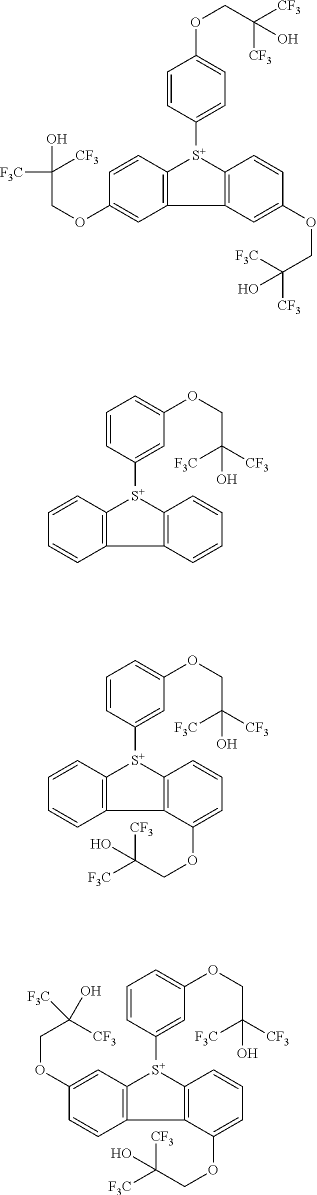 [SMILES: OC(COc1ccc(-[s+]2c3ccc(OCC(O)(C(F)(F)F)C(F)(F)F)cc3c3cc(OCC(O)(C(F)(F)F)C(F)(F)F)ccc32)cc1)(C(F)(F)F)C(F)(F)F ; OC(COc1cccc(-[s+]2c3ccccc3c3ccccc32)c1)(C(F)(F)F)C(F)(F)F ; OC(COc1cccc(-[s+]2c3ccccc3c3c(OCC(O)(C(F)(F)F)C(F)(F)F)cccc32)c1)(C(F)(F)F)C(F)(F)F ; OC(COc1cccc(-[s+]2c3cc(OCC(O)(C(F)(F)F)C(F)(F)F)ccc3c3c(OCC(O)(C(F)(F)F)C(F)(F)F)cccc32)c1)(C(F)(F)F)C(F)(F)F]

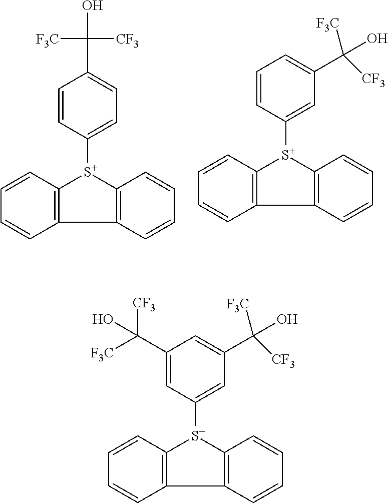 [SMILES: OC(c1ccc(-[s+]2c3ccccc3c3ccccc32)cc1)(C(F)(F)F)C(F)(F)F ; OC(c1cccc(-[s+]2c3ccccc3c3ccccc32)c1)(C(F)(F)F)C(F)(F)F ; OC(c1cc(-[s+]2c3ccccc3c3ccccc32)cc(C(O)(C(F)(F)F)C(F)(F)F)c1)(C(F)(F)F)C(F)(F)F]

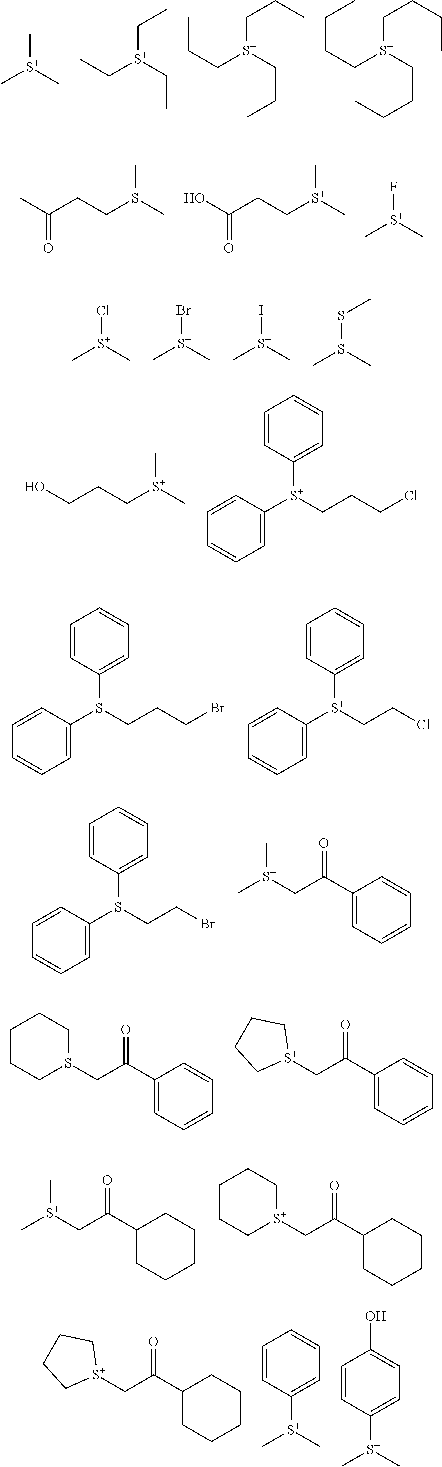

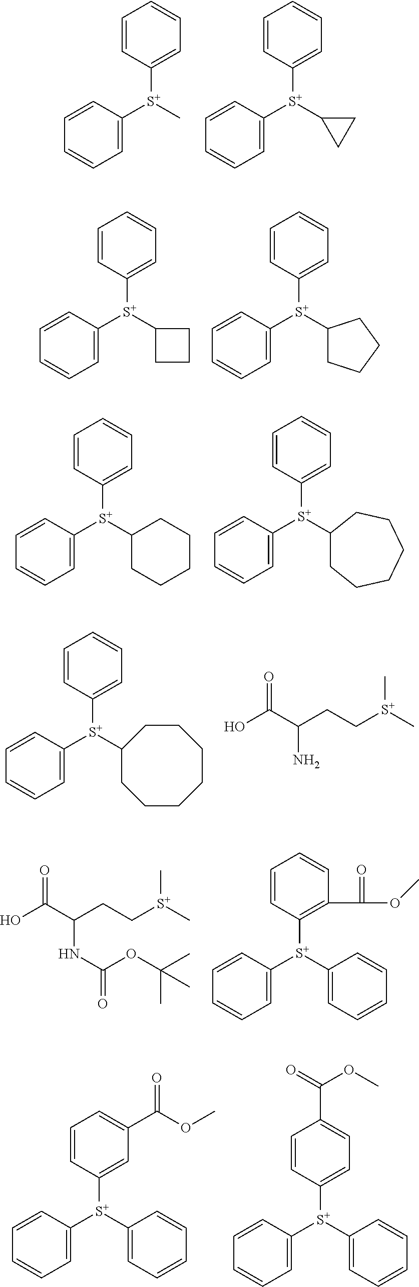

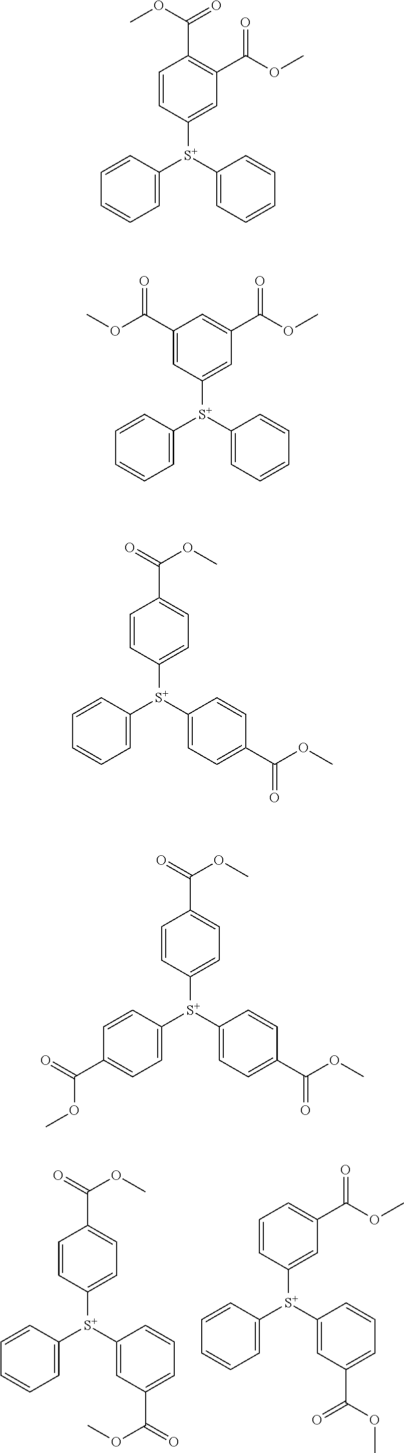

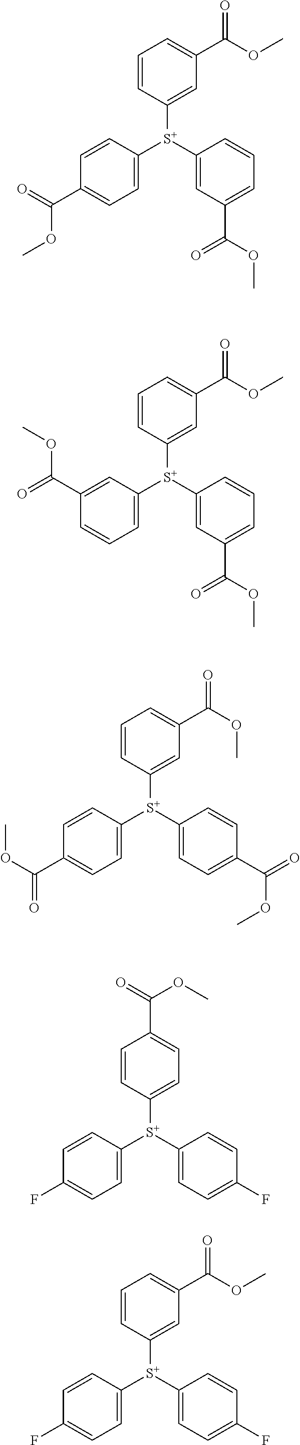 [SMILES: COC(=O)c1ccc([S+](c2cccc(C(=O)OC)c2)c2cccc(C(=O)OC)c2)cc1 ; COC(=O)c1cccc([S+](c2cccc(C(=O)OC)c2)c2cccc(C(=O)OC)c2)c1 ; COC(=O)c1ccc([S+](c2ccc(C(=O)OC)cc2)c2cccc(C(=O)OC)c2)cc1 ; COC(=O)c1ccc([S+](c2ccc(F)cc2)c2ccc(F)cc2)cc1 ; COC(=O)c1cccc([S+](c2ccc(F)cc2)c2ccc(F)cc2)c1]

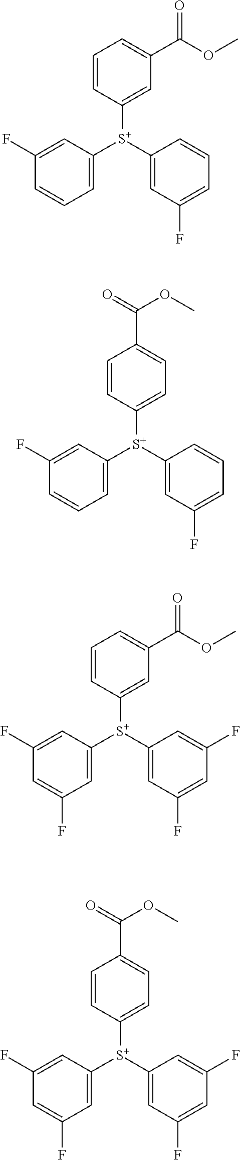 [SMILES: COC(=O)c1cccc([S+](c2cccc(F)c2)c2cccc(F)c2)c1 ; COC(=O)c1ccc([S+](c2cccc(F)c2)c2cccc(F)c2)cc1 ; COC(=O)c1cccc([S+](c2cc(F)cc(F)c2)c2cc(F)cc(F)c2)c1 ; COC(=O)c1ccc([S+](c2cc(F)cc(F)c2)c2cc(F)cc(F)c2)cc1]

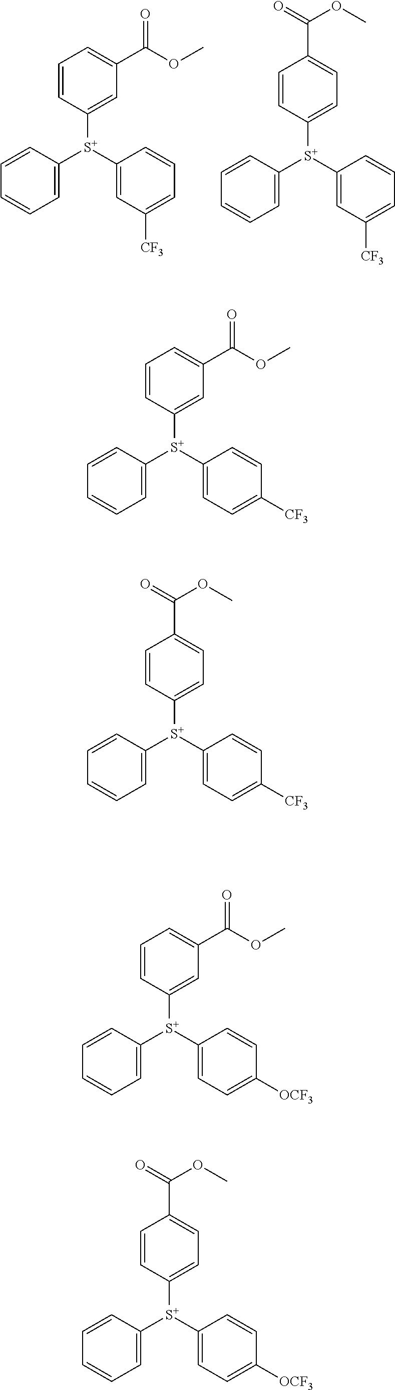 [SMILES: COC(=O)c1cccc([S+](c2ccccc2)c2cccc(C(F)(F)F)c2)c1 ; COC(=O)c1ccc([S+](c2ccccc2)c2cccc(C(F)(F)F)c2)cc1 ; COC(=O)c1cccc([S+](c2ccccc2)c2ccc(C(F)(F)F)cc2)c1 ; COC(=O)c1ccc([S+](c2ccccc2)c2ccc(C(F)(F)F)cc2)cc1 ; COC(=O)c1cccc([S+](c2ccccc2)c2ccc(OC(F)(F)F)cc2)c1 ; COC(=O)c1ccc([S+](c2ccccc2)c2ccc(OC(F)(F)F)cc2)cc1]

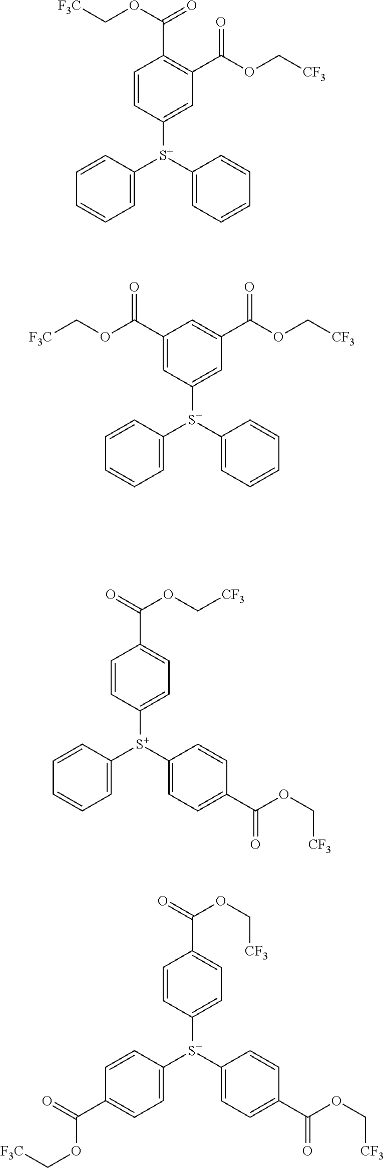 [SMILES: O=C(OCC(F)(F)F)c1ccc([S+](c2ccccc2)c2ccccc2)cc1C(=O)OCC(F)(F)F ; O=C(OCC(F)(F)F)c1cc(C(=O)OCC(F)(F)F)cc([S+](c2ccccc2)c2ccccc2)c1 ; O=C(OCC(F)(F)F)c1ccc([S+](c2ccccc2)c2ccc(C(=O)OCC(F)(F)F)cc2)cc1 ; O=C(OCC(F)(F)F)c1ccc([S+](c2ccc(C(=O)OCC(F)(F)F)cc2)c2ccc(C(=O)OCC(F)(F)F)cc2)cc1]

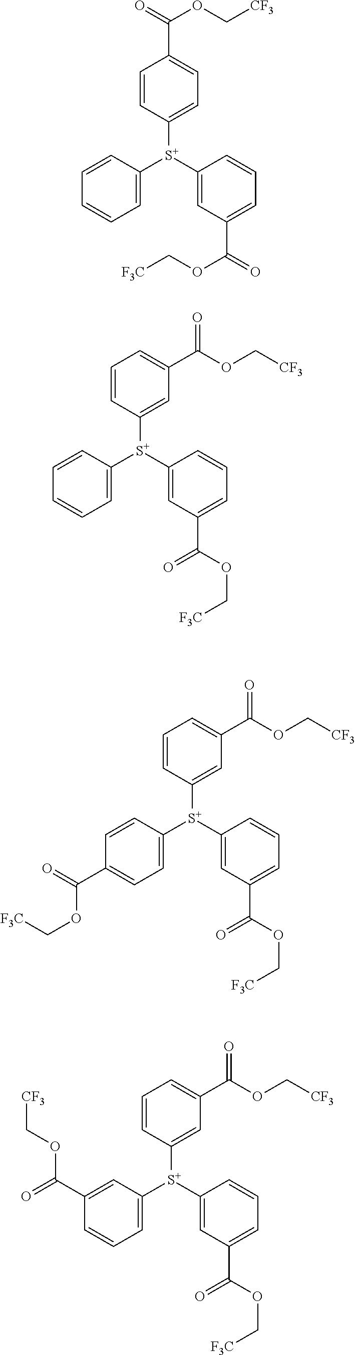 [SMILES: O=C(OCC(F)(F)F)c1ccc([S+](c2ccccc2)c2cccc(C(=O)OCC(F)(F)F)c2)cc1 ; O=C(OCC(F)(F)F)c1cccc([S+](c2ccccc2)c2cccc(C(=O)OCC(F)(F)F)c2)c1 ; O=C(OCC(F)(F)F)c1ccc([S+](c2cccc(C(=O)OCC(F)(F)F)c2)c2cccc(C(=O)OCC(F)(F)F)c2)cc1 ; O=C(OCC(F)(F)F)c1cccc([S+](c2cccc(C(=O)OCC(F)(F)F)c2)c2cccc(C(=O)OCC(F)(F)F)c2)c1]

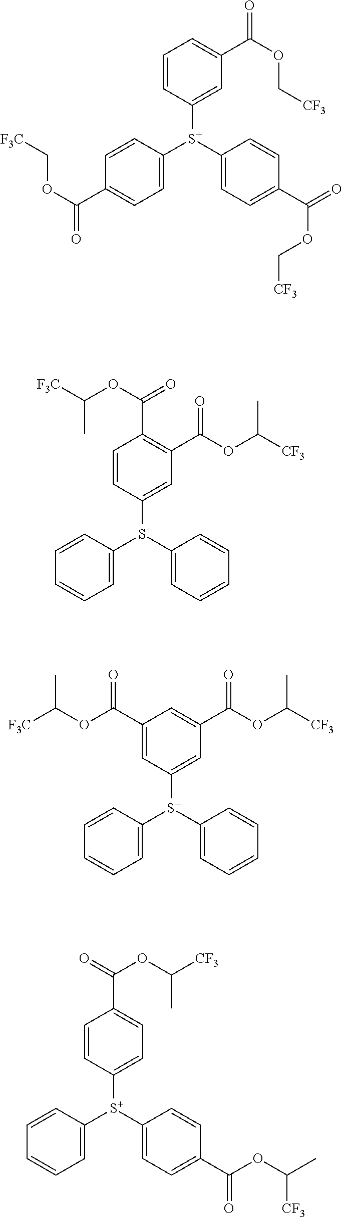 [SMILES: O=C(OCC(F)(F)F)c1ccc([S+](c2ccc(C(=O)OCC(F)(F)F)cc2)c2cccc(C(=O)OCC(F)(F)F)c2)cc1 ; CC(OC(=O)c1ccc([S+](c2ccccc2)c2ccccc2)cc1C(=O)OC(C)C(F)(F)F)C(F)(F)F ; CC(OC(=O)c1cc(C(=O)OC(C)C(F)(F)F)cc([S+](c2ccccc2)c2ccccc2)c1)C(F)(F)F ; CC(OC(=O)c1ccc([S+](c2ccccc2)c2ccc(C(=O)OC(C)C(F)(F)F)cc2)cc1)C(F)(F)F]

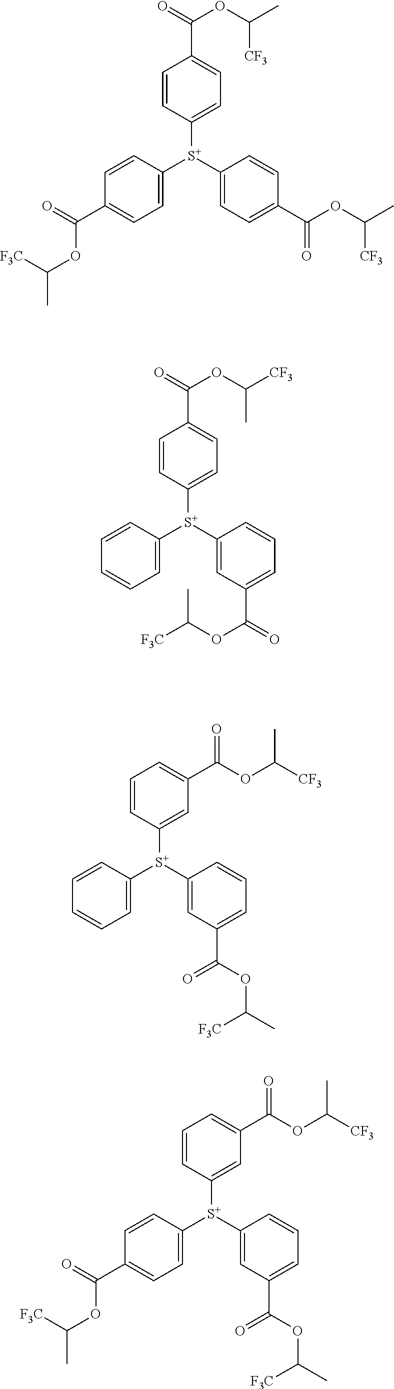 [SMILES: CC(OC(=O)c1ccc([S+](c2ccc(C(=O)OC(C)C(F)(F)F)cc2)c2ccc(C(=O)OC(C)C(F)(F)F)cc2)cc1)C(F)(F)F ; CC(OC(=O)c1ccc([S+](c2ccccc2)c2cccc(C(=O)OC(C)C(F)(F)F)c2)cc1)C(F)(F)F ; CC(OC(=O)c1cccc([S+](c2ccccc2)c2cccc(C(=O)OC(C)C(F)(F)F)c2)c1)C(F)(F)F ; CC(OC(=O)c1ccc([S+](c2cccc(C(=O)OC(C)C(F)(F)F)c2)c2cccc(C(=O)OC(C)C(F)(F)F)c2)cc1)C(F)(F)F]

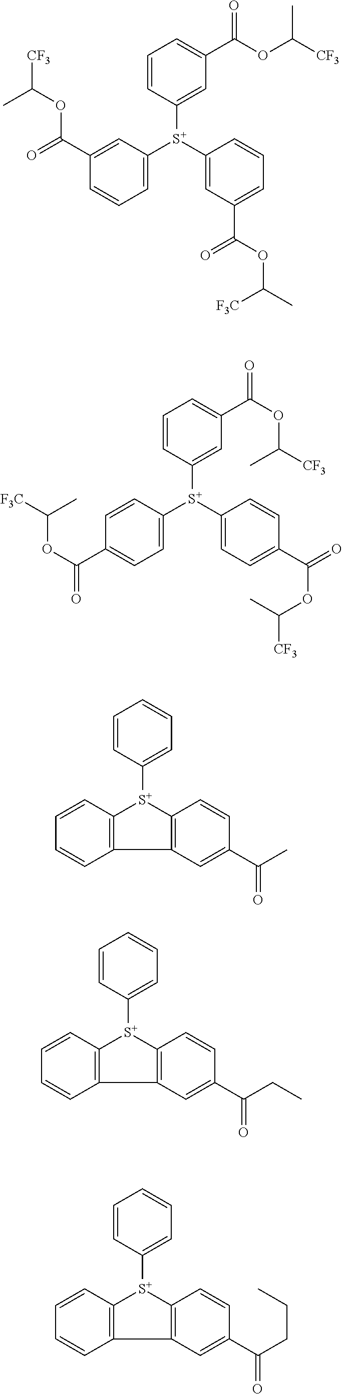 [SMILES: CC(OC(=O)c1cccc([S+](c2cccc(C(=O)OC(C)C(F)(F)F)c2)c2cccc(C(=O)OC(C)C(F)(F)F)c2)c1)C(F)(F)F ; CC(OC(=O)c1ccc([S+](c2ccc(C(=O)OC(C)C(F)(F)F)cc2)c2cccc(C(=O)OC(C)C(F)(F)F)c2)cc1)C(F)(F)F ; CC(=O)c1ccc2c(c1)c1ccccc1[s+]2-c1ccccc1 ; CCC(=O)c1ccc2c(c1)c1ccccc1[s+]2-c1ccccc1 ; CCCC(=O)c1ccc2c(c1)c1ccccc1[s+]2-c1ccccc1]

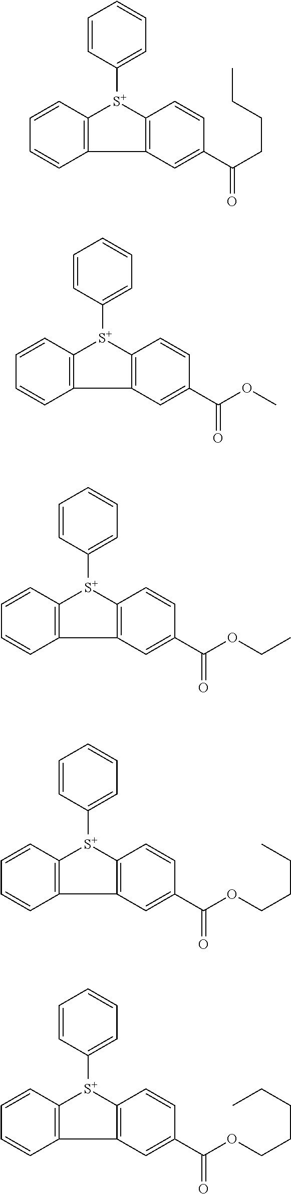 [SMILES: CCCCC(=O)c1ccc2c(c1)c1ccccc1[s+]2-c1ccccc1 ; COC(=O)c1ccc2c(c1)c1ccccc1[s+]2-c1ccccc1 ; CCOC(=O)c1ccc2c(c1)c1ccccc1[s+]2-c1ccccc1 ; CCCCOC(=O)c1ccc2c(c1)c1ccccc1[s+]2-c1ccccc1 ; CCCCCOC(=O)c1ccc2c(c1)c1ccccc1[s+]2-c1ccccc1]

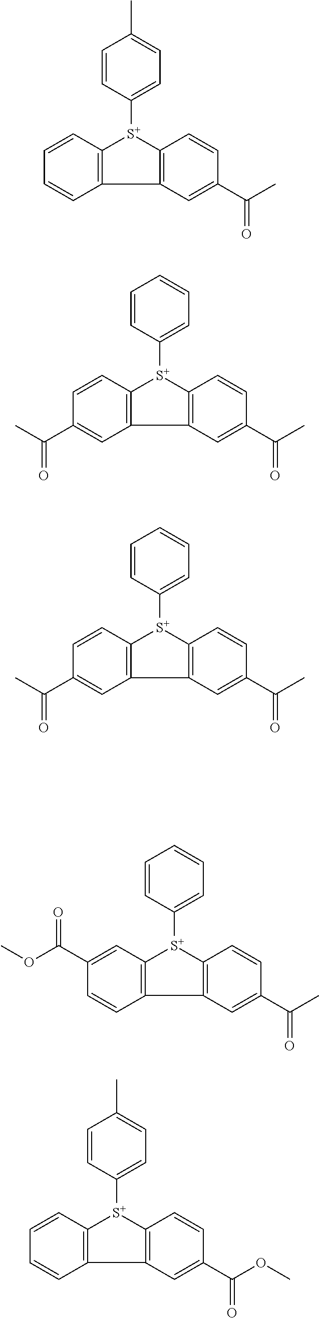

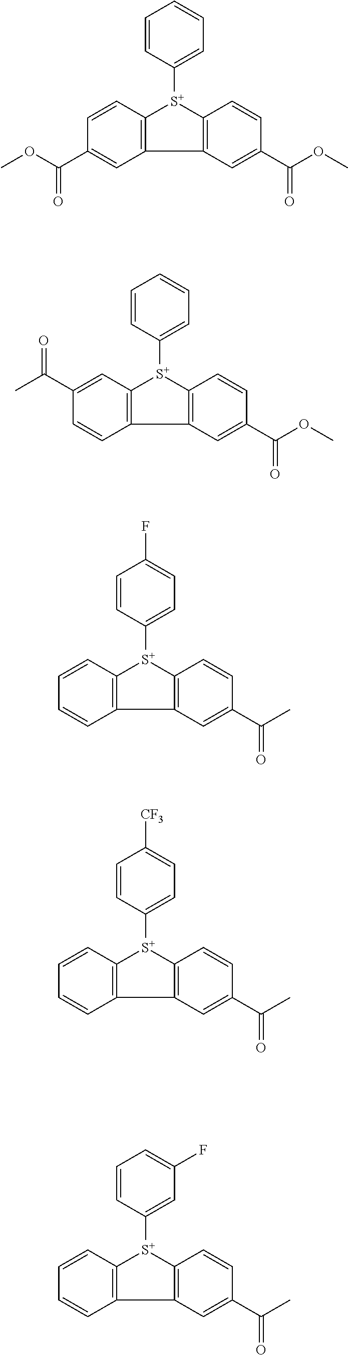

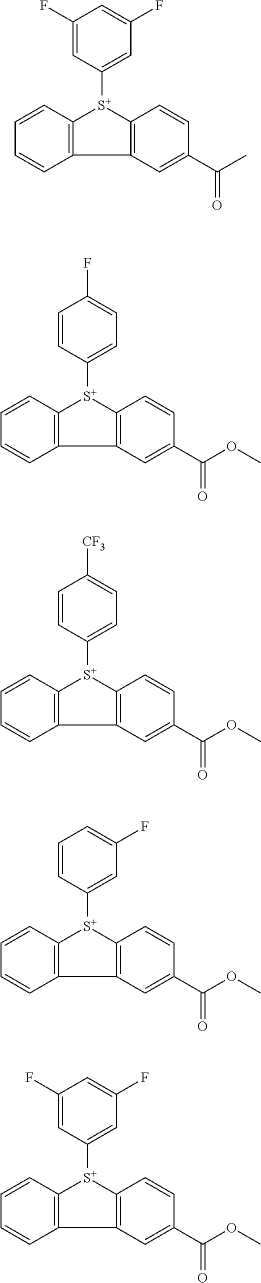 [SMILES: CC(=O)c1ccc2c(c1)c1ccccc1[s+]2-c1cc(F)cc(F)c1 ; COC(=O)c1ccc2c(c1)c1ccccc1[s+]2-c1ccc(F)cc1 ; COC(=O)c1ccc2c(c1)c1ccccc1[s+]2-c1ccc(C(F)(F)F)cc1 ; COC(=O)c1ccc2c(c1)c1ccccc1[s+]2-c1cccc(F)c1 ; COC(=O)c1ccc2c(c1)c1ccccc1[s+]2-c1cc(F)cc(F)c1]

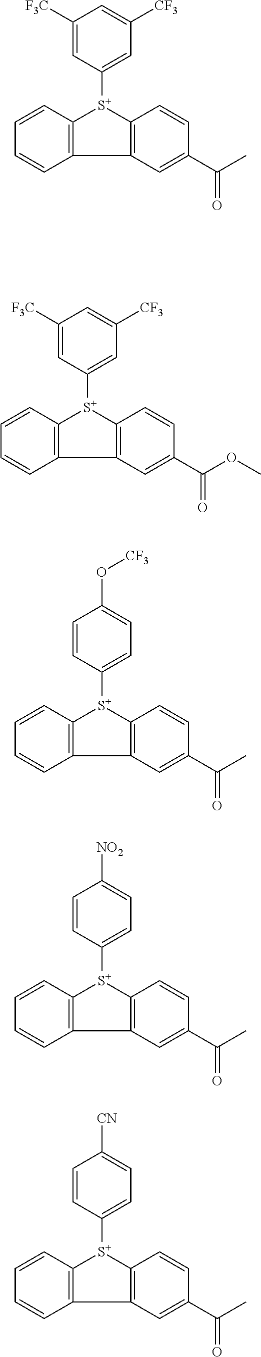 [SMILES: CC(=O)c1ccc2c(c1)c1ccccc1[s+]2-c1cc(C(F)(F)F)cc(C(F)(F)F)c1 ; COC(=O)c1ccc2c(c1)c1ccccc1[s+]2-c1cc(C(F)(F)F)cc(C(F)(F)F)c1 ; CC(=O)c1ccc2c(c1)c1ccccc1[s+]2-c1ccc(OC(F)(F)F)cc1 ; CC(=O)c1ccc2c(c1)c1ccccc1[s+]2-c1ccc([N+](=O)[O-])cc1 ; CC(=O)c1ccc2c(c1)c1ccccc1[s+]2-c1ccc(C#N)cc1]

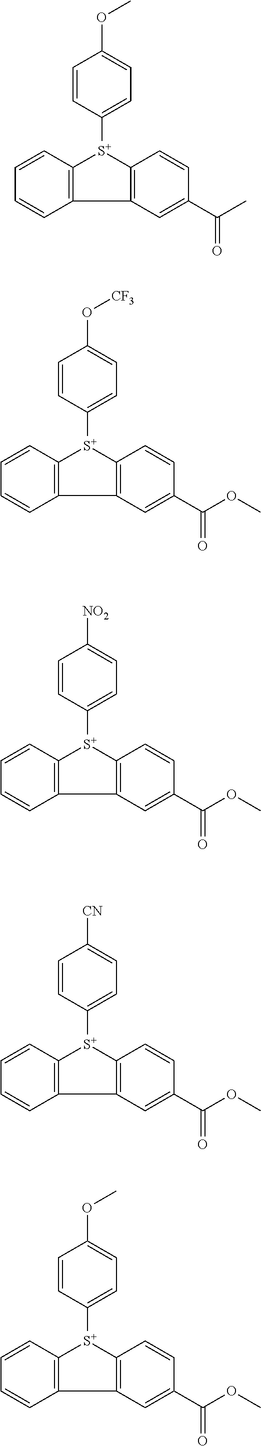 [SMILES: COc1ccc(-[s+]2c3ccccc3c3cc(C(C)=O)ccc32)cc1 ; COC(=O)c1ccc2c(c1)c1ccccc1[s+]2-c1ccc(OC(F)(F)F)cc1 ; COC(=O)c1ccc2c(c1)c1ccccc1[s+]2-c1ccc([N+](=O)[O-])cc1 ; COC(=O)c1ccc2c(c1)c1ccccc1[s+]2-c1ccc(C#N)cc1 ; COC(=O)c1ccc2c(c1)c1ccccc1[s+]2-c1ccc(OC)cc1]

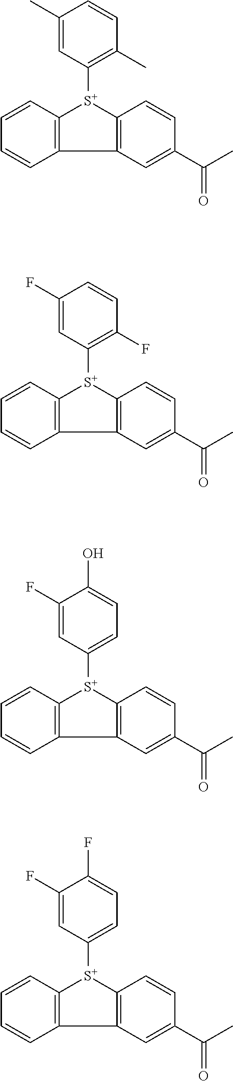 [SMILES: CC(=O)c1ccc2c(c1)c1ccccc1[s+]2-c1cc(C)ccc1C ; CC(=O)c1ccc2c(c1)c1ccccc1[s+]2-c1cc(F)ccc1F ; CC(=O)c1ccc2c(c1)c1ccccc1[s+]2-c1ccc(O)c(F)c1 ; CC(=O)c1ccc2c(c1)c1ccccc1[s+]2-c1ccc(F)c(F)c1]

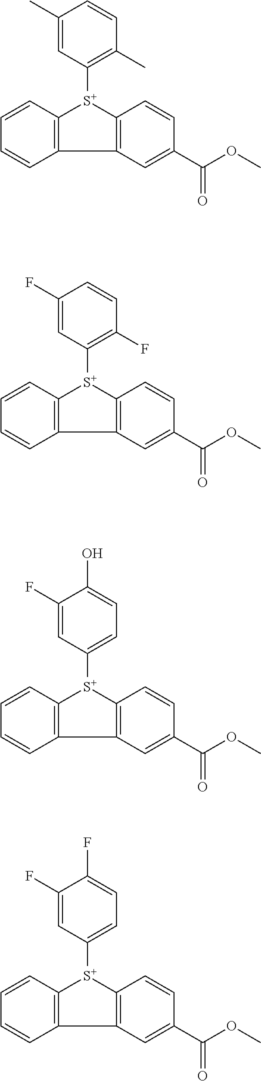 [SMILES: COC(=O)c1ccc2c(c1)c1ccccc1[s+]2-c1cc(C)ccc1C ; COC(=O)c1ccc2c(c1)c1ccccc1[s+]2-c1cc(F)ccc1F ; COC(=O)c1ccc2c(c1)c1ccccc1[s+]2-c1ccc(O)c(F)c1 ; COC(=O)c1ccc2c(c1)c1ccccc1[s+]2-c1ccc(F)c(F)c1]

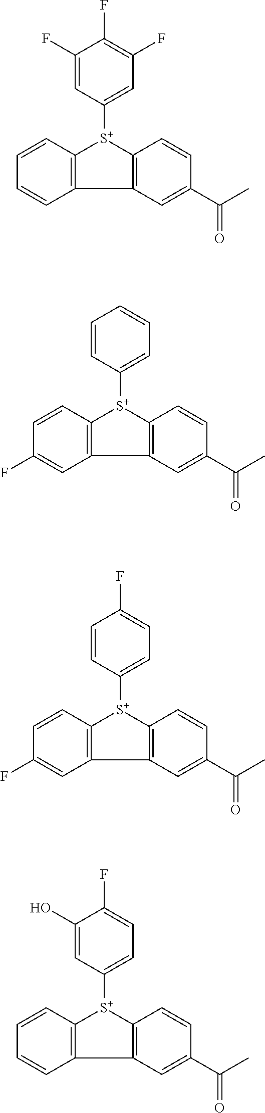 [SMILES: CC(=O)c1ccc2c(c1)c1ccccc1[s+]2-c1cc(F)c(F)c(F)c1 ; CC(=O)c1ccc2c(c1)c1cc(F)ccc1[s+]2-c1ccccc1 ; CC(=O)c1ccc2c(c1)c1cc(F)ccc1[s+]2-c1ccc(F)cc1 ; CC(=O)c1ccc2c(c1)c1ccccc1[s+]2-c1ccc(F)c(O)c1]

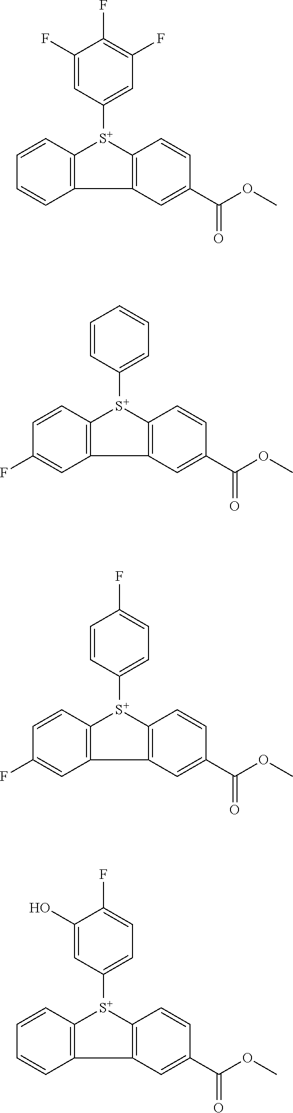 [SMILES: COC(=O)c1ccc2c(c1)c1ccccc1[s+]2-c1cc(F)c(F)c(F)c1 ; COC(=O)c1ccc2c(c1)c1cc(F)ccc1[s+]2-c1ccccc1 ; COC(=O)c1ccc2c(c1)c1cc(F)ccc1[s+]2-c1ccc(F)cc1 ; COC(=O)c1ccc2c(c1)c1ccccc1[s+]2-c1ccc(F)c(O)c1]

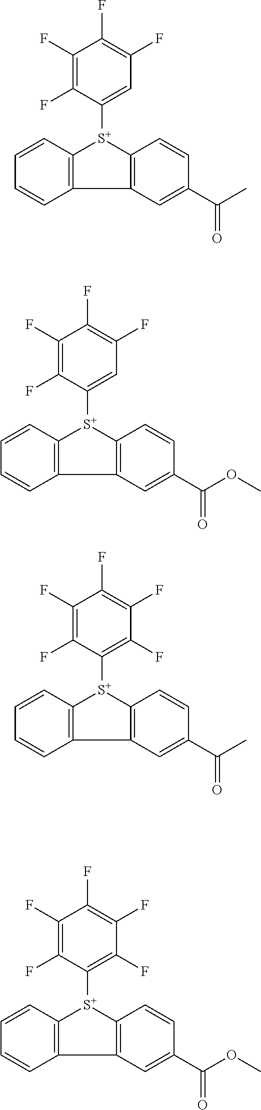 [SMILES: CC(=O)c1ccc2c(c1)c1ccccc1[s+]2-c1cc(F)c(F)c(F)c1F ; COC(=O)c1ccc2c(c1)c1ccccc1[s+]2-c1cc(F)c(F)c(F)c1F ; CC(=O)c1ccc2c(c1)c1ccccc1[s+]2-c1c(F)c(F)c(F)c(F)c1F ; COC(=O)c1ccc2c(c1)c1ccccc1[s+]2-c1c(F)c(F)c(F)c(F)c1F]

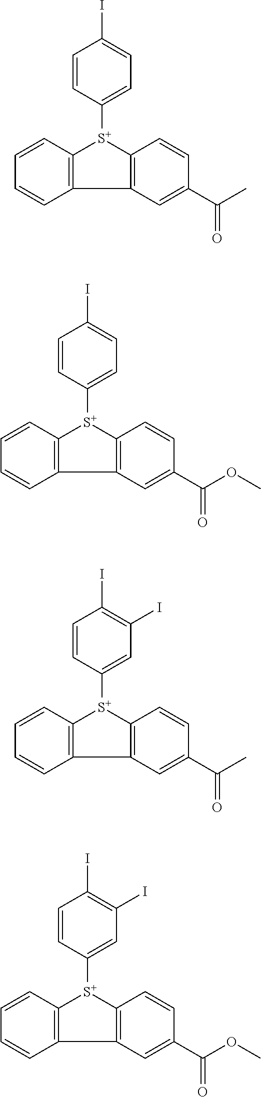 [SMILES: CC(=O)c1ccc2c(c1)c1ccccc1[s+]2-c1ccc(I)cc1 ; COC(=O)c1ccc2c(c1)c1ccccc1[s+]2-c1ccc(I)cc1 ; CC(=O)c1ccc2c(c1)c1ccccc1[s+]2-c1ccc(I)c(I)c1 ; COC(=O)c1ccc2c(c1)c1ccccc1[s+]2-c1ccc(I)c(I)c1]

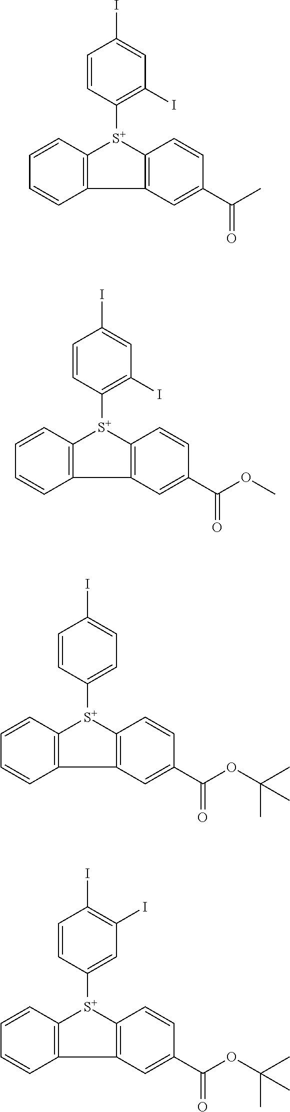 [SMILES: CC(=O)c1ccc2c(c1)c1ccccc1[s+]2-c1ccc(I)cc1I ; COC(=O)c1ccc2c(c1)c1ccccc1[s+]2-c1ccc(I)cc1I ; CC(C)(C)OC(=O)c1ccc2c(c1)c1ccccc1[s+]2-c1ccc(I)cc1 ; CC(C)(C)OC(=O)c1ccc2c(c1)c1ccccc1[s+]2-c1ccc(I)c(I)c1]

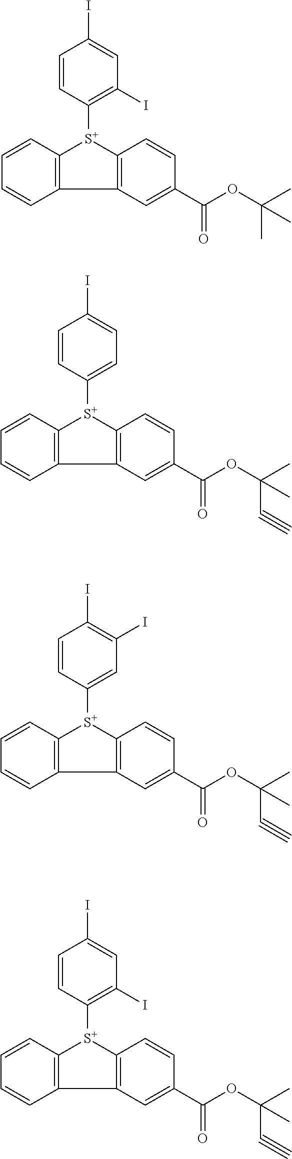 [SMILES: CC(C)(C)OC(=O)c1ccc2c(c1)c1ccccc1[s+]2-c1ccc(I)cc1I ; C#CC(C)(C)OC(=O)c1ccc2c(c1)c1ccccc1[s+]2-c1ccc(I)cc1 ; C#CC(C)(C)OC(=O)c1ccc2c(c1)c1ccccc1[s+]2-c1ccc(I)c(I)c1 ; C#CC(C)(C)OC(=O)c1ccc2c(c1)c1ccccc1[s+]2-c1ccc(I)cc1I]

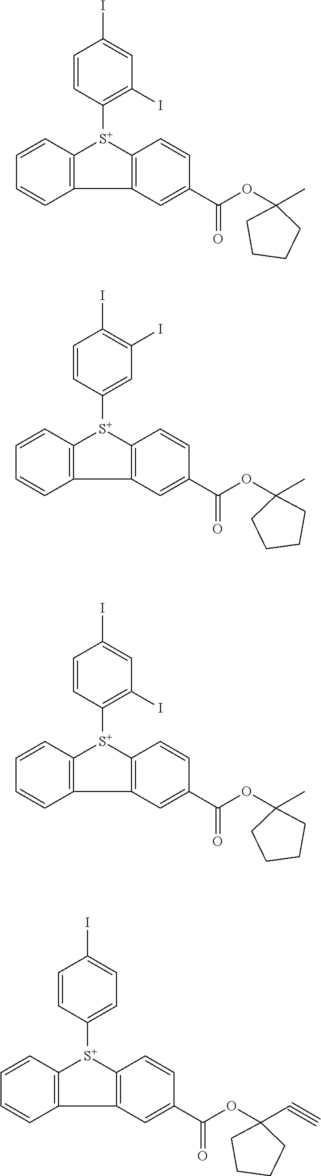 [SMILES: CC1(OC(=O)c2ccc3c(c2)c2ccccc2[s+]3-c2ccc(I)cc2I)CCCC1 ; CC1(OC(=O)c2ccc3c(c2)c2ccccc2[s+]3-c2ccc(I)c(I)c2)CCCC1 ; CC1(OC(=O)c2ccc3c(c2)c2ccccc2[s+]3-c2ccc(I)cc2I)CCCC1 ; C#CC1(OC(=O)c2ccc3c(c2)c2ccccc2[s+]3-c2ccc(I)cc2)CCCC1]

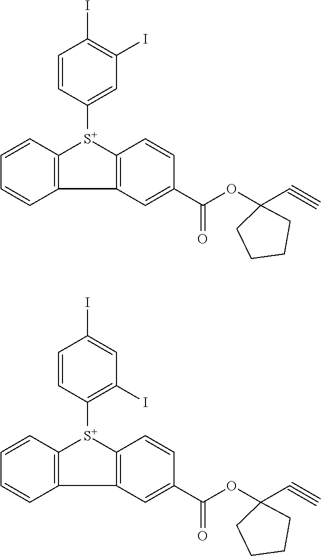 [SMILES: C#CC1(OC(=O)c2ccc3c(c2)c2ccccc2[s+]3-c2ccc(I)c(I)c2)CCCC1 ; C#CC1(OC(=O)c2ccc3c(c2)c2ccccc2[s+]3-c2ccc(I)cc2I)CCCC1]

Examples of the iodonium cation represented by M⁺ include those shown below, but are not limited thereto.

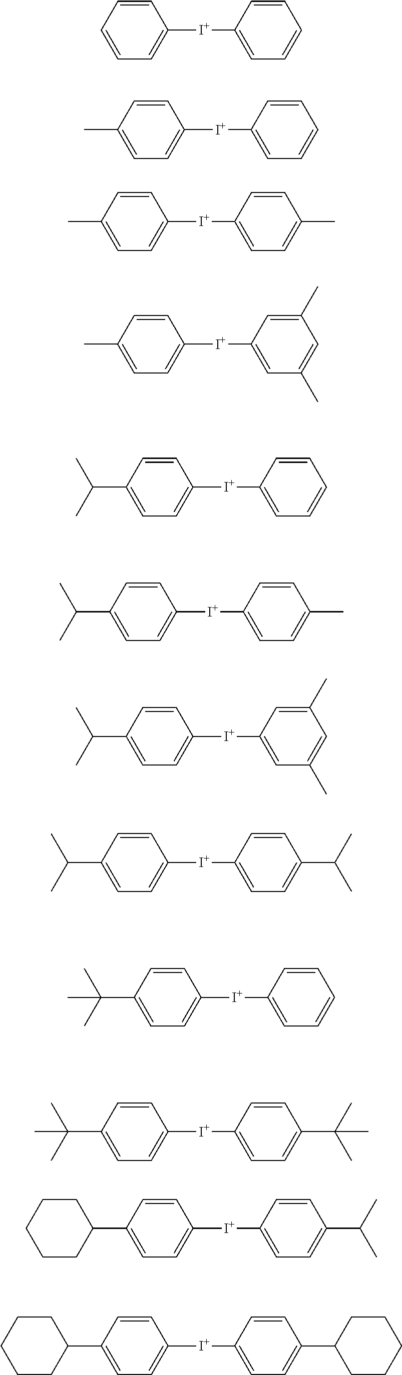

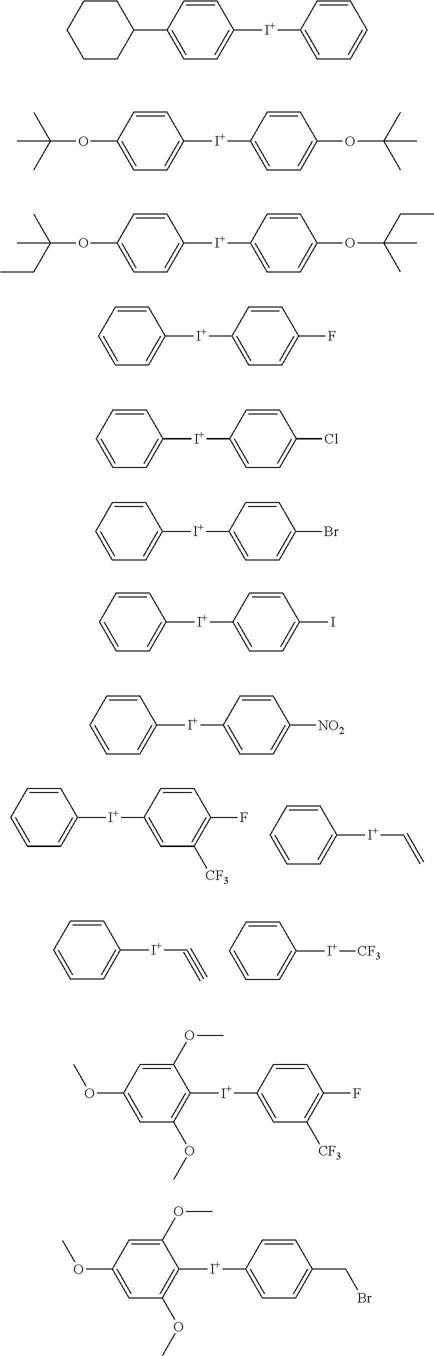

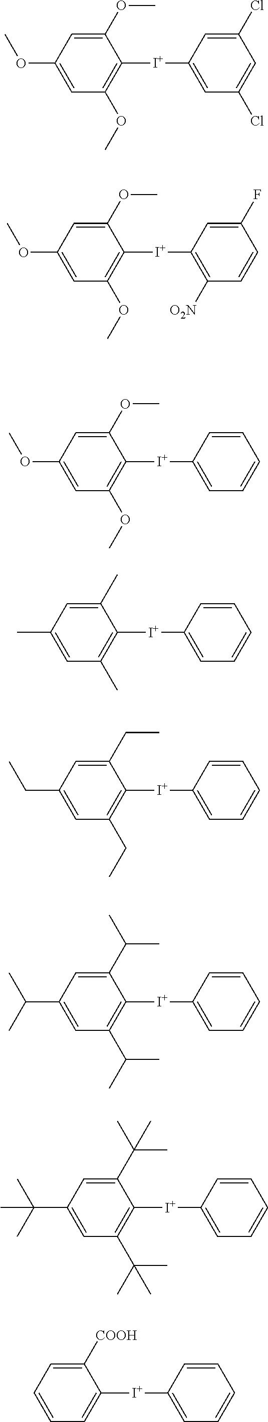

Examples of the method for synthesizing the sulfonium salt and the iodonium salt having formula (1) include a method in which a sulfonium salt or an iodonium salt containing a halide anion is subjected to salt exchange with an ammonium salt containing the sulfonate anion having an aromatic group substituted with a bromine atom or an iodine atom.

The sulfonium salt or iodonium salt having formula (1) is preferably added to the resist composition of the present invention in an amount of 0.01 to 1,000 parts by weight, and more preferably 0.05 to 500 parts by weight per 100 parts by weight of the base polymer described later, from the viewpoint of sensitivity and an effect of reducing acid diffusion.

### [Base Polymer]

When the resist composition of the present invention is of positive tone, the base polymer comprised in the resist composition contains repeat units containing an acid labile group. The repeat units containing an acid labile group are preferably repeat units having formula (a1) (hereinafter, the repeat units are also referred to as repeat units (a1)) or repeat units having formula (a2) (hereinafter, the repeat units are also referred to as repeat units (a2)).

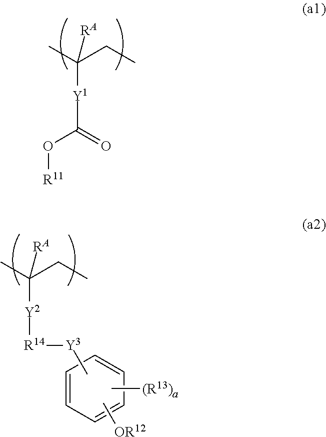

In formulae (a1) and (a2), R^{A} is each independently a hydrogen atom or a methyl group, Y¹ is a single bond, a phenylene group, a naphthylene group, or a C₁-C₁₂ linking group containing at least one moiety selected from an ester bond, an ether bond, and a lactone ring, Y² is a single bond or an ester bond, Y³ is a single bond, an ether bond, or an ester bond, R¹¹ and R¹² are each independently an acid labile group, R¹³ is a C₁-C₄ saturated hydrocarbyl group, a halogen atom, a C₂-C₅ saturated hydrocarbylcarbonyl group, a cyano group, or a C₂-C₅ saturated hydrocarbyloxycarbonyl group, R¹⁴ is a single bond or a C₁-C₆ alkanediyl group which may contain an ether bond or an ester bond, and a is an integer of 0 to 4.

Examples of the monomer from which the repeat units (a1) are derived include those shown below, but are not limited thereto. In the formulae, R^{A} and R¹¹ are as defined above.

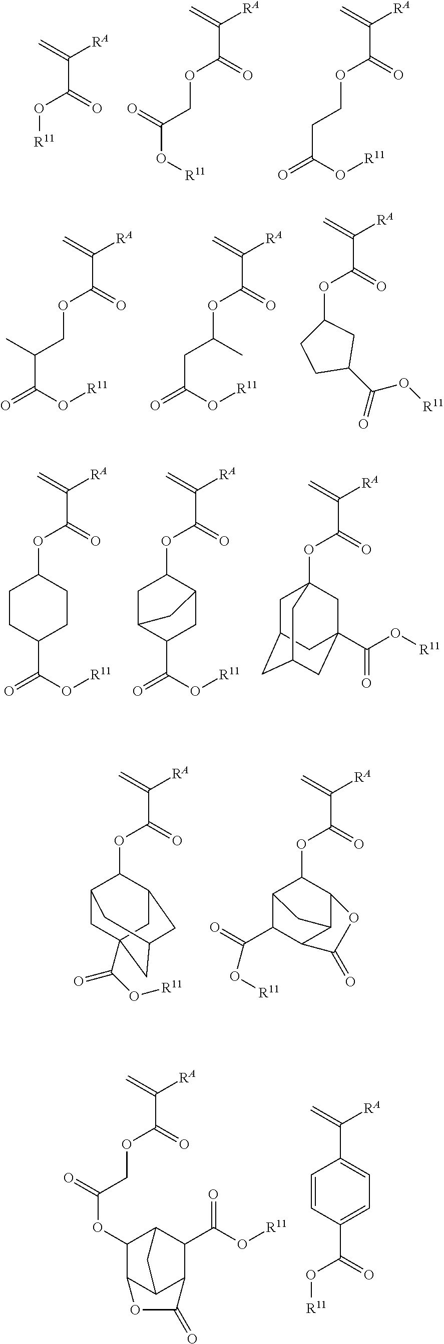

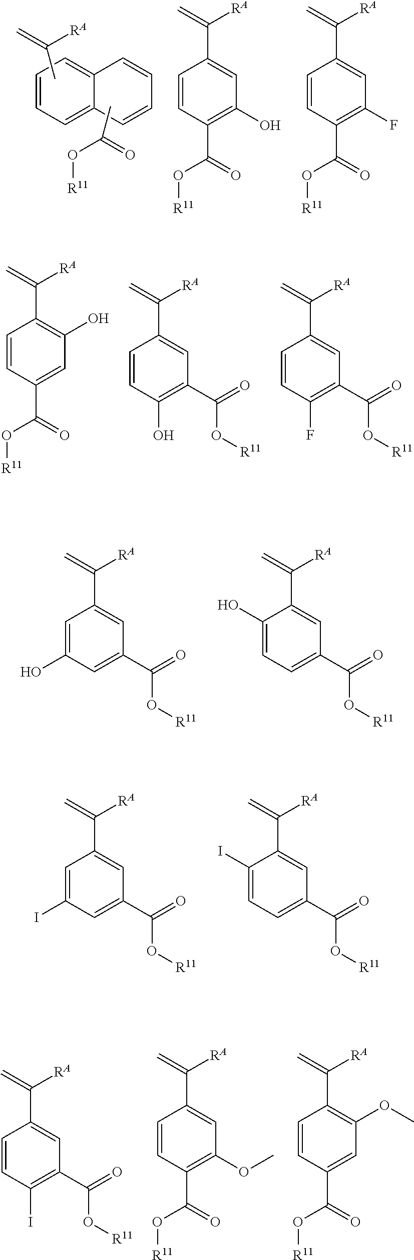

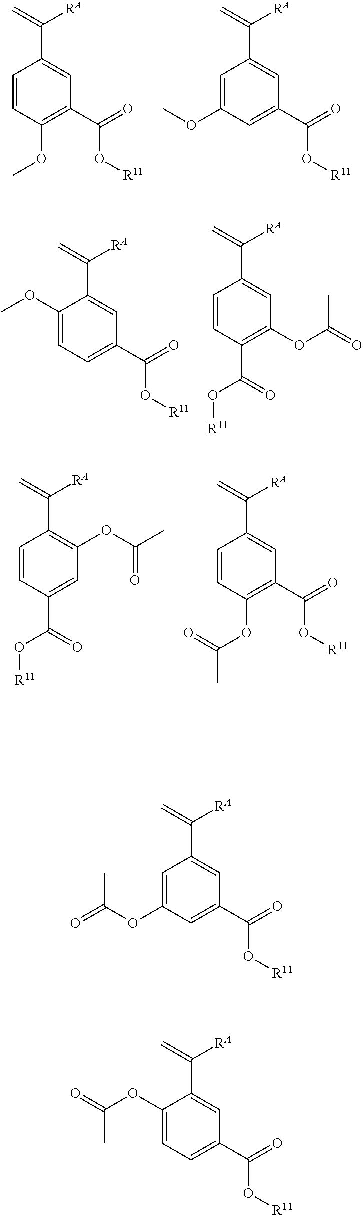

Examples of the monomer from which the repeat units (a2) are derived include those shown below, but are not limited thereto. In the formulae, R^{A} and R¹² are as defined above.

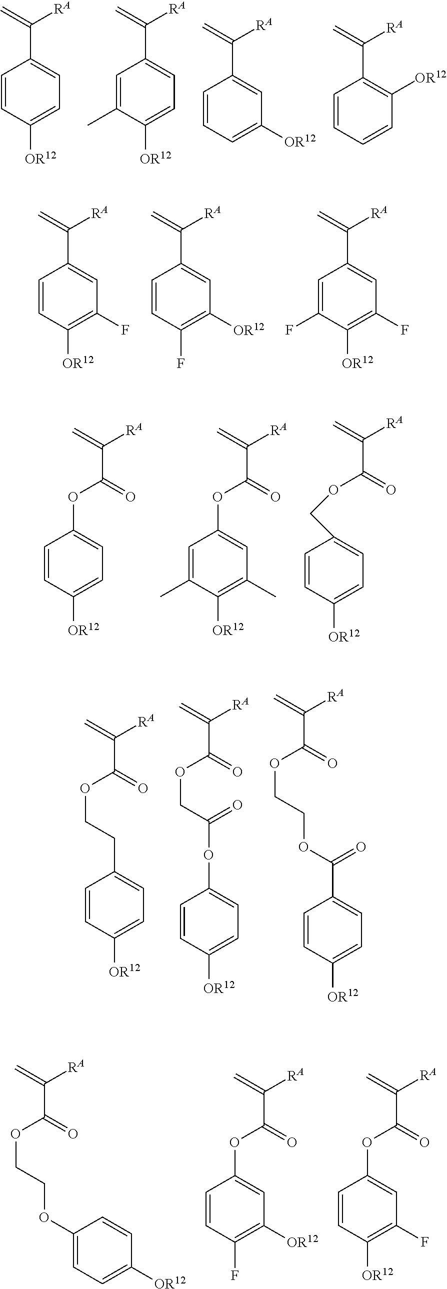

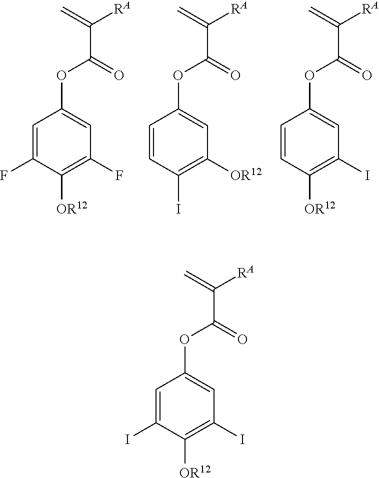

Examples of the acid labile groups represented by R¹¹ and R¹² in the repeat units (a1) and (a2) include those described in JP-A 2013-080033 and JP-A 2013-083821.

Typical examples of the acid labile groups include those represented by any of formulae (AL-1) to (AL-3).

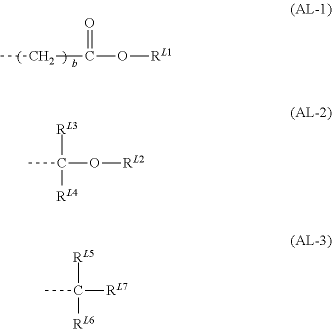

In the formulae, a broken line represents a bond.

In formulae (AL-1) and (AL-2), R^{L1} and R^{L2} are each independently a C₁-C₄₀ hydrocarbyl group which may contain a heteroatom such as an oxygen atom, a sulfur atom, a nitrogen atom, or a fluorine atom. The hydrocarbyl group may be saturated or unsaturated, and may be straight, branched, or cyclic. Preferred are C₁-C₄₀ saturated hydrocarbyl groups, especially C₁-C₂₀ saturated hydrocarbyl groups.

In formula (AL-1), b is an integer of 0 to 10, preferably an integer of 1 to 5.

In formula (AL-2), R^{L3} and R^{L4} are each independently a hydrogen atom or a C₁-C₂₀ hydrocarbyl group which may contain a heteroatom such as an oxygen atom, a sulfur atom, a nitrogen atom, or a fluorine atom. The hydrocarbyl group may be saturated or unsaturated, and may be straight, branched, or cyclic. Preferred are C₁-C₂₀ saturated hydrocarbyl groups. Any two of R^{L2}, R^{L3}, and R^{L4} may bond together to form a ring, typically an alicyclic ring, with the carbon atom or carbon and oxygen atoms to which they are bonded, the ring containing 3 to 20 carbon atoms. The ring is preferably a C₄-C₁₆ ring, and particularly preferably an alicyclic ring.

In formula (AL-3), R^{L5}, R^{L6}, and R^{L7} are each independently a C₁-C₂₀ hydrocarbyl group which may contain a heteroatom such as an oxygen atom, a sulfur atom, a nitrogen atom, or a fluorine atom. The hydrocarbyl group may be saturated or unsaturated, and may be straight, branched, or cyclic. Preferred are C₁-C₂₀ saturated hydrocarbyl groups. Any two of R^{L5}, R^{L6}, and R^{L7} may bond together to form a ring, typically an alicyclic ring, with the carbon atom to which they are bonded, the ring containing 3 to 20 carbon atoms. The ring is preferably a C₄-C₁₆ ring, and particularly preferably an alicyclic ring.

The base polymer may contain repeat units (b) having a phenolic hydroxy group as an adhesive group. Examples of the monomer from which the repeat units (b) are derived include those shown below, but are not limited thereto. In the formulae, R^{A} is as defined above.

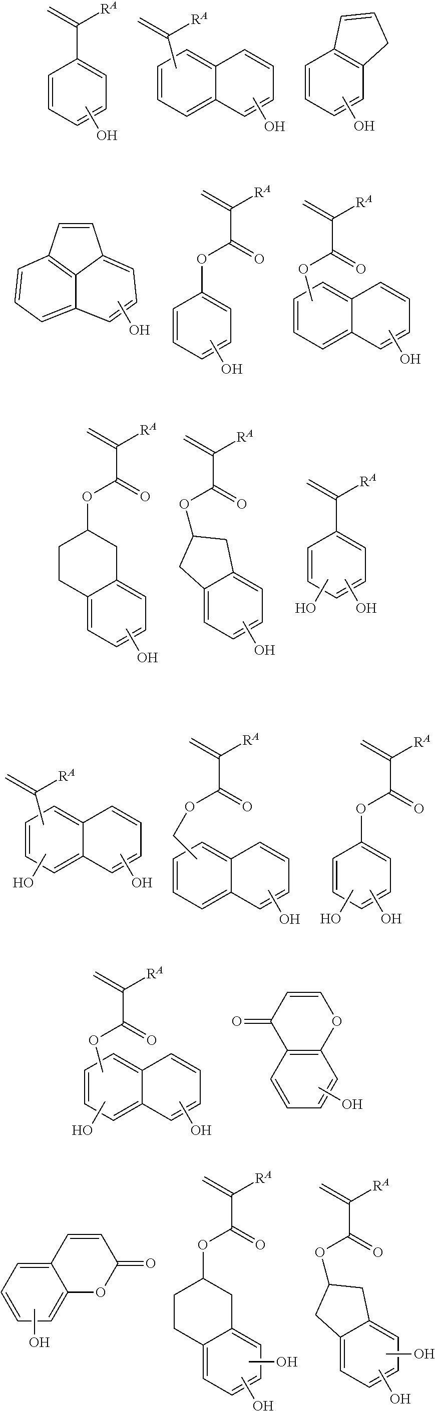

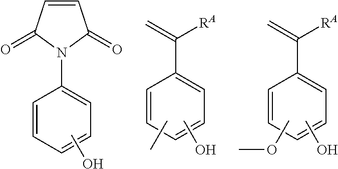

The base polymer may further contain repeat units (c) having another adhesive group selected from a hydroxy group other than the phenolic hydroxy group, a lactone ring, a sultone ring, an ether bond, an ester bond, a sulfonic acid ester bond, a carbonyl group, a sulfonyl group, a cyano group, and a carboxy group. Examples of the monomer from which the repeat units (c) are derived include those shown below, but are not limited thereto. In the formulae, R^{A} is as defined above.

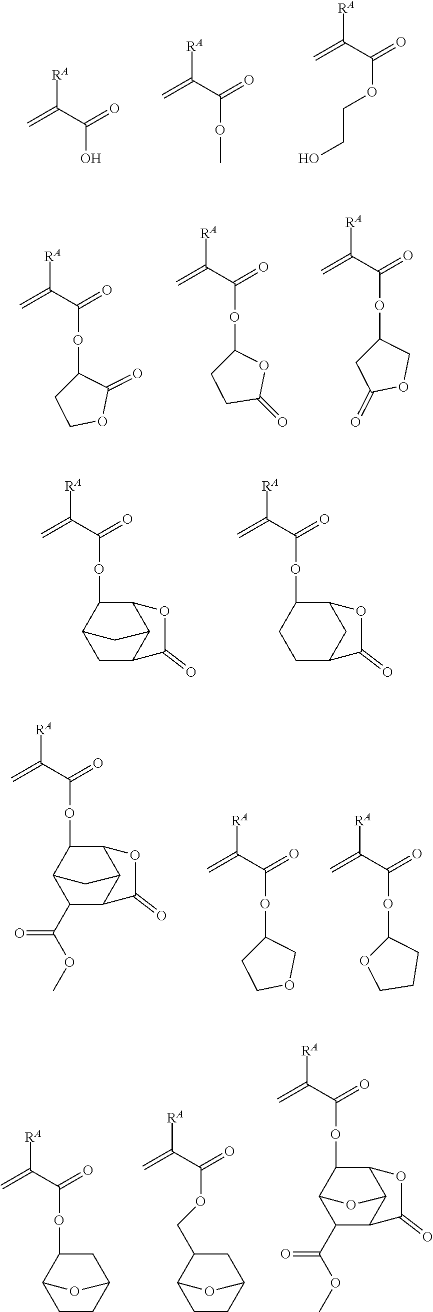

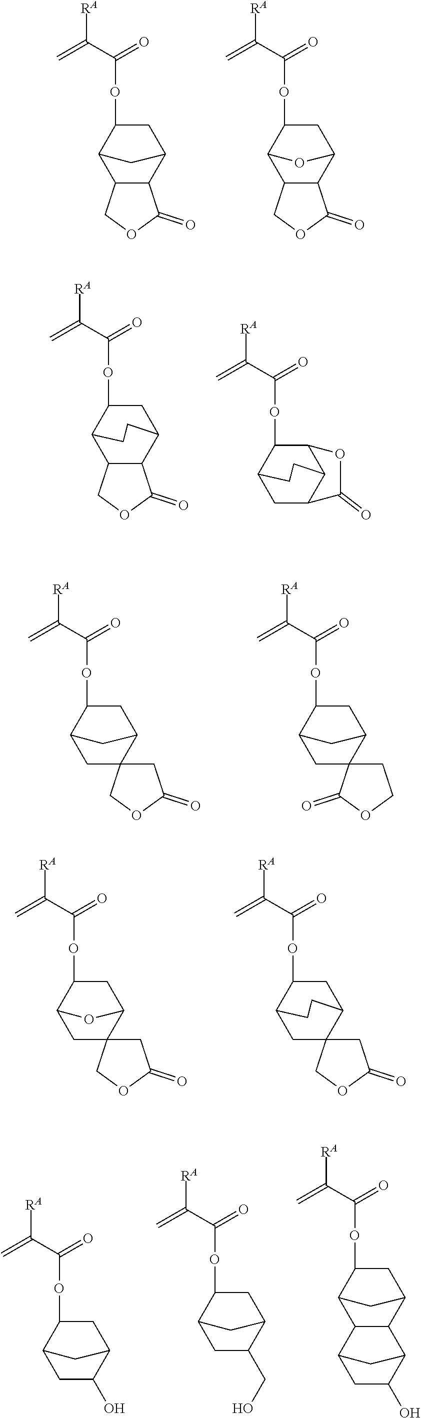

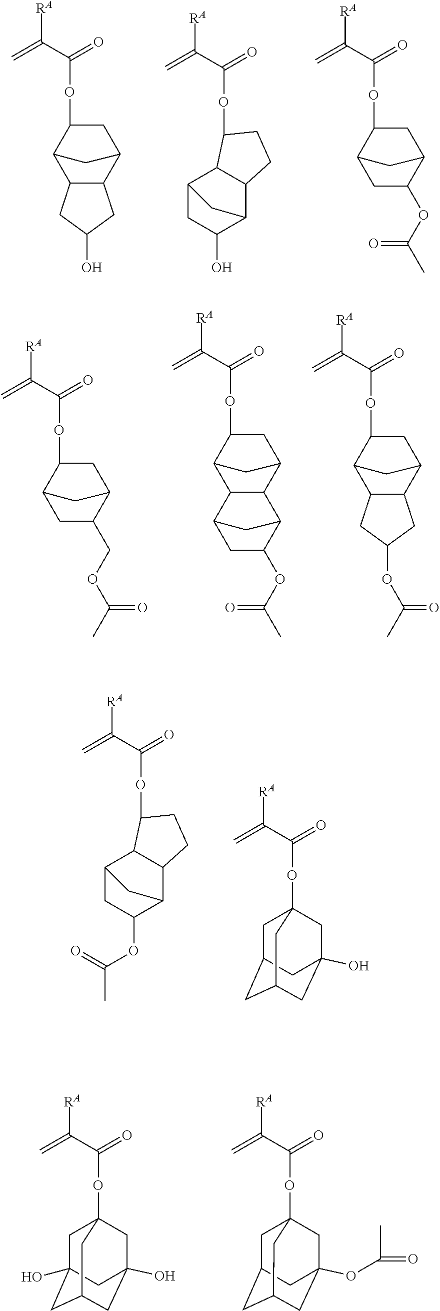

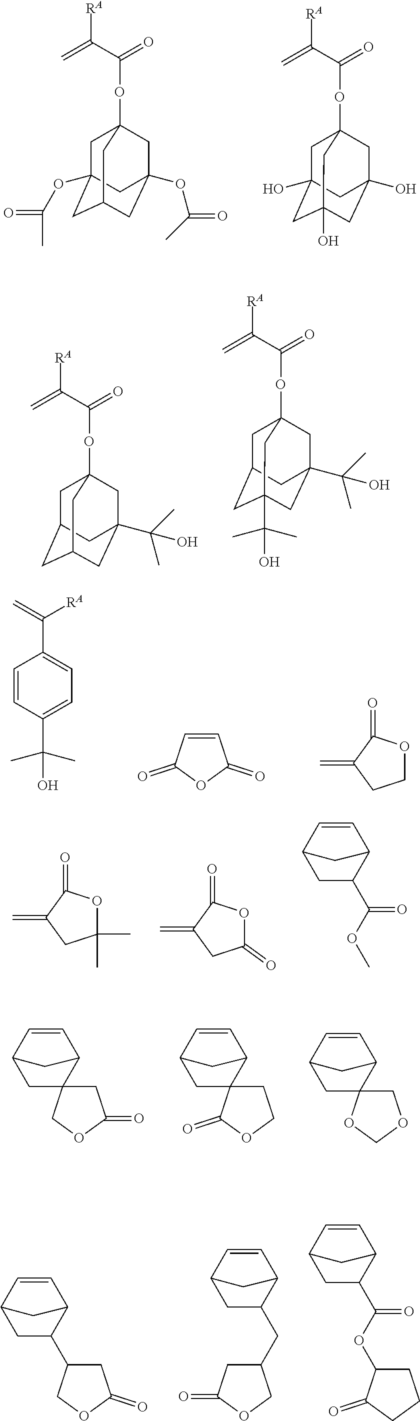

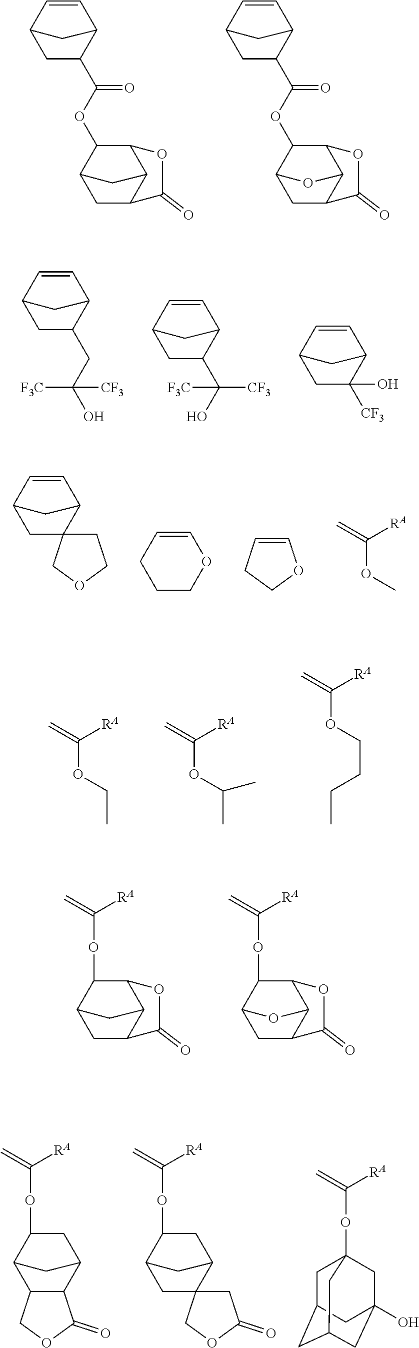

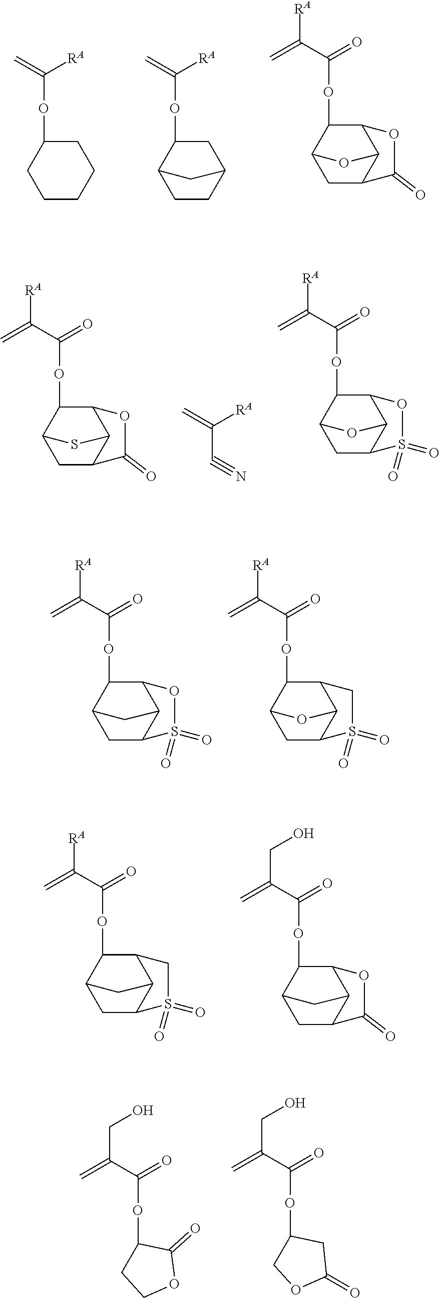

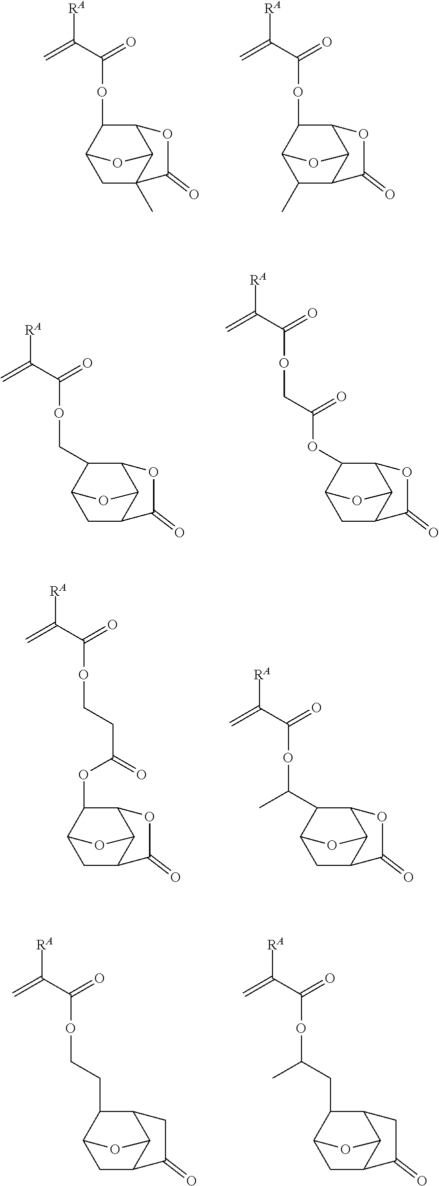

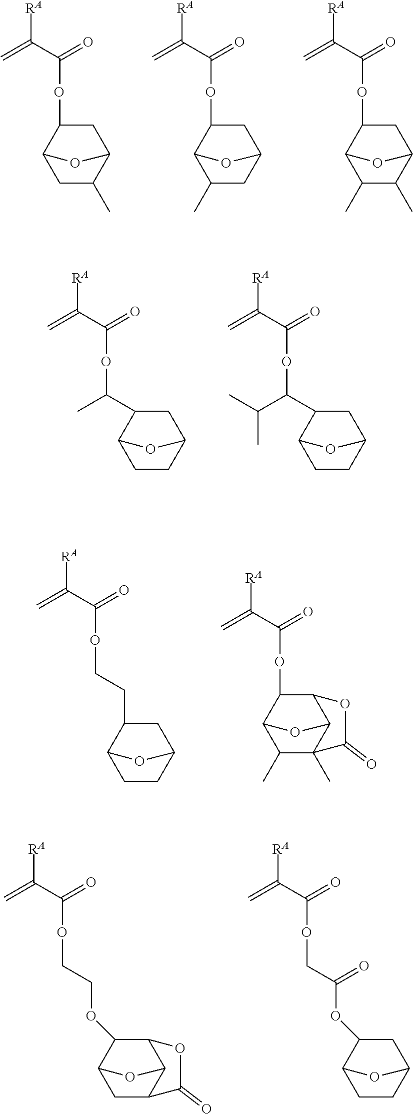

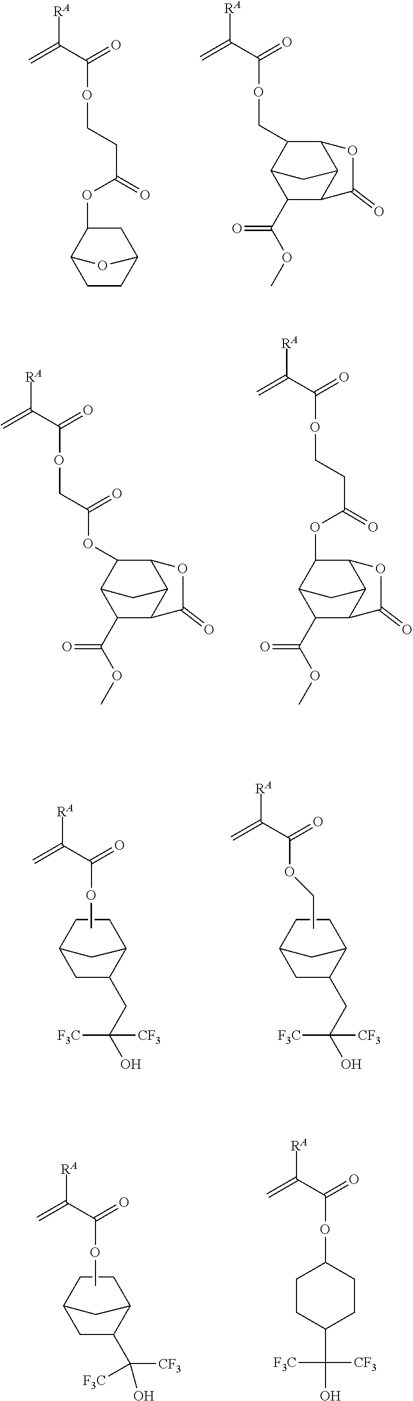

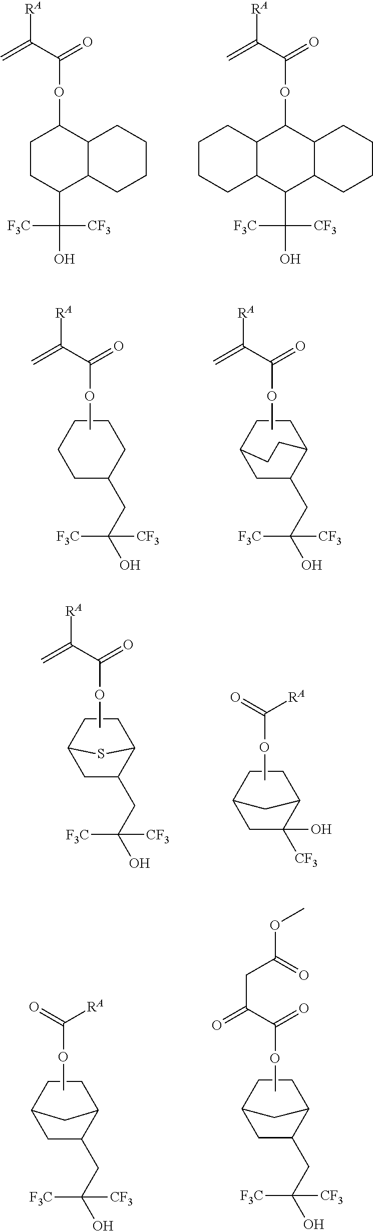

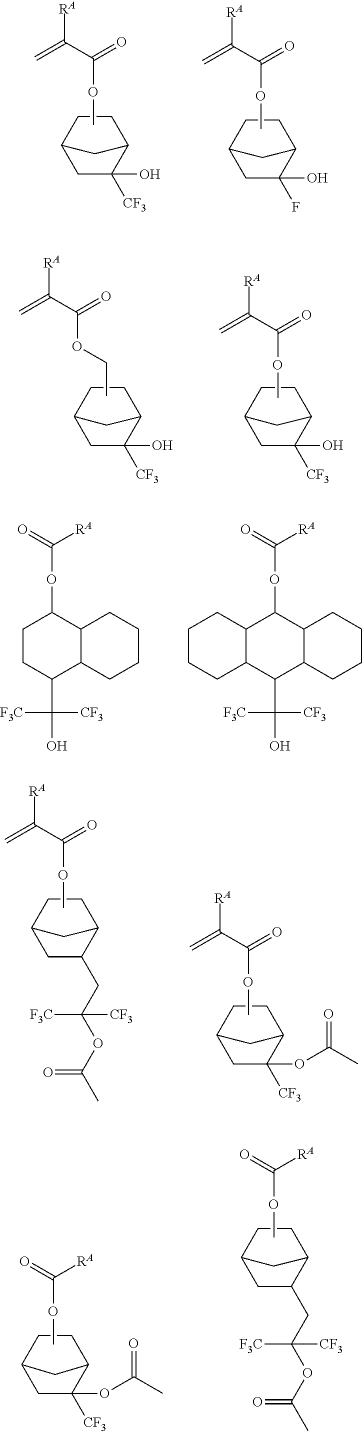

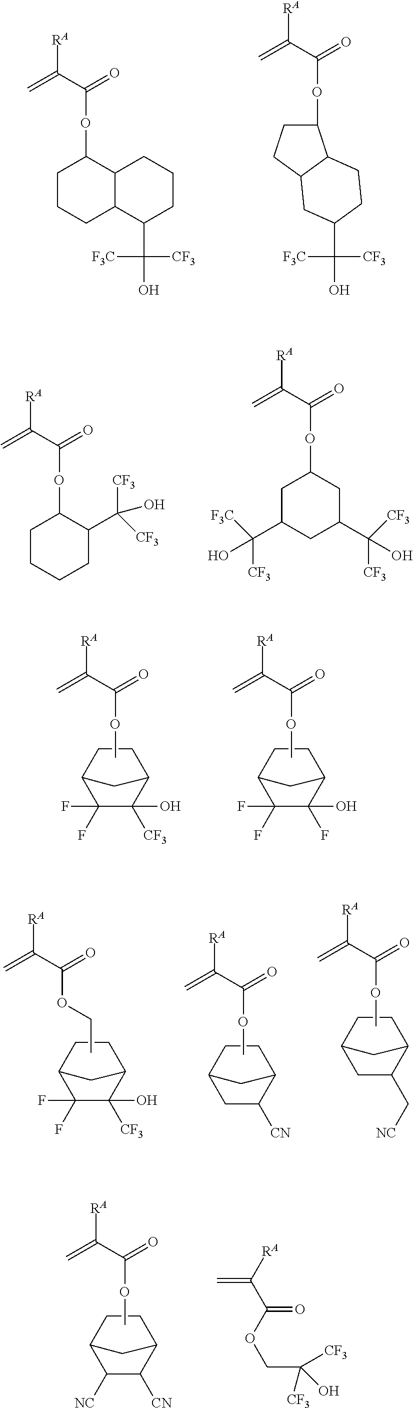

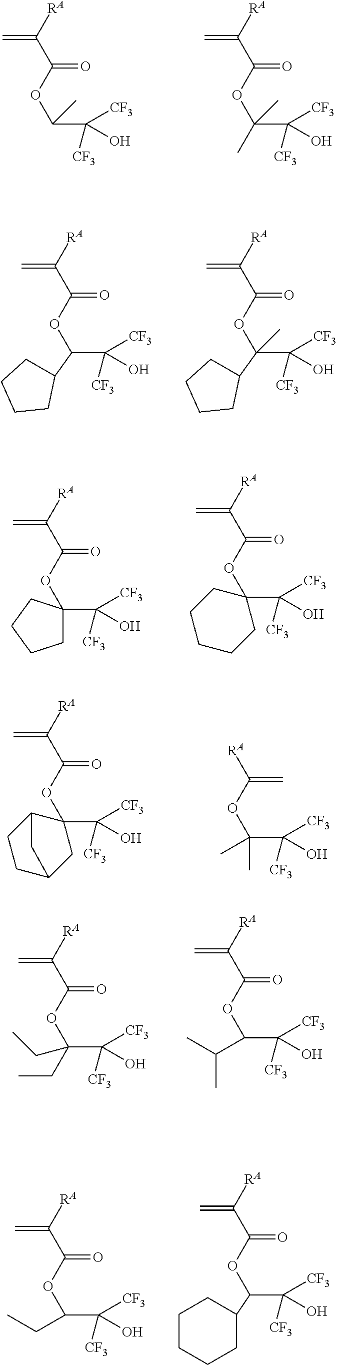

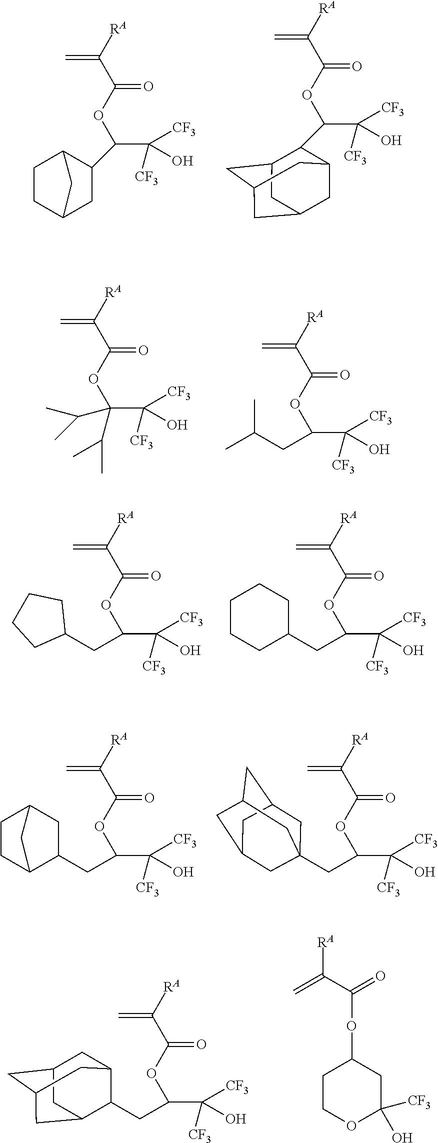

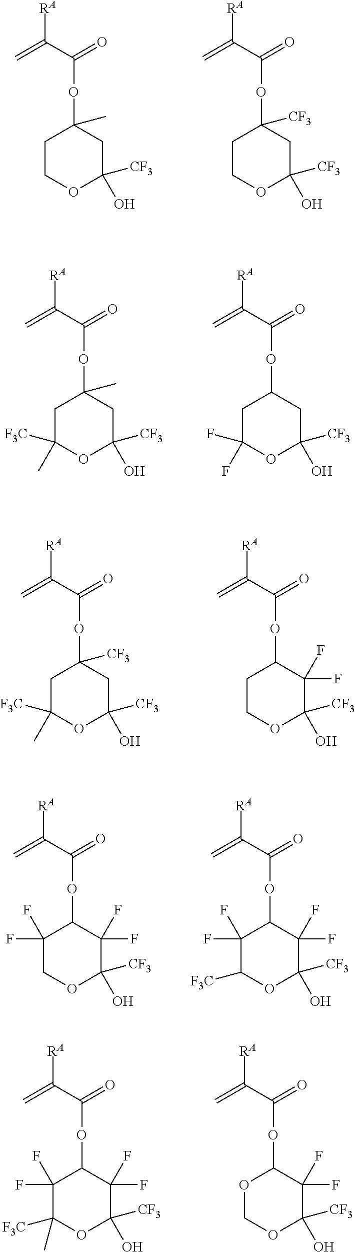

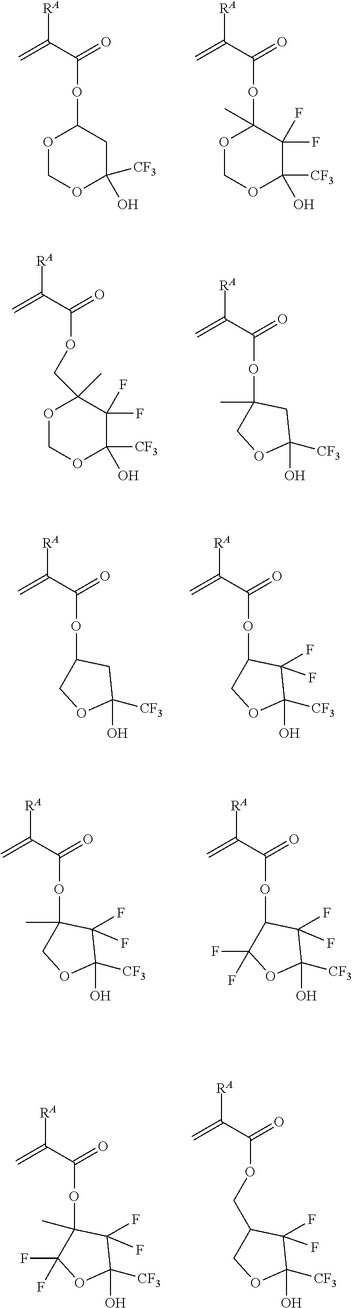

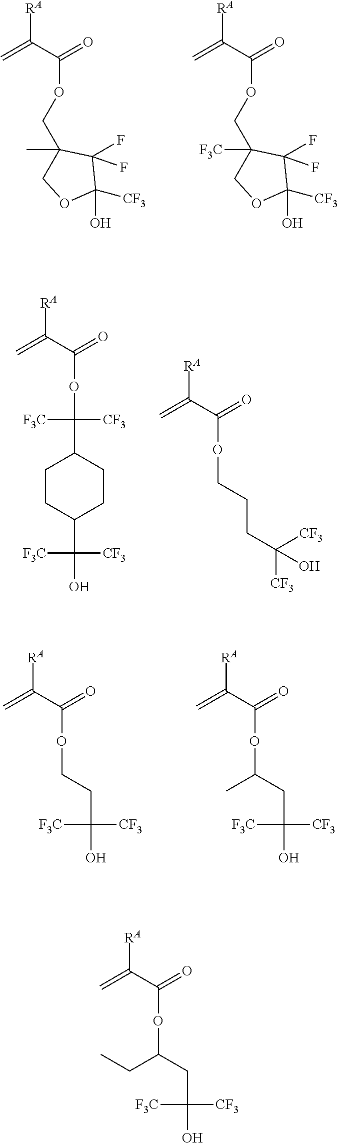

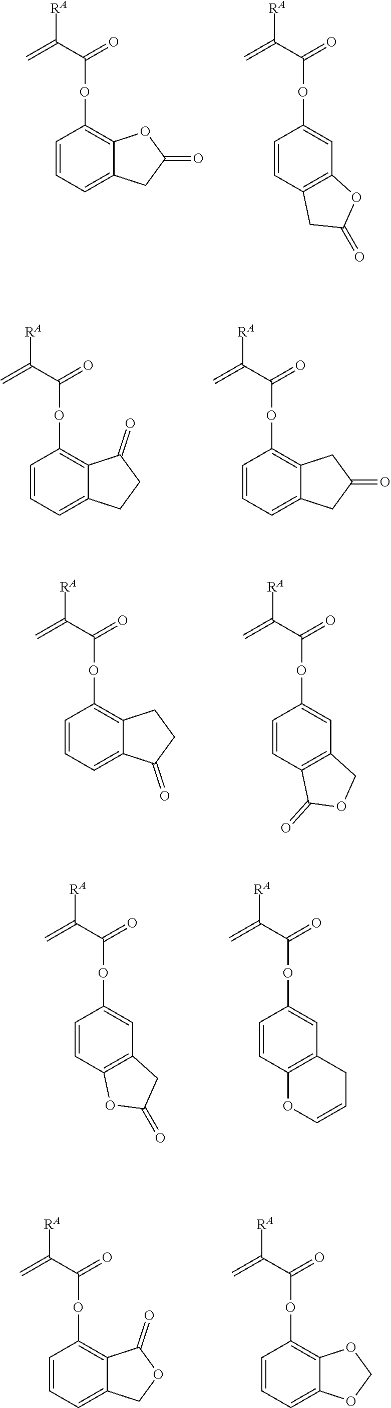

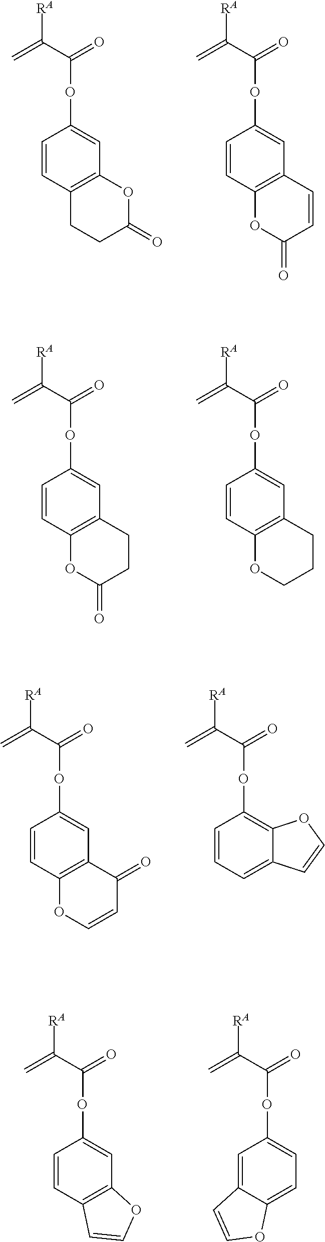

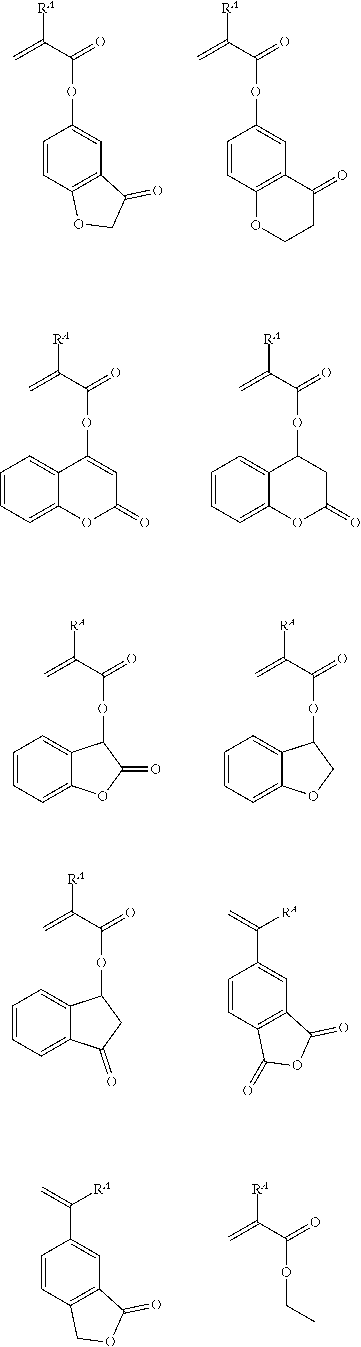

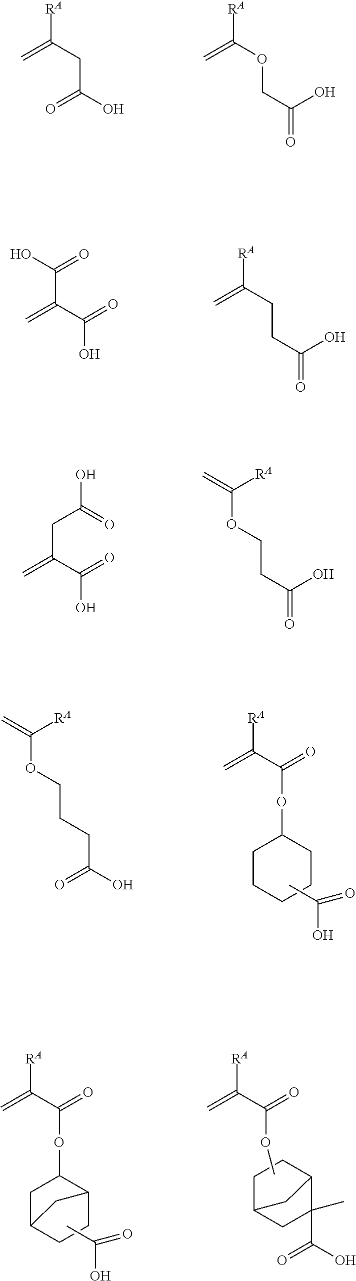

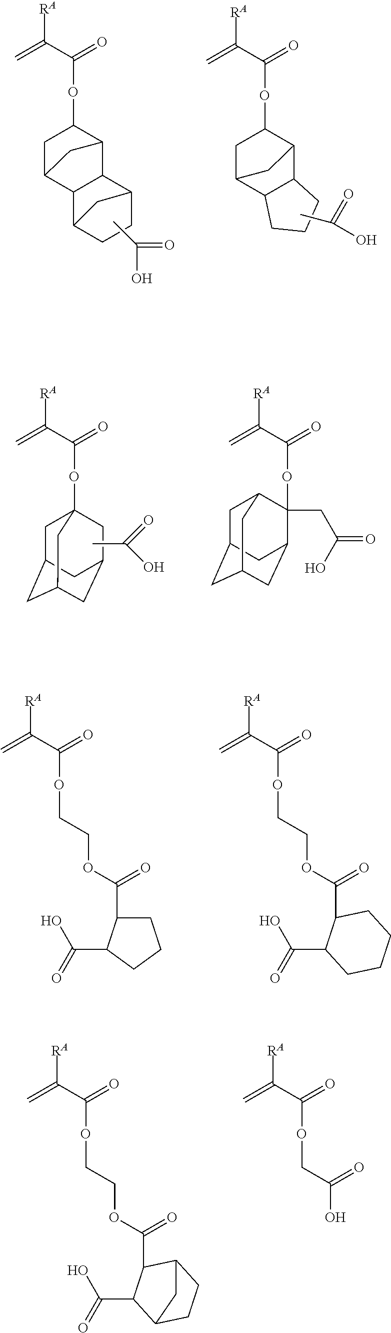

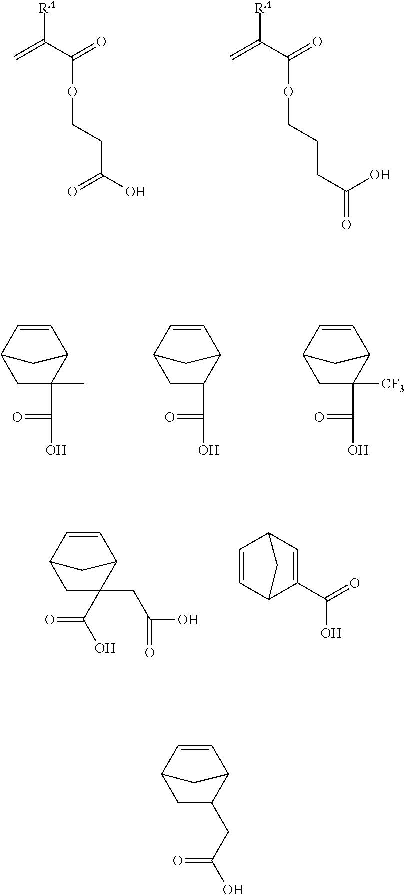

The base polymer may contain repeat units (d) derived from indene, benzofuran, benzothiophene, acenaphthylene, chromone, coumarin, norbornadiene, or a derivative thereof. Examples of the monomer from which the repeat units (d) are derived include those shown below, but are not limited thereto.

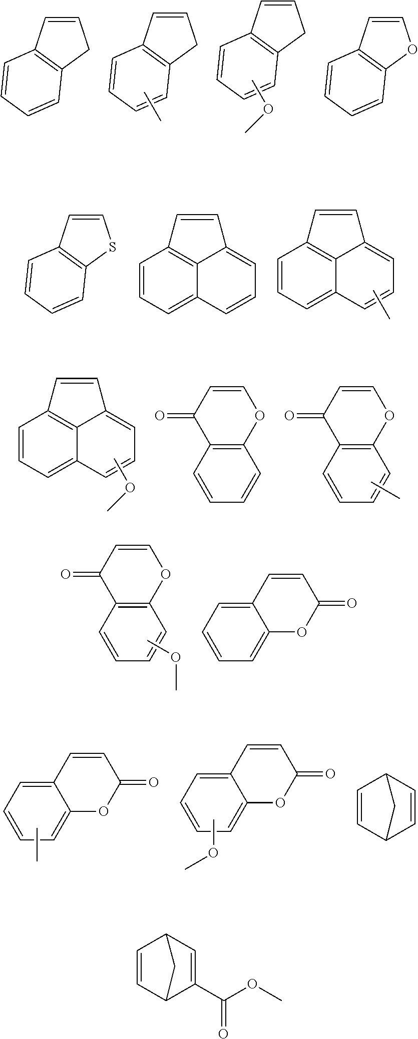

The base polymer may contain repeat units (e) derived from styrene, vinylnaphthalene, vinylanthracene, vinylpyrene, methyleneindane, vinylpyridine, or vinylcarbazole.

The base polymer may contain repeat units (f) derived from an onium salt containing a polymerizable unsaturated bond. JP-A 2005-084365 discloses a sulfonium or iodonium salt containing a polymerizable olefin and generating a specific sulfonic acid. JP-A 2006-178317 discloses a sulfonium salt having a sulfonic acid directly bonded to the backbone.

Examples of the preferred repeat units (f) include repeat units having formula (f1) (hereinafter, the repeat units are also referred to as repeat units (f1)), repeat units having formula (f2) (hereinafter, the repeat units are also referred to as repeat units (f2)), and repeat units having formula (f3) (hereinafter, the repeat units are also referred to as repeat units (f3)). The repeat units (f1) to (f3) may each be used singly or in combination of two or more kinds thereof.

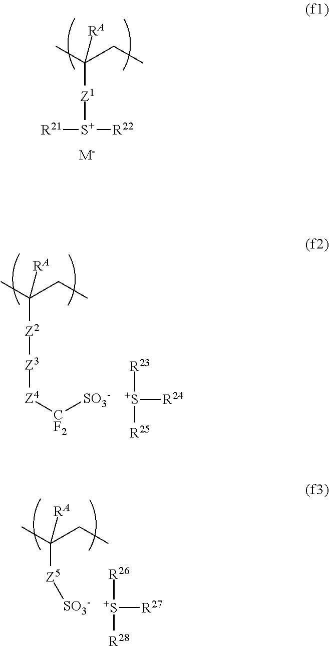

In formulae (f1) to (f3), R^{A} is each independently a hydrogen atom or a methyl group. Z¹ is a single bond, a C₁-C₆ aliphatic hydrocarbylene group, a phenylene group, a naphthylene group, a C₇-C₁₈ group obtained by combining the foregoing, —O—Z¹¹—, —C(═O)—O—Z¹—, or —C(═O)—NH—Z¹¹—. Z¹¹ is a C₁-C₆ aliphatic hydrocarbylene group, a phenylene group, a naphthylene group, or a C₇-C₁₈ group obtained by combining the foregoing, which may contain a carbonyl group, an ester bond, an ether bond, or a hydroxy group. Z² is a single bond or an ester bond. Z³ is a single bond, —Z³¹—C(═O)—O—, —Z³¹—O—, or —Z³¹—O—C(═O)—. Z³¹ is a C₁-C₁₂ aliphatic hydrocarbylene group, a phenylene group, or a C₇-C₁₈ group obtained by combining the foregoing, which may contain a carbonyl group, an ester bond, an ether bond, an iodine atom, or a bromine atom. Z⁴ is a methylene group, a 2,2,2-trifluoro-1,1-ethanediyl group, or a carbonyl group. Z⁵ is a single bond, a methylene group, an ethylene group, a phenylene group, a fluorinated phenylene group, a trifluoromethyl-substituted phenylene group, —O—Z⁵¹—, —C(═O)—O—Z⁵¹—, or —C(═O)—NH—Z⁵¹—. Z⁵¹ is a C₁-C₆ aliphatic hydrocarbylene group, a phenylene group, a fluorinated phenylene group, or a trifluoromethyl-substituted phenylene group, which may contain a carbonyl group, an ester bond, an ether bond, a hydroxy group, or a halogen atom.

In formulae (f1) to (f3), R²¹ to R²⁸ are each independently a halogen atom, or a C₁-C₂₀ hydrocarbyl group which may contain a heteroatom. Examples of the halogen atom include a fluorine atom, a chlorine atom, a bromine atom, and an iodine atom. The hydrocarbyl group may be saturated or unsaturated, and may be straight, branched, or cyclic. Examples thereof include those groups mentioned as the hydrocarbyl group represented by R⁴ to R⁸ in the description of formulae (2) to (3). Some or all of hydrogen atoms of the hydrocarbyl group may be substituted with a group containing a heteroatom such as an oxygen atom, a sulfur atom, a nitrogen atom, or a halogen atom, and some of —CH₂— of the hydrocarbyl group may be substituted with a group containing a heteroatom such as an oxygen atom, a sulfur atom, or a nitrogen atom, so that the group may contain a hydroxy group, a fluorine atom, a chlorine atom, a bromine atom, an iodine atom, a cyano group, a nitro group, a carbonyl group, an ether bond, an ester bond, a sulfonic acid ester bond, a carbonate bond, a lactone ring, a sultone ring, a carboxylic anhydride (—C(═O)—O—C(═O)—), or a haloalkyl group. R²³ and R²⁴ or R²⁶ and R²⁷ may bond together to form a ring together with the sulfur atom to which they are bonded. At this time, examples of the ring include those rings mentioned as the ring in which R⁴ and R⁵ bond together to form a ring together with the sulfur atom to which they are bonded in the description of formula (2).

In formula (f1), M⁻ is a non-nucleophilic counter ion. Examples of the non-nucleophilic counter ion include halide ions such as a chloride ion and a bromide ion; fluoroalkylsulfonate ions such as a triflate ion, a 1,1,1-trifluoroethanesulfonate ion, and a nonafluorobutanesulfonate ion; arylsulfonate ions such as a tosylate ion, a benzenesulfonate ion, a 4-fluorobenzenesulfonate ion, and a 1,2,3,4,5-pentafluorobenzenesulfonate ion; alkylsulfonate ions such as a mesylate ion and a butanesulfonate ion; imide ions such as a bis(trifluoromethylsulfonyl)imide ion, a bis(perfluoroethylsulfonyl)imide ion, and a bis(perfluorobutylsulfonyl)imide ion; and methide ions such as a tris(trifluoromethylsulfonyl)methide ion and a tris(perfluoroethylsulfonyl)methide ion.

Examples of the non-nucleophilic counter ion further include sulfonate ions having a fluorine atom substituted at the α-position as represented by formula (f1-1) and sulfonate ions having a fluorine atom substituted at the α-position and a trifluoromethyl group substituted at the β-position as represented by formula (f1-2).

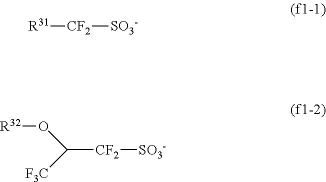

In formula (f1-1), R³¹ is a hydrogen atom, or a C₁-C₂₀ hydrocarbyl group which may contain at least one selected from an ether bond, an ester bond, a carbonyl group, a lactone ring, and a fluorine atom.

In formula (f1-2), R³² is a hydrogen atom, a C₁-C₃₀ hydrocarbyl group, or a C₂-C₃₀ hydrocarbylcarbonyl group, and may contain at least one selected from an ether bond, an ester bond, a carbonyl group, and a lactone ring.

In R³¹ and R³², the hydrocarbyl group and the hydrocarbyl moiety in the hydrocarbylcarbonyl group may be saturated or unsaturated, and may be straight, branched, or cyclic. Examples thereof include alkyl groups such as a methyl group, an ethyl group, a propyl group, an isopropyl group, a butyl group, an isobutyl group, a sec-butyl group, a tert-butyl group, a pentyl group, a neopentyl group, a hexyl group, a heptyl group, a 2-ethylhexyl group, a nonyl group, an undecyl group, a tridecyl group, a pentadecyl group, a heptadecyl group, and an icosyl group; cyclic saturated hydrocarbyl groups such as a cyclopentyl group, a cyclohexyl group, a 1-adamantyl group, a 2-adamantyl group, a 1-adamantylmethyl group, a norbornyl group, a norbornylmethyl group, a tricyclodecanyl group, a tetracyclododecanyl group, a tetracyclododecanylmethyl group, and a dicyclohexylmethyl group; alkenyl groups such as an allyl group; cyclic unsaturated hydrocarbyl groups such as a 3-cyclohexenyl group; aryl groups such as a phenyl group, a 1-naphthyl group, and a 2-naphthyl group; and aralkyl groups such as a benzyl group and a diphenylmethyl group.

Some or all of hydrogen atoms of these groups may be substituted with a heteroatom-containing group such as an oxygen atom, a sulfur atom, a nitrogen atom, or a halogen atom, and some of carbon atoms of these groups may be substituted with a heteroatom-containing group such as an oxygen atom, a sulfur atom, or a nitrogen atom, so that the group may contain a hydroxy group, a cyano group, a carbonyl group, an ether bond, an ester bond, a sulfonic acid ester bond, a carbonate group, a lactone ring, a sultone ring, a carboxylic anhydride, or a haloalkyl group. Examples of the hydrocarbyl group containing a heteroatom include a tetrahydrofuryl group, a methoxymethyl group, an ethoxymethyl group, a methylthiomethyl group, an acetamidomethyl group, a trifluoroethyl group, a (2-methoxyethoxy)methyl group, an acetoxymethyl group, a 2-carboxy-1-cyclohexyl group, a 2-oxopropyl group, a 4-oxo-1-adamantyl group, and a 3-oxocyclohexyl group.

Examples of the cation in the monomer from which the repeat units (f1) are derived include those shown below, but are not limited thereto. In the formulae, R^{A} is as defined above.

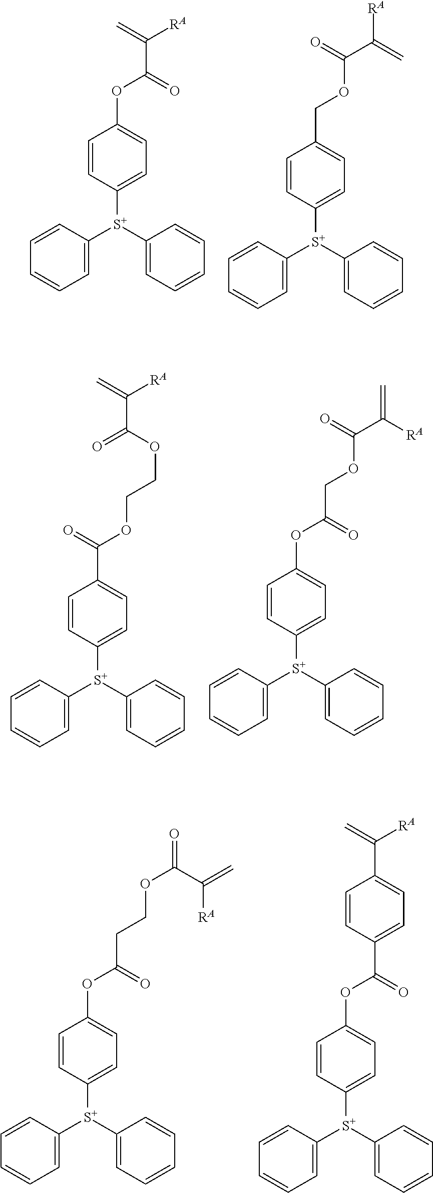

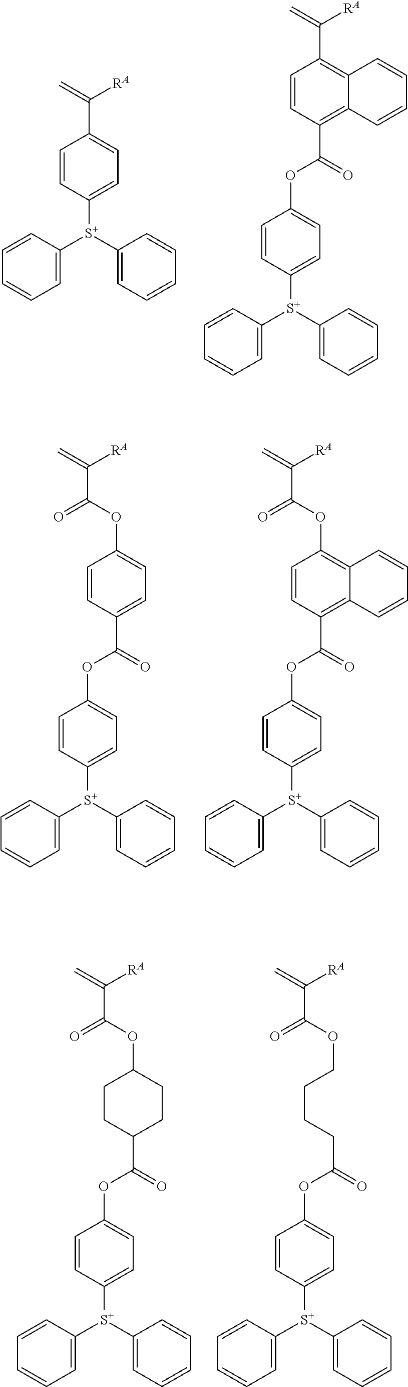

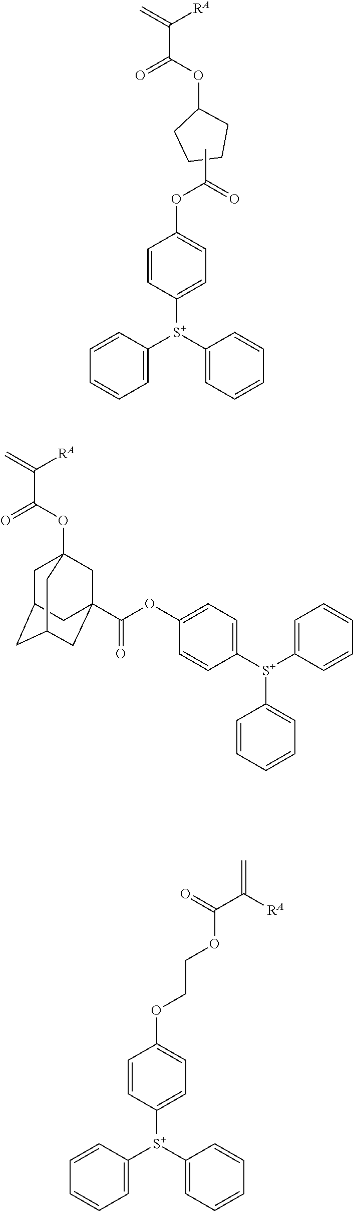

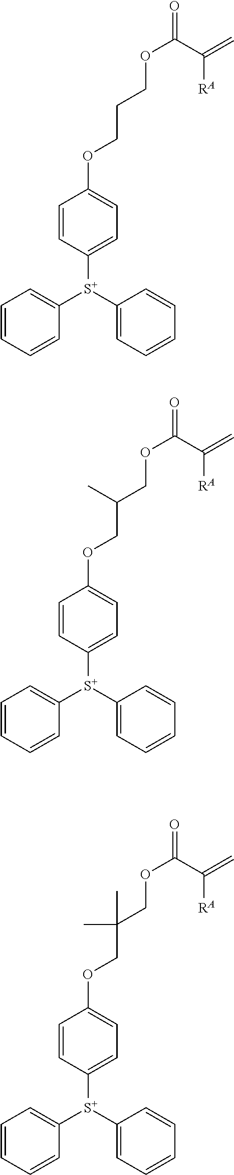

Examples of the cation in the monomer from which the repeat units (f2) or (f3) are derived include those groups mentioned as the sulfonium cation represented by M⁺ in the description of formula (1).

Examples of the monomer from which the repeat units (f2) are derived include those shown below, but are not limited thereto. In the formulae, R^{A} is as defined above.

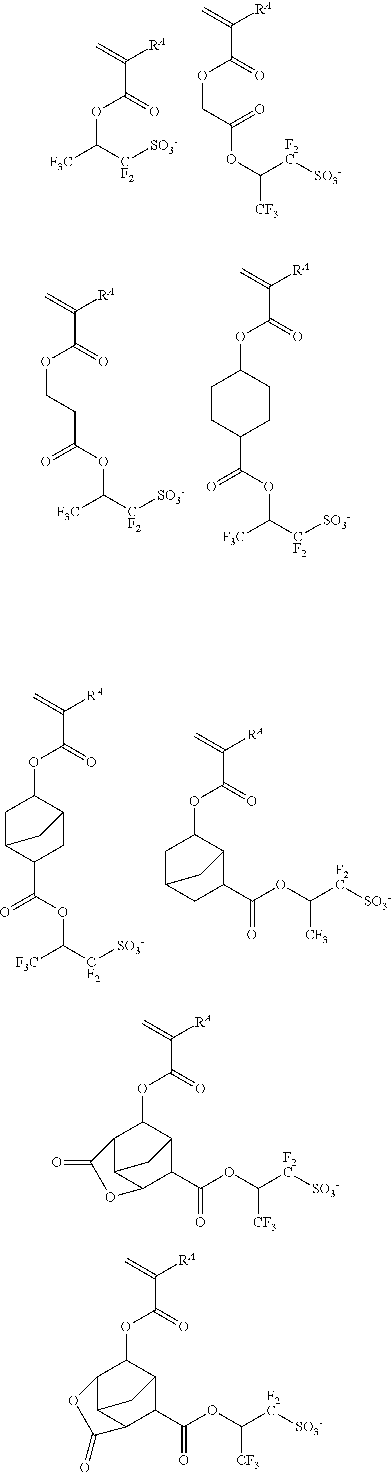

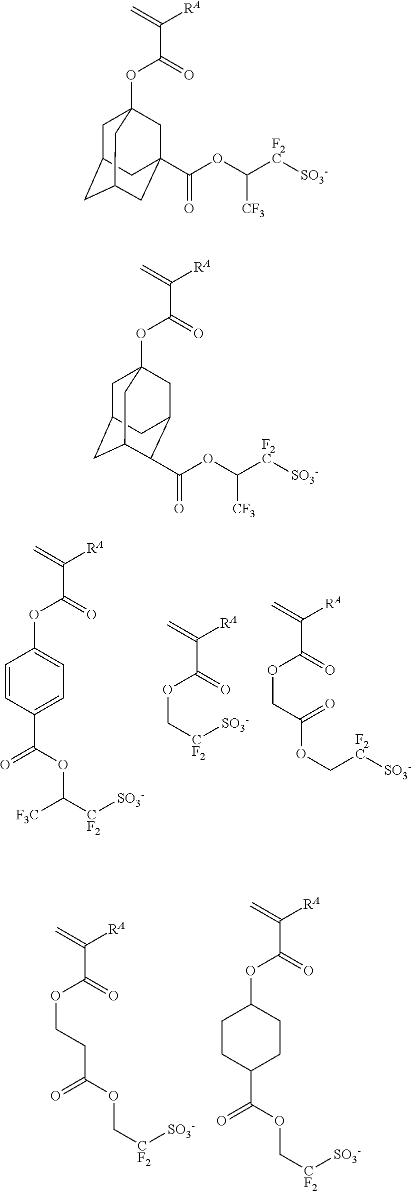

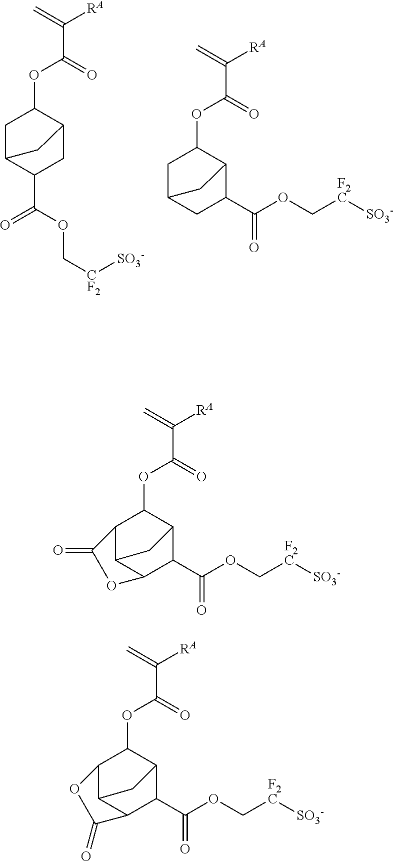

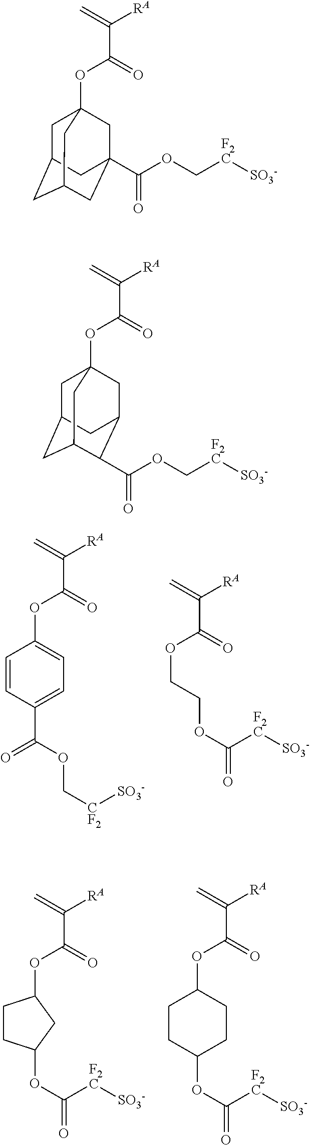

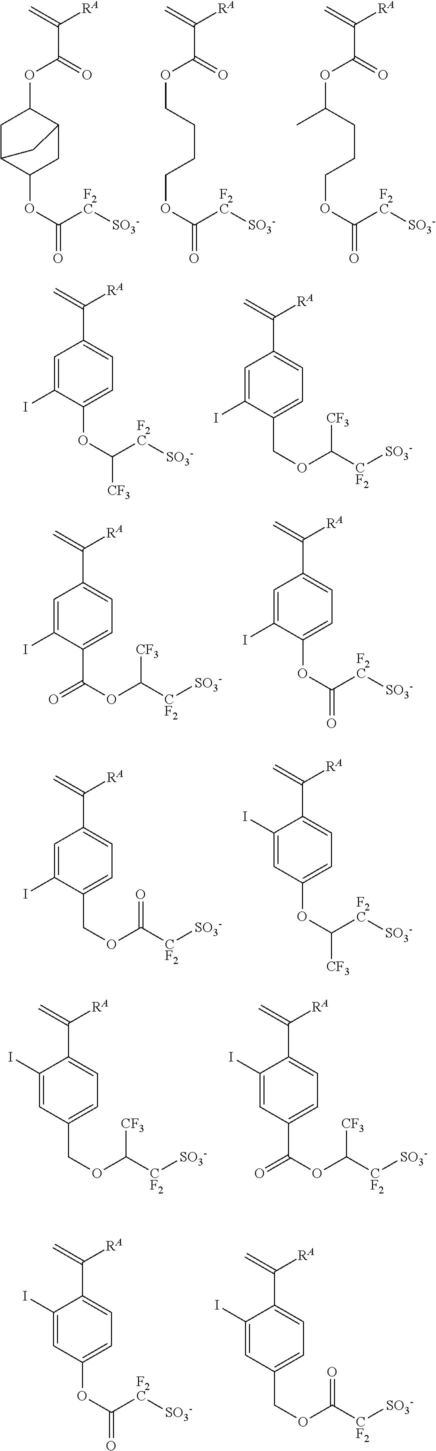

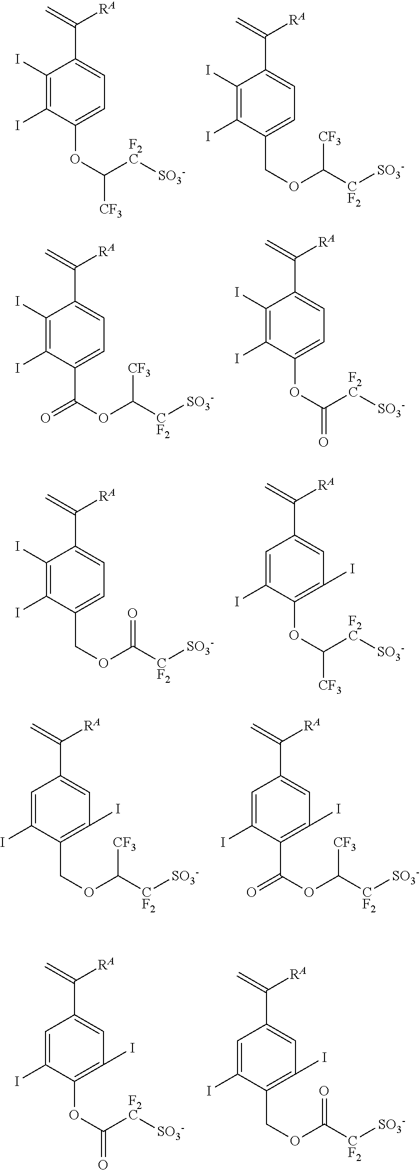

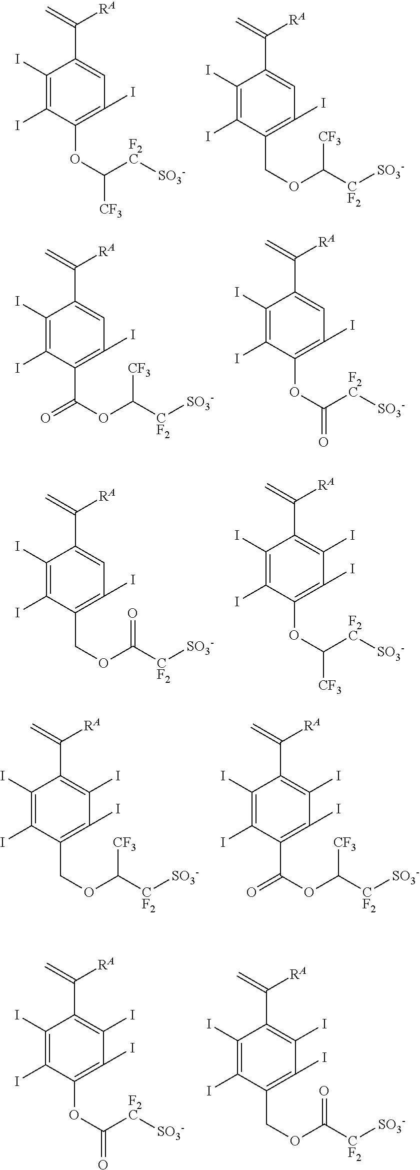

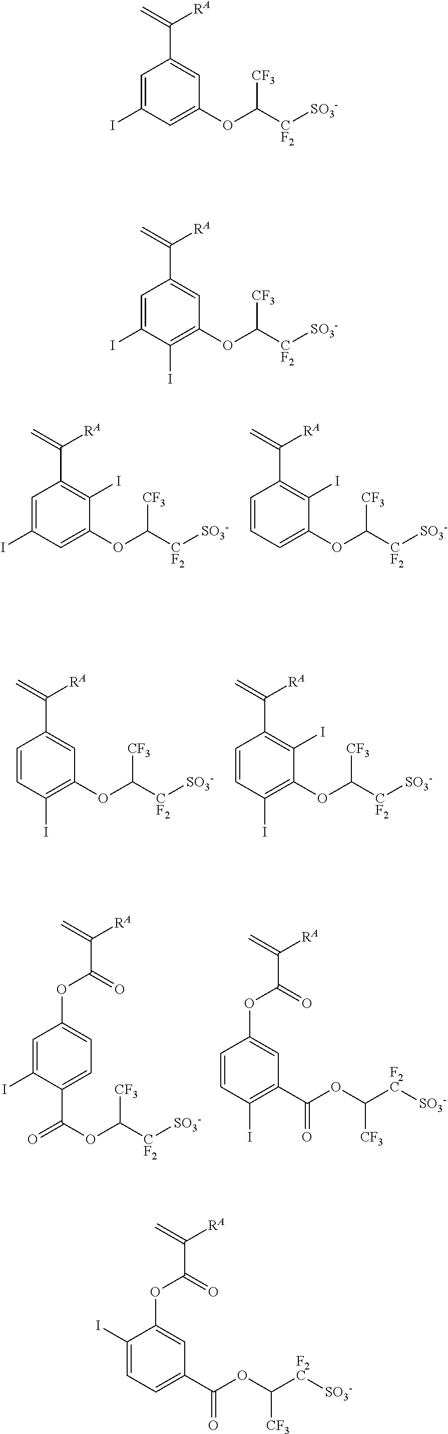

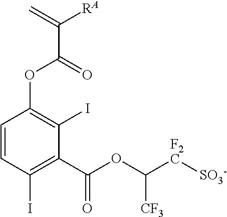

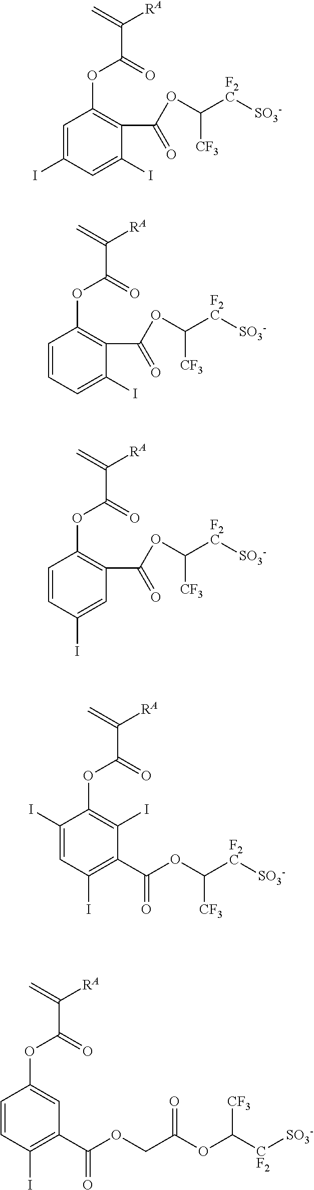

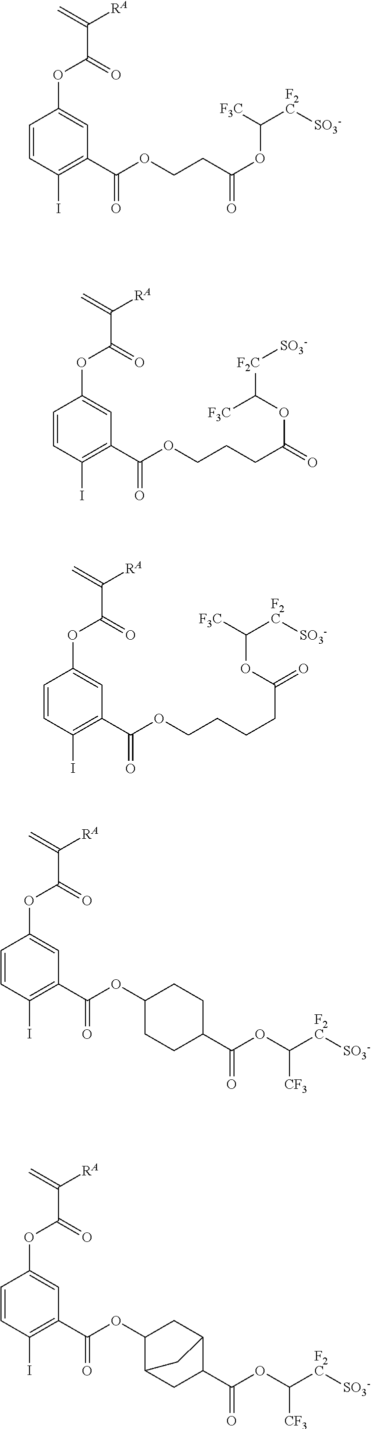

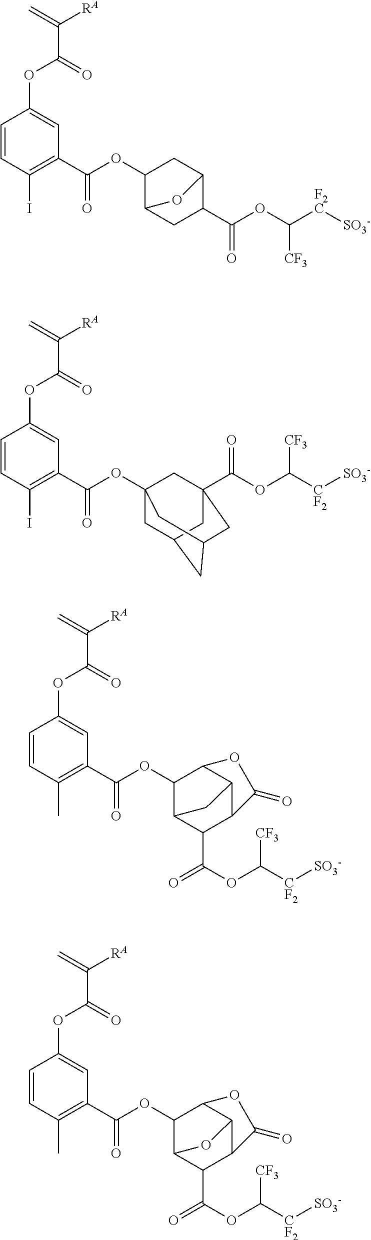

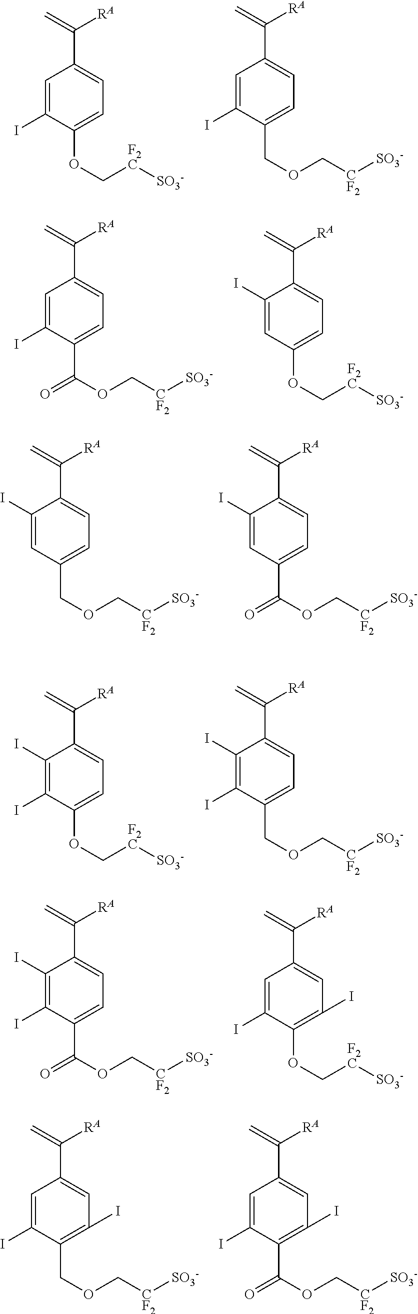

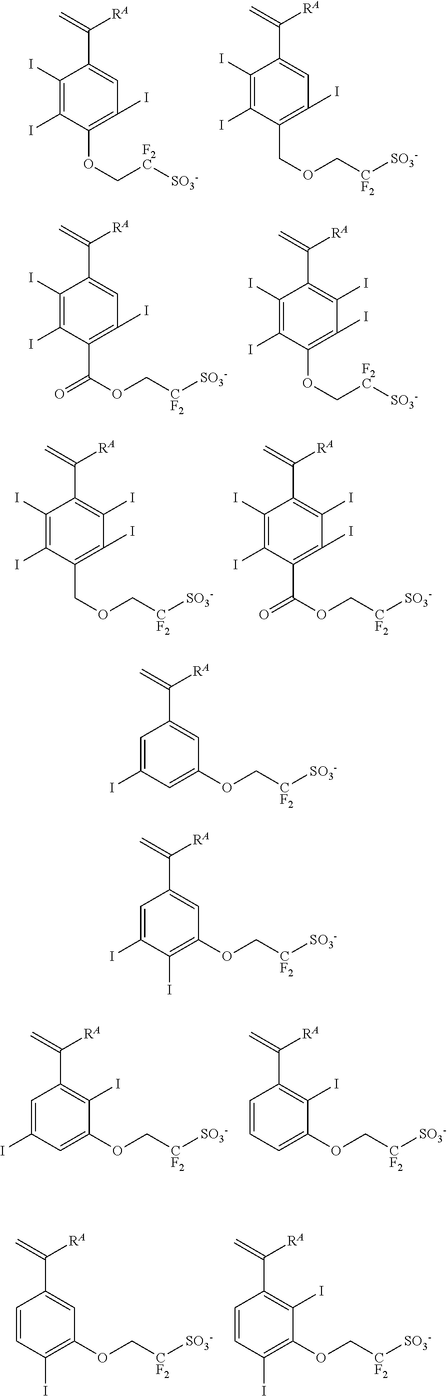

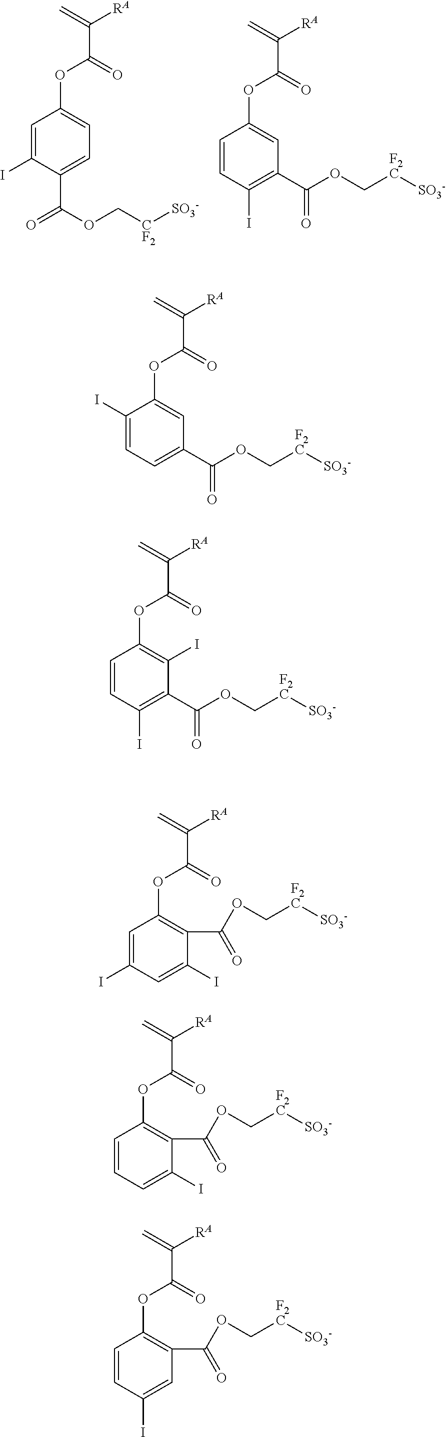

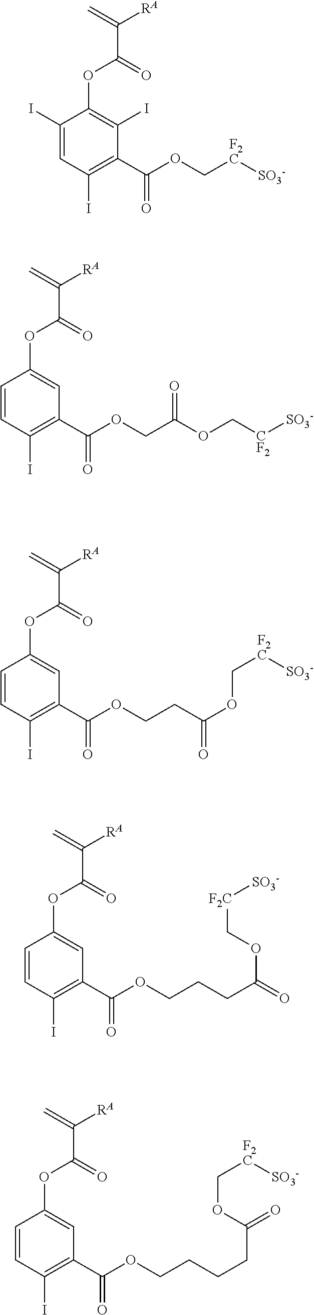

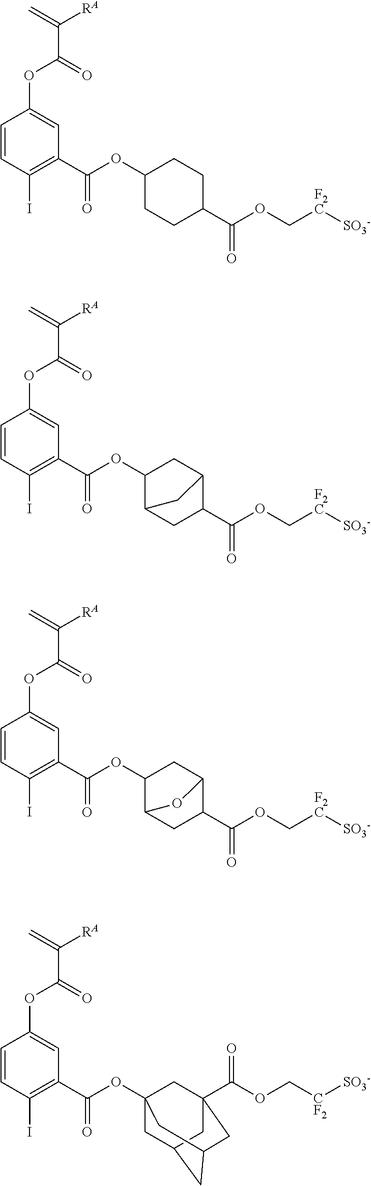

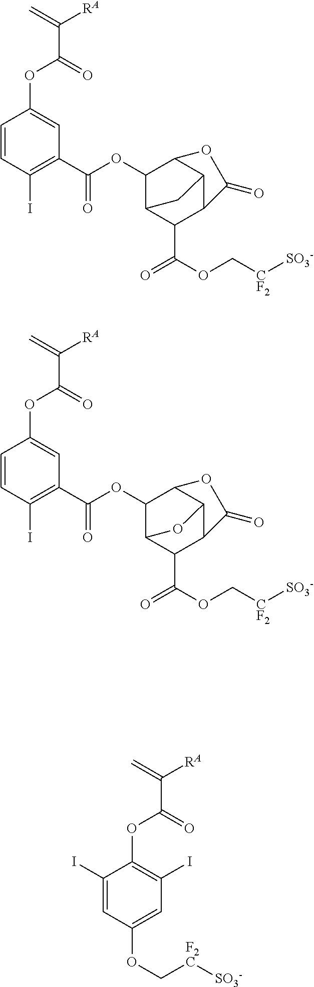

Examples of the monomer from which the repeat units (f3) are derived include those shown below, but are not limited thereto. In the formulae, R^{A} is as defined above.

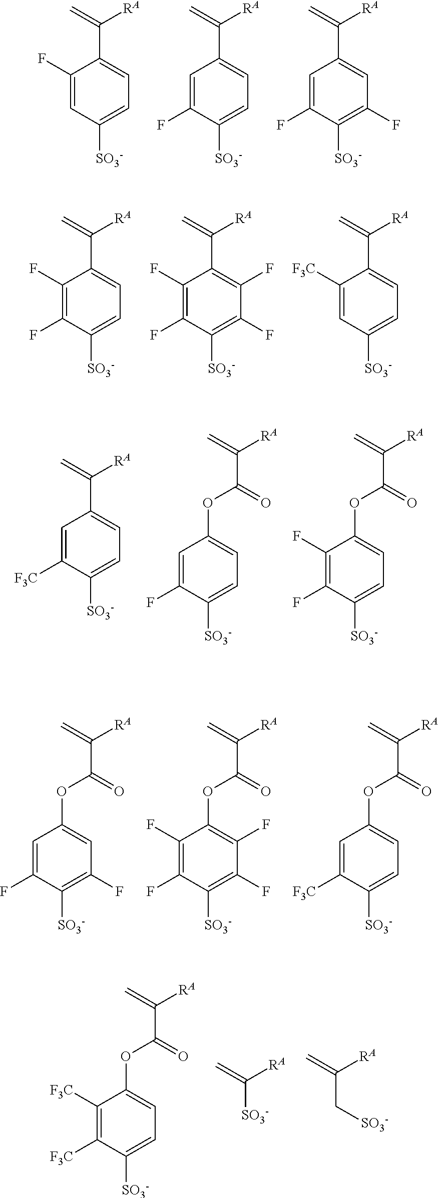

The repeating units (f1) to (f3) function as acid generators. Bonding an acid generator to the polymer backbone is effective in restraining acid diffusion, thereby preventing a reduction of resolution due to blur by acid diffusion. In addition, the LWR and CDU are improved since the acid generator is uniformly distributed.

The base polymer for a positive resist composition essentially contains the repeat units (a1) or (a2) each containing an acid labile group. In this case, the fractions of the repeat units (a1), (a2), (b), (c), (d), (e), and (f) are preferably 0≤(a1)<1.0, 0≤(a2)<1.0, 0<(a1)+(a2)<1.0, 0≤(b)≤0.9, 0≤(c)≤0.9, 0≤(d)≤0.8, 0≤(e)≤0.8, and 0≤(f)≤0.5, more preferably 0≤(a1)≤0.9, 0≤(a2)≤0.9, 0.1≤(a1)+(a2)≤0.9, 0≤(b)≤0.8, 0≤(c)≤0.8, 0≤(d)≤0.7, 0≤(e)≤0.7, and 0≤(f)≤0.4, and even more preferably 0≤(a1)≤0.8, 0≤(a2)≤0.8, 0.1≤(a1)+(a2)≤0.8, 0≤(b)≤0.75, 0≤(c)≤0.75, 0≤(d)≤0.6, 0≤(e)≤0.6, and 0≤(f)≤0.3. The repeat units (f) satisfy (f)=(f1)+(f2)+(f3) when they are at least one selected from the repeat units (f1) to (f3). In addition, (a1)+(a2)+(b)+(c)+(d)+(e)+(f)=1.0.

Meanwhile, the base polymer for a negative resist composition does not necessarily contain an acid labile group. Examples of such a base polymer include those containing the repeat units (b) and further containing the repeat units (c), (d), (e), and/or (f) as necessary. The fractions of these repeat units are preferably 0<(b)≤1.0, 0≤(c)≤0.9, 0≤(d)≤0.8, 0≤(e)≤0.8, and 0≤(f)≤0.5, more preferably 0.2≤(b)≤1.0, 0≤(c)≤0.8, 0≤(d)≤0.7, 0≤(e)≤0.7, and 0≤(f)≤0.4, and even more preferably 0.3≤(b)≤1.0, 0≤(c)≤0.75, 0≤(d)≤0.6, 0≤(e)≤0.6, and 0≤(f)≤0.3. The repeat units (f) satisfy (f)=(f1)+(f2)+(f3) when they are at least one selected from the repeat units (f1) to (f3). In addition, (b)+(c)+(d)+(e)+(f)=1.0.

The base polymer may be synthesized, for example, by heating a monomer from which the repeat units are derived in an organic solvent with the addition of a radical polymerization initiator to perform polymerization.

Examples of the organic solvent used in the polymerization include toluene, benzene, tetrahydrofuran (THF), diethyl ether, and dioxane. Examples of the polymerization initiator include 2,2′-azobisisobutyronitrile (AIBN), 2,2′-azobis(2,4-dimethylvaleronitrile), dimethyl-2,2-azobis(2-methylpropionate), benzoyl peroxide, and lauroyl peroxide. The temperature during polymerization is preferably 50 to 80° C. The reaction time is preferably 2 to 100 hours, and more preferably 5 to 20 hours.

When a monomer having a hydroxy group is copolymerized, the hydroxy group may be substituted with an acetal group susceptible to deprotection with an acid such as an ethoxyethoxy group prior to polymerization, and the polymerization be followed by deprotection with a weak acid and water. Alternatively, the hydroxy group may be substituted with an acetyl group, a formyl group, a pivaloyl group or the like prior to polymerization, and the polymerization be followed by alkaline hydrolysis.

When hydroxystyrene or hydroxyvinylnaphthalene is copolymerized, acetoxystyrene or acetoxyvinylnaphthalene may be used instead of hydroxystyrene or hydroxyvinylnaphthalene, and after polymerization, the acetoxy group may be deprotected by alkaline hydrolysis for converting the polymer product to hydroxystyrene or hydroxyvinylnaphthalene.

For alkaline hydrolysis, a base such as aqueous ammonia or triethylamine may be used. The reaction temperature is preferably −20 to 100° C., and more preferably 0 to 60° C. The reaction time is preferably 0.2 to 100 hours, and more preferably 0.5 to 20 hours.

The base polymer preferably has a weight average molecular weight (Mw) in the range of 1,000 to 500,000, and more preferably 2,000 to 30,000, as measured by gel permeation chromatography (GPC) versus polystyrene standards using a THF solvent. A Mw in the range ensures that the resist film has heat resistance and high solubility in an alkaline developer.

Further, if the base polymer has a wide molecular weight distribution (Mw/Mn), which indicates the presence of lower and higher molecular weight polymer fractions, there is a possibility that foreign matters are left on the pattern or the pattern profile is degraded after exposure. The influences of Mw and Mw/Mn tend to be stronger as the pattern rule becomes finer. Therefore, the base polymer should preferably have a narrow dispersity (Mw/Mn) of 1.0 to 2.0, especially 1.0 to 1.5, in order to provide a resist composition suitable for micropatterning to a small feature size.

The base polymer may contain two or more polymers having different compositional ratios, Mw, and Mw/Mn.

### [Organic Solvent]

The resist composition of the present invention may contain an organic solvent. The organic solvent is not particularly limited as long as the components described above and below are soluble therein. Examples of the organic solvent are described in paragraphs [0144] to [0145] of JP-A 2008-111103, and include ketones such as cyclohexanone, cyclopentanone, methyl-2-n-pentyl ketone, and 2-heptanone; alcohols such as 3-methoxybutanol, 3-methyl-3-methoxybutanol, 1-methoxy-2-propanol, 1-ethoxy-2-propanol, and diacetone alcohol (DAA); ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate (PGMEA), propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; and lactones such as γ-butyrolactone.

The organic solvent is preferably added to the resist composition of the present invention in an amount of 100 to 10,000 parts by weight, and more preferably 200 to 8,000 parts by weight per 100 parts by weight of the base polymer. The organic solvent may be used singly or as a mixture of two or more kinds thereof.

### [Quencher]

The resist composition of the present invention may contain a quencher. The quencher refers to a compound capable of trapping the acid generated from the acid generator in the resist composition to prevent the acid from diffusing to the unexposed region.

Examples of the quencher include conventional basic compounds. Examples of the conventional basic compounds include primary, secondary, and tertiary aliphatic amines, mixed amines, aromatic amines, heterocyclic amines, nitrogen-containing compounds having a carboxy group, nitrogen-containing compounds having a sulfonyl group, nitrogen-containing compounds having a hydroxy group, nitrogen-containing compounds having a hydroxyphenyl group, alcoholic nitrogen-containing compounds, amide derivatives, imide derivatives, and carbamate derivatives. In particular, primary, secondary, and tertiary amine compounds described in paragraphs [0146] to [0164] of JP-A 2008-111103, particularly amine compounds having a hydroxy group, an ether bond, an ester bond, a lactone ring, a cyano group, or a sulfonic acid ester bond, and compounds having a carbamate bond described in JP 3790649 are preferred. Addition of a basic compound may be effective for further reducing the diffusion rate of the acid in the resist film or correcting the pattern profile.

Examples of the quencher also include onium salts such as sulfonium salts, iodonium salts, and ammonium salts of sulfonic acids which are not fluorinated at the α-position, carboxylic acids, or fluorinated alkoxides, as described in JP-A 2008-158339. While a sulfonic acid which is fluorinated at the α-position, imide acid, or methide acid is necessary for deprotecting the acid labile group of a carboxylic acid ester, an α-non-fluorinated sulfonic acid, a carboxylic acid, or fluorinated alcohol is released by salt exchange with the onium salt. The α-non-fluorinated sulfonic acid, carboxylic acid, and fluorinated alcohol function as a quencher because they do not induce deprotection reaction.

Examples of such a quencher include a compound having formula (4) (an onium salt of an α-non-fluorinated sulfonic acid), a compound having formula (5) (an onium salt of a carboxylic acid), and a compound having formula (6) (an onium salt of an alkoxide).

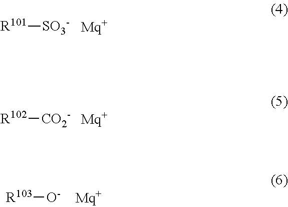

In formula (4), R¹⁰¹ is a hydrogen atom or a C₁-C₄₀ hydrocarbyl group which may contain a heteroatom, exclusive of a hydrocarbyl group in which a hydrogen atom bonded to the carbon atom at the α-position of the sulfo group is substituted with a fluorine atom or a fluoroalkyl group.

The C₁-C₄₀ hydrocarbyl group represented by R¹⁰¹ may be saturated or unsaturated, and may be straight, branched, or cyclic. Examples thereof include C₁-C₄₀ alkyl groups such as a methyl group, an ethyl group, a n-propyl group, an isopropyl group, a n-butyl group, an isobutyl group, a sec-butyl group, a tert-butyl group, a n-pentyl group, a tert-pentyl group, a n-hexyl group, a n-octyl group, a 2-ethylhexyl group, a n-nonyl group, and a n-decyl group; C₃-C₄₀ cyclic saturated hydrocarbyl groups such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.0^{2,6}]decyl group, an adamantyl group, and an adamantylmethyl group; C₂-C₄₀ alkenyl groups such as a vinyl group, an allyl group, a propenyl group, a butenyl group, and a hexenyl group; C₃-C₄₀ cyclic unsaturated aliphatic hydrocarbyl groups such as a cyclohexenyl group; C₆-C₄₀ aryl groups such as a phenyl group, a naphthyl group, alkylphenyl groups (e.g., a 2-methylphenyl group, a 3-methylphenyl group, a 4-methylphenyl group, a 4-ethylphenyl group, a 4-tert-butylphenyl group, and a 4-n-butylphenyl group), di- or tri-alkylphenyl groups (e.g., a 2,4-dimethylphenyl group and a 2,4,6-triisopropylphenyl group), alkylnaphthyl groups (e.g., a methylnaphthyl group and an ethylnaphthyl group), and dialkylnaphthyl groups (e.g., a dimethylnaphthyl group and a diethylnaphthyl group); and C₇-C₄₀ aralkyl groups such as a benzyl group, a 1-phenylethyl group, and a 2-phenylethyl group.

Some or all of hydrogen atoms of the hydrocarbyl group may be substituted with a group containing a heteroatom such as an oxygen atom, a sulfur atom, a nitrogen atom, or a halogen atom, and some of —CH₂— of the hydrocarbyl group may be substituted with a group containing a heteroatom such as an oxygen atom, a sulfur atom, or a nitrogen atom, so that the group may contain a hydroxy group, a cyano group, a carbonyl group, an ether bond, an ester bond, a sulfonic acid ester bond, a carbonate bond, a lactone ring, a sultone ring, a carboxylic anhydride (—C(═O)—O—C(═O)—), or a haloalkyl group. Examples of the hydrocarbyl group containing a heteroatom include heteroaryl groups such as a thienyl group; alkoxyphenyl groups such as a 4-hydroxyphenyl group, a 4-methoxyphenyl group, a 3-methoxyphenyl group, a 2-methoxyphenyl group, a 4-ethoxyphenyl group, a 4-tert-butoxyphenyl group, and a 3-tert-butoxyphenyl group; alkoxynaphthyl groups such as a methoxynaphthyl group, an ethoxynaphthyl group, a n-propoxynaphthyl group, and a n-butoxynaphthyl group; dialkoxynaphthyl groups such as a dimethoxynaphthyl group and a diethoxynaphthyl group; and aryloxoalkyl groups, typically 2-aryl-2-oxoethyl groups such as a 2-phenyl-2-oxoethyl group, a 2-(1-naphthyl)-2-oxoethyl group, and a 2-(2-naphthyl)-2-oxoethyl group.

In formula (5), R¹⁰² is a C₁-C₄₀ hydrocarbyl group which may contain a heteroatom. Examples of the hydrocarbyl group represented by R¹⁰² include those groups mentioned as the hydrocarbyl group represented by R¹⁰¹. Also included are fluorinated alkyl groups such as a trifluoromethyl group, a trifluoroethyl group, a 2,2,2-trifluoro-1-methyl-1-hydroxyethyl group, and a 2,2,2-trifluoro-1-(trifluoromethyl)-1-hydroxyethyl group; and fluorinated aryl groups such as a pentafluorophenyl group and a 4-trifluoromethylphenyl group.

In formula (6), R¹⁰³ is a C₁-C₈ saturated hydrocarbyl group containing at least 3 fluorine atoms or a C₆-C₁₀ aryl group containing at least 3 fluorine atoms, and may contain a nitro group.

In formulae (4), (5), and (6), Mq⁺ is an onium cation. The onium cation is preferably a sulfonium cation, an iodonium cation, or an ammonium cation, and more preferably a sulfonium cation. Examples of the sulfonium cation include those groups mentioned as the sulfonium cation represented by M⁺ in the description of formula (1).

A sulfonium salt of iodized benzene ring-containing carboxylic acid having formula (7) is also suitable for the quencher.

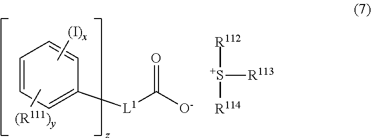

In formula (7), x is an integer of 1 to 5. y is an integer of 0 to 3. z is an integer of 1 to 3.

In formula (7), R¹¹¹ is a hydroxy group, a fluorine atom, a chlorine atom, a bromine atom, an amino group, a nitro group, a cyano group, or a C₁-C₆ saturated hydrocarbyl group, a C₁-C₆ saturated hydrocarbyloxy group, a C₂-C₆ saturated hydrocarbylcarbonyloxy group, or a C₁-C₄ saturated hydrocarbylsulfonyloxy group, in which some or all hydrogen atoms may be substituted with a halogen atom, —N(R^{111A})—C(═O)—R^{111B}, or N(R^{111A})—C(═O)—O—R^{111B}, wherein R^{111A} is a hydrogen atom or a C₁-C₆ saturated hydrocarbyl group, and R^{111B} is a C₁-C₆ saturated hydrocarbyl group or a C₂-C₈ unsaturated aliphatic hydrocarbyl group. Groups R¹¹¹ may be identical or different when y and/or z is 2 or more.

In formula (7), L¹ is a single bond, or a C₁-C₂₀ (z+1)-valent linking group which may contain at least one moiety selected from an ether bond, a carbonyl group, an ester bond, an amide bond, a sultone ring, a lactam ring, a carbonate bond, a halogen atom, a hydroxy group, and a carboxy group. The saturated hydrocarbyl group, the saturated hydrocarbyloxy group, the saturated hydrocarbylcarbonyloxy group, and the saturated hydrocarbylsulfonyloxy group may be straight, branched, or cyclic.

In formula (7), R¹¹², R¹¹³, and R¹¹⁴ are each independently a halogen atom, or a C₁-C₂₀ hydrocarbyl group which may contain a heteroatom. The hydrocarbyl group may be saturated or unsaturated, and may be straight, branched, or cyclic. Examples thereof include those groups mentioned as the hydrocarbyl group represented by R⁴ to R⁸ in the description of formulae (2) to (3).

Examples of the compound having formula (7) include compounds described in JP-A 2017-219836 and JP-A 2021-091666.

Another example of the quencher is a polymeric quencher described in JP-A 2008-239918. The polymeric quencher segregates at the resist film surface and thus enhances the rectangularity of the resist pattern. When a protective film is applied as is often the case in immersion lithography, the polymeric quencher is also effective for preventing a film thickness loss of the resist pattern or rounding of the pattern top.

Other useful quenchers include sulfonium salts of betaine structure as described in JP 6848776 and JP-A 2020-037544, fluorine-free methide acids as described in JP-A 2020-055797, sulfonium salts of sulfonamide as described in JP 5807552, sulfonium salts of iodized sulfonamide as described in JP-A 2019-211751, and an acid generator that generates phenol, halogen, and carbonic acid.

The quencher is preferably added to the resist composition of the present invention in an amount of 0 to 5 parts by weight, and more preferably 0 to 4 parts by weight per 100 parts by weight of the base polymer. The quencher may be used singly or in combination of two or more kinds thereof.

### [Other Components]

In addition to the foregoing components, the resist composition of the present invention may contain an acid generator other than the salt having formula (1) (hereinafter referred to as another acid generator), a surfactant, a dissolution inhibitor, a crosslinker, a water repellency improver, and an acetylene alcohol.

Examples of the other acid generator include a compound that generates an acid in response to actinic rays or radiation (the compound is referred to as photoacid generator, PAG). Although the PAG may be any compound capable of generating an acid upon exposure to high-energy radiation, those compounds capable of generating a sulfonic acid, imide acid (imidic acid), or methide acid are preferred. Suitable PAGs include sulfonium salts, iodonium salts, sulfonyldiazomethane, N-sulfonyloxyimide, and oxime-O-sulfonate acid generators. Examples of the acid generator include those described in paragraphs [0122] to [0142] of JP-A 2008-111103, JP-A 2018-005224, and JP-A 2018-025789. The other acid generator is preferably added to the resist composition of the present invention in an amount of 0 to 200 parts by weight, and more preferably 0.1 to 100 parts by weight per 100 parts by weight of the base polymer.

Examples of the surfactant include those described in paragraphs [0165] to [0166] of JP-A 2008-111103. Addition of a surfactant may improve or control the coating characteristics of the resist composition. The surfactant is preferably added to the resist composition of the present invention in an amount of 0.0001 to 10 parts by weight per 100 parts by weight of the base polymer. The surfactant may be used singly or in combination of two or more kinds thereof.

In the embodiment wherein the resist composition of the present invention is of positive tone, the addition of a dissolution inhibitor may lead to an increased difference in dissolution rate between the exposed region and the unexposed region and a further improvement in resolution. Examples of the dissolution inhibitor include a compound having at least two phenolic hydroxy groups in the molecule, in which 0 to 100 mol % of all the hydrogen atoms in the phenolic hydroxy groups are substituted with acid labile groups or a compound having at least one carboxy group in the molecule, in which an average of 50 to 100 mol % of all the hydrogen atoms in the carboxy group are substituted with acid labile groups, both the compounds preferably having a molecular weight of 100 to 1,000, and more preferably 150 to 800. Examples thereof include bisphenol A, trisphenol, phenolphthalein, cresol novolac, naphthalenecarboxylic acid, adamantanecarboxylic acid, and cholic acid derivatives, in which the hydrogen atom in the hydroxy or carboxy group is substituted with an acid labile group, as described in paragraphs [0155] to [0178] of JP-A 2008-122932.

The dissolution inhibitor is preferably added to the resist composition of positive tone of the present invention in an amount of 0 to 50 parts by weight, and more preferably 5 to 40 parts by weight per 100 parts by weight of the base polymer. The dissolution inhibitor may be used singly or in combination of two or more kinds thereof.

When the resist composition of the present invention is of negative tone, a negative pattern can be obtained by adding a crosslinker to reduce the dissolution rate of a resist film in the exposed region. Examples of the crosslinker include epoxy compounds, melamine compounds, guanamine compounds, glycoluril compounds, and urea compounds having substituted thereon at least one group selected from a methylol group, an alkoxymethyl group, and an acyloxymethyl group, isocyanate compounds, azide compounds, and compounds having a double bond such as an alkenyloxy group. These compounds may be used as an additive or introduced into a polymer side chain as a pendant group. In addition, a compound containing a hydroxy group can also be used as a crosslinker.

Examples of the epoxy compound include tris(2,3-epoxypropyl) isocyanurate, trimethylolmethane triglycidyl ether, trimethylolpropane triglycidyl ether, and triethylolethane triglycidyl ether.

Examples of the melamine compound include hexamethylol melamine, hexamethoxymethyl melamine, hexamethylol melamine compounds having 1 to 6 methylol groups methoxymethylated and mixtures thereof, hexamethoxyethyl melamine, hexaacyloxymethyl melamine, and hexamethylol melamine compounds having 1 to 6 methylol groups acyloxymethylated and mixtures thereof.

Examples of the guanamine compound include tetramethylol guanamine, tetramethoxymethyl guanamine, tetramethylol guanamine compounds having 1 to 4 methylol groups methoxymethylated and mixtures thereof, tetramethoxyethyl guanamine, tetraacyloxyguanamine, and tetramethylol guanamine compounds having 1 to 4 methylol groups acyloxymethylated and mixtures thereof.

Examples of the glycoluril compound include tetramethylol glycoluril, tetramethoxy glycoluril, tetramethoxymethyl glycoluril, tetramethylol glycoluril compounds having 1 to 4 methylol groups methoxymethylated and mixtures thereof, and tetramethylol glycoluril compounds having 1 to 4 methylol groups acyloxymethylated and mixtures thereof. Examples of the urea compound include tetramethylol urea, tetramethoxymethyl urea, tetramethylol urea compounds having 1 to 4 methylol groups methoxymethylated and mixtures thereof, and tetramethoxyethyl urea.

Examples of the isocyanate compound include tolylene diisocyanate, diphenylmethane diisocyanate, hexamethylene diisocyanate, and cyclohexane diisocyanate.

Examples of the azide compound include 1,1′-biphenyl-4,4′-bisazide, 4,4′-methylidenebisazide, and 4,4′-oxybisazide.

Examples of the alkenyloxy group-containing compound include ethylene glycol divinyl ether, triethylene glycol divinyl ether, 1,2-propanediol divinyl ether, 1,4-butanediol divinyl ether, tetramethylene glycol divinyl ether, neopentyl glycol divinyl ether, trimethylol propane trivinyl ether, hexanediol divinyl ether, 1,4-cyclohexanediol divinyl ether, pentaerythritol trivinyl ether, pentaerythritol tetravinyl ether, sorbitol tetravinyl ether, sorbitol pentavinyl ether, and trimethylol propane trivinyl ether.

The crosslinker is preferably added to the resist composition of negative tone of the present invention in an amount of 0.1 to 50 parts by weight, and more preferably 1 to 40 parts by weight per 100 parts by weight of the base polymer. The crosslinker may be used singly or in combination of two or more kinds thereof.

The water repellency improver improves the water repellency of the surface of the resist film, and can be used in the topcoatless immersion lithography. Examples of preferred water repellency improvers include polymers having a fluoroalkyl group and polymers having a specific structure with a 1,1,1,3,3,3-hexafluoro-2-propanol residue, and those described in JP-A 2007-297590 and JP-A 2008-111103, for example, are preferred. The water repellency improver should be soluble in alkaline developers and organic solvent developers. The water repellency improver having a specific structure with a 1,1,1,3,3,3-hexafluoro-2-propanol residue is well soluble in the developer. A polymer having an amino group or amine salt copolymerized as repeat units may serve as the water repellency improver and is effective for preventing evaporation of the acid during PEB, thus preventing any hole pattern opening failure after development. The water repellency improver is preferably added to the resist composition of the present invention in an amount of 0 to 20 parts by weight, and more preferably 0.5 to 10 parts by weight per 100 parts by weight of the base polymer. The water repellency improver may be used singly or in combination of two or more kinds thereof.

Examples of the acetylene alcohol include those described in paragraphs [0179] to [0182] of JP-A 2008-122932. The acetylene alcohol is preferably added to the resist composition of the present invention in an amount of 0 to 5 parts by weight per 100 parts by weight of the base polymer. The acetylene alcohol may be used singly or in combination of two or more kinds thereof.

### [Pattern Forming Process]

The resist composition of the present invention is used in the fabrication of various integrated circuits by a well-known lithography technique. Examples of the pattern forming process include a process comprising the steps of: applying the resist composition onto a substrate to form a resist film thereon, exposing the resist film to high-energy radiation, and developing the exposed resist film in a developer.

The resist composition of the present invention is first applied onto a substrate on which an integrated circuit is to be formed (e.g., Si, SiO₂, SiN, SiON, TiN, WSi, BPSG, SOG, or organic antireflective coating) or a substrate on which a mask circuit is to be formed (e.g., Cr, CrO, CrON, MoSi₂, or SiO₂) by a suitable coating technique such as spin coating, roll coating, flow coating, dipping, spraying, or doctor coating so that the coating may have a thickness of 0.01 to 2 μm. The coating is prebaked on a hotplate preferably at a temperature of 60 to 150° C. for 10 seconds to 30 minutes, and more preferably at 80 to 120° C. for 30 seconds to 20 minutes to form a resist film.

The resist film is then exposed to high-energy radiation. Examples of the high-energy radiation include UV, deep-UV, EB, EUV having a wavelength of 3 to 15 nm, X-rays, soft X-rays, excimer laser, γ-rays, and synchrotron radiation. When UV, deep-UV, EUV, X-rays, soft X-rays, excimer laser, γ-rays, or synchrotron radiation is used as the high-energy radiation, the resist film is exposed thereto directly or through a mask having a desired pattern preferably at a dose of about 1 to 200 mJ/cm², and more preferably about 10 to 100 mJ/cm². When EB is used as the high-energy radiation, the resist film is exposed thereto directly or through a mask having a desired pattern preferably at a dose of about 0.1 to 300 μC/cm², and more preferably about 0.5 to 200 μC/cm². It is appreciated that the resist composition of the present invention is suitable for micropatterning using high-energy radiation such as KrF excimer laser, ArF excimer laser, EB, EUV, X-rays, soft X-rays, 7-rays, or synchrotron radiation, especially for micropatterning using EB or EUV.

After the exposure, the resist film may be baked (PEB) on a hotplate or in an oven preferably at 30 to 150° C. for 10 seconds to 30 minutes, and more preferably at 50 to 120° C. for 30 seconds to 20 minutes.

After the exposure or PEB, the resist film is developed in a developer in the form of an alkaline aqueous solution for 3 seconds to 3 minutes, preferably 5 seconds to 2 minutes by conventional techniques such as dip, puddle, and spray techniques to form a desired pattern. A preferred developer is a 0.1 to 10 wt %, preferably 2 to 5 wt % aqueous solution of tetramethylammonium hydroxide (TMAH), tetraethylammonium hydroxide, tetrapropylammonium hydroxide, or tetrabutylammonium hydroxide. In the case of a positive resist composition, the resist film in the exposed region is dissolved in the developer whereas the resist film in the unexposed region is not dissolved. In this way, the desired positive pattern is formed on the substrate. In the case of a negative resist composition, inversely the resist film in the exposed region is insolubilized in the developer whereas the resist film in the unexposed region is dissolved.

A negative pattern can be obtained from the positive resist composition comprising a base polymer containing an acid labile group by effecting organic solvent development. Examples of the developer used herein include 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, benzyl formate, phenylethyl formate, methyl 3-phenylpropionate, benzyl propionate, ethyl phenylacetate, and 2-phenylethyl acetate. These organic solvent may be used singly or as a mixture of two or more kinds thereof.

At the end of development, the resist film is rinsed. The rinsing liquid is preferably a solvent which is miscible with the developer and does not dissolve the resist film. Examples of preferred solvents include alcohols having 3 to 10 carbon atoms, ether compounds having 8 to 12 carbon atoms, alkanes, alkenes, and alkynes each having 6 to 12 carbon atoms, and aromatic solvents.

Examples of the alcohols having 3 to 10 carbon atoms include n-propyl alcohol, isopropyl alcohol, 1-butyl alcohol, 2-butyl alcohol, isobutyl alcohol, tert-butyl alcohol, 1-pentanol, 2-pentanol, 3-pentanol, tert-pentyl alcohol, neopentyl alcohol, 2-methyl-1-butanol, 3-methyl-1-butanol, 3-methyl-3-pentanol, cyclopentanol, 1-hexanol, 2-hexanol, 3-hexanol, 2,3-dimethyl-2-butanol, 3,3-dimethyl-1-butanol, 3,3-dimethyl-2-butanol, 2-ethyl-1-butanol, 2-methyl-1-pentanol, 2-methyl-2-pentanol, 2-methyl-3-pentanol, 3-methyl-1-pentanol, 3-methyl-2-pentanol, 3-methyl-3-pentanol, 4-methyl-1-pentanol, 4-methyl-2-pentanol, 4-methyl-3-pentanol, cyclohexanol, and 1-octanol.

Examples of the ether compounds having 8 to 12 carbon atoms include di-n-butyl ether, diisobutyl ether, di-sec-butyl ether, di-n-pentyl ether, diisopentyl ether, di-sec-pentyl ether, di-tert-pentyl ether, and di-n-hexyl ether.

Examples of the alkanes having 6 to 12 carbon atoms include hexane, heptane, octane, nonane, decane, undecane, dodecane, methylcyclopentane, dimethylcyclopentane, cyclohexane, methylcyclohexane, dimethylcyclohexane, cycloheptane, cyclooctane, and cyclononane. Examples of the alkenes having 6 to 12 carbon atoms include hexene, heptene, octene, cyclohexene, methylcyclohexene, dimethylcyclohexene, cycloheptene, and cyclooctene. Examples of the alkynes having 6 to 12 carbon atoms include hexyne, heptyne, and octyne.

Examples of the aromatic solvents include toluene, xylene, ethylbenzene, isopropylbenzene, tert-butylbenzene, and mesitylene.

Rinsing is effective for reducing the resist pattern collapse and defect formation. However, rinsing is not essential. If rinsing is omitted, the amount of solvent used may be reduced.

A hole or trench pattern after development may be shrunk by the thermal flow, RELACS®, or DSA process. A hole pattern is shrunk by applying a shrink agent thereto, and baking the resist composition such that the shrink agent may undergo crosslinking at the resist film surface due to diffusion of the acid catalyst from the resist film during baking, and the shrink agent may attach to the sidewall of the hole pattern. The baking temperature is preferably 70 to 180° C., more preferably 80 to 170° C., and the baking time is preferably 10 to 300 seconds to remove the excess shrink agent and shrink the hole pattern.

### EXAMPLES

Hereinafter, the present invention is specifically described with reference to Synthesis Examples, Examples, and Comparative Examples, but the present invention is not limited to the following Examples.

Structures of acid generators of a sulfonium salt or iodonium salt PAG-1 to PAG-26 used in resist compositions are shown below.

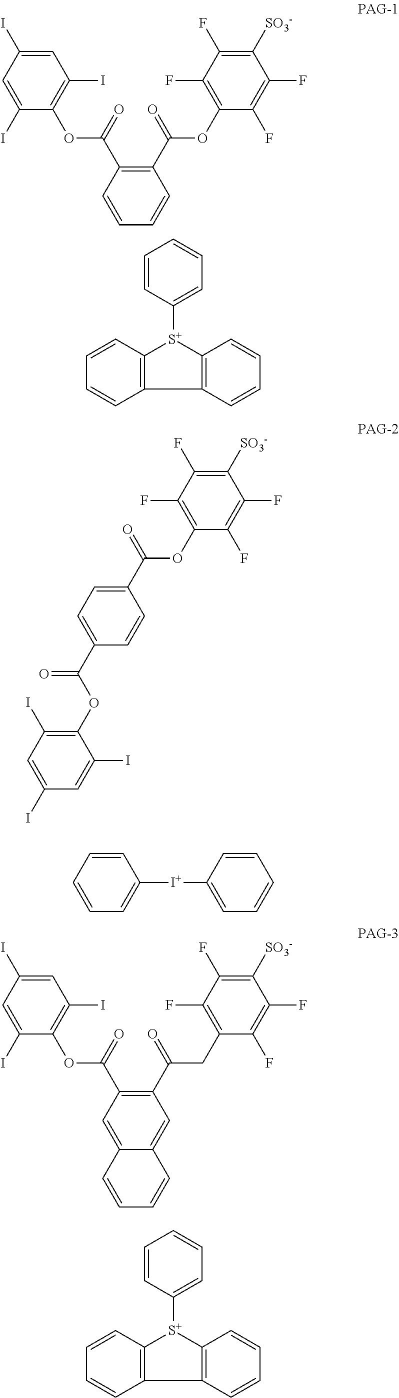

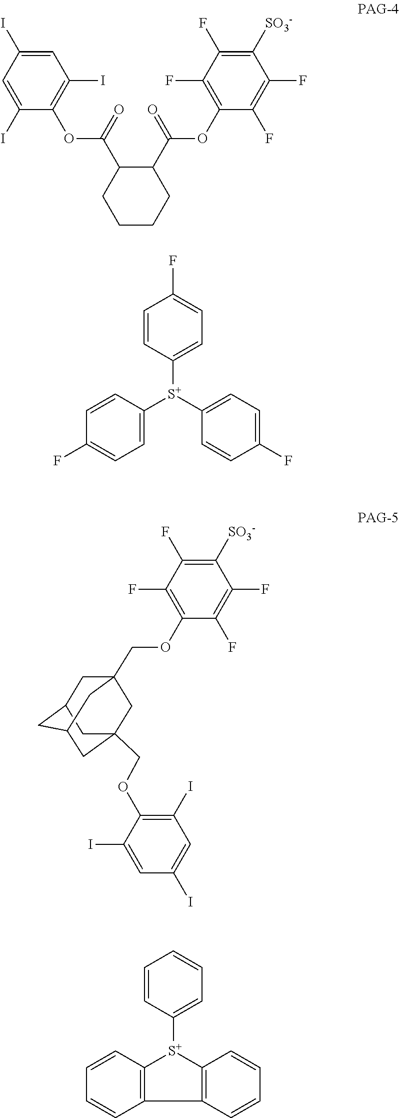

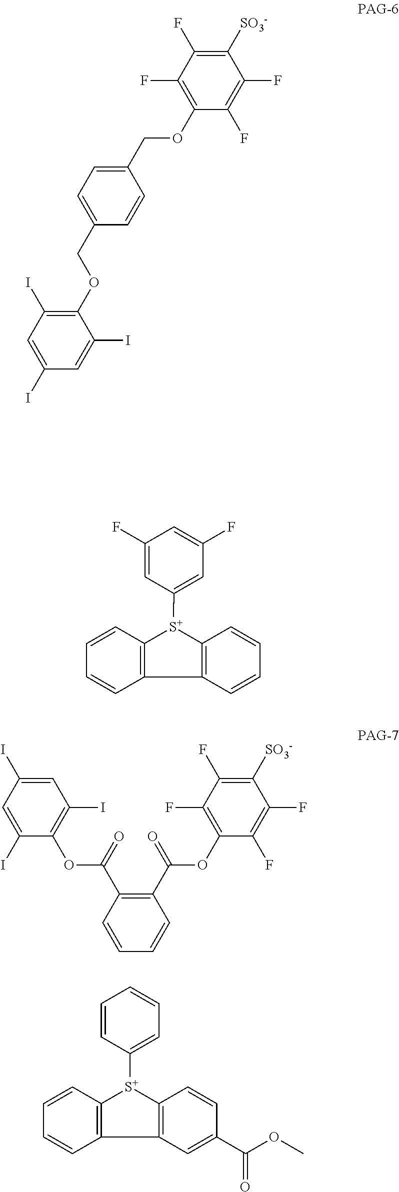

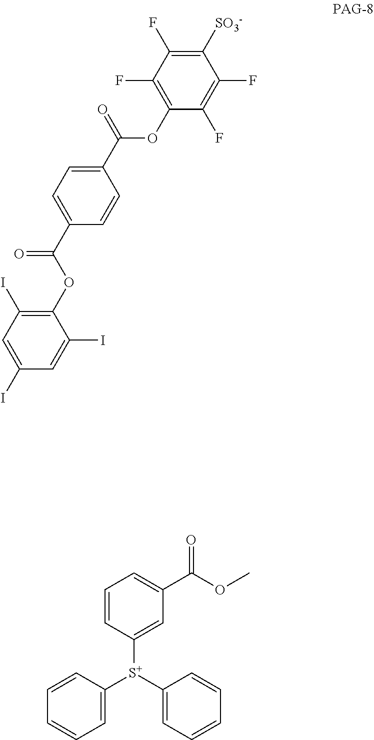

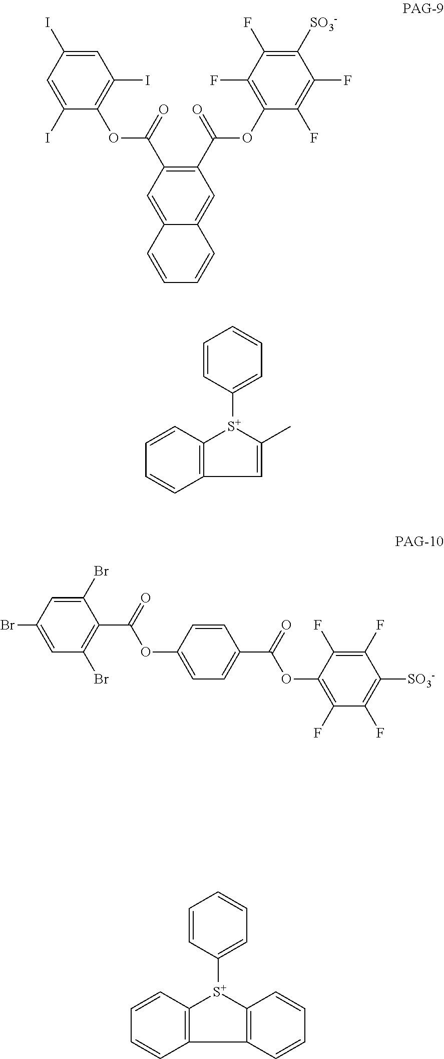

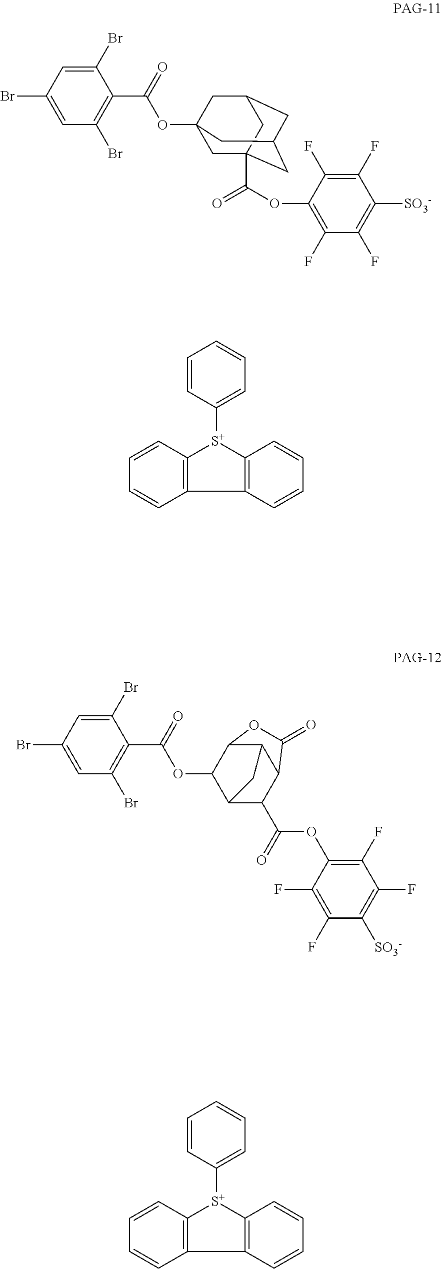

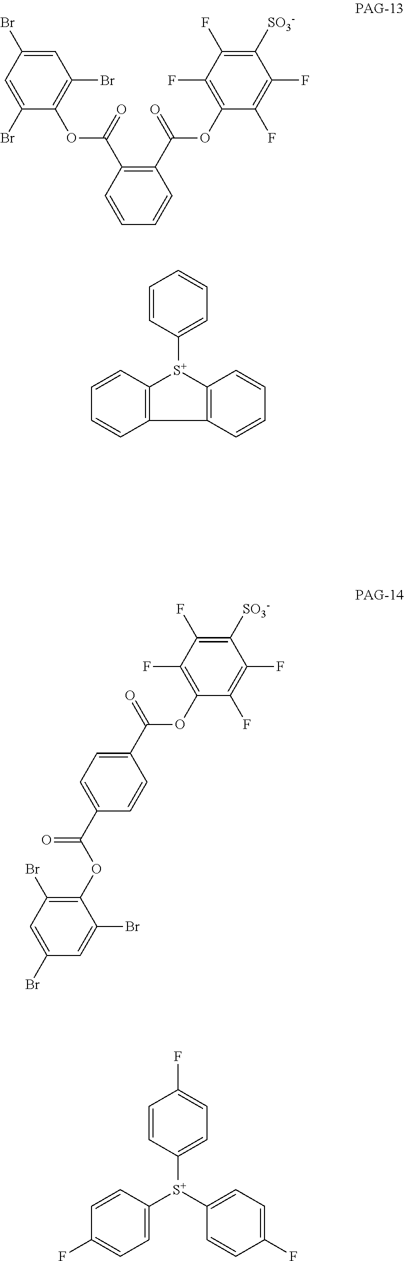

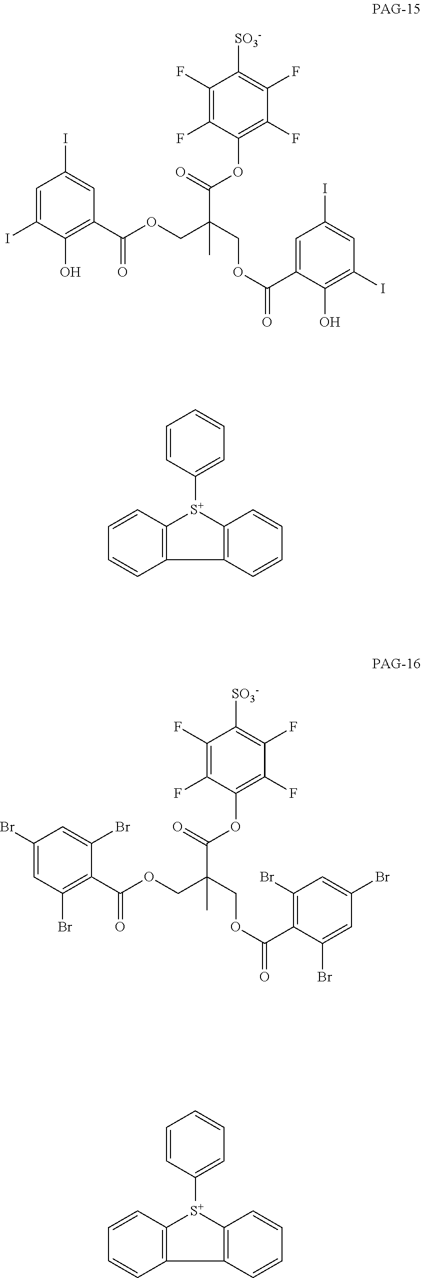

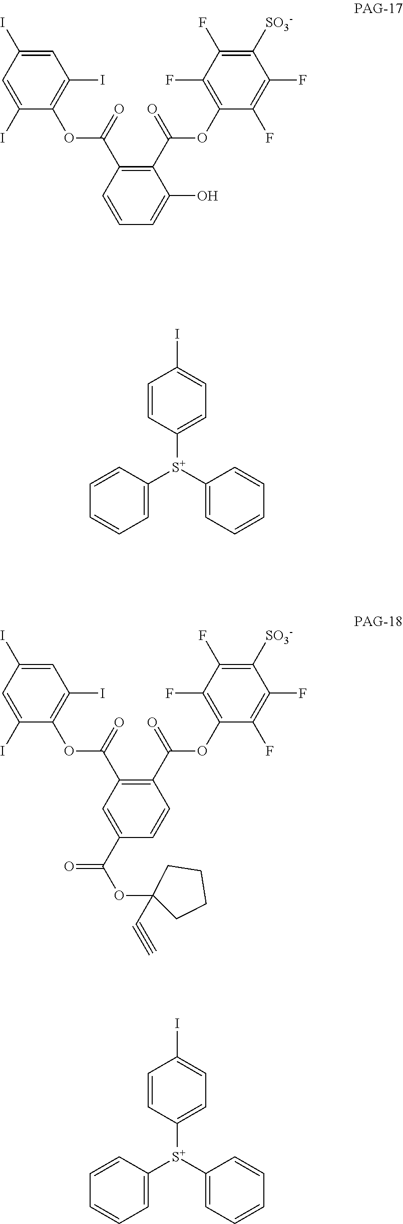

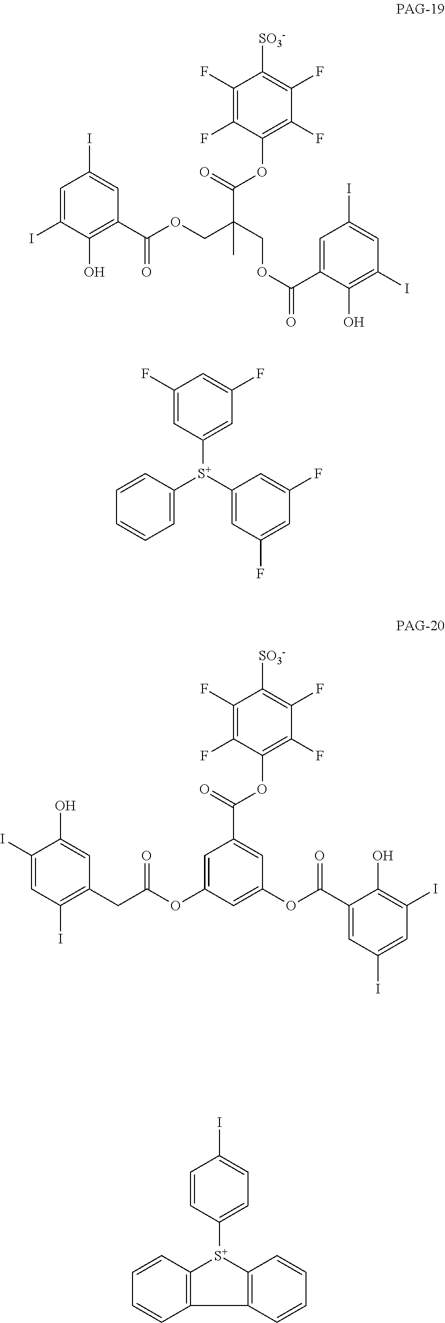

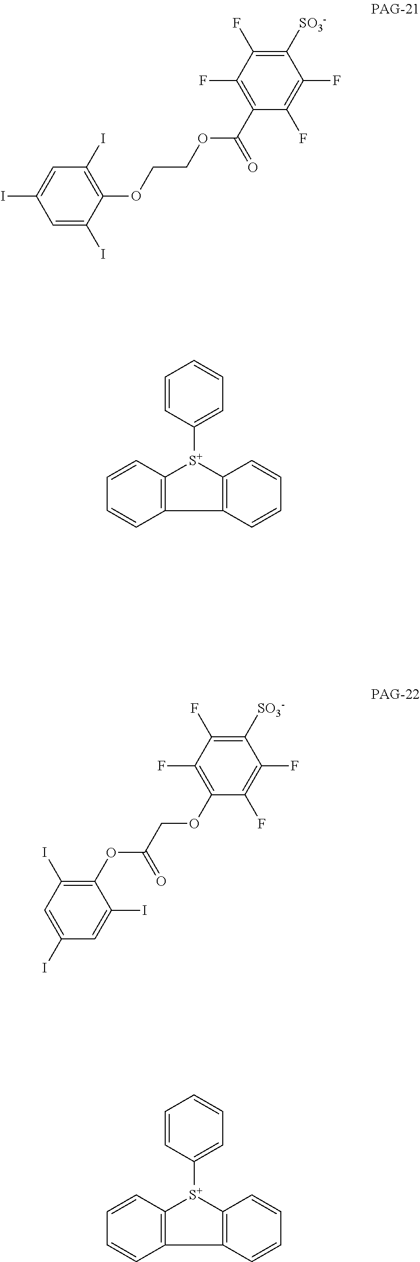

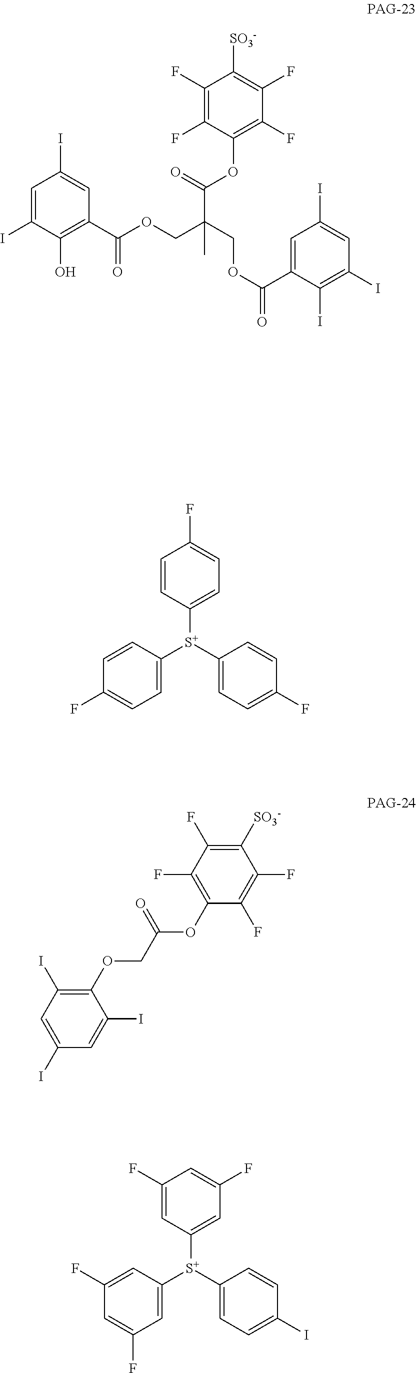

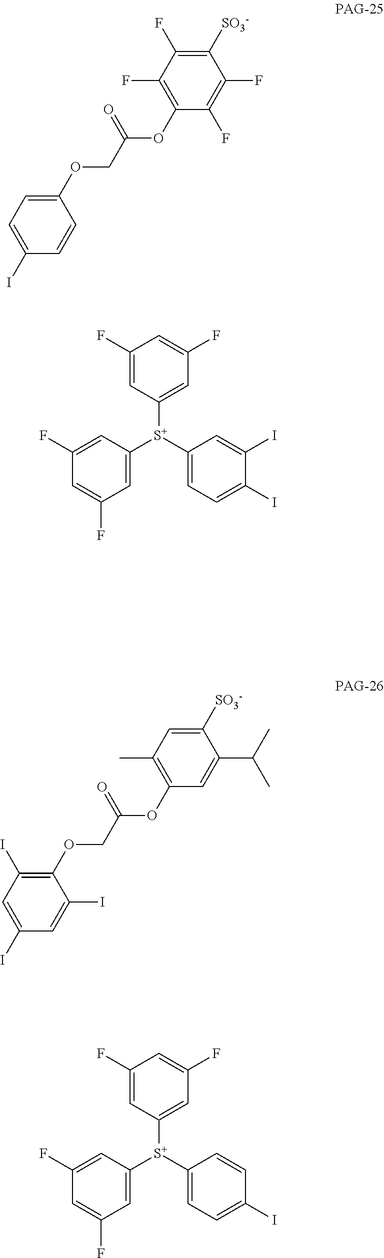

### [Synthesis Example] Synthesis of Base Polymers (Polymers P-1 to P-4)

Base polymers (Polymers P-1 to P-4) each having the composition shown below were synthesized by combining monomers, effecting copolymerization reaction in a THF solvent, pouring the reaction solution into methanol, washing the solid precipitate with hexane, isolation, and drying. The base polymers were analyzed for composition by ¹H-NMR spectroscopy and for Mw and Mw/Mn by GPC versus polystyrene standards using a THF solvent.

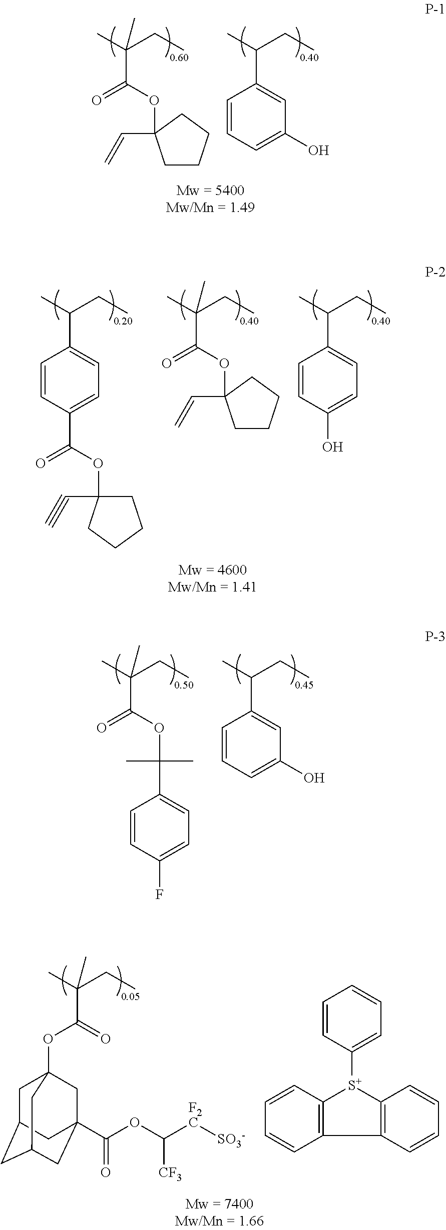

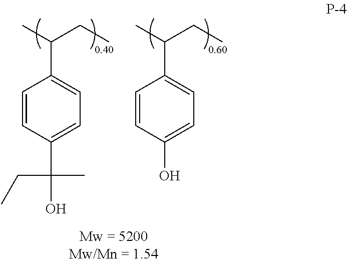

### [Examples 1 to 29 and Comparative Examples 1 to 3] Preparation and Evaluation of Resist Compositions

### (1) Preparation of Resist Compositions

A solution in which each component was dissolved with the composition shown in Tables 1 to 3 was filtered through a 0.2 μm-sized filter to prepare a resist composition.

The components in Tables 1 to 3 are as follows.

### Organic Solvents:

PGMEA (propylene glycol monomethyl ether acetate)

EL (ethyl lactate)

DAA (diacetone alcohol)

PGME (propylene glycol monomethyl ether)

### Comparative Acid Generators: cPAG-1 and cPAG-2

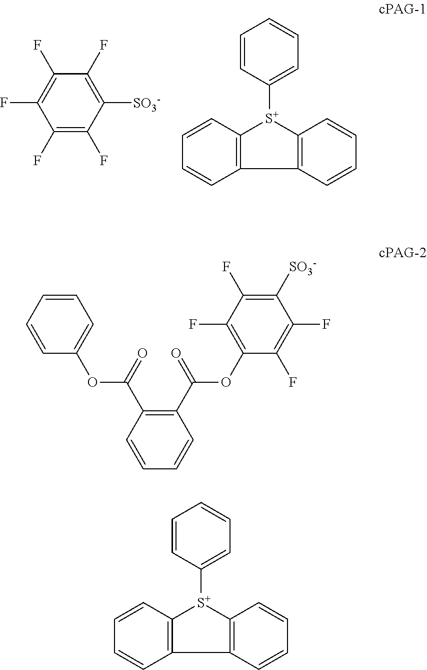

### Quenchers: Q-1 and Q-2

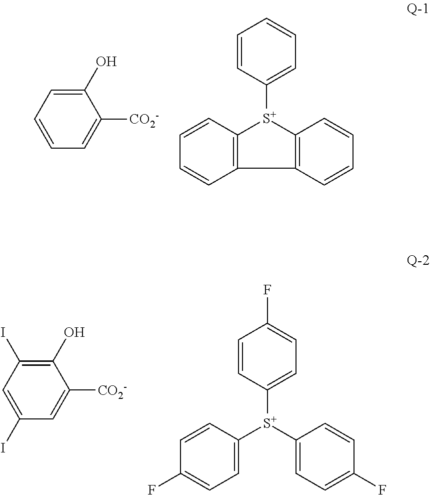

### (2) EUV Lithography Evaluation

Each of the resist compositions shown in Tables 1 to 3 was applied by spin coating to a silicon substrate having a 20-nm coating of silicon-containing spin-on hard mask SHB-A940 (Shin-Etsu Chemical Co., Ltd., Si content 43 wt %) and prebaked on a hotplate at 105° C. for 60 seconds to form a 50 nm-thick resist film. Using an EUV scanner NXE3400 (ASML, NA 0.33, σ 0.9/0.6, quadrupole illumination), the resist film was exposed to EUV through a mask bearing a hole pattern at a pitch of 40 nm (on-wafer size) and +20% bias. The resist film was baked (PEB) on a hotplate at the temperature shown in Tables 1 to 3 for 60 seconds and developed in a 2.38 wt % TMAH aqueous solution for 30 seconds to form a hole pattern having a size of 20 nm in Examples 1 to 28 and Comparative Examples 1 and 2, and a dot pattern having a size of 20 nm in Example 29 and Comparative Example 3.

The resist pattern was observed under CD-SEM (CG6300, Hitachi High-Technologies Corp.). The exposure dose that provides a hole pattern or a dot pattern having a size of 20 nm was reported as sensitivity. The size of 50 holes or dots printed at that dose was measured, from which a 3-fold value (3σ) of the standard deviation (σ) was computed and reported as CDU. Results are shown in Tables 1 to 3.

TABLE 1

| | Polymer | Acid generator | Quencher | Organic solvent | PEB | Sensitivity | CDU |
|---|---|---|---|---|---|---|---|
| Example | (pbw) | (pbw) | (pbw) | (pbw) | (° C.) | (mJ/cm²) | (nm) |
| 1 | P-1 | PAG-1 | Q-1 | PGMEA (500) | 90 | 32 | 3.1 |
| | (100) | (27.7) | (3.98) | EL (2000) | | | |
| 2 | P-1 | PAG-2 | Q-1 | PGMEA (500) | 90 | 31 | 3.2 |
| | (100) | (28.2) | (3.98) | EL (2000) | | | |
| 3 | P-1 | PAG-3 | Q-1 | PGMEA (500) | 90 | 29 | 3.2 |
| | (100) | (28.9) | (3.98) | EL (2000) | | | |
| 4 | P-1 | PAG-4 | Q-1 | PGMEA (2000) | 90 | 29 | 3.3 |
| | (100) | (28.8) | (3.98) | DAA (500) | | | |
| 5 | P-1 | PAG-5 | Q-1 | PGMEA (2000) | 90 | 31 | 3.1 |
| | (100) | (28.5) | (3.98) | DAA (500) | | | |
| 6 | P-1 | PAG-6 | Q-1 | PGMEA (2000) | 90 | 32 | 3.1 |
| | (100) | (27.9) | (3.98) | DAA (500) | | | |
| 7 | P-1 | PAG-7 | Q-1 | PGMEA (2000) | 90 | 30 | 3.0 |
| | (100) | (29.1) | (3.98) | DAA (500) | | | |
| 8 | P-1 | PAG-8 | Q-1 | PGMEA (2000) | 90 | 32 | 3.0 |
| | (100) | (29.2) | (3.98) | DAA (500) | | | |
| 9 | P-1 | PAG-9 | Q-1 | PGMEA (2000) | 90 | 32 | 3.2 |
| | (100) | (28.0) | (3.98) | DAA (500) | | | |
| 10 | P-1 | PAG-10 | Q-1 | PGMEA (2000) | 90 | 32 | 3.2 |
| | (100) | (24.2) | (3.98) | DAA (500) | | | |
| 11 | P-1 | PAG-11 | Q-1 | PGMEA (2000) | 90 | 32 | 3.3 |
| | (100) | (25.6) | (3.98) | DAA (500) | | | |
| 12 | P-1 | PAG-12 | Q-1 | PGMEA (2000) | 90 | 32 | 3.3 |
| | (100) | (25.6) | (3.98) | DAA (500) | | | |

TABLE 2

| | Polymer | Acid generator | Quencher | Organic solvent | PEB | Sensitivity | CDU |
|---|---|---|---|---|---|---|---|
| Example | (pbw) | (pbw) | (pbw) | (pbw) | (° C.) | (mJ/cm²) | (nm) |
| 13 | P-1 | PAG-13 | Q-1 | PGMEA (2000) | 90 | 30 | 3.2 |
| | (100) | (24.2) | (3.98) | DAA (500) | | | |
| 14 | P-1 | PAG-14 | Q-1 | PGMEA (2000) | 90 | 30 | 3.2 |
| | (100) | (25.5) | (3.98) | DAA (500) | | | |
| 15 | P-1 | PAG-15 | Q-1 | PGMEA (2000) | 90 | 32 | 3.0 |
| | (100) | (34.2) | (3.98) | DAA (500) | | | |
| 16 | P-1 | PAG-16 | Q-1 | PGMEA (2000) | 90 | 33 | 3.2 |
| | (100) | (32.6) | (3.98) | DAA (500) | | | |
| 17 | P-1 | PAG-17 | Q-1 | EL (2000) | 90 | 32 | 3.2 |
| | (100) | (31.3) | (3.98) | PGME (500) | | | |
| 18 | P-1 | PAG-18 | Q-1 | EL (2000) | 90 | 31 | 3.2 |
| | (100) | (34.3) | (3.98) | PGME (500) | | | |
| 19 | P-1 | PAG-19 | Q-1 | EL (2000) | 90 | 32 | 3.2 |
| | (100) | (36.0) | (3.98) | PGME (500) | | | |
| 20 | P-1 | PAG-20 | Q-1 | EL (2000) | 90 | 31 | 3.2 |
| | (100) | (37.8) | (3.98) | PGME (500) | | | |
| 21 | P-1 | PAG-21 | Q-1 | EL (2000) | 90 | 32 | 3.2 |
| | (100) | (25.8) | (3.98) | PGME (500) | | | |
| 22 | P-1 | PAG-22 | Q-1 | EL (2000) | 90 | 33 | 3.3 |
| | (100) | (25.5) | (3.98) | PGME (500) | | | |
| 23 | P-1 | PAG-23 | Q-1 | EL (2000) | 90 | 32 | 3.2 |
| | (100) | (41.0) | (3.98) | PGME (500) | | | |
| 24 | P-1 | PAG-24 | Q-1 | EL (2000) | 90 | 30 | 3.3 |
| | (100) | (30.5) | (3.98) | PGME (500) | | | |
| 25 | P-1 | PAG-25 | Q-1 | EL (2000) | 90 | 29 | 3.3 |
| | (100) | (27.3) | (3.98) | PGME (500) | | | |
| 26 | P-1 | PAG-26 | Q-1 | EL (2000) | 100 | 31 | 3.3 |
| | (100) | (30.1) | (3.98) | PGME (500) | | | |
| 27 | P-2 | PAG-1 | Q-2 | PGMEA (2000) | 90 | 33 | 3.1 |
| | (100) | (27.7) | (7.06) | DAA (500) | | | |
| 28 | P-3 | PAG-1 | Q-2 | PGMEA (2000) | 90 | 33 | 3.1 |
| | (100) | (27.7) | (7.06) | DAA (500) | | | |
| 29 | P-4 | PAG-1 | Q-1 | PGMEA (2000) | 130 | 32 | 3.2 |
| | (100) | (22.1) | (3.98) | DAA (500) | | | |

TABLE 3

| Comparative | Polymer | Acid generator | Quencher | Organic solvent | PEB | Sensitivity | CDU |
|---|---|---|---|---|---|---|---|
| Example | (pbw) | (pbw) | (pbw) | (pbw) | (° C.) | (mJ/cm²) | (nm) |
| 1 | P-1 | cPAG-1 | Q-1 | PGMEA (2000) | 90 | 35 | 4.9 |
| | (100) | (12.7) | (3.98) | DAA (500) | | | |
| 2 | P-1 | cPAG-2 | Q-1 | PGMEA (2000) | 90 | 38 | 4.2 |
| | (100) | (18.2) | (3.98) | DAA (500) | | | |
| 3 | P-4 | cPAG-1 | Q-1 | PGMEA (2000) | 130 | 45 | 5.1 |
| | (100) | (10.2) | (3.98) | DAA (500) | | | |

From the results shown in Tables 1 to 3, it was found that the resist composition of the present invention, which comprises, as an acid generator, a sulfonium salt or an iodonium salt containing an arylsulfonic acid anion having an aromatic group substituted with an iodine atom or a bromine atom, has high sensitivity and good CDU.

Japanese Patent Application Nos. 2023-113631 and 2024-001147 are incorporated herein by reference. Although some preferred embodiments have been described, many modifications and variations may be made thereto in light of the above teachings. It is therefore to be understood that the invention may be practiced otherwise than as specifically described without departing from the scope of the appended claims.

1. A resist composition comprising an acid generator containing an onium salt having the following formula (1): 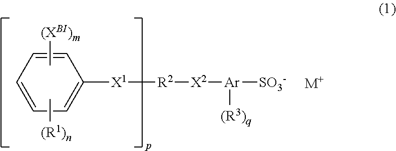
wherein m is an integer of 1 to 5, n is an integer of 0 to 4, provided that 1≤m+n≤5, p is an integer of 1 to 3, q is an integer of 1 to 4 when Ar is a (q+2)-valent group derived from benzene, and an integer of 1 to 6 when Ar is a (q+2)-valent group derived from naphthalene,
X^{BI} is a bromine atom or an iodine atom,
Ar is a (q+2)-valent group derived from benzene or naphthalene,
X¹ and X² are each a single bond, an ether bond, an ester bond, a sulfonic acid ester bond, an amide bond, a urethane bond, a urea bond, a carbonate bond, or a C₁-C₆ alkanediyl group, and the alkanediyl group may contain at least one selected from an ether bond and an ester bond, however, when p=1, X^{BI} is an iodine atom and Ar is a (q+2)-valent group derived from benzene, X¹ is not *—C(═O)—O—, and when p=1, X^{BI} is an iodine atom, X¹ and R² are each a single bond and Ar is a (q+2)-valent group derived from benzene, X² is not *—C(═O)—O—, and when p=1, X² and R² are each a single bond and Ar is a (q+2)-valent group derived from benzene, X¹ is not —O—C(═O)—** and a carbonate bond, and when p=1, X¹ is a single bond and Ar is a (q+2)-valent group derived from benzene, X² is not —O—C(═O)—** and a carbonate bond, * represents a bond with a benzene ring in the formula, and ** represents a bond with Ar,
R¹ is a hydroxy group, a carboxy group, a fluorine atom, a chlorine atom, an amino group, a C₁-C₂₀ hydrocarbyl group, a C₁-C₂₀ hydrocarbyloxy group, a C₂-C₂₀ hydrocarbyloxycarbonyl group, a C₂-C₂₀ hydrocarbylcarbonyloxy group, —N(R^{1A})—C(═O)—R^{1B}, N(R^{1A})—C(═O)—O—R^{1B}, or —N(R^{1A})—S(═O)₂—R^{1B}, and the hydrocarbyl group, the hydrocarbyloxy group, the hydrocarbyloxycarbonyl group, and the hydrocarbylcarbonyloxy group may contain at least one selected from a fluorine atom, a chlorine atom, a bromine atom, an iodine atom, a hydroxy group, an amino group, an ester bond, and an ether bond, R^{1A} is a hydrogen atom or a C₁-C₆ saturated hydrocarbyl group, and the saturated hydrocarbyl group may contain a halogen atom, a hydroxy group, a C₁-C₆ saturated hydrocarbyloxy group, a C₂-C₆ saturated hydrocarbylcarbonyl group, or a C₂-C₆ saturated hydrocarbylcarbonyloxy group, R^{1B} is a C₁-C₆ aliphatic hydrocarbyl group or a C₆-C₁₂ aryl group, which may contain a halogen atom, a hydroxy group, a C₁-C₆ saturated hydrocarbyloxy group, a C₂-C₆ saturated hydrocarbylcarbonyl group, or a C₂-C₆ saturated hydrocarbylcarbonyloxy group,
R² is a single bond or a C₁-C₂₈ (p+1)-valent hydrocarbon group, and the hydrocarbon group may contain at least one selected from an oxygen atom, a nitrogen atom, a sulfur atom, and a halogen atom,
R³ is a fluorine atom, a trifluoromethoxy group, a nitro group, a C₁-C₁₂ saturated hydrocarbyl group, or a C₆-C₁₀ aryl group, and
M⁺ is a sulfonium cation or an iodonium cation.

2. The resist composition of claim 1, wherein X¹ and X² are groups other than a single bond, and R² is a group other than a single bond.

3. The resist composition of claim 1, further comprising a base polymer.

4. The resist composition of claim 3, wherein the base polymer contains repeat units having the following formula (a1) or (a2): 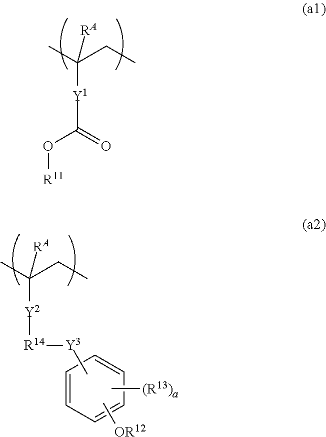
wherein R^{A} is each independently a hydrogen atom or a methyl group,
Y¹ is a single bond, a phenylene group, a naphthylene group, or a C₁-C₁₂ linking group containing at least one moiety selected from an ester bond, an ether bond, and a lactone ring,
Y² is a single bond or an ester bond,
Y³ is a single bond, an ether bond, or an ester bond,
R¹¹ and R¹² are each independently an acid labile group,
R¹³ is a C₁-C₄ saturated hydrocarbyl group, a halogen atom, a C₂-C₅ saturated hydrocarbylcarbonyl group, a cyano group, or a C₂-C₅ saturated hydrocarbyloxycarbonyl group,
R¹⁴ is a single bond or a C₁-C₆ alkanediyl group which may contain an ether bond or an ester bond, and
a is an integer of 0 to 4.

5. The resist composition of claim 4, which is a chemically amplified positive resist composition.

6. The resist composition of claim 3, wherein the base polymer is free of an acid labile group.

7. The resist composition of claim 6, which is a chemically amplified negative resist composition.

8. The resist composition of claim 1, further comprising an organic solvent.

9. The resist composition of claim 1, further comprising a quencher.

10. The resist composition of claim 1, further comprising a surfactant.

11. A pattern forming process comprising the steps of:
applying the resist composition of claim 1 onto a substrate to form a resist film thereon,
exposing the resist film to high-energy radiation, and
developing the exposed resist film in a developer.

12. The pattern forming process of claim 11, wherein the high-energy radiation is ArF excimer laser having a wavelength of 193 nm, KrF excimer laser having a wavelength of 248 nm, electron beam, or extreme ultraviolet having a wavelength of 3 to 15 nm.
